(19) United States
(12) Patent Application Publication
(10) Pub. No.: US 2026/0256689 A1
(43) Pub. Date: Sep. 3, 2026

(54) A CONTROLLED RELEASE IMPLANT FOR BIOLOGICS AND CORRESPONDING METHODS OF TREATMENT

(71) Applicant: OCULAR THERAPEUTIX, INC., Bedford, MA (US)

(72) Inventors: Rami Fahed ELHAYEK, Norwood, MA (US); Michael Harry GOLDSTEIN, Cambridge, MA (US); Peter Kendrick JARRETT, Corte Madera, CA (US); Steven LU, Somerville, MA (US); Meryem Oznur PEHLIVANER, Northborough, MA (US); Nelson J. BELLO, Lowell, MA (US)

(73) Assignee: OCULAR THERAPEUTIX, INC., Bedford, MA (US)

(21) Appl. No.: 18/834,967

(22) PCT Filed: Feb. 1, 2023
(86) PCT No.: PCT/US2023/061792

§ 371 Date: Jul. 31, 2024

Related U.S. Application Data
(60) Provisional application No. 63/267,419, filed on Feb. 1, 2022.
(60) Provisional application No. 63/343,912, filed on May 19, 2022.
(60) Provisional application No. 63/377,992, filed on Sep. 30, 2022.
(60) Provisional application No. 63/477,052, filed on Dec. 23, 2022.

(51) Int. Cl. A61K 9/00; A61K 45/06; A61K 47/26; A61K 47/32; A61K 47/34; A61K 48/00; A61P 27/02; C12N 15/86

(52) U.S. Cl. CPC A61K 9/0024; A61K 9/0048; A61K 45/06; A61K 47/26; A61K 47/32; A61K 47/34; A61K 48/005; A61K 48/0075; A61K 48/0083; A61P 27/02; C12N 15/86; C12N 2750/14143

(57) ABSTRACT

An implant, such as a pharmaceutically acceptable implant, comprising a xerogel, a biologic, and at least one dehydration stabilizer.

### FIELD OF THE INVENTION

The present invention relates to an implant for biologics. In particular, the present invention relates to a pharmaceutically acceptable implant for controlled release of a biologic such as a viral vector. The present invention also relates to corresponding methods of treatment and uses.

### BACKGROUND OF THE INVENTION

Controlled delivery of therapeutic agents is a large area of research and in the recent years extending in particular to biologics. A controlled delivery improves therapies, facilitates administration and leads to better compliance, less side effects and better therapeutic results.

However, delivery of ocular therapeutic agents to the eye remains a challenge. The effectiveness of these treatments is hampered by various parameters, most of them related to the eye being an immunologically privileged organ and the eye being of limited size.

While treating an ocular disorder may require a therapeutically effective dose, it may not be feasible to administer large volumes of a therapeutic agent to the eye or administer such therapeutic agents with a higher frequency without causing inflammation.

Gene therapy for example is a rather new but elegant mode of treatment and is also considered for treating eye diseases. The basic concept of gene therapy is to fix a genetic problem at its source. If, for instance, a mutation in a certain gene causes the production of a dysfunctional protein resulting in a disease, gene therapy can be used to deliver a copy of this gene that does not contain the deleterious mutation and thereby produces a functional protein. Not only genetic problems can be addressed, any kind of therapeutic protein can be produced by the patient through the vector to treat certain diseases.

In such therapies viral vectors coding for certain therapeutic agents are most often administered. These viral vectors have the ability to replicate and to lead to the expression of the therapeutic protein. Very often, expression of the therapeutic protein comprised in the viral vectors or the viral vectors themselves induce inflammation thereby leading to reduced expression of the therapeutic protein comprised in the viral vectors and thus, reduced gene therapy efficacy. Thus, these approaches suffer from a major bottle-neck of being unable to avoid inflammation when delivering gene therapy vectors. Several methods to solve this problem have been investigated including the use of steroids, a known anti-inflammatory agent. However, the administration of steroids in the eye may increase the intraocular eye pressure and therefore drastically increase the risk of developing eye pressure associated eye diseases like glaucoma.

Stabilizing complex biologics such as viral vectors in a more complex formulation is a challenge. Temperature, organic solvents, other chemicals and the elimination of water are major challenges for the formulation technology for biologics, in particular complex biologics.

### OBJECTS AND SUMMARY OF THE INVENTION

It is an object of the invention, and an aspect, to stabilize a biologic such as a viral vector in a pharmaceutically acceptable implant.

It is an object of the invention, and an aspect, to provide a pharmaceutically acceptable implant comprising a biologic.

It is an object of the invention, and an aspect, to stabilize a biologic such as a viral vector in a pharmaceutically acceptable implant with a polymeric network.

It is an object of the invention, and an aspect, to stabilize a biologic such as a viral vector during the manufacture of an implant, such as an implant with a polymeric network.

It is an object of the invention, and an aspect, to provide a pharmaceutically acceptable implant comprising a biologic in a polymeric network. It is an object of the invention to provide for a controlled release of a biologic, such as a viral vector from a pharmaceutically acceptable implant.

It is an object of the invention, and an aspect, to provide a pharmaceutically acceptable implant comprising a viral vector for gene therapy of the eye.

It is an object of the invention, and an aspect, to deliver a biologic, such as a viral vector to the eye while controlling inflammation.

It is an object of the invention, and an aspect, to deliver a viral vector to the eye for expressing a therapeutic protein while controlling inflammation.

It is an object of the invention, and an aspect, to deliver a viral vector to the eye for expressing a therapeutic protein while controlling the immune response.

It is an object of the invention, and an aspect, to deliver a viral vector to the eye for expressing a therapeutic protein while controlling the adaptive immune response such as humoral immune response. It is an object of the invention, and an aspect, to treat inflammation of the eye, in particular inflammation due to the adaptive immune response.

Some aspects of the present disclosure are directed to an implant such as a pharmaceutically acceptable implant, comprising a xerogel, a biologic and at least one dehydration stabilizer.

Some aspects of the present disclosure are in part directed to a pharmaceutically acceptable implant for controlled release of a biologic, and wherein the controlled release is characterized by: the amount of the biologic released on day 1 is from 0 to 50% of the total amount of the biologic, the amount of the biologic released per day from day 2 until the last day of release is from 0 to 50% of the total amount of the biologic, and/or the number of days required for 100% release of the total amount of the biologic is at least 2 days.

Some aspects of the present disclosure are directed to a method of treating an ocular disorder, such as an ocular genetic disorder comprising administering to a subject a pharmaceutically acceptable implant of the invention or corresponding use.

Some aspects of the present disclosure are directed to a method of controlling inflammation when treating an ocular disorder such as an ocular genetic disorder comprising administering to a subject a pharmaceutically acceptable implant of the invention or corresponding use.

Some aspects of the present disclosure are directed to a method of controlling an immune response such as an adaptive immune response when treating an ocular disorder such as an ocular genetic disorder comprising administering to a subject a pharmaceutically acceptable implant of the invention or corresponding use. Some aspects of the present disclosure are directed to providing an effective method when treating an ocular disorder such as an ocular genetic disorder comprising administering to a subject a pharmaceutically acceptable implant of the invention or corresponding use.

Some aspects of the present disclosure are directed to a use of at least one dehydration stabilizer for protecting a biologic against damage during a process wherein the biologic is directly exposed to an organic solvent.

Some aspects of the present disclosure are directed to a method for protecting a biologic against damage during a process wherein the biologic is directly exposed to an organic solvent, the method comprising mixing the biologic with at least one dehydration stabilizer before directly exposing the biologic to an organic solvent.

Some aspects of the present disclosure are directed to a method for manufacturing a pharmaceutically acceptable implant comprising a biologic comprising (A) forming an organogel including the biologic comprising forming a matrix comprising at least two multi-arm precursors that are covalently crosslinked in an organic solvent in the presence of the biologic, (B) forming a xerogel comprising removing the organic solvent.

The present invention is also directed to a method for manufacturing a pharmaceutically acceptable implant for controlled release of a total amount of a biologic comprising
(a) selecting the total (w/w) % of a carbohydrate, sugar alcohol or combination thereof,
(b) selecting the molecular weight between crosslinks in the xerogel,
(c) selecting the (w/w) % of the total particles comprising a mixture of the biologic and a carbohydrate, a sugar alcohol, or a combination thereof,
(d) selecting the (w/w) % of the total number of multi-arm precursors,
(e) selecting the ratio of (c) and (d), and/or
(f) selecting the molar ratio of:
(f-i) the first reactive group comprised in the second multi-arm precursor, and
(f-ii) the second reactive group comprised in the third multi-arm precursor, wherein the (w/w) % is based on the weight of the pharmaceutically acceptable implant.

The present invention is also directed to a method for manufacturing a pharmaceutically acceptable implant for controlled release of a total amount of a biologic comprising
(a) selecting the total (w/w) % of a carbohydrate, sugar alcohol or combination thereof,
(b) selecting the molecular weight between crosslinks in the xerogel,
(c) selecting the (w/w) % of the total particles comprising a mixture of a biologic and, a carbohydrate, a sugar alcohol, or a combination thereof,
(d) selecting the (w/w) % of the total number of multi-arm precursors,
(e) selecting the ratio of (c) and (d),
(f) selecting the D₉₀ particle size such as Dv₉₀ particle size, wherein particles comprise a mixture of the biologic and at least one dehydration stabilizer such as a carbohydrate, a sugar alcohol, or a combination thereof, and/or
(g) selecting the molar ratio of:
(g-i) the first reactive group comprised in the second multi-arm precursor, and
(g-ii) the second reactive group comprised in the third multi-arm precursor, wherein the (w/w) % is based on the weight of the pharmaceutically acceptable implant.

Some aspects of the present disclosure are directed to a method of treating inflammation of the eye comprising administering a therapeutically effective amount of a tyrosine kinase inhibitor to the eye of the subject in need thereof.

### Definitions

A “precursor” as used herein refers to any polymer that fulfils all of the following requirements (i) soluble in an organic solvent, (ii) able to react with another precursor, (iii) unreactive to a biologic.

The term “polymer network” describes a structure formed of at least two precursors (of the same or different molecular structure and of the same or different molecular weight) that are crosslinked with each other. The types of precursors suitable for the purpose of the present invention are disclosed herein. The term “polymer network” is used interchangeably with the term “matrix”.

“Organogel” as used herein refers to a three-dimensional polymer network of at least two precursors that are covalently cross-linked with each other in the presence of an organic solvent and still comprising the organic solvent (undried form). An “organic solvent” as used herein is a carbon-based substance that is liquid at room temperature and pressure. Such organic solvents can be methylene chloride, dimethyl carbonate, acetone, acetonitrile, ethyl acetate, and tetrahydrofuran.

“Xerogel” in its simplest meaning refers to a dried organogel. A “xerogel” is a three-dimensional polymer network of at least two multi-arm precursors that are covalently cross-linked with each other to form a polymer network and is in its dried state. Thus, in certain embodiments, a xerogel in the context of the present invention may contain no more than about 5%, 4%, 3%, 2% or 1% by weight water, such as less than 2%, such as less than 1% by weight water. The water content of an implant in its dry/dried state may be measured e.g., by means of a Karl Fischer coulometric method. Thus, in certain embodiments, a xerogel in the context of the present invention may contain no more than 2% by weight organic solvent, such as less than 1% by weight organic solvent.

“Hydrogel” as used herein refers to a hydrated xerogel. Once under physiological conditions such as pH 7.2-7.4 at 37° C., the xerogel is hydrated and thus, referred to as a hydrogel. Due to their high-water content, hydrogels are soft and flexible, which makes them very similar to natural tissue. In the present invention the term “hydrogel” is used to refer to a xerogel in the hydrated state when it contains water (e.g., under physiological conditions).

The term “implant” as used herein refers to a xerogel that has any pre-determined shape (such as disclosed herein). Thus, an implant is an object that comprises a xerogel, trapped within which is an active agent, specifically any biologic (as disclosed herein) that is in the form of a mixture with at least one dehydration stabilizer.

The “in-situ implant” according to the present invention refers to an implant formed in vivo from a hydrogel precursor composition comprising a biologic, when injected in vivo forms a hydrogel.

The term “pharmaceutically acceptable implant” is an implant that can be administered to a subject. The “pharmaceutically acceptable implant” when administered into the human or animal body, e.g., to the vitreous humor of the eye (also called “vitreous chamber” or “vitreous body”) remains for a certain period of time while it releases the active agent into the surrounding environment. An implant can have any predetermined shape (such as disclosed herein) before being injected, which shape is maintained to a certain degree upon placing the implant into the desired location, although dimensions of the implant (e.g. length and/or diameter) may change after administration due to hydration as further disclosed herein. In other words, what is injected into the eye is not a solution or suspension, but an already shaped, coherent object. The “pharmaceutically acceptable implant” has thus been completely formed as disclosed herein prior to being administered, and in the embodiments of the present invention is not created in situ at the desired location in the eye (as would generally also be possible with suitable formulations). Herein, the term “implant” or “pharmaceutically acceptable implant” is used to refer to an implant that comprises a xerogel and therefore, in its dried and/or dehydrated state, i.e., after the implant has been produced and dried and just prior to being loaded into a needle, or after having been loaded into a needle as disclosed herein, or wherein the implant has been manufactured in a dry state without the need for dehydration. Once the pharmaceutically acceptable implant has been administered to the eye or otherwise immersed into an aqueous environment (such as in vitro), it is hydrated under physiological conditions and then it is used to refer to an implant or pharmaceutically acceptable implant that comprises a hydrogel. Whenever dimensions of an implant or pharmaceutically acceptable implant (i.e., length, diameter, or volume) are reported herein in the hydrated state, these dimensions are measured at various indicated time points after the implant or pharmaceutically acceptable implant has been immersed in an aqueous solution under physiological conditions such as pH 7.2-7.4 at 37° C. Whenever dimensions of an implant or pharmaceutically acceptable implant are reported herein in the dry state, these dimensions are measured after it has been fully dried (and thus, in certain embodiments, the implant or pharmaceutically acceptable implant contains no more than about 5%, 4%, 3%, 2% or 1% by weight water or organic solvent) and it is in a state to be loaded into a needle for subsequent administration.

In certain embodiments of the present invention, the term “fiber” characterizes an object in the shape of which, the implant or pharmaceutically acceptable implant has been formed that in general has an elongated shape. Specific dimensions of implants of the present invention are disclosed herein. The fiber may have a cylindrical or essentially cylindrical shape. The cross-sectional area of the fiber or the implant may be either round or essentially round but may in certain embodiments also be oval or oblong, or may in other embodiments have different geometries, such as cross-shaped, star-shaped or other as disclosed herein.

The term “biodegradable” refers to a material or object (such as the ocular implant according to the present invention) which becomes degraded in vivo, i.e., when placed in the human or animal body or in vitro when immersed in an aqueous solution under physiological conditions such as pH 7.2-7.4 at 37° C. In the context of the present invention, as disclosed in detail herein below, the implant slowly biodegrades over time once deposited within the eye, e.g., within the vitreous humor. In certain embodiments biodegradation takes place at least in part via ester hydrolysis in the aqueous environment of the vitreous. The implant slowly dissolves until it is fully resorbed and is no longer visible in the vitreous.

As used herein, the term “adeno-associated virus” or “AAV” includes all serotypes such as but not limited to, AAV1, AAV2 such as AAV2.7m8, AAV3, AAV4, AAV5, AAV6, AAV7, AAV8, AAV9, AAV10, AAV11 derived from any species. Further details on AAV serotypes, clades and any other AAV can e.g., be found in Gao et al. (J. Virol. 78:6381 (2004), Moris et al. (Virol. 33:375 (2004), and FIELDS et al. VIROLOGY, volume 2, chapter 69 (4th ed., Lippincott-Raven Publishers). For example, the AAV can be an AAV derived from a naturally occurring “wild-type” virus, an AAV derived from a recombinant AAV (rAAV) genome packaged into a capsid derived from capsid proteins encoded by a naturally occurring cap gene and/or a rAAV genome packaged into a capsid derived from capsid proteins encoded by a non-natural capsid cap gene. As used herein, “AAV” can be used to refer to the virus itself or derivatives thereof. The term covers all subtypes and both naturally occurring and recombinant forms, except where specifically indicated otherwise. In some aspects, the AAV is a non-replicating AAV, e.g., a non-infectious AAV. In some embodiments, the AAV comprises a viral vector.

AAV vectors can include additional elements that function in cis or in trans. In particular embodiments, an AAV vector that includes a vector genome also has one or more ITR sequences that flank the 5′ or 3′ terminus of the donor sequence; an expression control element that drives transcription (e.g., a promoter or enhancer) of the donor sequence, such as a constitutive or regulatable control element, or tissue-specific expression control element; an intron sequence, a stuffer or filler polynucleotide sequence; and/or a poly-Adenine sequence located 3′ of the donor sequence.

The terms “polypeptide,” “peptide”, “protein,” “protein fragment” are used interchangeably herein to refer to at least two amino acids or amino acid analogs which are covalently linked by a peptide bond or an analog of a peptide bond. Thus, these terms include any protein having a primary, secondary, tertiary, or quaternary structure, fragments thereof, and fusions thereof.

The term “release” (and accordingly the terms “released”, “releasing” etc.) as used herein refers to the provision of agents such as a biologic from an implant of the present invention to the surrounding environment. The surrounding environment may be an in vitro or in vivo environment as described herein. In certain specific embodiments, the surrounding environment is the vitreous humor and/or ocular tissue, such as the retina and the choroid. Thus, whenever it is herein stated that the implant or “pharmaceutically acceptable implant” “releases” or “provides for (controlled) release” of a biologic such as an AAV or a recombinant protein, this not only refers to the provision of said biologic directly from the implant while the hydrogel has not yet (fully) biodegraded, but also refers to the continued provision of said biologic to the surrounding environment following full degradation of the hydrogel when remaining biologic is still present in this surrounding environment for an extended period of time and continues to exert its therapeutic effect.

The term “controlled release” refers to release of an active agent specifically a biologic such as an AAV or a recombinant protein from the implant or pharmaceutically acceptable implant in a predetermined way and is in contrast to an immediate release like a bolus injection. The controlled release refers to the amount of the biologic release on day 1, per day from day 2 onwards, and the total number of days required for 100% release of the biologic in an aqueous solution under physiological conditions such as at pH 7.2-7.4 and 37° C. Thus, the “controlled release” as measured under these conditions is considered to be the same when the pharmaceutically acceptable implant is administered in vivo to a subject.

The term “100% release of the biologic” should be construed as from 95% to 100%. The way this controlled release is achieved is by a number of parameters that are characteristics of the pharmaceutically acceptable implant as disclosed herein. Each such characteristic feature of the pharmaceutically acceptable implant alone or in combination with each other can be responsible for the controlled release.

“Total amount of the biologic” as used herein refers to the total amount of the biologic that is comprised and/or included in the pharmaceutically acceptable implant. A skilled artisan is able to assess the total amount of the biologic before including it in the method of manufacturing the pharmaceutically acceptable implant of the invention. For example, if the biologic is a virus, a skilled artisan may use polymerase chain reaction (PCR) or Enzyme-Linked Immunosorbent Assay (ELISA) to assess the total amount of the virus.

As used herein the terms “heterologous” or “exogenous” refer to such molecules that are not normally found in a given context, e.g., in a cell or in a polypeptide. For example, an exogenous or heterologous molecule can be introduced into a cell and are only present after manipulation of the cell, e.g., by transfection or other forms of genetic engineering or a heterologous amino acid sequence can be present in a protein in which it is not naturally found.

A “zero order” release or “substantially zero order” release or “near zero order” release is defined as exhibiting a relatively straight line in a graphical representation of percent of the biologic released versus time. In certain embodiments of the present invention, substantially zero order release is defined as the amount of the biologic released which is proportional within 20% to elapsed time.

A “dehydration stabilizer” as used herein is an excipient and/or an additive that protects and stabilizes a biologic or (a non-biologic comprising a biologic) in dry form or in the absence of water. For example, if a biologic is a protein, a dehydration stabilizer prevents denaturation or aggregation by preserving the tertiary or quaternary structure of said proteins.

The term “ocular” as used in the present invention refers to the eye in general, or any part or portion of the eye (as an “ocular implant” according to the invention can in principle be administered to any part or portion of the eye) or any disease of the eye (as in one aspect the present invention generally refers to treating any diseases of the eye (“ocular diseases”), of various origin and nature. The present invention in certain embodiments is directed to intravitreal injection of an ocular implant (in this case the “ocular implant” is thus an “intravitreal implant”).

“Controlling inflammation” according to the present invention refers to limiting inflammation to an acceptable level such that the treatment can be continued.

The term “patient” herein includes both human and animal patients. The pharmaceutically acceptable implants according to the present invention are therefore suitable for human or veterinary medicinal applications. Generally, a “subject” is a (human or animal) individual to which an implant according to the present invention is administered. A “patient” is a subject in need of treatment due to a particular physiological or pathological condition. A “patient” does not necessarily have a diagnosis of the particular physiological or pathological condition prior to receiving an implant.

The molecular weight of a polymer precursor as used for the purposes of the present invention and as disclosed herein may be determined by analytical methods known in the art. The molecular weight of polyethylene glycol may for example be determined by any method known in the art, including gel electrophoresis such as SDS-PAGE (sodium dodecyl sulphate-polyacrylamide gel electrophoresis), gel permeation chromatography (GPC), including GPC with dynamic light scattering (DLS), liquid chromatography (LC), as well as mass spectrometry such as matrix-assisted laser desorption/ionization-time of flight (MALDI-TOF) spectrometry or electrospray ionization (ESI) mass spectrometry. The molecular weight of a polymer, including a polyethylene glycol precursor as disclosed herein, is an average molecular weight (based on the polymer's molecular weight distribution), and may therefore be indicated by means of various average values, including the weight average molecular weight (Mw) and the number average molecular weight (Mn). In the case of polyethylene glycol precursors as used in the present invention, the molecular weight indicated herein is the number average molecular weight (Mn).

The term “day 1” as used herein refers to a time point that immediately follows after “day 0”. Thus, whenever “day 1” is used, it refers to an already elapsed time period of one day or about 24 hours.

“anti-drug antibody (ADA) titer” as used herein is depicted as reciprocal dilution. ADA as used herein encompasses any ADA known to the skilled in the art including ADA that are neutralizing antibodies (Nab).

“D₉₀ particle size” as used herein refers to a numerical value representing diameter of a particle and indicates that 90% of the particle distribution comprised in the implant of the invention has a diameter below said numerical value.

“Dn₉₀ particle size” as used herein refers to a numerical value representing diameter of a particle and indicates that 90% of the particle distribution by number comprised in the implant of the invention has a diameter below said numerical value.

“Dv₉₀ particle size” as used herein refers to a numerical value representing diameter of a particle and indicates that 90% of the particle distribution by volume comprised in the implant of the invention has a diameter below said numerical value.

As used herein, the term “about” in connection with a measured quantity, refers to the normal variations in that measured quantity, as expected by one of ordinary skill in the art in making the measurement and exercising a level of care commensurate with the objective of measurement and the precision of the measuring equipment.

The term “at least about” in connection with a measured quantity refers to the normal variations in the measured quantity, as expected by one of ordinary skill in the art in making the measurement and exercising a level of care commensurate with the objective of measurement and precisions of the measuring equipment and any quantities higher than that.

The term “average” as used herein refers to a central or typical value in a set of data (points), which is calculated by dividing the sum of the data (points) in the set by their number (i.e., the mean value of a set of data).

As used herein, the singular forms “a,” “an”, and “the” include plural references unless the context clearly indicates otherwise.

The term “and/or” as used in a phrase such as “A and/or B” herein is intended to include both “A and B” and “A or B”.

Open terms such as “include,” “including,” “contain,” “containing” and the like as used herein mean “comprising” and are intended to refer to open-ended lists or enumerations of elements, method steps, or the like and are thus not intended to be limited to the recited elements, method steps or the like but are intended to also include additional, unrecited elements, method steps or the like.

The term “up to” when used herein together with a certain value or number is meant to include the respective value or number.

The terms “from A to B”, “of from A to B”, and “of A to B” are used interchangeably herein and all refer to a range from A to B, including the upper and lower limits A and B.

Throughout this disclosure, various aspects of this invention are presented in a range format. It should be understood that the description in range format is merely for convenience and brevity and should not be construed as an inflexible limitation on the scope of the invention. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6, etc., as well as individual numbers within that range, for example, 1, 2, 3, 4, 5, and 6. This applies regardless of the breadth of the range. Numeric ranges recited are inclusive of the numbers defining the range and include each integer within the defined range.

The abbreviation “PBS” when used herein means phosphate-buffered saline. The abbreviation “PEG” when used herein means polyethylene glycol.

### BRIEF DESCRIPTION OF FIGURES

FIG. 1—Analysis of the effect of AAV2 formulation on (A) % transduction efficiency and (B) MFI of GFP+ cells at MOI 1×10⁵

FIG. 2—Analysis of the effect of AAV2 formulation at MOI 1×10⁵ on (A) % transduction efficiency and (B) MFI of GFP+ cells

FIG. 3—Subgroup analysis of the effect of AAV2 formulation at MOI 1×10⁵ on (A) % transduction efficiency and (B) MFI of GFP+ cells

FIG. 4—Formulation Effects on AAV8 Transduction Efficiency in HEK293T cells at MOI=1.00E+06 (% GFP Expression by FACS)

FIG. 5—Formulation Effects on AAV8 Transduction Efficiency in HEK293T cells at MOI=1.00E+06 (MFI Analysis by FACS)

FIG. 6—Formulation Effects on AAV2.7m8 Transduction Efficiency in HEK293T cells at MOI=5.00E+05 (% GFP Expression by FACS)

FIG. 7—Formulation Effects on AAV2.7m8 Transduction Efficiency in HEK293T cells at MOI=5.00E+05 (MFI Analysis by FACS)

FIG. 8—Fiber Fabrication Process A

FIG. 9—Fiber Fabrication Process B

FIG. 10—SS:SG Ratios can be selected to control the release profile (A) Groups 3.1A and 3.1B (B) Groups 3.1C and 3.1D (C) Groups 3.1E and 3.1F (D) Groups 3.1G and 3.1H

FIG. 11—SS:SG Ratios can selected to control the release profile (A) Groups 3.1I and 3.1J (B) Groups 3.1K, 3.1L, and 3.1M (C) Groups 3.1N, 3.10, and 3.1P (D) Groups 3.1Q, 3.1R, and 3.1S

FIG. 12—SS:SG Ratios can be selected to control the release profile—Groups 3.1T, 3.1U, 3.1V, and 3.1W.

FIG. 13—SS:SG Ratios can be selected to control the release profile—Groups 3.1X and 3.1Y.

FIG. 14—SS:SG Ratios can be selected to control the release profile—Groups 3.1Z and 3.1AA

FIG. 15—SS:SG Ratios can be selected to control the release profile—Groups 3.1AB and 3.1AC

FIG. 16—SS:SG Ratios can be selected to control the release profile—Groups 3.1AD, 3.1AE and 3.1AF

FIG. 17—Molecular weight between crosslinks can be selected to control the release profile—Groups 3.2A and 3.2B

FIG. 18—Molecular weight between crosslinks can be selected to control the release profile—Groups 3.2C and 3.2D

FIG. 19A—LDP:maPEG ratio can be used to control the release profile—Groups 3.3A and 3.3B

FIG. 19B—LDP:maPEG ratio can be used to control the release profile—Groups according to Table 19AA

FIG. 20—LDP:maPEG ratio can be used to control the release profile—Groups 3.3C and 3.3D

FIG. 21—LDP:maPEG ratio can be used to control the release profile—Groups 3.3E and 3.3F

FIG. 22—LDP:maPEG ratio can be used to control the release profile—Groups 3.3G and 3.3H

FIG. 23—LDP:maPEG ratio can be used to control the release profile—Groups 3.3I and 3.3J

FIG. 24—Number of days required for 100% release can be tuned—at pH=8.0 & 37° C. (A) 8% and 10% PEG and (B) 6% PEG (C) 6% and 8% PEG

FIG. 25—Number of days required for 100% release can be tuned—6% maPEG cumulative release with varying SS:SG ratios at pH=7.2 & 37° C.—Groups 56-1, 56-2, 56-3, 54-4 and 54-3

FIG. 26—Number of days required for 100% release can be tuned—8% maPEG cumulative release with varying SS:SG ratios at pH=7.2 & 37° C.—Groups 56-4, 56-5, 56-6 and 54-6

FIG. 27—High sugar content leads to 100% release within about 2 days irrespective of other factors.

FIG. 28—AAV release profile is reproducible at varying doses

FIG. 29—AuNP release profiles can be reproduced with AAV

FIG. 29C—Dv₉₀ particle size of groups 1 and 2 according to Tables 27A and Table 27B

FIG. 29D—Dv₉₀ particle size can be used to control the release profile (Groups 1 and 2 according to Tables 27A and Table 27B)

FIG. 29E—Dv₉₀ particle size can be used to control the release profile (Group 3 using microemulsion and Group 1 of Tables 27A and 27B).

FIG. 30—In vivo AAV2 transduction and GFP expression with the formulated implants on Days 0, 4, 7, 10, 14, 17, 21, 24, and 28. The top to bottom listed days should be read from left to right on the graphs

FIG. 31—(A) Change in diameter of Fibers over time according to Example 5 (B) Change in length of Fibers over time according to Example 5. The top to bottom listed groups should be read from left to right on the graphs

FIG. 32—AAV2 Dose comparison Theoretical versus PCR versus ELISA-PCR and ELISA Results are comparable. The top to bottom listed groups should be read from left to right on the graphs

FIG. 33—AAV2 Fibers—Release profiles used for in vivo administration according to Example 5

FIG. 34—In vivo administration of AAV2 Implant according to Example 5: Inflammation scores—Aqueous Cells

FIG. 35—In vivo administration of AAV2 Implant according to Example 5: Inflammation scores—Aqueous Flare

FIG. 36—In vivo administration of AAV2 Implant according to Example 5: Inflammation scores—Vitreous Cells

FIG. 37—In vivo administration of AAV2 Implant according to Example 5: FAF images and Inflammation scores of G1 Placebo on days 2, 14, 29, 44 and 56. VH=vitreous haze, VC=vitreous cells, AC=aqueous cells, and AF=aqueous flare.

FIG. 38—In vivo administration of AAV2 Implant according to Example 5: FAF images and Inflammation scores of G2 AAV2 Liquid on days 2, 14, 29, 44 and 56. VH=vitreous haze, VC=vitreous cells, AC=aqueous cells, and AF=aqueous flare

FIG. 39—In vivo administration of AAV2 Implant according to Example 5: FAF images and Inflammation scores of G3 AAV2 Implant on days 2, 14, 29, 44 and 56. VH=vitreous haze, VC=vitreous cells, AC=aqueous cells, and AF=aqueous flare.

FIG. 40—In vivo administration of AAV2 Implant according to Example 5: FAF images and Inflammation scores of G4 AAV2 Liquid+TA on days 2, 14, 29, 44 and 56. VH=vitreous haze, VC=vitreous cells, AC=aqueous cells, and AF=aqueous flare.

FIG. 41—In vivo administration of AAV2 Implant according to Example 5: FAF images and Inflammation scores of G5 AAV2 Implant+TA on days 2, 14, 29, 44 and 56. VH=vitreous haze, VC=vitreous cells, AC=aqueous cells, and AF=aqueous flare.

FIG. 42—In vivo administration of AAV2 Implant according to Example 5: FAF images and Inflammation scores of G6 AAV2 Liquid+TKI on days 2, 14, 29, 44 and 56. VH=vitreous haze, VC=vitreous cells, AC=aqueous cells, and AF=aqueous flare.

FIG. 43—In vivo administration of AAV2 Implant according to Example 5: FAF images and Inflammation scores of G7 AAV2 Implant+TKI on days 2, 14, 29, 44 and 56. VH=vitreous haze, VC=vitreous cells, AC=aqueous cells, and AF=aqueous flare.

FIG. 44—In vivo biodistribution of AAV2 following AAV2 implant or AAV2 bolus according to Example 5: bar graph illustrating AAV vector copy number in the aqueous humor and vitreous humor of rabbits administered an AAV2 bolus or an AAV implant, as indicated.

FIG. 45—(A) Change in diameter of Fibers over time according to Example 6 (B) Change in length of Fibers over time according to Example 6

FIG. 46—AAV2.7m8 Fibers Release profiles used for in vivo administration according to Example 6

FIG. 47A—In vivo administration of AAV2.7m8 Implant according to Example 6: Inflammation scores-Aqueous Cells

FIG. 47B—In vivo administration of AAV2.7m8 Implant according to Example 6: Inflammation scores-Aqueous Flare

FIG. 48—In vivo administration of AAV2.7m8 Implant according to Example 6: FAF images and Inflammation scores of Bolus, Fast Release and Medium Release Groups on Day 9. VH=vitreous haze, VC=vitreous cells, AC=aqueous cells, and AF=aqueous flare.

FIG. 49—In vivo administration of AAV2.7m8 Implant according to Example 6: FAF images and Inflammation scores of Bolus, Fast Release and Medium Release Groups on Day 30. VH=vitreous haze, VC=vitreous cells, AC=aqueous cells, and AF=aqueous flare.

FIG. 50—In vivo administration of AAV2.7m8 Implant according to Example 6: FAF images and Inflammation scores of Bolus, Fast Release and Medium Release Groups on Day 72. VH=vitreous haze, VC=vitreous cells, AC=aqueous cells, and AF=aqueous flare.

FIG. 51—AAV2 and AAV2.7m8 release over time based on ELISA results from implants used for in vivo administration according to Example 5 (1^{st} Rabbit study) and Example 6 (2^{nd} Rabbit study)

FIG. 52—Results of the anti-drug antibody (ADA) assay against AAV2.7m8 in the serum of bolus, fast release and medium release groups from week 0 up to week 13 according to Example 6 (2^{nd} rabbit study). The sample titer value is the highest dilution at which a sample has a mean OD value equal to or above the assay cutoff, with the next highest dilution being below the assay cutoff. Values are depicted as reciprocal dilution.

FIG. 53—Vector shedding quantified as copies of the heterologous nucleic acid sequence detected in the plasma on day 0, 2, 4, 7, and Week 2 post administration in bolus, fast release and medium release groups according to Example 6 (2^{nd} rabbit study). LLOQ=lower limit of quantification (5000 VG/mL).

FIG. 54—Serum ADA titer versus Vector shedding in bolus, fast release and medium release groups according to Example 6 (2^{nd} Rabbit Study). Vector copies per mL refers to the copies of the heterologous nucleic acid sequence in the AAV, in the present case, eGFP.

FIG. 55—Results of GFP quantification at week 14 in ocular tissues of bolus, fast release and medium release groups according to Example 6 (2^{nd} rabbit study).

FIG. 56—Vector copies in plasma at Day 2 versus aqueous cell scores (at week 3) in bolus, fast release and medium release groups according to Example 6 (2^{nd} Rabbit Study). Vector copies per mL refers to the copies of the heterologous nucleic acid sequence in the AAV, in the present case, eGFP.

FIG. 57—In vivo administration of AAV2.7m8 Implant according to Example 7: Inflammation scores—Aqueous Cell (A) Overall, (B) Placebo, (C) bolus, (D) fast release and (E) medium release. * indicates treatment of IVT dose of TA due to severe ocular inflammation.

FIG. 58—In vivo administration of AAV2.7m8 Implant according to Example 7: Inflammation scores—Aqueous Flare in bolus, fast release and medium release groups.

FIG. 59—In vivo administration of AAV2.7m8 Implant according to Example 7: FAF images and Inflammation scores of Bolus, Fast Release and Medium Release Groups at Week 8. VH=vitreous haze, VC=vitreous cells, AC=aqueous cells, and AF=aqueous flare.

FIG. 60—In vivo administration of AAV2.7m8 Implant according to Example 7: FAF images and Inflammation scores of Bolus, Fast Release and Medium Release Groups at Week 12. VH=vitreous haze, VC=vitreous cells, AC=aqueous cells, and AF=aqueous flare.

FIG. 61—Vector shedding quantified as copies of the heterologous nucleic acid sequence (here eGFP) detected in the plasma by qPCR at predose, on day 2, 4, 7, 10, at Week 2 and Week 3 post administration in placebo, bolus, fast release and medium release groups according to Example 7 (NHP study). LLOQ=lower limit of quantification (5000 VG/mL).

FIG. 62—In vitro release profile of AAV2.7m8 from the medium and fast release implants used in the NHP study according to Example 7

### DETAILED DESCRIPTION OF THE INVENTION

### Xerogel

According to some aspects of the invention, a xerogel is a dehydrated gel comprising a matrix and/or a polymer network comprising at least two covalently crosslinked multi-arm precursors. In some embodiments, a xerogel is a dehydrated organogel comprising a matrix and/or a polymer network comprising at least two covalently crosslinked multi-arm precursors.

Thus, a precursor is always a “functional polymer” that is able to participate in the crosslinking reaction with another precursor to form a polymer network or matrix. Thus, the term “non-functional polymer” refers to a polymer that may be present in the organogel, xerogel, hydrogel and/or implant (or pharmaceutically acceptable implant) of the present invention but does not participate in the crosslinking reaction with the precursors to form a polymer network or matrix.

The precursor used in the invention may be any polymer as long as it is soluble in an organic solvent, is able to react with another precursor, and is unreactive to a biologic. The polymer may be selected from a natural, synthetic or biosynthetic polymer.

Natural polymers may include glycosaminoglycans, polysaccharides (e.g., dextran), polyaminoacids and proteins or mixtures or combinations thereof.

In some aspects, synthetic precursors are preferred. Synthetic refers to a molecule not found in nature or not normally found in a human. Synthetic polymer may generally be any polymer that is synthetically produced by different types of polymerization, including free radical polymerization, anionic or cationic polymerization, chain-growth or addition polymerization, condensation polymerization, ring-opening polymerization etc. The polymerization may be initiated by certain initiators, by light and/or heat, and may be mediated by catalysts.

Generally, for the purposes of the present invention one or more synthetic polymers of the group comprising one or more units of polyalkylene glycol, such as polyethylene glycol (PEG), polypropylene glycol, poly(ethylene glycol)-block-poly(propylene glycol) copolymers, or polyethylene oxide, polypropylene oxide, polyvinyl alcohol, poly(vinylpyrrolidinone), polylactic acid, polylactic-co-glycolic acid, random or block copolymers or combinations/mixtures of any of these can be used, while this list is not intended to be limiting.

The precursors have functional groups that react with each other. The functional groups react with each other in electrophile-nucleophile reactions or are configured to participate in other polymerization reactions. Thus, according to the invention, each precursor comprises at least one nucleophile or at least one electrophile.

Nucleophiles that can be used for the present invention may comprise an amine such as a primary amine, a thiol, an azide or a hydrazide. In certain embodiments, at least one precursor comprises a nucleophile preferably a primary amine.

Electrophiles that can be used for the present invention may comprise succinimidyl esters, succinimidyl carbonates, nitrophenyl carbonates, aldehydes, ketones, acrylates, acrylamides, maleimides, vinylsulfones, iodoacetamides, alkenes, alkynes, dibenzocyclooctynes, norbornenes, epoxides, mesylates, tosylates, tresyls, cyanurates, orthopyridyl disulfides, or halides. These electrophiles may comprise reactive groups that participate in the electrophile-nucleophile reaction. For example, in an embodiment of the invention, a succinimidyl ester may comprise a reactive group such as succinimidyl succinate (SS), succinimidyl glutarate (SG), succinimidyl adipate (SAP), succinimidyl azelate (SAZ), or succinimidyl glutaramide.

The term “multi-arm” precursors means that the precursors are branched. In the case of a multi-arm polymer, a core refers to a contiguous portion of a molecule joined to arms that extend from the core, with the arms having a nucleophile or electrophile, which is often at the terminus of the branch. Precursors may have, e.g., 2-100 arms, with each arm having a terminus, bearing in mind that some precursors may be dendrimers or other highly branched materials. An arm on a precursor refers to a linear chain of chemical groups that connect a cross linkable group to a polymer core. Some embodiments are precursors with between 3 and 300 arms; artisans will immediately appreciate that all the ranges and values within the explicitly stated ranges are contemplated, e.g., 4, 6, 8, 10, 12, 4 to 16, 8 to 100, 6, 8, 10, 12, or at least 4 arms.

In some embodiments, when each precursor is multi-arm, it comprises two or more arms and thus, two or more same or different electrophiles or nucleophiles, such that each nucleophile may react with another electrophile (within the same precursor or another precursor) in an electrophilic-nucleophilic reaction to form a crosslinked polymeric product. Thus, for example, in some aspects, the precursor has 4 arms, wherein each arm terminates with either a nucleophile or an electrophile that may or may not be the same as its other arms.

According to an aspect of the invention, the xerogel comprises at least two multi-arm precursors comprising a first multi-arm precursor comprising nucleophiles and/or electrophiles, and a second multi-arm precursor comprising nucleophiles and/or electrophiles. In this embodiment, the first multi-arm precursor and the second multi-arm precursor are covalently cross-linked with each other in an electrophile-nucleophile reaction. In this context, the multi-arm refers to at least 10 arms, at least 8 arms, such as at least 4 arms.

In one embodiment, if the xerogel comprises two multi-arm precursors, it may comprise a first multi-arm precursor comprising a nucleophile such as an amine such as a primary amine, a thiol, an azide or a hydrazide, and a second multi-arm precursor comprising an electrophile such as succinimidyl esters, succinimidyl carbonates, nitrophenyl carbonates, aldehydes, ketones, acrylates, acrylamides, maleimides, vinylsulfones, iodoacetamides, alkenes, alkynes, dibenzocyclooctynes, norbornenes, epoxides, mesylates, tosylates, tresyls, cyanurates, orthopyridyl disulfides, or halides. The nucleophile and electrophile are covalently cross-linked to each other in an electrophile-nucleophile reaction. In some embodiments, the first multi-arm precursor is a primary amine, and the second multi-arm precursor is a succinimidyl ester.

According to the invention, the xerogel comprises at least three multi-arm precursors comprising a first multi-arm precursor comprising nucleophiles and/or electrophiles, and a second multi-arm precursor comprising nucleophiles and/or electrophiles and a third multi-arm precursor comprising nucleophiles and/or electrophiles. In this embodiment, the first multi-arm precursor, the second multi-arm precursor, and the third multi-arm precursors are covalently cross-linked with each other in an electrophile-nucleophile reaction. In this context, the multi-arm refers to at least 10 arms, at least 8 arms, such as at least 4 arms.

According to the invention, the xerogel comprises at least three multi-arm precursors comprising a first multi-arm precursor comprising a nucleophile, a second multiarm precursor comprising an electrophile and a third multi-arm precursor comprising an electrophile. In this embodiment, the first multi-arm precursor and the second multi-arm precursor, and the first multi-arm precursor and the third multi-arm precursors are covalently cross-linked with each other in an electrophile-nucleophile reaction. In this context, the multi-arm refers to at least 10 arms, at least 8 arms, such as at least 4 arms. In this embodiment, the nucleophile can be an amine such as a primary amine, a thiol, an azide or a hydrazide, and the electrophiles can be succinimidyl esters, succinimidyl carbonates, nitrophenyl carbonates, aldehydes, ketones, acrylates, acrylamides, maleimides, vinylsulfones, iodoacetamides, alkenes, alkynes, dibenzocyclooctynes, norbornenes, epoxides, mesylates, tosylates, tresyls, cyanurates, orthopyridyl disulfides, or halides. For example, in an embodiment of the invention, a succinimidyl ester may comprise a reactive group such as succinimidyl succinate (SS), succinimidyl glutarate (SG), succinimidyl adipate (SAP), succinimidyl azelate (SAZ), or succinimidyl glutaramide.

Some precursors may have a longer hydrolysis half-life as compared to others. This means that the time required for them to degrade may be longer. This may, in part, be due to the reactive group comprised in that precursor. For example, a PEG polymer comprising an electrophile group such as a succinimidyl ester group that comprises a reactive group such as a succinimidyl succinate (SS) has a shorter hydrolysis half-life as compared to a PEG polymer comprising an electrophile group such as a succinimidyl ester group that comprises a reactive group such as a succinimidyl glutarate (SG). For example, a PEG polymer comprising an electrophile group such as a succinimidyl ester group that comprises a reactive group such as a succinimidyl succinate (SG) has a shorter hydrolysis half-life as compared to a PEG polymer comprising an electrophile group such as a succinimidyl ester group that comprises a reactive group such as a succinimidyl glutarate (SAP). For example, a PEG polymer comprising an electrophile group such as a succinimidyl ester group that comprises a reactive group such as a succinimidyl succinate (SAP) has a shorter hydrolysis half-life as compared to a PEG polymer comprising an electrophile group such as a succinimidyl ester group that comprises a reactive group such as a succinimidyl glutarate (SAZ).

According to the invention, if the xerogel comprises three multi-arm precursors, it may comprise a first multi-arm precursor comprising a nucleophile such as an amine, and a second multi-arm precursor comprising an electrophile such as a succinimidyl ester, and a third precursor also comprising an electrophile that may or may not be a succinimidyl ester. Thus, in another embodiment, if the xerogel comprises three precursors, it may comprise a first multi-arm precursor comprising a nucleophile such as an amine such as a primary amine, and a second multi-arm precursor comprising an electrophile such as a succinimidyl ester comprising a first reactive group and a third precursor comprising an electrophile that is a succinimidyl ester comprising a second reactive group. In this embodiment, the reactive group is selected from succinimidyl succinate (SS), succinimidyl glutarate (SG), succinimidyl adipate (SAP) or a succinimidyl azelate (SAZ).

In some embodiments, precursors are polyethylene glycol precursors. Thus, in some embodiments, the polymer network or matrix of covalently cross-linked precursors is made of polyethylene glycol-containing precursor. Polyethylene glycol (PEG, also referred to as polyethylene oxide) refers to a polymer with a repeat group (CH2CH20) n, with n being at least 3.

A polymeric precursor having a polyethylene glycol thus has at least three of these repeat groups connected to each other in a linear series. A PEG polymer that terminates in a hydroxyl group or a methoxy group that does not participate in the crosslinking reaction between the precursors is referred to as a “non-functional PEG” described herein above and thus, not used as one of the precursors. Thus, a PEG polymer that terminates in a nucleophile selected from a primary amine, a thiol, an azide or a hydrazide is considered as a “functional PEG” and can be used as one of the precursors. Further, a PEG polymer that terminates in an electrophile selected from succinimidyl esters, succinimidyl carbonates, nitrophenyl carbonates, aldehydes, ketones, acrylates, acrylamides, maleimides, vinylsulfones, iodoacetamides, alkenes, alkynes, dibenzocyclooctynes, norbornenes, epoxides, mesylates, tosylates, tresyls, cyanurates, orthopyridyl disulfides, or halides is considered as a “functional PEG” and can be used as one of the precursors.

The polymer network of the hydrogel implants of the present invention may comprise one or more multi-arm PEG units having from 2 to 10 arms, or 4 to 8 arms, or 4, 5, 6, 7 or 8 arms. The PEG units may have a different or the same number of arms. In certain embodiments, the PEG units used in the hydrogel of the present invention have 4 and/or 8 arms. In certain particular embodiments, a combination of 4- and 8-arm PEG units is utilized.

In certain embodiments of the present invention, polyethylene glycol units used as precursors have an average molecular weight in the range from about 2,000 to about 100,000 Daltons, or in a range from about 10,000 to about 60,000 Daltons, or in a range from about 15,000 to about 50,000 Daltons. In certain particular embodiments the polyethylene glycol units have an average molecular weight in a range from about 10,000 to about 40,000 Daltons, or of about 20,000 Daltons. PEG precursors of the same average molecular weight may be used, or PEG precursors of different average molecular weight may be combined with each other. The average molecular weight of the PEG precursors used in the present invention is given as the number average molecular weight (Mn), which, in certain embodiments, may be determined by MALDI.

In a 4-arm PEG, each of the arms may have an average arm length (or molecular weight) of the total molecular weight of the PEG divided by 4. A 4a20kPEG precursor, which is one precursor that can be utilized in the present invention thus has 4 arms with an average molecular weight of about 5,000 Daltons each. An 8a20k PEG precursor, which may be used in addition to the 4a20kPEG precursor in the present invention, thus has 8 arms each having an average molecular weight of 2,500 Daltons.

When referring to a PEG precursor having a certain average molecular weight, such as a 15kPEG- or a 20kPEG-precursor, the indicated average molecular weight (i.e., a Mn of 15,000 or 20,000, respectively) refers to the PEG part of the precursor, before end groups are added (“20k” here means 20,000 Daltons, and “15k” means 15,000 Daltons—the same abbreviation is used herein for other average molecular weights of PEG precursors). In certain embodiments, the Mn of the PEG part of the precursor is determined by MALDI. The degree of substitution with end groups as disclosed herein may be determined by means of H-NMR after end group functionalization.

In various embodiments of the invention, the xerogel comprises at least two multi-arm precursors, the first precursor is a multi-arm PEG precursor comprising a nucleophile such as an amine, such as a primary amine. In this embodiment, the second multi-arm precursor is a multi-arm PEG precursor comprising an electrophile such as a succinimidyl ester.

In various embodiments of the invention, the xerogel comprises three multiarm precursors, the first multi-arm precursor is a multi-arm PEG precursor comprising a nucleophile such as an amine, such as a primary amine. In this embodiment, the second multi-arm precursor is a multi-arm PEG precursor comprising an electrophile such as a succinimidyl ester comprising a first reactive group. In this embodiment, the third multiarm precursor is a multi-arm PEG precursor comprising an electrophile such as a succinimidyl ester comprising a second reactive group. In this embodiment, the first and the second reactive groups can be selected from succinimidyl succinate (SS), succinimidyl glutarate (SG), succinimidyl adipate (SAP) or a succinimidyl azelate (SAZ). SS, SG, SAP and SAZ are all reactive groups of succinimidyl esters that have an ester group that degrades by hydrolysis in water. In some embodiments, the first multi-arm precursor is succinimidyl succinate (SS) and the second multi-arm precursor is succinimidyl glutarate (SG).

Each and any combination of electrophilic- and nucleophilic-group containing PEG precursors disclosed herein may be used for preparing the implant according to the present invention. For example, any 4-arm or 8-arm PEG precursor (e.g., having a succinimidyl ester comprising a SS, SG, SAP, or SAZ reactive group) may be combined with any 4-arm or 8-arm PEG precursor (e.g., having a NH2 group or another nucleophile). Furthermore, the PEG units of the electrophile- and the nucleophile group-containing precursors may have the same or may have a different average molecular weight.

One such combination is a PEG amine precursor and two PEG succinimidyl ester precursors, one comprising an SS reactive group and another comprising an SG reactive group. The inventors have found that by keeping the molar ratio of PEG amine to PEG succinimidyl ester at 1:1 and by varying the molar ratio of the reactive groups of the succinimidyl esters SS and SG, the time taken by the polymeric network to degrade in an aqueous solution under physiological conditions can be controlled. The amount of PEG SS and SG to be used to reach a particular molar ratio of the two reactive groups can be calculated by a skilled artisan and described as follows.

The amount of PEG amine and PEG esters (SS and SG) to be used is calculated through stoichiometric equations of molar proportion and converting moles to grams. First, the reactive end group molar ratio between the amine, the succinimidyl succinate, and succinimidyl glutarate is determined. In an example formulation, 4a20k PEG NH2, 4a20k PEG SS, and 4a40k PEG SG are used. The molar ratio between amine and succinimidyl ester groups is 1:1, and the molar ratio between SS and SG is 80:20. The final end group molar ratio between the 4a20k NH2: 4a20k SS: 4a40k SG is 1.0:0.8:0.2. Next, gram to mole stoichiometric conversions, and vice versa, are used to determine mass amounts. Below outlines an example calculation of 4a20k SS at the molar ratios above with 100 g of 4a20k NH2:

$$100 g 4 a 20 k PEG NH 2 \times \frac{1 mol PEG NH 2}{20000 g PEG NH 2} \times \frac{4 mol NH 2}{1 mol PEG NH 2} \times \frac{0.8 mol SS}{1 mol NH 2} \times \frac{1 mol PEG SS}{4 mol SS} \times \frac{20000 g PEG SS}{1 mol PEG SS} = 80 g 4 a 20 k PEG SS$$

Alternatively, the amounts of PEGs can be determined by calculating the “molecular weight between crosslinks” (MWc) and the arm length ratio. The MWc can be calculated through the sum of the average arm length of each multi-arm PEG precursor.

$$PEG Arm Length = \frac{PEG MW}{\# arms} \times PEG molar ratio$$

$$MWc = PEG NH 2 Arm Length + PEG SS Arm Length + PEG SG Arm Length$$

The arm length ratio is calculated by dividing the PEG Arm Length over the MWc. By multiplying the arm length ratio for a particular multi-arm precursor with a total PEG batch size, the amount of multi-arm precursor can be determine. Below outlines an example calculation for the amount of 4a20k PEG SS with a total batch size of 100 g PEG:

$$PEG NH 2 Arm Length = \frac{20000 Da}{4} \times 1. = 5000 Da$$

$$PEG SS Arm Length = \frac{20000 Da}{4} \times 0.8 = 4000 Da$$

$$PEG SG Arm Length = \frac{40000 Da}{4} \times 0.2 = 2000 Da$$

$$MWc = 5000 Da + 4000 Da + 2000 Da = 11000 Da$$

$$PEG SS Arm Length Ratio = \frac{4000 Da}{11000 Da} = 0.364$$

$$Mass PEG SS = 0.364 \times 100 g = 36.4 g$$

In certain preferred embodiments, 4-arm PEGs with an average molecular weight of about 20,000 Daltons and 4-arm PEGs with an average molecular weight of about 40,000 Daltons can be used for forming the polymer network and thus the xerogel according to the present invention.

Thus, the first precursor, the second precursor and/or the third precursor may be a 4a20k precursor, wherein 4 denotes the arms and 20k denotes the Mn. Thus, for example, the first, second and/or the third precursor may be a 4a40k precursor. Thus, for example, the first and/or the second precursor may be a 4a20k precursor and the third precursor may be a 4a40k precursor.

In certain embodiments, the nucleophile-containing crosslinking agent may be bound to or conjugated with a visualization agent. A visualization agent is an agent that contains a fluorophoric or other visualization-enabling group. Fluorophores such as fluorescein, rhodamine, coumarin, and cyanine may for example be used as visualization agents. The visualization agent may be conjugated with the crosslinking agent e.g. through some of the nucleophiles of the crosslinking agent. Since a sufficient amount of the nucleophiles are necessary for crosslinking, “conjugated” or “conjugation” in general includes partial conjugation, meaning that only part of the nucleophiles are used for conjugation with the visualization agent, such as about 1% to about 20%, or about 5% to about 10%, or about 8% of the nucleophiles of the crosslinking agent may be conjugated with a visualization agent. In other embodiments, a visualization agent may also be conjugated with the polymer precursor, e.g. through certain reactive (such as electrophile) of the polymer precursors.

### Active Agent: Biologic

The active agent according to the invention can be a plurality of the same or different biologics. A biologic can be, for example, a polypeptide, a protein encapsulating a nucleic acid, a virus, or a lipid encapsulating a nucleic acid.

As used herein, peptide is any compound containing two or more amino acid residues joined by an amide bond formed from the carboxyl group of one amino acid residue and the amino group of the adjacent amino acid residue. The amino acid residues may have the L-form as well as the D-form, and may be naturally occurring or synthetic, linear as well as cyclic. Also included within the meaning of peptides are polypeptides and peptide dimers which can be peptides linked C-terminus to N-terminus (tandem repeats) or peptides linked C-terminus to C-terminus (parallel repeats).

A protein fragment is any section of the polypeptide sequence that has been separated from the rest of the protein and takes a form of primary, secondar or tertiary structure on its own. In some embodiments, these fragments are at least 8 amino acids long and are at least 40% to 99% identical to the reference protein, more preferably 70%, 80% or 90% or 99% identical to the reference protein.

The structure of a protein or a polypeptide is typically described by its primary, secondary, tertiary, and quaternary structures. The amino acid sequence of the protein defines the primary structure. Thus, according to the invention, a biologic may be a polypeptide comprising a primary structure.

Proteins seldom form random coils and the high specificity of their function depends on a defined conformation of the polypeptide chain, in a secondary structure. The most common types of secondary structures are α-helices and β-sheets. Thus, according to the invention, a biologic may be a polypeptide comprising a secondary structure.

The elements of secondary structure may be connected via loops and turns of various types into a larger tertiary structure. Polypeptide tertiary structure is the three-dimensional shape of a protein. The tertiary structure will have a single polypeptide chain “backbone” with one or more secondary structures. The interactions and bonds of side chains within a particular protein determine its tertiary structure. Thus, according to the invention, a biologic may be a polypeptide comprising a tertiary structure.

The quaternary structure of a protein is the association of several polypeptide chains or subunits into a closely packed arrangement. Each of the subunits has its own primary, secondary, and tertiary structure. The subunits are held together by hydrogen bonds and van der Waals forces between nonpolar side chains. Thus, according to the invention, a biologic may be a polypeptide comprising a quaternary structure.

In some embodiments, the biologic according to the present invention is a recombinant protein or recombinant polypeptide used interchangeably here. In some embodiments, a recombinant protein refers to an antibody, an antigen binding fragment, a fusion protein, a cytokine, a transcription factor, an enzyme or a hormone.

The term “antibody” or “antibodies” as used herein refers to monoclonal or polyclonal antibodies. The term “antibody” or “antibodies” as used herein includes but is not limited to recombinant antibodies that are generated by recombinant technologies as known in the art. “Antibody” or “antibodies” include antibodies' of any species, in particular of mammalian species; such as human antibodies of any isotype, including IgA1, IgA2, IgD, IgG1, IgG2a, IgG2b, IgG3, IgG4 IgE and IgM and modified variants thereof, non-human primate antibodies, e.g. from chimpanzee, baboon, rhesus or cynomolgus monkey; rodent antibodies, e.g. from mouse, rat or rabbit; goat or horse antibodies; and camelid antibodies (e.g. from camels or llamas such as Nanobodies) and derivatives thereof; or of bird species such as chicken antibodies or of fish species such as shark antibodies. The term “antibody” or “antibodies” also refers to “chimeric” antibodies in which a first portion of at least one heavy and/or light chain antibody sequence is from a first species and a second portion of the heavy and/or light chain antibody sequence is from a second species. The term “antibody” or “antibodies” further includes antigen-binding portions or antigen-binding fragments of an antibody. The term “antigen binding fragment” also refers to an antibody that comprises at least one heavy or light chain immunoglobulin domain as known in the art and binds to one or more antigen(s). Examples of antibody fragments that can be used as biologics include Fab, Fab′, F(ab′) 2, and Fv and scFv fragments; as well as diabodies, triabodies, tetrabodies, minibodies, domain antibodies, single-chain antibodies, bispecific, trispecific, tetraspecific or multispecific antibodies formed from antibody fragments or antibodies, including but not limited to Fab-Fv constructs. In some aspects, the antibody is a vNAR, a camelid antibody, a VHH antibody, or an antigen-binding portion thereof. Antibody fragments as defined above are known in the art.

A fusion protein is a protein consisting of at least two domains that are encoded by separate genes that have been joined so that they are transcribed and translated as a single unit, producing a single polypeptide. Fusion proteins may also be used interchangeably with chimeric proteins.

By the term “cytokine” as used herein is meant a molecule which modulates interactions between cells in the immune or inflammatory response. A cytokine includes but is not limited to monokines and lymphokines.

“Hormones” as used herein refer to any signalling molecule that exerts its effect on specific cell types. The term “hormones” encompasses any type of hormone such as endocrine, paracrine, autocrine and intracrine. In some embodiments, hormone as used herein refers to a polypeptide hormone.

“Transcription factor” as used herein should be construed in the broadest possible sense as any protein involved in the process of converting, or transcribing, DNA into RNA. Transcription factors include a wide number of proteins that initiate and regulate the transcription of genes. One distinct feature of transcription factors is that they have DNA-binding domains that give them the ability to bind to specific sequences of DNA called enhancer or promoter sequences. Some transcription factors bind to a DNA promoter sequence near the transcription start site and help form the transcription initiation complex. Other transcription factors bind to regulatory sequences, such as enhancer sequences, and can either stimulate or repress transcription of the related gene.

Common examples of a recombinant protein that can be considered as a biologic comprised in the pharmaceutically acceptable implant of the present invention include but are not limited to RPE65, REP1, RPGR, BEST1, anti-VEGF inhibitors such as aflibercept, ranibizumab, brolucizumab, or bevacizumab, 31tructure31 sodium, adalimumab, Infliximab, hRS1, hCNGB3, ABCR, MYO7A, endostatin, angiostatin, TNF [alpha] receptor, the TGF [beta]2 receptor, IRS-1, IGF-1, Angiogenin, Angiopoietin-1, DeM, acidic or basic Fibroblast Growth Factors (aFGF and bFGF), FGF-2, Follistatin, Granulocyte Colony-Stimulating factor (G-CSF), Hepatocyte Growth Factor (HGF), Scatter Factor (SF), Leptin, Midkine, Placental Growth Factor (PGF), Platelet-Derived Endothelial Cell Growth Factor (PD-ECGF), Platelet-Derived Growth Factor-BB (PDGF-BB), Pleiotrophin (PTN), RdCVF (Rod-derived Cone Viability Factor), Progranulin, Proliferin, Transforming Growth Factor-alpha (TGF-alpha), PEDF, Transforming Growth Factor-beta (TGF-beta), Vascular Permeability Factor (VPF), CNTF, BDNF, GDNF, PEDF, NT3, BFGF, ephrin, EPO, NGF, GMF, aFGF, NT5, Gax, a growth hormone, [alpha]-1-antitrypsin, calcitonin, leptin, an apolipoprotein, an enzyme for the biosynthesis of vitamins, hormones or neuromediators, chemokines, cytokines such as IL-1, IL-8, IL-10, IL-12, IL-13, a receptor thereof, an antibody blocking any one of said receptors, TIMP such as TIMP-1, TIMP-2, TIMP-3, TIMP-4, angioarrestin, endostatin such as endostatin XVIII and endostatin XV, ATF, a fusion protein of endostatin and angiostatin, the C-terminal hemopexin domain of matrix metalloproteinase-2, the kringle 5 domain of human plasminogen, a fusion protein of endostatin and the kringle 5 domain of human plasminogen, the placental ribonuclease inhibitor, the plasminogen activator inhibitor, the Platelet Factor-4 (PF4), a prolactin fragment, the Proliferin-Related Protein (PRP), the antiangiogenic antithrombin III, the Cartilage-Derived Inhibitor (CDI), a CD59 complement fragment, C3a and C5a inhibitors, complex attack membrane inhibitors, Factor H, ICAM, VCAM, caveolin, PKC zeta, junction proteins, JAMs, CD36, MERTK vasculostatin, vasostatin (calreticulin fragment), thrombospondin, fibronectin, in particular fibronectin fragment gro-beta, an heparinase, human chorionic gonadotropin (hCG), interferon alpha/beta/gamma, interferon inducible protein (IP-10), the monokine-induced by interferon-gamma (Mig), the interferon-alpha inducible protein 10 (IP10), a fusion protein of Mig and IP10, soluble Fms-Like Tyrosine kinase 1 (FLT-1) receptor, Kinase insert Domain Receptor (KDR), regulators of apoptosis such as Bcl-2, Bad, Bak, Bax, Bik, BcI-X short isoform and Gax, alpha-1 antitrypsin, factor IX, factor VIII, CI-esterase inhibitor, β-globin or γ-globin. The recombinant protein can also be a Cas9 polypeptide, a zinc-finger nuclease, a TALEN polypeptide, or any combination thereof.

The biologic can be a lipid encapsulating a nucleic acid. The nucleic acid can be any nucleic acid selected from a DNA and RNA. Some examples include ssDNA (single-strand DNA), dsDNA (double-stranded DNA), plasmid DNA, diploid RNA, small interfering RNA (siRNA), micro-RNA, dsRNA, mRNA, lncRNA, piRNA, rmRNA, sRNA, tiRNA, eRNA, snoRNA, snRNA, circRNA (circular RNA), a micro-RNA, a long non-coding RNA, RNA aptamer, antisense oligonucleotide, a guide RNA, a tRNA or any combination thereof. In some embodiments, a lipid encapsulating a nucleic acid is in the form of lipid nanoparticles. Lipid nanoparticles are spherical vesicles made of ionizable lipids, which are positively charged at low pH (enabling nucleic acid complexation) and neutral at physiological pH. In some aspects, the nucleic acid is encapsulated in a micro vesicle, a nanovesicle, an exosome, or an endosome.

A biologic can be a virus such as retrovirus, adenovirus, adeno-associated virus (AAV), lentivirus and herpes simplex virus. The term “virus” can also be used interchangeably with the term “protein encapsulating a nucleic acid(s)” since most viruses comprise at least an outer protein shell and an endogenous nucleic acid. In some embodiments, when the biologic is a virus, it comprises not only its endogenous nucleic acid but also a heterologous nucleic acid. A heterologous nucleic acid is any nucleic acid that does not belong to the virus.

The heterologous nucleic acid can be a DNA or RNA. The heterologous nucleic acid can be in the form of ssDNA (single-strand DNA), dsDNA (double-stranded DNA), plasmid DNA, diploid RNA, small interfering RNA (siRNA), micro-RNA, dsRNA, mRNA, lncRNA, piRNA, rmRNA, sRNA, tiRNA, eRNA, snoRNA, snRNA, circRNA (circular RNA), a micro-RNA, a long non-coding RNA, RNA aptamer, antisense oligonucleotide, a guide RNA, a tRNA or any combination thereof.

The heterologous nucleic acid can be a coding nucleic acid or a non-coding nucleic acid. In certain embodiments, the heterologous nucleic acid(s) code(s) for a therapeutic protein, which is absent in a subject, or present at a reduced level in a subject as compared to the same but healthy subject.

When the heterologous nucleic acid is a non-coding nucleic acid it may be selected from a group consisting of a ssDNA (single-strand DNA), dsDNA (double-stranded DNA), small interfering RNA (siRNA), micro-RNA, dsRNA, lncRNA, piRNA, rmRNA, sRNA, tiRNA, eRNA, snoRNA, snRNA, circRNA (circular RNA), RNA aptamer, antisense oligonucleotide, a guide RNA, a tRNA or any combination thereof.

When the heterologous nucleic acid is a coding nucleic acid, it preferably codes for a therapeutic protein. In some embodiments, the therapeutic protein can be but is not limited to RPE65, REP1, RPGR, BEST1, anti-VEGF inhibitors such as aflibercept, ranibizumab, brolucizumab, or bevacizumab, 33tructure33 sodium, adalimumab, Infliximab, hRS1, hCNGB3, ABCR, MYO7A, endostatin, angiostatin, TNF [alpha] receptor, the TGF [beta]2 receptor, IRS-1, IGF-1, Angiogenin, Angiopoietin-1, DeM, acidic or basic Fibroblast Growth Factors (aFGF and bFGF), FGF-2, Follistatin, Granulocyte Colony-Stimulating factor (G-CSF), Hepatocyte Growth Factor (HGF), Scatter Factor (SF), Leptin, Midkine, Placental Growth Factor (PGF), Platelet-Derived Endothelial Cell Growth Factor (PDECGF), Platelet-Derived Growth Factor-BB (PDGF-BB), Pleiotrophin (PTN), RdCVF (Rod-derived Cone Viability Factor), Progranulin, Proliferin, Transforming Growth Factor-alpha (TGF-alpha), PEDF, Transforming Growth Factor-beta (TGF-beta), Vascular Permeability Factor (VPF), CNTF, BDNF, GDNF, PEDF, NT3, BFGF, ephrin, EPO, NGF, GMF, aFGF, NT5, Gax, a growth hormone, [alpha]-1-antitrypsin, calcitonin, leptin, an apolipoprotein, an enzyme for the biosynthesis of vitamins, hormones or neuromediators, chemokines, cytokines such as IL-1, IL-8, IL-10, IL-12, IL-13, a receptor thereof, an antibody blocking any one of said receptors, TIMP such as TIMP-1, TIMP-2, TIMP-3, TIMP-4, angioarrestin, endostatin such as endostatin XVIII and endostatin XV, ATF, a fusion protein of endostatin and angiostatin, the C-terminal hemopexin domain of matrix metalloproteinase-2, the kringle 5 domain of human plasminogen, a fusion protein of endostatin and the kringle 5 domain of human plasminogen, the placental ribonuclease inhibitor, the plasminogen activator inhibitor, the Platelet Factor-4 (PF4), a prolactin fragment, the Proliferin-Related Protein (PRP), the antiangiogenic antithrombin III, the Cartilage-Derived Inhibitor (CDI), a CD59 complement fragment, C3a and C5a inhibitors, complex attack membrane inhibitors, Factor H, ICAM, VCAM, caveolin, PKC zeta, junction proteins, JAMs, CD36, MERTK vasculostatin, vasostatin (calreticulin fragment), thrombospondin, fibronectin, in particular fibronectin fragment gro-beta, an heparinase, human chorionic gonadotropin (hCG), interferon alpha/beta/gamma, interferon inducible protein (IP-10), the monokine-induced by interferon-gamma (Mig), the interferon-alpha inducible protein 10 (IP10), a fusion protein of Mig and IP10, soluble Fms-Like Tyrosine kinase 1 (FLT-1) receptor, Kinase insert Domain Receptor (KDR), regulators of apoptosis such as Bcl-2, Bad, Bak, Bax, Bik, BcI-X short isoform and Gax, alpha-1 antitrypsin, factor IX, factor VIII, CI-esterase inhibitor, β-globin or γ-globin. In some embodiments, the therapeutic protein is RPE65, REP1, RPGR, BEST1, anti-VEGF inhibitors like aflibercept, ranibizumab, or bevacizumab, hRS1, hCNGB3, ABCR, MYO7A, endostatin, angiostatin.

In some embodiments, the biologic is a virus, and the virus is adeno-associated virus (AAV) selected from a group consisting of AAV1, AAV2 such as AAV2.7m8, AAV3, AAV4, AAV5, AAV6, AAV7, AAV8, AAV9, AAV10, AAV11 and a mutant, hybrid or variant thereof. In other preferred embodiments, the virus is AAV2, AAV2.7m8, or AAV8. In these embodiments, the AAV comprises at least one heterologous nucleic acid sequence. In these embodiments, the heterologous nucleic acid sequence can be either a coding nucleic acid sequence or non-coding nucleic acid sequence. When the heterologous nucleic acid sequence is a coding nucleic acid sequence, it may code for a therapeutic protein described herein above.

The total amount or concentration of the biologic comprised in the pharmaceutically acceptable implant would depend on the type of biologic.

In one embodiment, the biologic is a virus and is comprised in the pharmaceutically acceptable implant at a total amount of at least 10⁹ vg. In certain embodiments, the virus is comprised in the pharmaceutically acceptable implant at a total amount from 10⁹ to 10¹⁵ vg. In other preferred embodiments, the virus is comprised in the pharmaceutically acceptable implant at a total amount from 10⁹ to 10¹⁵ vg, from 10⁹ to 10¹³ vg, or from 10⁹ to 10¹² vg. In some embodiments, the virus is comprised in the pharmaceutically acceptable implant at a total concentration of at least 10¹³ vg/cm³, such as at least 10¹⁴ vg/cm³.

In one embodiment, the biologic is an adeno-associated virus (AAV) selected from a group consisting of AAV1, AAV2 such as AAV2.7m8, AAV3, AAV4, AAV5, AAV6, AAV7, AAV8, AAV9, AAV10, AAV11 and a mutant, hybrid or variant thereof and is comprised in the pharmaceutically acceptable implant at a total amount of at least 10⁹ vg. In certain embodiments, the AAV is comprised in the pharmaceutically acceptable implant at a total amount from 10⁹ to 10¹⁵ vg. In other preferred embodiments, the AAV is comprised in the pharmaceutically acceptable implant at a total amount from 10⁹ to 10¹⁵ vg, from 10⁹ to 10¹³ vg, or from 10⁹ to 10¹² vg. In some embodiments, the AAV is comprised in the pharmaceutically acceptable implant at a total concentration of at least 10¹³ vg/cm³, such as at least 10¹⁴ vg/cm³.

In one embodiment, the biologic is an adeno-associated virus (AAV) selected from a group consisting of AAV1, AAV2 such as AAV2.7m8, AAV3, AAV4, AAV5, AAV6, AAV7, AAV8, AAV9, AAV10, AAV11 and a mutant, hybrid or variant thereof and is comprised in the pharmaceutically acceptable implant at a total amount of at least 10¹⁰ vg. In certain embodiments, the AAV is comprised in the pharmaceutically acceptable implant at a total amount from 10¹⁰ to 10¹⁵ vg. In other preferred embodiments, the AAV is comprised in the pharmaceutically acceptable implant at a total amount from 10¹⁰ to 10¹⁵ vg, or from 10¹⁰ to 10¹⁴ vg, or from 10¹⁰ to 10¹³ vg, or from 10¹⁰ to 10¹² vg, or from 10¹⁰ to 10¹¹ vg. In some embodiments, the AAV is comprised in the pharmaceutically acceptable implant at a total concentration of at least 10¹³ vg/cm³, such as at least 10¹⁴ vg/cm³.

In some embodiments, the AAV is comprised in the pharmaceutically acceptable implant at a total amount from 10¹⁰ to 10¹⁵ vg. In some embodiments, the AAV is comprised in the pharmaceutically acceptable implant at a total amount from 10¹⁰ to 10¹⁴ vg. In some embodiments, the AAV is comprised in the pharmaceutically acceptable implant at a total amount from 10¹⁰ to 10¹³ vg. In some embodiments, the AAV is comprised in the pharmaceutically acceptable implant at a total amount from 10¹⁰ to 10¹² vg. In some embodiments, the AAV is comprised in the pharmaceutically acceptable implant at a total amount from 10¹⁰ to 10¹¹ vg.

In one embodiment, the biologic is a recombinant protein and is comprised in the pharmaceutically acceptable implant at a total amount of at least 10 μg. In certain embodiments, the recombinant protein is comprised in the pharmaceutically acceptable implant at a total amount from 10 to 3000 μg. In other preferred embodiments, the recombinant protein is comprised in the pharmaceutically acceptable implant at a total amount from 10 to 2,500 μg, or from 10 to 2000 μg.

In one embodiment, the biologic is an antibody and is comprised in the pharmaceutically acceptable implant at a total amount of at least 100 μg. In certain embodiments, the antibody is comprised in the pharmaceutically acceptable implant at a total amount from 100 to 3000 μg. In other preferred embodiments, the antibody is comprised in the pharmaceutically acceptable implant at a total amount from 300 to 3000 μg.

In one embodiment, the biologic is an anti-VEGF antibody such as ranibizumab and is comprised in the pharmaceutically acceptable implant at a total amount of at least 500 μg. In certain embodiments, ranibizumab is comprised in the pharmaceutically acceptable implant at a total amount from 500 to 1000 μg. In other preferred embodiments, ranibizumab is comprised in the pharmaceutically acceptable implant at a total amount from 300 to 1000 μg.

In one embodiment, the biologic is an anti-VEGF antibody such as bevacizumab and is comprised in the pharmaceutically acceptable implant at a total amount of at least 1,500 μg. In certain embodiments, bevacizumab is comprised in the pharmaceutically acceptable implant at a total amount from 1,500 to 3000 μg. In other preferred embodiments, bevacizumab is comprised in the pharmaceutically acceptable implant at a total amount from 1,500 to 2,000 μg, or 1,250 μg.

In one embodiment, the biologic is a fusion protein such as aflibercept and is comprised in the pharmaceutically acceptable implant at a total amount of at least 2000 μg. In certain embodiments, aflibercept is comprised in the pharmaceutically acceptable implant at a total amount from 2,000 to 3,000 μg. In other preferred embodiments, aflibercept is comprised in the pharmaceutically acceptable implant at a total amount of 2,000 μg.

### Dehydration Stabilizer

A dehydration stabilizer is an excipient and/or an additive that protects and stabilizes a biologic or (a non-biologic comprising a biologic) in dry form or in an essentially water-free environment. A dehydration stabilizer is an excipient and/or an additive that protects and stabilizes a biologic against damage. The term “protect the biologic against damage” means that the dehydration stabilizer prevents denaturation, aggregation or agglomeration of the biologic and thus, preserves its functional activity. Thus, a dehydration stabilizer according to the invention is an excipient and/or an additive that preserves the37tructuree and/or the functional activity of the biologic. Thus, a biologic is considered to have preserved its structure and/or functional activity if, and when measured by an appropriate analytical method, it retains about 90%, such as about 80%, 70%, 60%, or at least 50% of its functional activity as measured by an appropriate analytical method.

Such analytical methods are known in the art. For example, if a biologic is a virus comprising a heterologous nucleic acid coding for a marker protein such as a green fluorescence protein (GFP) and said virus has been subjected to steps of dehydration, a skilled artisan can assess the functional activity of the virus by assessing the infectivity commonly known as transduction efficiency of said virus. One such method includes mixing said virus with one or more dehydration stabilizers and then subjecting particles comprising a mixture of said dehydration stabilizer and the virus to one or more dehydration steps. Thereafter, said particles can be used to infect cells in vitro and the transduction efficiency of the virus can be assessed by way of expression of the marker protein. In this way, a skilled artisan can conclude that the dehydration stabilizer was able to preserve the functional activity of the biologic, in this case a virus by about 90%, such as about 80%, 70%, 60%, or at least 50%. Detailed methods of how a dehydration stabilizer is able to preserve the functional activity of a biologic are also described in the Examples.

In another example, if a biologic is an antibody, the method includes mixing said antibody with one or more dehydration stabilizers and then subjecting particles comprising a mixture of said dehydration stabilizer and the antibody to one or more dehydration steps. Thereafter, a skilled person can assess the functional activity of the antibody by various analytical methods known in the art. One such powerful method for quantitative and/or qualitative assessment of antibodies is Enzyme Linked ImmunoAssay (ELISA).

The term “dehydration stabilizer” is not to be construed as being limited to a certain step or process of dehydration such as the process of lyophilization. Instead, the inventors of the present invention have found that a dehydration stabilizer is able to protect the biologic against damage during any dehydration process. Such dehydration steps include but are not limited to lyophilization, spray drying, sterilization and exposure to an organic solvent such as when forming an organogel.

Furthermore, the term “dehydration stabilizer” is not to be construed as being limited to the process of dehydration. Instead, the inventors of the present invention have found that a dehydration stabilizer is able to protect the biologic against damage also after the dehydrations steps and/or process has been completed and the biologic remains in the dehydrated form for a period of time.

The total concentration of one or more dehydration stabilizers that is/are mixed with the biologic before the biologic is exposed to an organic solvent can be 5 mg/ml or more. Thus, the inventors have found that such lower total concentrations of one or more dehydration stabilizers can be used. The inventors have found that the total concentration of one or more dehydration stabilizers that is/are mixed with the biologic before exposing the biologic to one or more dehydration steps can be from 200 mg/mL to 5 mg/ml, 100 to 5 mg/mL, 55 mg/ml to 5 mg/mL, 85 mg/mL to 5 mg/mL, 30 mg/ml to 5 mg/mL or about 5 mg/mL. The inventors have found that these concentrations are sufficient to protect the biologic from damage. In this context, dehydration steps can be any one or combination of lyophilization, spray drying, sterilization and exposure to an organic solvent such as when forming an organogel.

The total concentration of one or more dehydration stabilizers that is/are mixed with the biologic before the biologic is exposed to an organic solvent can be 5 mg/ml or more. Thus, the inventors have found that lower total concentrations of one or more dehydration stabilizers can be used. Thus, the total concentration of one or more dehydration stabilizers that is/are mixed with the biologic before the biologic is exposed to an organic solvent can be from 200 to 5 mg/ml, from 100 mg/mL to 5 mg/ml, from 85 mg/ml to 5 mg/mL, 55 mg/mL to 5 mg/ml, 30 mg/ml to 5 mg/ml or about 5 mg/mL. This concentration is sufficient to protect the biologic from damage in a process where the biologic is directly exposed to an organic solvent. In this context, the biologic is substantially insoluble in the organic solvent. The organic solvent can be any organic solvent that is carbon based and is liquid at room temperature and pressure. Such organic solvents can be methylene chloride, dimethyl carbonate, acetone, acetonitrile, ethyl acetate, and tetrahydrofuran. In some embodiments, the organic solvent is a dimethyl carbonate. The term “substantially insoluble” generally refers to a solubility of 0.1 mg/ml or less, such as for example, 0.01 mg/ml or less, 0.001 mg/ml or less.

In the following, several classes of dehydration stabilizers are discussed. Each of these classes can be individually replaced by the term “dehydration stabilizer”. Thus, each of the following classes represent an embodiment of the invention.

### Lyoprotectant as a Dehydration Stabilizer

A dehydration stabilizer can be a lyoprotectant. A lyoprotectant is an excipient and/or an additive that protects and stabilizes a biologic against damage. The word “lyoprotectant” is not to be construed as being limited to the process of lyophilization. Instead, lyoprotectants should be construed in their broadest possible sense to encompass any excipient, e.g., that forms hydrogen bonds with the biologic, and other mechanisms such as alterations in reaction kinetics, and mobility inhibition, in order to protect the biologic against damage.

Thus, according to the invention, a lyoprotectant is an excipient and/or additive that protects or preserves the functional activity of the biologic either during the process in which the biologic is dehydrated such as, but not limited to, when converting the biologic into a dried particulate form or after said process has been completed and the biologic remains in the dried particulate form for a period of time. A skilled artisan would readily understand that any excipient that is able to form hydrogen bonds with the biologic would be able to protect the biologic against damage. Thus, in one embodiment of the invention, throughout the present disclosure, wherever a dehydration stabilizer is mentioned, it may be construed as a lyoprotectant as defined above. Thus, according to the invention, a dehydration stabilizer is a carbohydrate, a sugar alcohol or a combination thereof.

Carbohydrates are a preferred group of compounds that may be used as a lyoprotectant in the present invention. Carbohydrates are compounds with the general chemical formula Cx(H2O)y, made up of molecules of carbon ©, hydrogen (H), and oxygen (O). Carbohydrates may be naturally occurring or synthetic and may be selected from monosaccharides, disaccharides, oligosaccharides, and polysaccharides. Each of these sub-classes represent a separate embodiment of the invention. Thus, each of these subclasses of carbohydrates can be used as a dehydration stabilizer for the purpose of the invention.

Monosaccharides are simple sugars, the most common of which is glucose. In monosaccharides, the number of carbons usually ranges from three to seven. If the sugar has an aldehyde group (the functional group with the structure R-CHO), it is an aldose, and if it has a ketone group (the functional group with the structure RC(═O) R′), it is a ketose. Depending on the number of carbons in the sugar, they can be trioses (three carbons), pentoses (five carbons), and/or hexoses (six carbons). Galactose and fructose are other common monosaccharides. Glucose, galactose, and fructose are isomeric monosaccharides (hexoses), meaning they have the same chemical formula but have slightly different structures. Glucose and galactose are aldoses, and fructose is a ketose. Each of these examples of monosaccharides represent a separate embodiment of the invention. Thus, each of these monosaccharides can be used as a dehydration stabilizer for the purpose of the invention.

Disaccharides form when two monosaccharides undergo a dehydration reaction (or a condensation reaction or dehydration synthesis). During this process, one monosaccharide's hydroxyl group combines with another monosaccharide's hydrogen, releasing a water molecule and forming a covalent bond. This is called a glycosidic bond. Glycosidic bonds (or glycosidic linkages) can be alpha or beta type. An alpha bond is formed when the OH group on the carbon-1 of the first glucose is below the ring plane, and a beta bond is formed when the OH group on the carbon-1 is above the ring plane. The most common disaccharide is sucrose, or table sugar, which is composed of glucose and fructose monomers. Other common disaccharides include trehalose, or trehalose dihydrates. Each of these examples of disaccharides represent a separate embodiment of the invention. Thus, each of these disaccharides can be used as a dehydration stabilizer for the purpose of the invention.

Oligosaccharides are carbohydrates of from three to six units of simple sugars (monosaccharides). A large number of oligosaccharides can be prepared by partially breaking down more complex carbohydrates (polysaccharides). Some non-limiting examples of oligosaccharides are raffinose, gentianose, maltotriose, polyalditol and cyclodextrins.

A long chain of monosaccharides linked by glycosidic bonds is a polysaccharide. The chain may be branched or unbranched, and it may contain different types of monosaccharides. The molecular weight may be 100,000 Daltons or more depending on the number of joined monomers. Starch, glycogen, cellulose, and chitin are examples of polysaccharides. Dextran, chitosan, cellulose derivatives (CMC, HPC, etc.), glycosaminoglycans (HA), Ficoll/poly-sucrose. Each of these examples of polysaccharides represent a separate embodiment of the invention. Thus, each of these polysaccharides can be used as a dehydration stabilizer for the purpose of the invention.

Thus, according to the invention, a dehydration stabilizer is one or more lyoprotectant(s) that can be used alone or in combination with other dehydration stabilizers for the purpose of the present invention.

Thus, the dehydration stabilizer/lyoprotectant can be one or more carbohydrate(s). Thus, the dehydration stabilizer/lyoprotectant can be a sugar. When the dehydration stabilizer/lyoprotectant is a sugar, it can be selected from a group consisting of sucrose, trehalose, raffinose, stachyose, verbascose, hydrates thereof and a combination thereof. Preferably sucrose, trehalose, trehalose dihydrate or a combination thereof.

Thus, the dehydration stabilizer/lyoprotectant can be a sugar alcohol. When the dehydration stabilizer/lyoprotectant is a sugar alcohol, it can be selected from a group consisting of erythritol, glycerol, isomalt, lactitol, maltitol, mannitol, sorbitol, xylitol, and a combination thereof. In some embodiments, the dehydration stabilizer is mannitol.

Thus, the dehydration stabilizer/lyoprotectant can be one or more sugar(s) and one or more sugar alcohol(s). In some embodiments, the dehydration stabilizer/lyoprotectant can be sucrose and mannitol. In some embodiments, the dehydration stabilizer/lyoprotectant can be trehalose dihydrate and mannitol.

The total concentration of the lyoprotectant mixed with the biologic before the biologic is exposed to a dehydration step can be 5 mg/ml or more. Thus, the inventors have also found that lower total concentrations of lyoprotectant can be used. The inventors have found that the total concentration lyoprotectant mixed with the biologic before exposing the biologic to one or more dehydration steps can be from 200 mg/ml to 5 mg/mL, from 100 to 5 mg/mL, 85 mg/ml to 5 mg/ml, 30 mg/mL to 5 mg/ml or about 5 mg/mL. The inventors have found that these concentrations are sufficient to protect the biologic from damage. In this context, dehydration steps can be any one or combination of lyophilization, spray drying, sterilization and exposure to an organic solvent such as when forming an organogel. In embodiments where more than one dehydration stabilizer is used and the lyoprotectant is one of the dehydration stabilizers used, then the total concentration of the lyoprotectants can be lower than 5 mg/ml as long as the total concentration of the dehydration stabilizer mixed with the biologic before the biologic is exposed to dehydration steps is within the concentration ranges discussed above.

The total concentration of lyoprotectant mixed with the biologic before the biologic is exposed to an organic solvent can be 5 mg/ml or more. Thus, the inventors have found that lower total concentrations of lyoprotectant can be used. Thus, the total concentration of lyoprotectant mixed with the biologic before the biologic is exposed to an organic solvent can be from 200 to 5 mg/ml, from 85 mg/ml to 5 mg/ml 30 mg/ml to 5 mg/ml or about 5 mg/mL. This concentration is sufficient to protect the biologic from damage in a process where the biologic is directly exposed to an organic solvent. The organic solvent can be any organic solvent that is carbon based and is liquid at room temperature and pressure. Such organic solvents can be methylene chloride, dimethyl carbonate, acetone, acetonitrile, ethyl acetate, and tetrahydrofuran. In some embodiments, the organic solvent is a dimethyl carbonate. In this context, the biologic is substantially insoluble in the organic solvent. The term “substantially insoluble” generally refers to a solubility of 0.1 mg/ml or less, such as for example, 0.01 mg/ml or less, 0.001 mg/mL or less.

In embodiments where more than one dehydration stabilizer is used and the lyoprotectant is one of the dehydration stabilizers used, then the total concentration of the lyoprotectants can be lower than 5 mg/mL as long as the total concentration of the dehydration stabilizer mixed with the biologic before the biologic is exposed to an organic solvent is within the concentration ranges discussed above.

Thus, in one embodiment, a carbohydrate can be used as a dehydration stabilizer at a total concentration of at least 5 mg/mL. In some embodiments, a carbohydrate can be used as a dehydration stabilizer at a total concentration from 5 mg/ml to 200 mg/mL, 5 mg/mL to 85 mg/mL, or as low as 5 mg/ml to 20 mg/mL.

Thus, in one embodiment, a sugar alcohol can be used as a dehydration stabilizer at a total concentration of at least 5 mg/mL. In some embodiments, sugar alcohol can be used as a dehydration stabilizer at a total concentration from 5 mg/ml to 200 mg/ml, 5 mg/mL to 85 mg/mL, or as low as 5 mg/ml to 20 mg/mL.

Thus, in one embodiment, a carbohydrate and sugar alcohol can be used in combination as dehydration stabilizers at a total concentration of at least 5 mg/ml. In some embodiments, a carbohydrate and sugar alcohol can be used in combination as dehydration stabilizers at a total concentration from 5 mg/ml to 200 mg/mL, 5 mg/mL to 85 mg/mL, or as low as 5 mg/ml to 20 mg/mL.

Thus, in one embodiment, a sugar can be used in combination as dehydration stabilizers at a total concentration of at least 5 mg/mL. In some embodiments, a sugar can be used as a dehydration stabilizer at a total concentration from 5 mg/ml to 200 mg/mL, 5 mg/mL to 85 mg/mL, or as low as 5 mg/ml to 20 mg/mL.

Thus, in one embodiment, sucrose can be used as a dehydration stabilizer at a total concentration of at least 5 mg/mL. In some embodiments, sucrose can be used as a dehydration stabilizer at a total concentration from 5 mg/ml to 200 mg/ml, 5 mg/ml to 85 mg/mL, or as low as 5 mg/ml to 20 mg/mL.

Thus, in one embodiment, trehalose dihydrate can be used as a dehydration stabilizer at a total concentration of at least 5 mg/mL. In some embodiments, trehalose dihydrate can be used as a dehydration stabilizer at a total concentration from 5 mg/mL to 200 mg/mL, 5 mg/mL to 85 mg/mL, or as low as 5 mg/ml to 20 mg/mL.

Thus, in one embodiment, a combination of trehalose dihydrate and sucrose can be used as dehydration stabilizers at a total concentration of at least 5 mg/ml. In some embodiments, a combination of trehalose dihydrate and sucrose can be used as dehydration stabilizers at a total concentration from 5 mg/mL to 200 mg/mL, 5 mg/mL to 85 mg/mL, or as low as 5 mg/ml to 20 mg/mL.

Thus, in one embodiment, a combination of trehalose dihydrate and mannitol or sucrose and mannitol can be used as dehydration stabilizers at a total concentration of at least 5 mg/mL. In some embodiments, a combination of trehalose dihydrate and mannitol or sucrose and mannitol can be used as dehydration stabilizers at a total concentration of from 5 mg/ml to 200 mg/mL, 5 mg/mL to 85 mg/mL, or as low as 5 mg/mL to 20 mg/mL.

### Synthetic Polymer as a Dehydration Stabilizer

Synthetic polymers can also be used as a dehydration stabilizer according to the invention.

One or more synthetic polymers of the group comprising one or more units of polyalkylene glycol, such as polyethylene glycol (PEG), polypropylene glycol, poly(ethylene glycol)-block-poly(propylene glycol) copolymers, or polyethylene oxide, polypropylene oxide, polyvinyl alcohol, poly(vinylpyrrolidinone), polylactic acid, polylactic-co-glycolic acid, random or block copolymers or combinations/mixtures of any of these can be used, while this list is not intended to be limiting.

The polymers used as dehydration stabilizers can be branched (multi-arm) or linear. In the case of a branched polymer, a core refers to a contiguous portion of a molecule joined to arms that extend from the core, with the arms having a functional group, which is often at the terminus of the branch. These polymers may have, e.g., 2-100 arms, with each arm having a terminus, bearing in mind that some precursors may be dendrimers or other highly branched materials. An arm refers to a linear chain of chemical groups that connect a cross linkable functional group to a polymer core. In some embodiments polymers that can be used as dehydration stabilizers can comprise between 3 and 300 arms; artisans will immediately appreciate that all the ranges and values within the explicitly stated ranges are contemplated, e.g., 4, 6, 8, 10, 12, 4 to 16, 8 to 100, 6, 8, 10, 12, or at least 4 arms.

The polymers used as dehydration stabilizers may or may not be “functional polymers”. “Functional polymers” and “non-functional polymers” have been defined earlier in the present disclosure. The functional groups generally have reactive groups for polymerization or react with each other in electrophile-nucleophile reactions or are configured to participate in other polymerization reactions. In some embodiments, the polymers used as dehydration stabilizers comprise a nucleophile or an electrophile.

Thus, according to the invention, a dehydration stabilizer is one or more polymer(s) that can be used alone or in combination with other dehydration stabilizers for the purpose of the present invention. In preferred embodiments, the polymer is a poly(ethylene) oxide commonly known as polyethylene glycol.

In certain embodiments, if the dehydration stabilizer is a polyethylene glycol, it may have an average molecular weight in the range from about 2,000 to about 100,000 Daltons, or in a range from about 10,000 to about 60,000 Daltons, or in a range from about 15,000 to about 50,000 Daltons. In certain particular embodiments, it may have an average molecular weight in a range from about 10,000 to about 40,000 Daltons, or of about 20,000 Daltons. The average molecular weight is given as the number average molecular weight (Mn), which, in certain embodiments, may be determined by MALDI.

The total concentration of the polymer mixed with the biologic before the biologic is exposed to a dehydration step can be 5 mg/ml or more. Thus, the inventors have also found that lower total concentrations of the polymer can be used. The inventors have found that the total concentration of polymer mixed with the biologic before exposing the biologic to one or more dehydration steps can be from 200 mg/ml to 5 mg/mL, from 100 to 5 mg/mL, 55 mg/ml to 5 mg/ml, 30 mg/ml to 5 mg/ml or about 5 mg/ml. The inventors have found that these concentrations are sufficient to protect the biologic from damage. In this context, dehydration steps can be any one or combination of lyophilization, spray drying, sterilization and exposure to an organic solvent such as when forming an organogel. In embodiments where more than one dehydration stabilizer is used and the polymer is one of the dehydration stabilizers used, then the total concentration of the polymer can be as low as 5-10 mg/mL as long as the total concentration of the dehydration stabilizer mixed with the biologic before the biologic is exposed to dehydration steps is within the concentration ranges discussed above.

The total concentration of polymer mixed with the biologic before the biologic is exposed to an organic solvent can be 5 mg/ml or more. Thus, the inventors have found that lower total concentrations of polymer can be used. Thus, the total concentration of polymer mixed with the biologic before the biologic is exposed to an organic solvent can be from 200 mg/ml to 5 mg/mL, from 100 to 5 mg/mL, 55 mg/ml to 5 mg/ml, 30 mg/ml to 5 mg/ml or about 5 mg/mL. This concentration is sufficient to protect the biologic from damage in a process where the biologic is directly exposed to an organic solvent. The organic solvent can be any organic solvent that is carbon based and is liquid at room temperature and pressure. Such organic solvents can be methylene chloride, dimethyl carbonate, acetone, acetonitrile, ethyl acetate, and tetrahydrofuran. In some embodiments, the organic solvent is a dimethyl carbonate. In this context, the biologic is substantially insoluble in the organic solvent. The term “substantially insoluble” generally refers to a solubility of 0.1 mg/ml or less, such as for example, 0.01 mg/ml or less, 0.001 mg/ml or less. In embodiments where more than one dehydration stabilizer is used and the lyoprotectant is one of the dehydration stabilizers used, then the total concentration of the lyoprotectants can be as lower than 5 mg/ml as long as the total concentration of the dehydration stabilizer mixed with the biologic before the biologic is exposed to an organic solvent is within the concentration ranges discussed above.

Thus, in one embodiment, a synthetic polymer can be used as a dehydration stabilizer at a total concentration of at least 5 mg/mL. In some embodiments, a synthetic polymer can be used as a dehydration stabilizer at a total concentration from 5 mg/ml to 200 mg/mL, 5 mg/ml to 55 mg/mL, 5 mg/ml to 85 mg/mL, or as low as 5 mg/ml to 20 mg/mL.

Thus, in one embodiment, a PEG polymer can be used as a dehydration stabilizer at a total concentration of at least 5 mg/mL. In some embodiments, a PEG polymer can be used as a dehydration stabilizer at a total concentration from 5 mg/ml to 200 mg/mL, 5 mg/ml to 55 mg/mL 5 mg/ml to 85 mg/mL, or as low as 5 mg/ml to 20 mg/mL.

Thus, in one embodiment, a multi-arm PEG polymer can be used as a dehydration stabilizer at a total concentration of at least 5 mg/mL. In some embodiments, a multiarm PEG polymer can be used as a dehydration stabilizer at a total concentration from 5 mg/ml to 200 mg/ml, 5 mg/ml to 55 mg/ml 5 mg/ml to 85 mg/ml, or as low as 5 mg/ml to 20 mg/mL.

Thus, in one embodiment, a multi-arm PEG polymer comprising a nucleophile, or an electrophile group can be used as a dehydration stabilizer at a total concentration of at least 5 mg/mL. In some embodiments, a multi-arm PEG polymer comprising a nucleophile, or an electrophile group can be used as a dehydration stabilizer at a total concentration from 5 mg/ml to 200 mg/ml, 5 mg/ml to 55 mg/mL 5 mg/ml to 85 mg/mL, or as low as 5 mg/ml to 20 mg/mL.

Thus, in one embodiment, a multi-arm PEG polymer comprising a nucleophile, or an electrophile group can be used as a dehydration stabilizer at a total concentration of at least 5 mg/mL. In some embodiments, a multi-arm PEG polymer comprising a nucleophile, or an electrophile group can be used as a dehydration stabilizer at a total concentration from 5 mg/ml to 200 mg/ml, 5 mg/ml to 55 mg/mL 5 mg/ml to 85 mg/mL, or as low as 5 mg/ml to 20 mg/mL. In some embodiments, the nucleophile is an amine such as a primary amine.

### Combination of Dehydration Stabilizers

A skilled artisan would understand that any of the above-described dehydration stabilizers can be used for any of the dehydration steps at any of the concentration ranges. Thus, for example, in one embodiment, a combination of a carbohydrate such as sucrose or trehalose dihydrate and PEG polymer such as a multi-arm PEG polymer comprising an amine may be used as dehydration stabilizers. In this embodiment, the total concentration of all dehydration stabilizers can be from 5 mg/ml to 200 mg/ml, 5 mg/ml to 55 mg/mL 5 mg/ml to 85 mg/mL, or as low as 5 mg/mL to 20 mg/mL.

### Other Stabilizers

According to the invention, other stabilizers may also be used in combination with the dehydration stabilizers discussed above and can be selected from buffers, salts, amino acids, surfactants, and antioxidants at known concentration ranges that are within the knowledge of a skilled artisan. In some embodiments, the at least one dehydration stabilizer can be used with at least one or at least two stabilizers. For examples, at least one dehydration stabilizer is used in combination with a buffer such as PBS, or in combination with a surfactant such as a non-ionic surfactant or both.

The term “non-ionic surfactant” means a surfactant that contains neither positively nor negatively charged functional groups. In contrast to anionic and cationic surfactants, non-ionic surfactants do not ionize in solution. The non-ionic surfactant can be a poloxamer. Poloxamers are non-ionic triblock copolymers composed of a central hydrophobic chain of poly(propyleneoxide) flanked by two hydrophilic chains of poly(ethylene oxide). The length of the polymer blocks can be customized, leading to different poloxamers with slightly different properties. Accordingly, the non-ionic surfactant can be Pluronic F127 (poloxamer 407), Pluronic F123 (poloxamer 403), Pluronic F-68 (poloxamer 188), Pluronic P123, Pluronic P85, or other polyethylene oxide-polypropylene oxide (EOPO) block copolymers of greater than 3,000-4,000 MW or combinations thereof.

Thus, in one embodiment, at least one dehydration stabilizer may be used at any given concentration as discussed above, with at least one or at least two stabilizers such as for example, a buffer comprising at least two or at least three salts, and a nonionic surfactant such as for example F-68, F-127 or F123.

### Particles in the Implant of the Invention

In some embodiments, particles, or total particles in the implant of the invention such as a pharmaceutically acceptable implant of the invention comprise a mixture of the biologic as discussed above and at least one dehydration stabilizer as discussed above.

In some embodiments, particles, or total particles in the implant of the invention such as a pharmaceutically acceptable implant of the invention comprise a mixture of the biologic, at least one dehydration stabilizer, and at least one other stabilizer such as a buffer and a surfactant.

In some embodiments, particles, or total particles in the implant of the invention such as a pharmaceutically acceptable implant of the invention comprise a mixture of the biologic, at least one dehydration stabilizer, and at least one other stabilizer such as a buffer such as PBS comprising at least two or at least three salts, and a non-ionic surfactant such as for example F-68, F-127 or F123.

In some embodiments, particles, or total particles in the implant of the invention such as a pharmaceutically acceptable implant of the invention consist of a mixture of the biologic, at least one dehydration stabilizer, and at least one other stabilizer such as a buffer and a surfactant.

In some embodiments, particles, or total particles in the implant of the invention such as a pharmaceutically acceptable implant of the invention consist of a mixture of the biologic, at least one dehydration stabilizer, and at least one other stabilizer such as a buffer such as PBS comprising at least two or at least three salts, and a non-ionic surfactant such as for example F-68, F-127 or F123.

In some embodiments, particle size such as Dv₉₀ particle size refers to particles in the implant of the invention such as a pharmaceutically acceptable implant of the invention comprising a mixture of the biologic as discussed above and at least one dehydration stabilizer as discussed above.

In some embodiments, particle size such as Dv₉₀ particle size refers to particles in the implant of the invention such as a pharmaceutically acceptable implant of the invention comprising a mixture of the biologic, at least one dehydration stabilizer, and at least one other stabilizer such as a buffer and a surfactant.

In some embodiments, particle size such as Dv₉₀ particle size refers to particles in the implant of the invention such as a pharmaceutically acceptable implant of the invention comprising a mixture of the biologic, at least one dehydration stabilizer, and at least one other stabilizer such as a buffer such as PBS comprising at least two or at least three salts, and a non-ionic surfactant such as for example F-68, F-127 or F123.

In some embodiments, particle size such as Dv₉₀ particle size refers to particles in the implant of the invention such as a pharmaceutically acceptable implant of the invention consisting of a mixture of the biologic, at least one dehydration stabilizer, and at least one other stabilizer such as a buffer and a surfactant.

In some embodiments, particle size such as Dv₉₀ particle size refers to particles in the implant of the invention such as a pharmaceutically acceptable implant of the invention consisting of a mixture of the biologic, at least one dehydration stabilizer, and at least one other stabilizer such as a buffer such as PBS comprising at least two or at least three salts, and a non-ionic surfactant such as for example F-68, F-127 or F123.

### Method of Protecting a Biologic in a Process where the Biologic is Directly Exposed to an Organic Solvent

The invention also provides a method for protecting a biologic against damage during a process wherein the biologic is directly exposed to an organic solvent. The method comprises mixing the biologic with at least one dehydration stabilizer before directly exposing the biologic to an organic solvent. The at least one dehydration stabilizer is described in detail the previous section.

In one embodiment, the dehydration stabilizer and the biologic are substantially insoluble in the organic solvent such as, a solubility of 0.1 mg/ml or less such as for example, 0.01 mg/ml or less, 0.001 mg/ml or less.

In some embodiments, before the biologic is exposed to an organic solvent, the biologic is mixed with at least one dehydration stabilizer that is a carbohydrate, a sugar alcohol, or a combination thereof. In another preferred embodiment, the at least one dehydration stabilizer is selected from a sugar, a sugar alcohol, or a combination thereof. Such sugars can be sucrose, trehalose, raffinose, stachyose, verbascose, hydrates thereof and a combination thereof, such as sucrose, trehalose, trehalose dihydrate and a combination thereof. Such sugar alcohols can be erythritol, glycerol, isomalt, lactitol, maltitol, mannitol, sorbitol, xylitol, and a combination thereof. In this embodiment, the mixture may comprise further stabilizers such as for example a buffer and a non-ionic surfactant.

In another embodiment, before the biologic is exposed to an organic solvent, the biologic is mixed with at least one dehydration stabilizer that is a synthetic polymer. For this purpose, any synthetic polymer may be used as defined in the previous section. Such synthetic polymers may be polyalkylene oxide such as polyethylene glycol, polyvinyl pyrrolidinone, and polyvinyl alcohol. In this embodiment, the mixture may comprise further stabilizers such as for example a buffer and a non-ionic surfactant.

In another embodiment, before the biologic is exposed to an organic solvent, the biologic is mixed with at least two dehydration stabilizers selected from a carbohydrate and a synthetic polymer as discussed above and in the previous section. In this embodiment, the mixture may comprise further stabilizers such as for example a buffer and a non-ionic surfactant.

In such embodiments, the biologic is a recombinant protein, a lipid encapsulating a nucleic acid or a virus comprising at least one heterologous nucleic acid sequence. In some embodiments, the virus is selected from a group consisting of AAV1, AAV2 such as AAV2.7m8, AAV3, AAV4, AAV5, AAV6, AAV7, AAV8, AAV9, AAV10, AAV11 and a mutant, hybrid or variant thereof. In these embodiments, the AAV comprises at least one heterologous nucleic acid sequence. In these embodiments, the heterologous nucleic acid sequence can be either a coding nucleic acid sequence or non-coding nucleic acid sequence. When the heterologous nucleic acid sequence is a coding nucleic acid sequence, it may code for a therapeutic protein as described in the section under the heading of biologic.

In another preferred embodiment, a recombinant protein is selected from a group consisting of an antibody, an antigen binding fragment, a fusion protein, a cytokine, a transcription factor, an enzyme or a hormone.

In one embodiment, the biologic is a virus and the total amount of the virus that is mixed with at least one dehydration stabilizer is at least 10⁹ vg. In certain embodiments, the total amount of the virus that is mixed with at least one dehydration stabilizer is from 10⁹ to 10¹⁵ vg. In other preferred embodiments, the total amount of the virus that is mixed with at least one dehydration stabilizer is from 10⁹ to 10¹⁵ vg, from 10⁹ to 10¹³ vg, or from 10⁹ to 10¹² vg.

In one embodiment, the biologic that is protected from damage is an adenoassociated virus (AAV) selected from a group consisting of AAV1, AAV2 such as AAV2.7m8, AAV3, AAV4, AAV5, AAV6, AAV7, AAV8, AAV9, AAV10, AAV11 and a mutant, hybrid or variant thereof. In one embodiment, the total amount of the AAV that is mixed with at least one dehydration stabilizer is at least 10⁹ vg. In certain embodiments, the total amount of the AAV that is mixed with at least one dehydration stabilizer is from 10⁹ to 10¹⁵ vg. In other preferred embodiments the total amount of the AAV that is mixed with at least one dehydration stabilizer is from 10⁹ to 10¹⁵ vg, from 10⁹ to 10¹³ vg, or from 10⁹ to 10¹² vg.

In one embodiment, the biologic that is protected from damage is an adenoassociated virus (AAV) selected from a group consisting of AAV1, AAV2 such as AAV2.7m8, AAV3, AAV4, AAV5, AAV6, AAV7, AAV8, AAV9, AAV10, AAV11 and a mutant, hybrid or variant thereof. In one embodiment, the total amount of the AAV that is mixed with at least one dehydration stabilizer is at least 10⁹ vg. In certain embodiments, the total amount of the AAV that is mixed with at least one dehydration stabilizer is from 10¹⁰ to 10¹⁵ vg. In other preferred embodiments the total amount of the AAV that is mixed with at least one dehydration stabilizer is from 10¹⁰ to 10¹⁵ vg, or from 10¹⁰ to 10¹⁴ vg, or from 10¹⁰ to 10¹³ vg, or from 10¹⁰ to 10¹² vg, or from 10¹⁰ to 10¹¹ vg.

In one embodiment, the biologic is a recombinant protein and the total amount of the recombinant protein that is mixed with at least one dehydration stabilizer is at least at least 10 μg. In certain embodiments, the total amount of the recombinant protein that is mixed with at least one dehydration stabilizer is from 10 to 3000 μg. In other preferred embodiments, the total amount of the recombinant protein that is mixed with at least one dehydration stabilizer is from 10 to 2,500 μg, or from 10 to 2000 μg.

In one embodiment, the invention as disclosed herein does not concern adsorbing the biologic to silica particles such as mesoporous silica particles or equivalent thereof. This means that the biologic is not protected by mesoporous silica particles at any time point during the method of manufacturing the implant such as a pharmaceutically acceptable implant of the present invention.

In one embodiment, the invention as disclosed herein does not concern adsorbing the biologic to a fatty acid component or equivalent thereof. This means that the biologic is not protected by fatty acid components at any time point during the method of manufacturing the implant such as a pharmaceutically acceptable implant of the present invention.

In one embodiment, the invention as disclosed herein does not concern adsorbing the AAV to silica particles such as mesoporous silica particles or equivalent thereof. This means that the AAV is not protected by mesoporous silica particles at any time point during the method of manufacturing the implant such as a pharmaceutically acceptable implant of the present invention.

In one embodiment, the invention as disclosed herein does not concern adsorbing the AAV to a fatty acid component or equivalent thereof. This means that the AAV is not protected by fatty acid components at any time point during the method of manufacturing the implant such as a pharmaceutically acceptable implant of the present invention.

### Method of Manufacturing a Pharmaceutically Acceptable Implant

According to the invention, a method of manufacturing a pharmaceutically acceptable implant comprising a biologic is provided. The method for manufacturing a pharmaceutically acceptable implant comprising a biologic comprises forming an organogel including the biologic comprising forming a matrix comprising at least two multi-arm precursors that are covalently crosslinked in an organic solvent in the presence of the biologic followed by forming a xerogel comprising removing the organic solvent.

### (a) Providing a Mixture of a Biologic and at Least One Dehydration Stabilizer

The method of manufacturing a pharmaceutically acceptable implant requires first providing a mixture of the biologic and at least one dehydration stabilizer.

In some embodiments, the biologic is a recombinant protein, a lipid encapsulating a nucleic acid, or a virus comprising a heterologous nucleic acid sequence. In some embodiments, the virus is selected from a group consisting of AAV1, AAV2 such as AAV2.7m8, AAV3, AAV4, AAV5, AAV6, AAV7, AAV8, AAV9, AAV10, AAV11 and a mutant, hybrid or variant thereof. In these embodiments, the AAV comprises at least one heterologous nucleic acid sequence. In these embodiments, the heterologous nucleic acid sequence can be either a coding nucleic acid sequence or non-coding nucleic acid sequence. When the heterologous nucleic acid sequence is a coding nucleic acid sequence, it may code for a therapeutic protein as described in the section under the heading of biologic. In certain embodiments, the AAV may be at a total concentration of at least 10¹³ vg/cm³ in the pharmaceutically acceptable implant. In some embodiments, the AAV is at a concentration of at least 10¹⁴ vg/cm³ in the pharmaceutically acceptable implant. In one embodiment, the total amount of the AAV is in the order from 10⁹ to 10¹⁵ vg. In some embodiments, the total amount of AAV is in the order from 10¹⁰ to 10¹³ vg in the pharmaceutically acceptable implant. In some embodiments, the total amount of AAV is in the order from 10¹⁰ to 10¹⁵ vg, or from 10¹⁰ to 10¹⁴ vg, or from 10¹⁰ to 10¹³ vg, or from 10¹⁰ to 10¹² vg, or from 10¹⁰ to 10¹¹ vg in the pharmaceutically acceptable implant

In another preferred embodiment, the recombinant protein is selected from a group consisting of an antibody, an antigen binding fragment, a fusion protein, a cytokine, a transcription factor, an enzyme or a hormone. In one embodiment, the total amount of the recombinant protein in the pharmaceutically acceptable implant is at least at least 10 μg. In certain embodiments, the total amount of the recombinant protein in the pharmaceutically acceptable implant is from 10 to 3000 μg. In other preferred embodiments, the total amount of the recombinant protein in the pharmaceutically acceptable implant is from 5 to 2,500 μg, or from 5 to 2000 μg.

In some embodiments, the at least dehydration stabilizer is a carbohydrate, a sugar alcohol, and a combination thereof. In another embodiment, the at least one dehydration stabilizer is a synthetic polymer. In another embodiment, at least one dehydration stabilizer are two dehydration stabilizers selected from a carbohydrate and a synthetic polymer. The dehydration stabilizer may be selected from a sugar, a sugar alcohol, or a combination thereof. Such sugars can be sucrose, trehalose, raffinose, stachyose, verbascose, hydrates thereof and a combination thereof, such as sucrose, trehalose, trehalose dihydrate and a combination thereof. Such sugar alcohols can be erythritol, glycerol, isomalt, lactitol, maltitol, mannitol, sorbitol, xylitol, and a combination thereof. When the dehydration stabilizer is a synthetic polymer, it may be polyalkylene oxide such as polyethylene glycol, polyvinyl pyrrolidinone, and polyvinyl alcohol. The total concentration of the dehydration stabilizer that is mixed with the total amount of the biologic is at least 5 mg/ml such as 5 mg/ml to 200 mg/mL, 5 mg/ml to 100 mg/mL, 5 mg/ml to 85 mg/mL, 5 mg/ml to 55 mg/mL, 5 mg/mL to 30 mg/mL, or 5 mg/mL to 15 mg/mL.

In this step, in some embodiments, the mixture may comprise other stabilizers such as for example a buffer and a non-ionic surfactant.

### (b) Providing at Least One Multi-Arm Precursor

In one embodiment, at least one multi-arm precursor is provided. Precursors and multi-arm precursors used in the present invention have been described in detail in the section under the heading “xerogel”. In some embodiments, the at least one multiarm precursor comprises at least 8 arms, or at least 4 arms. The at least one multi-arm precursor comprises an electrophile or a nucleophile.

In another embodiment, the at least one multi-arm precursor comprises at least two multi-arm precursors. In such embodiments, one multi-arm precursor comprises an electrophile and another multi-arm precursor comprises a nucleophile. In another embodiment, the at least one multi-arm precursor comprises at least two multi-arm precursors each comprising an electrophile.

In these embodiments, the nucleophile can be an amine such as a primary amine, a thiol, an azide or a hydrazide, and the electrophile can be succinimidyl esters, succinimidyl carbonates, nitrophenyl carbonates, aldehydes, ketones, acrylates, acrylamides, maleimides, vinylsulfones, iodoacetamides, alkenes, alkynes, dibenzocyclooctynes, norbornenes, epoxides, mesylates, tosylates, tresyls, cyanurates, orthopyridyl disulfides, or halides. In an embodiment of the invention, if the electrophile is a succinimidyl ester, it may may comprise a reactive group such as succinimidyl succinate (SS), succinimidyl glutarate (SG), succinimidyl adipate (SAP), succinimidyl azelate (SAZ), or succinimidyl glutaramide.

In some embodiments, the at least one multi-arm precursor is a first multi-arm PEG precursors comprising a primary amine.

### (c) Providing at Least One Further Multi-Arm Precursor

In one embodiment, at least one further multi-arm precursor is provided. Precursors and multi-arm precursors used in the present invention have been described in detail in the section under the heading “xerogel”. In some embodiments, at least one further multi-arm precursor comprises at least 8 arms, or at least 4 arms. The at least one further multi-arm precursor may comprise an electrophile or a nucleophile.

In another embodiment, the at least one further multi-arm precursor comprises at least two multi-arm precursors. In such embodiments, one multi-arm precursor comprises an electrophile and another multi-arm precursor comprises a nucleophile. In another embodiment, the at least one further multi-arm precursor comprises at least two multi-arm precursors each comprising an electrophile.

In these embodiments, the nucleophile can be an amine such as a primary amine, a thiol, an azide or a hydrazide, and the electrophiles can be succinimidyl esters, succinimidyl carbonates, nitrophenyl carbonates, aldehydes, ketones, acrylates, acrylamides, maleimides, vinylsulfones, iodoacetamides, alkenes, alkynes, dibenzocyclooctynes, norbornenes, epoxides, mesylates, tosylates, tresyls, cyanurates, orthopyridyl disulfides, or halides.

In some embodiments, the at least one further multi-arm precursor comprises at least two further multi-arm precursors comprising a first multi-arm precursor comprising an electrophile comprising a first reactive group and a second multi-arm precursor comprising an electrophile comprising a second reactive group. In an embodiment of the invention, if the electrophile is a succinimidyl ester, the first and the second reactive groups are selected from succinimidyl succinate (SS), succinimidyl glutarate (SG), succinimidyl adipate (SAP), succinimidyl azelate (SAZ), or succinimidyl glutaramide.

### Processing Steps

Each of (a) (b) and (c) above are then processed to obtain (d), (c) and (f).

### (d) Particles Comprising a Mixture of a Biologic and at Least One Dehydration Stabilizer

Processing of (a) to obtain (d) may comprise one or more dehydration steps comprising forming dried particulates of the mixture of a biologic and at least one dehydration stabilizer. Such methods are known in the art and include but are not limited to lyophilization, spray drying, or vacuum drying. In one embodiment, the mixture of the biologic and at least one dehydration stabilizer is converted into dried particulate form. According to the invention, the dehydration stabilizer protects the biologic from damage during the one or more dehydration steps.

(c) and (f)—Processed (b) and (c)

In one embodiment, processing of (b) and (c) may or may not comprise one or more dehydration steps such as lyophilization, spray drying or vacuum drying to convert the multi-arm precursors into dried particulate form. In one embodiment, when these dehydration steps are not employed, a step of sterilization such as by gamma sterilization, e-beam sterilization, or ethylene oxide sterilization may be employed.

(g) and (h)—Adding and Organic Solvent to (c) and (f)

In one embodiment, an organic solvent is added to each of @ and (f) to obtain (g) and (h). In this embodiment, the organic solvent can be any organic solvent that is carbon based and is liquid at room temperature and pressure. Such organic solvents can be methylene chloride, dimethyl carbonate, acetone, acetonitrile, ethyl acetate, and tetrahydrofuran. In some embodiments, the organic solvent is a dimethyl carbonate.

### Forming an Organogel

In one embodiment, (g) is mixed with (d) to obtain (i) and then mixed with (h). In another embodiment, (h) is mixed with (d) to obtain (i) and then mixed with (g). At this step, the biologic is directly exposed to an organic solvent. According to the invention, the biologic is in the form of particles comprising a mixture of the biologic and at least one dehydration stabilizer, and thus, the biologic from damage by the at least one dehydration stabilizer. At this step, the organic solvent and the dehydration stabilizer are substantially insoluble in the organic solvent such as, a solubility of 0.1 mg/ml or less such as for example, 0.01 mg/ml or less, 0.001 mg/ml or less.

At this step, at least two multi-arm precursors react in an electrophile-nucleophile reaction to form a covalently cross-linked matrix that is an organogel. In some embodiments, at least three multi-arm precursors react in an electrophile-nucleophile reaction to form a covalently cross-linked matrix that is an organogel.

### Forming a Xerogel

According to the invention, forming a xerogel from an organogel comprises a step of drying. A skilled artisan understands that any known methods of drying can be used. Potential processes include, e.g., precipitation with non-solvent, nitrogen sweep drying, vacuum drying, freeze-drying, a combination of heat and vacuum, and lyophilization. In some embodiments, the organogel is dried in a nitrogen gas flow at a temperature of 35° C. to 37° C., and for 1-5 days, or at least 4 days, or at least 3 days. In some embodiments, before the step of drying, the organogel is casted in a tube with pre-determined dimensions to form it into a particular shape. In some embodiments, the pharmaceutically acceptable implant is in the form a fiber, and the fiber is characterized by a diameter of about 0.1 mm or more and/or a length of about 2.0 mm or more.

In one embodiment, (b) is part of (a) and thus, is a dehydration stabilizer for the biologic. In this embodiment (d) comprises particles comprising a mixture of the biologic and at least one dehydration stabilizer, said dehydration stabilizer being (b).

In one embodiment, (b) is part of (a) and thus, is a dehydration stabilizer for the biologic. In this embodiment (d) comprises particles comprising a mixture of the biologic and at least one dehydration stabilizer. The at least one dehydration stabilizer may be at least two dehydration stabilizers comprising (b) as the first dehydration stabilizer and another dehydration stabilizer such as a lyoprotectant, such as a carbohydrate such as sugar such as sucrose and trehalose, or a sugar alcohol such as mannitol.

In certain embodiments, (b) or (c) further comprises a polymer such as a synthetic polymer that is a “non-functional polymer”. A “non-functional” polymer as described previously in the present disclosure is a polymer that does not participate in the crosslinking reaction between the multi-arm precursors.

This polymer can be further defined as any polymer that is soluble in both organic solvent and water. In one embodiment, the further polymer is a non-functional polymer used as a bulking agent in the pharmaceutically acceptable implant. The MW of this polymer can be from 1,000 to 35,000 Da, for example from 5,000 to 35,000 Da such as from 5,000 to 10,000 Da, from 7,000 to 10,000 Da, from 8,000 to 15,000 Da, from 8,000 to 25,000 Da, or 5,000 Da or more.

The further polymer can be branched (multi-arm) or linear. In the case of a branched polymer, a core refers to a contiguous portion of a molecule joined to arms that extend from the core, with the arms having a functional group, which is often at the terminus of the branch. These polymers may have, e.g., 2-100 arms, with each arm having a terminus, bearing in mind that some precursors may be dendrimers or other highly branched materials. An arm refers to a linear chain of chemical groups that connect a cross linkable functional group to a polymer core. In some embodiments, these further polymers can comprise between 3 and 300 arms; artisans will immediately appreciate that all the ranges and values within the explicitly stated ranges are contemplated, e.g., 4, 6, 8, 10, 12, 4 to 16, 8 to 100, 6, 8, 10, 12, or at least 4 arms.

In various embodiments, this further polymer is selected from a group consisting of polyalkylene oxide such as polyethylene glycol, polyvinyl pyrrolidinone, and polyvinyl 59lcohol.

In one embodiment, the invention as disclosed herein does not concern adsorbing the biologic to silica particles such as mesoporous silica particles or equivalent thereof at any time point during the method of manufacturing the implant such as a pharmaceutically acceptable implant of the present invention.

In one embodiment, the invention as disclosed herein does not concern adsorbing the biologic to a fatty acid component or equivalent thereof at any time point during the method of manufacturing the implant such as a pharmaceutically acceptable implant of the present invention.

In one embodiment, the invention as disclosed herein does not concern adsorbing the AAV to silica particles such as mesoporous silica particles or equivalent thereof at any time point during the method of manufacturing the implant such as a pharmaceutically acceptable implant of the present invention.

In one embodiment, the invention as disclosed herein does not concern adsorbing the AAV to a fatty acid component or equivalent thereof at any time point during the method of manufacturing the implant such as a pharmaceutically acceptable implant of the present invention.

### Method of Manufacturing a Pharmaceutically Acceptable Implant for Controlled Release of a Biologic

According to the invention, a method of manufacturing a pharmaceutically acceptable implant for controlled release of the total amount of the biologic is provided. The method is essentially the same as the previous section. The additional considerations are necessary for steps (a), (b) and (c) in the previous section.

Throughout this section (w/w) % is based on the weight of the pharmaceutically acceptable implant.

Throughout this section, in all of the embodiments including all of the parameters disclosed in this section, “particles” or “total particles” or “Dv₉₀ particle size” is each described as pertaining to particles comprising a mixture of the biologic and at least one dehydration stabilizer such as a carbohydrate, sugar alcohol or combination thereof.

Throughout this section, in all of the embodiments including all of the parameters disclosed in this section, “particles”, or “total particles” or “Dv₉₀ particle size” can also refer to a mixture according to the following paragraphs.

In some embodiments, particles, or total particles in the implant of the invention such as a pharmaceutically acceptable implant of the invention comprise a mixture of the biologic as discussed above and at least one dehydration stabilizer as discussed above.

In some embodiments, particles, or total particles in the implant of the invention such as a pharmaceutically acceptable implant of the invention comprise a mixture of the biologic, at least one dehydration stabilizer, and at least one other stabilizer such as a buffer and a surfactant.

In some embodiments, particles, or total particles in the implant of the invention such as a pharmaceutically acceptable implant of the invention comprise a mixture of the biologic, at least one dehydration stabilizer, and at least one other stabilizer such as a buffer such as PBS comprising at least two or at least three salts, and a non-ionic surfactant such as for example F-68, F-127 or F123.

In some embodiments, particles, or total particles in the implant of the invention such as a pharmaceutically acceptable implant of the invention consist of a mixture of the biologic, at least one dehydration stabilizer, and at least one other stabilizer such as a buffer and a surfactant.

In some embodiments, particles, or total particles in the implant of the invention such as a pharmaceutically acceptable implant of the invention consist of a mixture of the biologic, at least one dehydration stabilizer, and at least one other stabilizer such as a buffer such as PBS comprising at least two or at least three salts, and a non-ionic surfactant such as for example F-68, F-127 or F123.

In some embodiments, particle size such as Dv₉₀ particle size refers to particles in the implant of the invention such as a pharmaceutically acceptable implant of the invention comprising a mixture of the biologic as discussed above and at least one dehydration stabilizer as discussed above.

In some embodiments, particle size such as Dv₉₀ particle size refers to particles in the implant of the invention such as a pharmaceutically acceptable implant of the invention comprising a mixture of the biologic, at least one dehydration stabilizer, and at least one other stabilizer such as a buffer and a surfactant.

In some embodiments, particle size such as Dv₉₀ particle size refers to particles in the implant of the invention such as a pharmaceutically acceptable implant of the invention comprising a mixture of the biologic, at least one dehydration stabilizer, and at least one other stabilizer such as a buffer such as PBS comprising at least two or at least three salts, and a non-ionic surfactant such as for example F-68, F-127 or F123.

In some embodiments, particle size such as Dv₉₀ particle size refers to particles in the implant of the invention such as a pharmaceutically acceptable implant of the invention consisting of a mixture of the biologic, at least one dehydration stabilizer, and at least one other stabilizer such as a buffer and a surfactant.

In some embodiments, particle size such as Dv₉₀ particle size refers to particles in the implant of the invention such as a pharmaceutically acceptable implant of the invention consisting of a mixture of the biologic, at least one dehydration stabilizer, and at least one other stabilizer such as a buffer such as PBS comprising at least two or at least three salts, and a non-ionic surfactant such as for example F-68, F-127 or F123.

Some aspects of the present disclosure are directed to a method for manufacturing a pharmaceutically acceptable implant for controlled release of a biologic comprises forming a xerogel comprising at least two covalently cross-linked precursors within which particles comprising the biologic and at least one dehydration stabilizer are dispersed. In one embodiment, the at least one dehydration stabilizer is selected from a carbohydrate such as a sugar, such as sucrose and trehalose, a sugar alcohol such as mannitol, or a combination thereof. The inventors have found that the total (w/w) % of said dehydration stabilizer comprised in the pharmaceutically acceptable implant can be selected to be no greater than 60%, no greater than 55%, no greater than 50%, no greater than 45%, or no greater than 40%, or no greater than 30% to provide for the controlled release of the biologic. In a related or separate embodiment, the molecular weight between crosslinks in the xerogel can be selected from 7 to 25 kDa, 9 to 20 kDa, or 10 to 15 kDa to provide for the controlled release of the biologic. In another related or separate embodiment, the (w/w) % of the total particles comprising the mixture of the biologic and said dehydration stabilizer can be selected to be no greater than 80%, or no greater than 70%, or no greater than 60%, or no greater than no greater than 50% such as from 20% to 40%, from 20% to 30% to provide for the controlled release of the biologic. In another related or separate embodiment, the (w/w) % of the total number of multi-arm precursors can be selected from 20% to 80% such as from 35% to 75%, such as from 35% to 65%, from 35% to 55%, from 35% to 45% to provide for the controlled release of the biologic. In another related or separate embodiment, the ratio of (1) the (w/w) % of the total particles comprising a mixture of said dehydration stabilizer and (2) the (w/w) % of the total number of multi-arm precursors can be selected from 0.3 to 4.0, such as 0.3 to 3.5, from 0.3 to 3.0, from 0.3 to 2.5, from 0.3 to 2.0, from 0.3 to 1.5, from 0.3 to 1.0 or from 0.5 to 4.0, such as 0.5 to 3.5, from 0.5 to 3.0, from 0.5 to 2.5, from 0.5 to 2.0, from 0.5 to 1.5, from 0.5 to 1.0, or from 0.6 to 4.0, 0.6 to 3.5, from 0.6 to 3.0, from 0.6 to 2.5, from 0.6 to 2.0, from 0.6 to 1.5, or from 0.6 to 1.0. to provide for the controlled release of the biologic. In another related or separate embodiment, the Dv₉₀ particle size can be selected to be from 10 μm to 200 μm such as 35 μm to 75 μm such as 35 μm to 100 μm, or 35 μm to 150 μm to provide for the controlled release of the biologic, wherein particles comprise the mixture of the biologic and said dehydration stabilizer such as a carbohydrate, a sugar alcohol, or a combination thereof. Each of these individual parameters can be selected alone or in combination with each other to provide for the controlled release of the biologic.

A method of providing a pharmaceutically acceptable implant for controlled release of a biologic comprises forming a xerogel comprising at least three multi-arm precursors comprising a first multi-arm precursor comprising a nucleophile, a second multi-arm precursor comprising an electrophile comprising a first reactive group and a third multi-arm precursor comprising an electrophile comprising a second reactive group within which particles comprising the biologic and at least one dehydration stabilizer are dispersed. In one embodiment, the hydrolysis half-life of the third multi-arm precursor is longer than the second precursor. The inventors have found that the molar ratio of the the first reactive group comprised in the second multi-arm precursor, and the second reactive group comprised in the third multi-arm precursor can be selected from 30-90:70-10, 40-80:60-20, 50-70:50-30, or 40-60:60-40 to provide for the controlled release of the biologic. In some embodiments, the nucleophile is a primary amine, and the electrophiles are succinimidyl esters In some embodiments, the first reactive group is succinimidyl succinate and the second reactive group is succinimidyl glutarate. In one embodiment, the at least one dehydration stabilizer is selected from a carbohydrate such as a sugar, such as sucrose and trehalose, a sugar alcohol such as mannitol, or a combination thereof. The inventors have found that the total (w/w) % of said dehydration stabilizer comprised in the pharmaceutically acceptable implant can be selected to be no greater than 60%, no greater than 55%, no greater than 50%, no greater than 45%, or no greater than 40%, or no greater than 30% to provide for the controlled release of the biologic. In a related or separate embodiment, the molecular weight between crosslinks in the xerogel can be selected from 7 to 25 kDa, 9 to 20 kDa, or 10 to 15 kDa to provide for the controlled release of the biologic. In another related or separate embodiment, the (w/w) % of the total particles comprising the mixture of the biologic and said dehydration stabilizer can be selected to be no greater than no greater than 50% such as from 20% to 40%, from 20% to 30% to provide for the controlled release of the biologic. In another related or separate embodiment, the (w/w) % of the total number of multi-arm precursors can be selected from 20% to 80% such as from 35% to 75%, such as from 35% to 65%, from 35% to 55%, from 35% to 45% to provide for the controlled release of the biologic. In another related or separate embodiment, the ratio of (1) the (w/w) % of the total particles comprising a mixture of said dehydration stabilizer and (2) the (w/w) % of the total number of multi-arm precursors can be selected from 0.3 to 4.0, such as 0.3 to 3.5, from 0.3 to 3.0, from 0.3 to 2.5, from 0.3 to 2.0, from 0.3 to 1.5, from 0.3 to 1.0 or from 0.5 to 4.0, such as 0.5 to 3.5, from 0.5 to 3.0, from 0.5 to 2.5, from 0.5 to 2.0, from 0.5 to 1.5, from 0.5 to 1.0, or from 0.6 to 4.0, 0.6 to 3.5, from 0.6 to 3.0, from 0.6 to 2.5, from 0.6 to 2.0, from 0.6 to 1.5, or from 0.6 to 1.0. to provide for the controlled release of the biologic. In another related or separate embodiment, the Dv₉₀ particle size can be selected to be from 10 μm to 200 μm such as 35 μm to 75 μm such as 35 μm to 100 μm, or 35 μm to 150 μm to provide for the controlled release of the biologic, wherein particles comprise the mixture of the biologic and said dehydration stabilizer such as a carbohydrate, a sugar alcohol, or a combination thereof. Each of these individual parameters can be selected alone or in combination with each other to provide for the controlled release of the biologic.

Some aspects of the present disclosure are directed to a method for manufacturing a pharmaceutically acceptable implant for controlled release of a biologic comprises forming an organogel comprising at least two covalently cross-linked precursors within which particles comprising the biologic and at least one dehydration stabilizer are dispersed. In one embodiment, organogel is formed by adding an organic solvent to each of the multi-arm precursors and mixing them together. In one embodiment, the total (w/v) % of multi-arm precursors dissolved in the organic solvent can be selected to provide for the controlled release of the biologic. In one embodiment, the at least one dehydration stabilizer is selected from a carbohydrate such as a sugar, such as sucrose and trehalose, a sugar alcohol such as mannitol, or a combination thereof. The inventors have found that the total (w/w) % of said dehydration stabilizer comprised in the pharmaceutically acceptable implant can be selected to be no greater than 60%, no greater than 55%, no greater than 50%, no greater than 45%, or no greater than 40%, or no greater than 30% to provide for the controlled release of the biologic. In a related or separate embodiment, the molecular weight between crosslinks in the xerogel can be selected from 7 to 25 kDa, 9 to 20 kDa, or 10 to 15 kDa to provide for the controlled release of the biologic. In another related or separate embodiment, the (w/w) % of the total particles comprising the mixture of the biologic and said dehydration stabilizer can be selected to be no greater than 80%, or no greater than 70%, or no greater than 60%, or no greater than no greater than 50% such as from 20% to 40%, from 20% to 30% to provide for the controlled release of the biologic. In another related or separate embodiment, the (w/w) % of the total number of multi-arm precursors can be selected from 20% to 80% such as from from 35% to 75%, such as from 35% to 65%, from 35% to 55%, from 35% to 45% to provide for the controlled release of the biologic. In another related or separate embodiment, the ratio of (1) the (w/w) % of the total particles comprising a mixture of said dehydration stabilizer and (2) the (w/w) % of the total number of multi-arm precursors can be selected from 0.3 to 4.0, such as 0.3 to 3.5, from 0.3 to 3.0, from 0.3 to 2.5, from 0.3 to 2.0, from 0.3 to 1.5, from 0.3 to 1.0 or from 0.5 to 4.0, such as 0.5 to 3.5, from 0.5 to 3.0, from 0.5 to 2.5, from 0.5 to 2.0, from 0.5 to 1.5, from 0.5 to 1.0, or from 0.6 to 4.0, 0.6 to 3.5, from 0.6 to 3.0, from 0.6 to 2.5, from 0.6 to 2.0, from 0.6 to 1.5, or from 0.6 to 1.0. to provide for the controlled release of the biologic. In one embodiment, the organogel and the xerogel comprise at least three multi-arm precursors comprising a first multi-arm precursor comprising a nucleophile, a second multi-arm precursor comprising an electrophile comprising a first reactive group and a third multi-arm precursor comprising an electrophile comprising a second reactive group. In one embodiment, the hydrolysis half-life of the third multi-arm precursor is longer than the second precursor. The inventors have found that the molar ratio of the first reactive group comprised in the second multi-arm precursor, and the second reactive group comprised in the third multi-arm precursor can be selected from 30-90:70-10, 40-80:60-20, 50-70:50-30, or 40-60:60-40 to provide for the controlled release of the biologic. In some embodiments, the nucleophile is a primary amine, and the electrophiles are succinimidyl esters. In some embodiments, the first reactive group is succinimidyl succinate and the second reactive group is succinimidyl glutarate. In another related or separate embodiment, the Dv₉₀ particle size can be selected to be from 10 μm to 200 μm such as 35 μm to 75 μm such as 35 μm to 100 μm, or 35 μm to 150 μm to provide for the controlled release of the biologic, wherein particles comprise the mixture of the biologic and said dehydration stabilizer such as a carbohydrate, a sugar alcohol, or a combination thereof. Each of these individual parameters can be selected alone or in combination with each other to provide for the controlled release of the biologic.

In one embodiment, the controlled release of the biologic is characterized by the amount of the biologic released on day 1 is from 0 to 50% of the total amount of the biologic, the amount of the biologic released per day from day 2 until the last day of release is from 0 to 50% of the total amount of the biologic, and/or the number of days required for 100% release of the total amount of the biologic is at least 2 days. In another embodiment, the amount of the biologic released on day 1 is from 0 to 25%, 0 to 20% 0 to 10%, 0 to 5%, or about 0% of the total amount of the biologic, the amount of the biologic released per day from day 2 until the last day of release is from 0 to 50% of the total amount of the biologic, and/or the number of days required for 100% release of the total amount of the biologic is at least 3 days but no greater than 30 days, 25 days, or no greater than 16 days.

In one embodiment, when the controlled release of the biologic is characterized by the number of days required for 100% release of the biologic such that the number of days are at least 2 days, at least 3 days, or at least 4 days, the total (w/w) % of the carbohydrate, sugar alcohol or combination thereof is selected to be no greater than 60%, no greater than 55%, no greater than 50%, no greater than 45%, or no greater than 40%, or no greater than 30%.

In one embodiment, when the controlled release of the biologic is characterized by the number of days required for 100% release of the biologic is at least 2 days, at least 3 days, or at least 4 days, the total (w/w) % of the carbohydrate, sugar alcohol or combination thereof is selected to be no greater than 60%.

In one embodiment, when the controlled release of the biologic is characterized by the number of days required for 100% release of the biologic such that the number of days are at least 2 days, at least 3 days, or at least 4 days, the total (w/w) % of the carbohydrate, sugar alcohol or combination thereof is selected to be no greater than 55%.

In one embodiment, when the controlled release of the biologic is characterized by the number of days required for 100% release of the biologic such that the number of days are at least 2 days, at least 3 days, or at least 4 days, the total (w/w) % of the carbohydrate, sugar alcohol or combination thereof is selected to be no greater than 50%.

In one embodiment, when the controlled release of the biologic is characterized by the number of days required for 100% release of the biologic such that the number of days are at least 2 days, at least 3 days, or at least 4 days, the total (w/w) % of the carbohydrate, sugar alcohol or combination thereof is selected to be no greater than 45%.

In one embodiment, when the controlled release of the biologic is characterized by the number of days required for 100% release of the biologic such that the number of days are at least 2 days, at least 3 days, or at least 4 days, the total (w/w) % of the carbohydrate, sugar alcohol or combination thereof is selected to be no greater than 40%.

In one embodiment, when the controlled release of the biologic is characterized by the number of days required for 100% release of the biologic is at least 3 days, the the total (w/w) % of the carbohydrate, sugar alcohol or combination thereof is selected to be no greater than 60%.

In one embodiment, when the controlled release of the biologic is characterized by the number of days required for 100% release of the biologic is at least 3 days, the total (w/w) % of the carbohydrate, sugar alcohol or combination thereof is selected to be no greater than 55%.

In one embodiment, when the controlled release of the biologic is characterized by the number of days required for 100% release of the biologic is at least 3 days, the the total (w/w) % of the carbohydrate, sugar alcohol or combination thereof is selected to be no greater than 50%.

In one embodiment, when the controlled release of the biologic is characterized by the number of days required for 100% release of the biologic is at least 3 days, the the total (w/w) % of the carbohydrate, sugar alcohol or combination thereof is selected to be no greater than 45%.

In one embodiment, when the controlled release of the biologic is characterized by the number of days required for 100% release of the biologic is at least 3 days, the the total (w/w) % of the carbohydrate, sugar alcohol or combination thereof is selected to be no greater than 40%.

In one embodiment, when the controlled release of the biologic is characterized by the number of days required for 100% release of the biologic is at least 4 days, the the total (w/w) % of the carbohydrate, sugar alcohol or combination thereof is selected to be no greater than 60%.

In one embodiment, when the controlled release of the biologic is characterized by the number of days required for 100% release of the biologic is at least 4 days, the total (w/w) % of the carbohydrate, sugar alcohol or combination thereof is selected to be no greater than 55%.

In one embodiment, when the controlled release of the biologic is characterized by the number of days required for 100% release of the biologic is at least 4 days, the the total (w/w) % of the carbohydrate, sugar alcohol or combination thereof is selected to be no greater than 50%.

In one embodiment, when the controlled release of the biologic is characterized by the number of days required for 100% release of the biologic is at least 4 days, the the total (w/w) % of the carbohydrate, sugar alcohol or combination thereof is selected to be no greater than 45%.

In one embodiment, when the controlled release of the biologic is characterized by the number of days required for 100% release of the biologic is at least 4 days, the the total (w/w) % of the carbohydrate, sugar alcohol or combination thereof is selected to be no greater than 40%.

In one embodiment, when the controlled release of the biologic is characterized by the amount of the biologic released on day 1 is from 0 to 50% of the total amount of the biologic, the Dv₉₀ particle size can be selected to be from 10 μm to 200 μm such as 35 μm to 75 μm such as 35 μm to 100 μm, or 35 μm to 150 μm, wherein particles comprise a mixture of the biologic and at least one dehydration stabilizer such as a carbohydrate, a sugar alcohol, or a combination thereof. The higher the Dv₉₀ particle size, the higher the percentage release of the total amount of the biologic on day 1.

In some embodiments, when the amount of the biologic released on day 1 is from 0 to 50%, 0 to 45%, 0 to 40%, 0 to 35%, 0 to 30%, 0 to 25%, 0 to 20%, 0 to 15%, 0 to 10%, 0 to 5% of the total amount of the biologic, the Dv₉₀ particle size can be selected to be from 10 to 20 μm, from 10 to 30 μm, from 10 to 40 μm, from 10 to 50 μm, from 10 to 60 μm, from 10 to 70 μm, from 10 to 100 μm, from 10 to 110 μm, from 10 to 120 μm, from 10 to 130 μm, from 10 to 140 μm, from 10 to 150 μm, from 10 to 160 μm, from 10 to 170 μm, from 10 to 180 μm, from 10 to 190 μm, from 10 to 200 μm, wherein particles comprise a mixture of the biologic and at least one dehydration stabilizer such as a carbohydrate, a sugar alcohol, or a combination thereof. The higher the Dv₉₀ particle size, the higher the percentage release of the total amount of the biologic on day 1.

In one embodiment, when the controlled release of the biologic is characterized by the number of days required for 100% release of the biologic such that the number of days are at least 2 days, at least 3 days, or at least 4 days, the Dv₉₀ particle size can be selected to be from 10 μm to 200 μm such as 35 μm to 75 μm such as 35 μm to 100 μm, or 35 μm to 150 μm, wherein particles comprise a mixture of the biologic and at least one dehydration stabilizer such as a carbohydrate, a sugar alcohol, or a combination thereof. The higher the Dv₉₀ particle size, the higher the percentage release of the total amount of the biologic on day 1.

In some embodiments, when the controlled release of the biologic is characterized by the number of days required for 100% release of the biologic such that the number of days are at least 2 days, at least 3 days, or at least 4 days, the Dv₉₀ particle size can be selected to be from 10 to 20 μm, from 10 to 30 μm, from 10 to 40 μm, from 10 to 50 μm, from 10 to 60 μm, from 10 to 70 μm, from 10 to 100 μm, from 10 to 110 μm, from 10 to 120 μm, from 10 to 130 μm, from 10 to 140 μm, from 10 to 150 μm, from 10 to 160 μm, from 10 to 170 μm, from 10 to 180 μm, from 10 to 190 μm, from 10 to 200 μm, wherein particles comprise a mixture of the biologic and at least one dehydration stabilizer such as a carbohydrate, a sugar alcohol, or a combination thereof. The higher the Dv₉₀ particle size, the higher the percentage release of the total amount of the biologic on day 1.

In such embodiments, the biologic is a recombinant protein, a lipid encapsulating a nucleic acid or a virus comprising at least one heterologous nucleic acid sequence. In some embodiments, the virus is selected from a group consisting of AAV1, AAV2 such as AAV2.7m8, AAV3, AAV4, AAV5, AAV6, AAV7, AAV8, AAV9, AAV10, AAV11 and a mutant, hybrid or variant thereof. In these embodiments, the AAV comprises at least one heterologous nucleic acid sequence. In these embodiments, the heterologous nucleic acid sequence can be either a coding nucleic acid sequence or non-coding nucleic acid sequence. When the heterologous nucleic acid sequence is a coding nucleic acid sequence, it may code for a therapeutic protein as described in the section under the heading of biologic.

In another preferred embodiment, a recombinant protein is selected from a group consisting of an antibody, an antigen binding fragment, a fusion protein, a cytokine, a transcription factor, an enzyme or a hormone.

In one embodiment, the biologic is a virus and the total amount of the virus comprised in the pharmaceutically acceptable implant for controlled release is at least 10⁹ vg. In certain embodiments, the total amount of the virus is from 10⁹ to 10¹⁵ vg. In other preferred embodiments, the total amount of the virus is from 10⁹ to 10¹⁵ vg, from 10⁹ to 10¹³ vg, or from 10⁹ to 10¹² vg.

In one embodiment, the biologic is an adeno-associated virus (AAV) selected from a group consisting of AAV1, AAV2 such as AAV2.7m8, AAV3, AAV4, AAV5, AAV6, AAV7, AAV8, AAV9, AAV10, AAV11 and a mutant, hybrid or variant thereof. In one embodiment, the total amount of the AAV comprised in the pharmaceutically acceptable implant for controlled release is at least 10⁹ vg. In certain embodiments, the total amount of the AAV is from 10⁹ to 10¹⁵ vg. In other preferred embodiments the total amount of the AAV is from 10⁹ to 10¹⁵ vg, from 10⁹ to 10¹³ vg, or from 10⁹ to 10¹² vg.

In one embodiment, the biologic is an adeno-associated virus (AAV) selected from a group consisting of AAV1, AAV2 such as AAV2.7m8, AAV3, AAV4, AAV5, AAV6, AAV7, AAV8, AAV9, AAV10, AAV11 and a mutant, hybrid or variant thereof. In one embodiment, the total amount of the AAV comprised in the pharmaceutically acceptable implant for controlled release is at least 10¹⁰ vg. In certain embodiments, the total amount of the AAV is from 10¹⁰ to 10¹⁵ vg. In other preferred embodiments the total amount of the AAV is from 10¹⁰ to 10¹⁵ vg. In other preferred embodiments the total amount of the AAV is from 10¹⁰ to 10¹⁴ vg, In other preferred embodiments the total amount of the AAV is from 10¹⁰ to 10¹³ vg. In other preferred embodiments the total amount of the AAV is from 10¹⁰ to 10¹² vg. In other preferred embodiments the total amount of the AAV is from 10¹⁰ to 10¹¹ vg.

In this embodiment, the controlled release can be characterized as the amount of the AAV released on day 1 is no greater 0 to 50% such as 0 to 25%, 0 to 20% 0 to 10%, 0 to 5%, or about 0% of the total amount of the AAV on day 1, no greater than 50% of the total amount of the AAV released per day from day 2 until the last day of the controlled release, and/or number of days required for 100% release of the AAV is not less than 4 days. In these embodiments, the AAV comprises at least one heterologous nucleic acid sequence. In these embodiments, the heterologous nucleic acid sequence can be either a coding nucleic acid sequence or non-coding nucleic acid sequence. When the heterologous nucleic acid sequence is a coding nucleic acid sequence, it may code for a therapeutic protein as described in the section under the heading of biologic.

In this embodiment, the controlled release can be characterized as the amount of the AAV released on day 1 is no greater than 9.0×10⁹ to 1.5×10¹⁰ AAV vg released on day 1, no greater than in the order 10¹¹ vg AAV per day such as in the order 10⁸, or 10⁹ or 10¹⁰ such as 5.0×10⁹ to 1.5×10¹⁰ AAV vg released per day from day 2 until the last day of the controlled release, and/or number of days required for 100% release of the AAV is not less than 4 days. In these embodiments, the AAV comprises at least one heterologous nucleic acid sequence. In these embodiments, the heterologous nucleic acid sequence can be either a coding nucleic acid sequence or non-coding nucleic acid sequence. When the heterologous nucleic acid sequence is a coding nucleic acid sequence, it may code for a therapeutic protein as described in the section under the heading of biologic.

In one embodiment, the biologic is a recombinant protein and the total amount of the recombinant protein comprised in the pharmaceutically acceptable implant for controlled release is at least at least 100 μg. In certain embodiments, the total amount of the recombinant protein is from 100 to 3000 μg. In other preferred embodiments, the total amount of the recombinant protein that is mixed with at least one dehydration stabilizer is from 100 to 2,500 μg, or from 100 to 2000 μg.

In another preferred embodiment, the biologic is a recombinant protein such as an antibody, an antigen binding fragment, a fusion protein or a hormone.

### Pharmaceutically Acceptable Implant

According to the invention, a pharmaceutically acceptable implant is provided that comprises a xerogel as described in the previous section, a biologic as also described in the previous section, and at least one dehydration stabilizer as also described in the previous section.

Throughout this section, reference to (w/w) % is to be construed as based on the weight of the pharmaceutically acceptable implant.

Throughout this section, in all of the embodiments including all of the parameters disclosed in this section, “particles” or “total particles” or “Dv₉₀ particle size” is each described as pertaining to particles comprising a mixture of the biologic and at least one dehydration stabilizer such as a carbohydrate, sugar alcohol or combination thereof.

Throughout this section, in all of the embodiments including all of the parameters disclosed in this section, “particles”, or “total particles” or “Dv₉₀ particle size” can also refer to a mixture according to the following paragraphs.

In some embodiments, particles, or total particles in the implant of the invention such as a pharmaceutically acceptable implant of the invention comprise a mixture of the biologic as discussed above and at least one dehydration stabilizer as discussed above.

In some embodiments, particles, or total particles in the implant of the invention such as a pharmaceutically acceptable implant of the invention comprise a mixture of the biologic, at least one dehydration stabilizer, and at least one other stabilizer such as a buffer and a surfactant.

In some embodiments, particles, or total particles in the implant of the invention such as a pharmaceutically acceptable implant of the invention comprise a mixture of the biologic, at least one dehydration stabilizer, and at least one other stabilizer such as a buffer such as PBS comprising at least two or at least three salts, and a non-ionic surfactant such as for example F-68, F-127 or F123.

In some embodiments, particles, or total particles in the implant of the invention such as a pharmaceutically acceptable implant of the invention consist of a mixture of the biologic, at least one dehydration stabilizer, and at least one other stabilizer such as a buffer and a surfactant.

In some embodiments, particles, or total particles in the implant of the invention such as a pharmaceutically acceptable implant of the invention consist of a mixture of the biologic, at least one dehydration stabilizer, and at least one other stabilizer such as a buffer such as PBS comprising at least two or at least three salts, and a non-ionic surfactant such as for example F-68, F-127 or F123.

In some embodiments, particle size such as Dv₉₀ particle size refers to particles in the implant of the invention such as a pharmaceutically acceptable implant of the invention comprising a mixture of the biologic as discussed above and at least one dehydration stabilizer as discussed above.

In some embodiments, particle size such as Dv₉₀ particle size refers to particles in the implant of the invention such as a pharmaceutically acceptable implant of the invention comprising a mixture of the biologic, at least one dehydration stabilizer, and at least one other stabilizer such as a buffer and a surfactant.

In some embodiments, particle size such as Dv₉₀ particle size refers to particles in the implant of the invention such as a pharmaceutically acceptable implant of the invention comprising a mixture of the biologic, at least one dehydration stabilizer, and at least one other stabilizer such as a buffer such as PBS comprising at least two or at least three salts, and a non-ionic surfactant such as for example F-68, F-127 or F123.

In some embodiments, particle size such as Dv₉₀ particle size refers to particles in the implant of the invention such as a pharmaceutically acceptable implant of the invention consisting of a mixture of the biologic, at least one dehydration stabilizer, and at least one other stabilizer such as a buffer and a surfactant.

In some embodiments, particle size such as Dv₉₀ particle size refers to particles in the implant of the invention such as a pharmaceutically acceptable implant of the invention consisting of a mixture of the biologic, at least one dehydration stabilizer, and at least one other stabilizer such as a buffer such as PBS comprising at least two or at least three salts, and a non-ionic surfactant such as for example F-68, F-127 or F123.

A pharmaceutically acceptable implant is provided according to the invention that comprises; a xerogel comprising a matrix comprising covalently crosslinked multiarm precursors comprising a first multi-arm precursor comprising a nucleophile, a second multi-arm precursor comprising an electrophile, and optionally, a third multi-arm precursor comprising an electrophile. It also comprises particles comprising a mixture of a biologic and at least one dehydration stabilizer selected from a carbohydrate such as a sugar, such as sucrose and trehalose, a sugar alcohol such as mannitol, or a combination thereof, wherein the particles are dispersed within the xerogel. The total (w/w) % of said dehydration stabilizer is no greater than 60%, no greater than 55%, no greater than 50%, no greater than 45%, or no greater than 40%, or no greater than 30%.

A pharmaceutically acceptable implant is provided according to the invention that comprises; a xerogel comprising a matrix comprising covalently crosslinked multiarm precursors comprising a first multi-arm precursor comprising a nucleophile, a second multi-arm precursor comprising an electrophile, and optionally, a third multi-arm precursor comprising an electrophile. The molecular weight between crosslinks in the xerogel is from 7 to 25 kDa, 9 to 20 kDa, or 10 to 15 kDa. It also comprises particles comprising a mixture of a biologic and at least one dehydration stabilizer selected from a carbohydrate such as a sugar, such as sucrose and trehalose, a sugar alcohol such as mannitol, or a combination thereof, wherein the particles are dispersed within the xerogel.

A pharmaceutically acceptable implant is provided according to the invention that comprises; a xerogel comprising a matrix comprising covalently crosslinked multiarm precursors comprising a first multi-arm precursor comprising a nucleophile, a second multi-arm precursor comprising an electrophile, and optionally, a third multi-arm precursor comprising an electrophile. It also comprises particles comprising a mixture of a biologic and at least one dehydration stabilizer selected from a carbohydrate such as a sugar, such as sucrose and trehalose, a sugar alcohol such as mannitol, or a combination thereof, wherein the particles are dispersed within the xerogel. The (w/w) % of the total particles comprising a mixture of the biologic and said dehydration stabilizer is no greater than 80%, or no greater than 70%, or no greater than 60%, or no greater than 50% such as from 20% to 40%, from 20% to 30%.

A pharmaceutically acceptable implant is provided according to the invention that comprises; a xerogel comprising a matrix comprising covalently crosslinked multiarm precursors comprising a first multi-arm precursor comprising a nucleophile, a second multi-arm precursor comprising an electrophile, and optionally, a third multi-arm precursor comprising an electrophile. It also comprises particles comprising a mixture of a biologic and at least one dehydration stabilizer selected from a carbohydrate such as a sugar, such as sucrose and trehalose, a sugar alcohol such as mannitol, or a combination thereof, wherein the particles are dispersed within the xerogel. The (w/w) % of the total number of multi-arm precursors is from 20% to 80% such as from 35% to 75%, such as from 35% to 65%, from 35% to 55%, from 35% to 45%.

A pharmaceutically acceptable implant is provided according to the invention that comprises; a xerogel comprising a matrix comprising covalently crosslinked multiarm precursors comprising a first multi-arm precursor comprising a nucleophile, a second multi-arm precursor comprising an electrophile, and optionally, a third multi-arm precursor comprising an electrophile. It also comprises particles comprising a mixture of a biologic and at least one dehydration stabilizer selected from a carbohydrate such as a sugar, such as sucrose and trehalose, a sugar alcohol such as mannitol, or a combination thereof, wherein the particles are dispersed within the xerogel. The ratio of (1) the (w/w) % of the total particles comprising a mixture of the biologic and said dehydration stabilizer and (2) the (w/w) % of the total number of multi-arm precursors is from 0.3 to 4.0, such as 0.3 to 3.5, from 0.3 to 3.0, from 0.3 to 2.5, from 0.3 to 2.0, from 0.3 to 1.5, from 0.3 to 1.0 or from 0.5 to 4.0, such as 0.5 to 3.5, from 0.5 to 3.0, from 0.5 to 2.5, from 0.5 to 2.0, from 0.5 to 1.5, from 0.5 to 1.0, or from 0.6 to 4.0, 0.6 to 3.5, from 0.6 to 3.0, from 0.6 to 2.5, from 0.6 to 2.0, from 0.6 to 1.5, or from 0.6 to 1.0. In this context, the (w/w) % is based on the weight of the pharmaceutically acceptable implant.

A pharmaceutically acceptable implant is provided according to the invention that comprises; a xerogel comprising a matrix comprising covalently crosslinked multiarm precursors comprising a first multi-arm precursor comprising a nucleophile, a second multi-arm precursor comprising an electrophile, and optionally, a third multi-arm precursor comprising an electrophile. It also comprises particles comprising a mixture of a biologic and at least one dehydration stabilizer selected from a carbohydrate such as a sugar, such as sucrose and trehalose, a sugar alcohol such as mannitol, or a combination thereof, wherein the particles are dispersed within the xerogel. In certain embodiments, the Dv₉₀ particle size is from 10 μm to 200 μm such as 35 μm to 75 μm such as 35 μm to 100 μm, or 35 μm to 150 μm, wherein particles comprise the mixture of the biologic and said dehydration stabilizer such as a carbohydrate, a sugar alcohol, or a combination thereof.

A pharmaceutically acceptable implant is provided according to the invention that comprises a xerogel comprising a matrix comprising covalently crosslinked multi-arm precursors comprising a first multi-arm precursor comprising a nucleophile, a second multi-arm precursor comprising an electrophile comprising a first reactive group, and a third multi-arm precursor comprising an electrophile comprising a second reactive group. It also comprises particles comprising a mixture of a biologic and at least one dehydration stabilizer selected from a carbohydrate such as a sugar, such as sucrose and trehalose, a sugar alcohol such as mannitol, or a combination thereof. In one embodiment, the third multi-arm precursor has a longer hydrolysis half-life as compared to the second multi-arm precursor. In this embodiment, the molar ratio of the first reactive group comprised in the second multi-arm precursor, and the second reactive group comprised in the third multi-arm precursor is 30-90:70-10, 40-80:60-20, 50-70:50-30, or 40-60:60-40.

A pharmaceutically acceptable implant is provided according to the invention that comprises a xerogel comprising a matrix comprising covalently crosslinked multi-arm precursors comprising a first multi-arm precursor comprising a comprising a primary amine, a second multi-arm precursor comprising succinimidyl succinate, and a third multiarm precursor comprising succinimidyl glutarate. It also comprises particles comprising a mixture of a biologic and at least one dehydration stabilizer selected from a carbohydrate such as a sugar, such as sucrose and trehalose, a sugar alcohol such as mannitol, or a combination thereof. In this embodiment, molar ratio of the succinimidyl succinate group comprised in the second multi-arm precursor, and the succinimidyl glutarate group comprised in the third multi-arm precursor is 30-90:70-10, 40-80:60-20, 50-70:50-30, or 40-60:60-40.

A pharmaceutically acceptable implant is provided according to the invention that comprises a xerogel comprising a matrix comprising covalently crosslinked multi-arm precursors comprising a first multi-arm precursor comprising a nucleophile, a second multi-arm precursor comprising an electrophile comprising a first reactive group, and a third multi-arm precursor comprising an electrophile comprising a second reactive group. It also comprises particles comprising a mixture of a biologic and at least one dehydration stabilizer selected from a carbohydrate such as a sugar, such as sucrose and trehalose, a sugar alcohol such as mannitol, or a combination thereof. The total (w/w) % of said dehydration stabilizer is no greater than 60%, no greater than 55%, no greater than 50%, no greater than 45%, or no greater than 40%, or no greater than 30%. The molecular weight between crosslinks in the xerogel is from 7 to 25 kDa, 9 to 20 kDa, or 10 to 15 kDa. The (w/w) % of the total particles comprising a mixture of the biologic and said dehydration stabilizer is no greater than 80%, or no greater than 70%, or no greater than 60%, or no greater than 50% such as from 20% to 40%, from 20% to 30%. The (w/w) % of the total number of multi-arm precursors is from 20% to 80% such as from 35% to 75%, such as from 35% to 65%, from 35% to 55%, from 35% to 45%. The ratio of (1) the (w/w) % of the total particles comprising a mixture of the biologic and said dehydration stabilizer and (2) the (w/w) % of the total number of multi-arm precursors is from 0.3 to 4.0, such as 0.3 to 3.5, from 0.3 to 3.0, from 0.3 to 2.5, from 0.3 to 2.0, from 0.3 to 1.5, from 0.3 to 1.0 or from 0.5 to 4.0, such as 0.5 to 3.5, from 0.5 to 3.0, from 0.5 to 2.5, from 0.5 to 2.0, from 0.5 to 1.5, from 0.5 to 1.0, or from 0.6 to 4.0, 0.6 to 3.5, from 0.6 to 3.0, from 0.6 to 2.5, from 0.6 to 2.0, from 0.6 to 1.5, or from 0.6 to 1.0. In one embodiment, the third multi-arm precursor has a longer hydrolysis half-life as compared to the second multi-arm precursor. In this embodiment, the molar ratio of the first reactive group comprised in the second multi-arm precursor, and the second reactive group comprised in the third multi-arm precursor is 30-90:70-10, 40-80:60-20, 50-70:50-30, or 40-60:60-40. In certain embodiments, the Dv₉₀ particle size is from 10 μm to 200 μm such as 35 μm to 75 μm such as 35 μm to 100 μm, or 35 μm to 150 μm, wherein particles comprise the mixture of the biologic and said dehydration stabilizer such as a carbohydrate, a sugar alcohol, or a combination thereof.

A pharmaceutically acceptable implant is provided according to the invention that comprises a xerogel comprising a matrix comprising covalently crosslinked multi-arm precursors comprising a first multi-arm precursor comprising a comprising a primary amine, a second multi-arm precursor comprising succinimidyl succinate, and a third multiarm precursor comprising succinimidyl glutarate. It also comprises particles comprising a mixture of a biologic and at least one dehydration stabilizer selected from a carbohydrate such as a sugar, such as sucrose and trehalose, a sugar alcohol such as mannitol, or a combination thereof. The total (w/w) % of said dehydration stabilizer is no greater than 60%, no greater than 55%, no greater than 50%, no greater than 45%, or no greater than 40%, or no greater than 30%. The molecular weight between crosslinks in the xerogel is from 7 to 25 kDa, 9 to 20 kDa, or 10 to 15 kDa. The (w/w) % of the total particles comprising a mixture of the biologic and said dehydration stabilizer is no greater than 80%, or no greater than 70%, or no greater than 60%, or no greater than 50% such as from 20% to 40%, from 20% to 30%. The (w/w) % of the total number of multi-arm precursors is from 20% to 80% such as from 35% to 75%, such as from 35% to 65%, from 35% to 55%, from 35% to 45%. The ratio of (1) the (w/w) % of the total particles comprising a mixture of the biologic and said dehydration stabilizer and (2) the (w/w) % of the total number of multi-arm precursors is from 0.3 to 4.0, such as 0.3 to 3.5, from 0.3 to 3.0, from 0.3 to 2.5, from 0.3 to 2.0, from 0.3 to 1.5, from 0.3 to 1.0 or from 0.5 to 4.0, such as 0.5 to 3.5, from 0.5 to 3.0, from 0.5 to 2.5, from 0.5 to 2.0, from 0.5 to 1.5, from 0.5 to 1.0, or from 0.6 to 4.0, 0.6 to 3.5, from 0.6 to 3.0, from 0.6 to 2.5, from 0.6 to 2.0, from 0.6 to 1.5, or from 0.6 to 1.0. In one embodiment, the third multi-arm precursor has a longer hydrolysis half-life as compared to the second multi-arm precursor. In this embodiment, molar ratio of the succinimidyl succinate group comprised in the second multi-arm precursor, and the succinimidyl glutarate group comprised in the third multi-arm precursor is 30-90:70-10, 40-80:60-20, 50-70:50-30, or 40-60:60-40. In certain embodiments, the Dv₉₀ particle size is from 10 μm to 200 μm such as 35 μm to 75 μm such as 35 μm to 100 μm, or 35 μm to 150 μm, wherein particles comprise the mixture of the biologic and said dehydration stabilizer such as a carbohydrate, a sugar alcohol, or a combination thereof.

A pharmaceutically acceptable implant is provided according to the invention that comprises; a xerogel comprising a matrix comprising covalently crosslinked multiarm precursors comprising a first multi-arm precursor comprising a nucleophile, a second multi-arm precursor comprising an electrophile, and optionally, a third multi-arm precursor comprising an electrophile. It also comprises particles comprising a mixture of AAV selected from a group consisting of AAV1, AAV2 such as AAV2.7m8, AAV3, AAV4, AAV5, AAV6, AAV7, AAV8, AAV9, AAV10, AAV11 and a mutant, hybrid or variant thereof at a total amount in the order from 10⁹ to 10¹⁵ vg, such as in the order from 10⁹ to 10¹³ vg and at least one dehydration stabilizer selected from a carbohydrate such as a sugar, such as sucrose or trehalose, a sugar alcohol such as mannitol, or a combination thereof, wherein the particles are dispersed within the xerogel. The total (w/w) % of said dehydration stabilizer is no greater than 60%, no greater than 55%, no greater than 50%, no greater than 45%, or no greater than 40%, or no greater than 30%. In these embodiments, the AAV comprises at least one heterologous nucleic acid sequence. In these embodiments, the heterologous nucleic acid sequence can be either a coding nucleic acid sequence or non-coding nucleic acid sequence. When the heterologous nucleic acid sequence is a coding nucleic acid sequence, it may code for a therapeutic protein as described in the section under the heading of biologic. In some embodiments, the total amount of the AAV is in the order from 10¹⁰ to 10¹⁵ vg. In some embodiments, the total amount of the AAV is in the order from 10¹⁰ to 10¹⁴ vg. In some embodiments, the total amount of the AAV is in the order from 10¹⁰ to 10¹³ vg. In some embodiments, the total amount of the AAV is in the order from 10¹⁰ to 10¹² vg. In some embodiments, the total amount of the AAV is in the order from 10¹⁰ to 10¹¹ vg.

A pharmaceutically acceptable implant is provided according to the invention that comprises; a xerogel comprising a matrix comprising covalently crosslinked multiarm precursors comprising a first multi-arm precursor comprising a nucleophile, a second multi-arm precursor comprising an electrophile, and optionally, a third multi-arm precursor comprising an electrophile. It also comprises particles comprising a mixture of AAV selected from a group consisting of AAV1, AAV2 such as AAV2.7m8, AAV3, AAV4, AAV5, AAV6, AAV7, AAV8, AAV9, AAV10, AAV11 and a mutant, hybrid or variant thereof at a total amount in the order from 10⁹ to 10¹⁵ vg, such as in the order from 10⁹ to 10¹³ vg and at least one dehydration stabilizer, selected from a carbohydrate such as a sugar, such as sucrose or trehalose, a sugar alcohol such as mannitol, or a combination thereof, wherein the particles are dispersed within the xerogel. In certain embodiments, the Dv₉₀ particle size is from 10 μm to 200 μm such as 35 μm to 75 μm such as 35 μm to 100 μm, or 35 μm to 150 μm, wherein particles comprise the mixture of the AAV and said dehydration stabilizer such as a carbohydrate, a sugar alcohol, or a combination thereof. In these embodiments, the AAV comprises at least one heterologous nucleic acid sequence. In these embodiments, the heterologous nucleic acid sequence can be either a coding nucleic acid sequence or non-coding nucleic acid sequence. When the heterologous nucleic acid sequence is a coding nucleic acid sequence, it may code for a therapeutic protein as described in the section under the heading of biologic. In some embodiments, the total amount of the AAV is in the order from 10¹⁰ to 10¹⁵ vg. In some embodiments, the total amount of the AAV is in the order from 10¹⁰ to 10¹⁴ vg. In some embodiments, the total amount of the AAV is in the order from 10¹⁰ to 10¹³ vg. In some embodiments, the total amount of the AAV is in the order from 10¹⁰ to 10¹² vg. In some embodiments, the total amount of the AAV is in the order from 10¹⁰ to 10¹¹ vg.

A pharmaceutically acceptable implant is provided according to the invention that comprises; a xerogel comprising a matrix comprising covalently crosslinked multiarm precursors comprising a first multi-arm precursor comprising a nucleophile, a second multi-arm precursor comprising an electrophile, and optionally, a third multi-arm precursor comprising an electrophile. The molecular weight between crosslinks in the xerogel is from 7 to 25 kDa, 9 to 20 kDa, or 10 to 15 kDa. It also comprises particles comprising a mixture of AAV selected from a group consisting of AAV1, AAV2 such as AAV2.7m8, AAV3, AAV4, AAV5, AAV6, AAV7, AAV8, AAV9, AAV10, AAV11 and a mutant, hybrid or variant thereof at a total amount in the order from 10⁹ to 10¹⁵ vg, such as in the order from 10⁹ to 10¹³ vg and at least one dehydration stabilizer selected from a carbohydrate such as a sugar, such as sucrose and trehalose, a sugar alcohol such as mannitol, or a combination thereof, wherein the particles are dispersed within the xerogel. In these embodiments, the AAV comprises at least one heterologous nucleic acid sequence. In these embodiments, the heterologous nucleic acid sequence can be either a coding nucleic acid sequence or non-coding nucleic acid sequence. When the heterologous nucleic acid sequence is a coding nucleic acid sequence, it may code for a therapeutic protein as described in the section under the heading of biologic. In some embodiments, the total amount of the AAV is in the order from 10¹⁰ to 10¹⁵ vg. In some embodiments, the total amount of the AAV is in the order from 10¹⁰ to 10¹⁴ vg. In some embodiments, the total amount of the AAV is in the order from 10¹⁰ to 10¹³ vg. In some embodiments, the total amount of the AAV is in the order from 10¹⁰ to 10¹² vg. In some embodiments, the total amount of the AAV is in the order from 10¹⁰ to 10¹¹ vg.

A pharmaceutically acceptable implant is provided according to the invention that comprises; a xerogel comprising a matrix comprising covalently crosslinked multiarm precursors comprising a first multi-arm precursor comprising a nucleophile, a second multi-arm precursor comprising an electrophile, and optionally, a third multi-arm precursor comprising an electrophile. It also comprises particles comprising a mixture of AAV selected from a group consisting of AAV1, AAV2 such as AAV2.7m8, AAV3, AAV4, AAV5, AAV6, AAV7, AAV8, AAV9, AAV10, AAV11 and a mutant, hybrid or variant thereof at a total amount in the order from 10⁹ to 10¹⁵ vg, such as in the order from 10⁹ to 10¹³ vg and at least one dehydration stabilizer selected from a carbohydrate such as a sugar, such as sucrose and trehalose, a sugar alcohol such as mannitol, or a combination thereof, wherein the particles are dispersed within the xerogel. The (w/w) % of the total particles comprising a mixture of said AAV and said dehydration stabilizer is no greater than 80%, or no greater than 70%, or no greater than 60%, or no greater than 50% such as from 20% to 40%, from 20% to 30%. In this context, the (w/w) % is based on the weight of the pharmaceutically acceptable implant. In these embodiments, the AAV comprises at least one heterologous nucleic acid sequence. In these embodiments, the heterologous nucleic acid sequence can be either a coding nucleic acid sequence or non-coding nucleic acid sequence. When the heterologous nucleic acid sequence is a coding nucleic acid sequence, it may code for a therapeutic protein as described in the section under the heading of biologic. In some embodiments, the total amount of the AAV is in the order from 10¹⁰ to 10¹⁵ vg. In some embodiments, the total amount of the AAV is in the order from 10¹⁰ to 10¹⁴ vg. In some embodiments, the total amount of the AAV is in the order from 10¹⁰ to 10¹³ vg. In some embodiments, the total amount of the AAV is in the order from 10¹⁰ to 10¹² vg. In some embodiments, the total amount of the AAV is in the order from 10¹⁰ to 10¹¹ vg.

A pharmaceutically acceptable implant is provided according to the invention that comprises; a xerogel comprising a matrix comprising covalently crosslinked multiarm precursors comprising a first multi-arm precursor comprising a nucleophile, a second multi-arm precursor comprising an electrophile, and optionally, a third multi-arm precursor comprising an electrophile. It also comprises particles comprising a mixture of AAV selected from a group consisting of AAV1, AAV2 such as AAV2.7m8, AAV3, AAV4, AAV5, AAV6, AAV7, AAV8, AAV9, AAV10, AAV11 and a mutant, hybrid or variant thereof at a total amount in the order from 10⁹ to 10¹⁵ vg, such as in the order from 10⁹ to 10¹³ vg and at least one dehydration stabilizer selected from a carbohydrate such as a sugar, such as sucrose and trehalose, a sugar alcohol such as mannitol, or a combination thereof, wherein the particles are dispersed within the xerogel. The (w/w) % of the total number of multi-arm precursors is from 20% to 80% such as from 35% to 75%, such as from 35% to 65%, from 35% to 55%, from 35% to 45%. In this context, the (w/w) % is based on the weight of the pharmaceutically acceptable implant. In these embodiments, the AAV comprises at least one heterologous nucleic acid sequence. In these embodiments, the heterologous nucleic acid sequence can be either a coding nucleic acid sequence or non-coding nucleic acid sequence. When the heterologous nucleic acid sequence is a coding nucleic acid sequence, it may code for a therapeutic protein as described in the section under the heading of biologic. In some embodiments, the total amount of the AAV is in the order from 10¹⁰ to 10¹⁵ vg. In some embodiments, the total amount of the AAV is in the order from 10¹⁰ to 10¹⁴ vg. In some embodiments, the total amount of the AAV is in the order from 10¹⁰ to 10¹³ vg. In some embodiments, the total amount of the AAV is in the order from 10¹⁰ to 10¹² vg. In some embodiments, the total amount of the AAV is in the order from 10¹⁰ to 10¹¹ vg.

A pharmaceutically acceptable implant is provided according to the invention that comprises; a xerogel comprising a matrix comprising covalently crosslinked multiarm precursors comprising a first multi-arm precursor comprising a nucleophile, a second multi-arm precursor comprising an electrophile, and optionally, a third multi-arm precursor comprising an electrophile. It also comprises particles comprising a mixture of AAV selected from a group consisting of AAV1, AAV2 such as AAV2.7m8, AAV3, AAV4, AAV5, AAV6, AAV7, AAV8, AAV9, AAV10, AAV11 and a mutant, hybrid or variant thereof at a total amount in the order from 10⁹ to 10¹⁵ vg, such as in the order from 10⁹ to 10¹³ vg and at least one dehydration stabilizer selected from a carbohydrate such as a sugar, such as sucrose and trehalose, a sugar alcohol such as mannitol, or a combination thereof, wherein the particles are dispersed within the xerogel. The ratio of (1) the (w/w) % of the total particles comprising a mixture of said AAV and said dehydration stabilizer and (2) the (w/w) % of the total number of multi-arm precursors is from 0.3 to 4.0, such as 0.3 to 3.5, from 0.3 to 3.0, from 0.3 to 2.5, from 0.3 to 2.0, from 0.3 to 1.5, from 0.3 to 1.0 or from 0.5 to 4.0, such as 0.5 to 3.5, from 0.5 to 3.0, from 0.5 to 2.5, from 0.5 to 2.0, from 0.5 to 1.5, from 0.5 to 1.0, or from 0.6 to 4.0, 0.6 to 3.5, from 0.6 to 3.0, from 0.6 to 2.5, from 0.6 to 2.0, from 0.6 to 1.5, or from 0.6 to 1.0. In these embodiments, the AAV comprises at least one heterologous nucleic acid sequence. In these embodiments, the heterologous nucleic acid sequence can be either a coding nucleic acid sequence or non-coding nucleic acid sequence. When the heterologous nucleic acid sequence is a coding nucleic acid sequence, it may code for a therapeutic protein as described in the section under the heading of biologic. In some embodiments, the total amount of the AAV is in the order from 10¹⁰ to 10¹⁵ vg. In some embodiments, the total amount of the AAV is in the order from 10¹⁰ to 10¹⁴ vg. In some embodiments, the total amount of the AAV is in the order from 10¹⁰ to 10¹³ vg. In some embodiments, the total amount of the AAV is in the order from 10¹⁰ to 10¹² vg. In some embodiments, the total amount of the AAV is in the order from 10¹⁰ to 10¹¹ vg.

A pharmaceutically acceptable implant is provided according to the invention that comprises a xerogel comprising a matrix comprising covalently crosslinked multi-arm precursors comprising a first multi-arm precursor comprising a nucleophile, a second multi-arm precursor comprising an electrophile comprising a first reactive group, and a third multi-arm precursor comprising an electrophile comprising a second reactive group. It also comprises particles comprising a mixture of AAV selected from a group consisting of AAV1, AAV2 such as AAV2.7m8, AAV3, AAV4, AAV5, AAV6, AAV7, AAV8, AAV9, AAV10, AAV11 and a mutant, hybrid or variant thereof at a total amount in the order from 10⁹ to 10¹⁵ vg, such as in the order from 10⁹ to 10¹³ vg, such as in the order from 10⁹ to 10¹³ vg and at least one dehydration stabilizer selected from a carbohydrate such as a sugar, such as sucrose and trehalose, a sugar alcohol such as mannitol, or a combination thereof. In one embodiment, the third multi-arm precursor has a longer hydrolysis half-life as compared to the second multi-arm precursor. In this embodiment, the molar ratio of the first reactive group comprised in the second multi-arm precursor, and the second reactive group comprised in the third multi-arm precursor is 30-90:70-10, 40-80:60-20, 50-70:50-30, or 40-60:60-40. In these embodiments, the AAV comprises at least one heterologous nucleic acid sequence. In these embodiments, the heterologous nucleic acid sequence can be either a coding nucleic acid sequence or non-coding nucleic acid sequence. When the heterologous nucleic acid sequence is a coding nucleic acid sequence, it may code for a therapeutic protein as described in the section under the heading of biologic. In some embodiments, the total amount of the AAV is in the order from 10¹⁰ to 10¹⁵ vg. In some embodiments, the total amount of the AAV is in the order from 10¹⁰ to 10¹⁴ vg. In some embodiments, the total amount of the AAV is in the order from 10¹⁰ to 10¹³ vg. In some embodiments, the total amount of the AAV is in the order from 10¹⁰ to 10¹² vg. In some embodiments, the total amount of the AAV is in the order from 10¹⁰ to 10¹¹ vg.

A pharmaceutically acceptable implant is provided according to the invention that comprises a xerogel comprising a matrix comprising covalently crosslinked multi-arm precursors comprising a first multi-arm precursor comprising a comprising a primary amine, a second multi-arm precursor comprising succinimidyl succinate, and a third multiarm precursor comprising succinimidyl glutarate. It also comprises particles comprising a mixture of AAV selected from a group consisting of AAV1, AAV2 such as AAV2.7m8, AAV3, AAV4, AAV5, AAV6, AAV7, AAV8, AAV9, AAV10, AAV11 and a mutant, hybrid or variant thereof at a total amount in the order from 10⁹ to 10¹⁵ vg, such as in the order from 10⁹ to 10¹³ vg one dehydration stabilizer selected from a carbohydrate such as a sugar, such as sucrose and trehalose, a sugar alcohol such as mannitol, or a combination thereof. In this embodiment, molar ratio of the succinimidyl succinate group comprised in the second multi-arm precursor, and the succinimidyl glutarate group comprised in the third multiarm precursor is 30-90:70-10, 40-80:60-20, 50-70:50-30, or 40-60:60-40. In these embodiments, the AAV comprises at least one heterologous nucleic acid sequence. In these embodiments, the heterologous nucleic acid sequence can be either a coding nucleic acid sequence or non-coding nucleic acid sequence. When the heterologous nucleic acid sequence is a coding nucleic acid sequence, it may code for a therapeutic protein as described in the section under the heading of biologic. In some embodiments, the total amount of the AAV is in the order from 10¹⁰ to 10¹⁵ vg. In some embodiments, the total amount of the AAV is in the order from 10¹⁰ to 10¹⁴ vg. In some embodiments, the total amount of the AAV is in the order from 10¹⁰ to 10¹³ vg. In some embodiments, the total amount of the AAV is in the order from 10¹⁰ to 10¹² vg. In some embodiments, the total amount of the AAV is in the order from 10¹⁰ to 10¹¹ vg.

A pharmaceutically acceptable implant is provided according to the invention that comprises a xerogel comprising a matrix comprising covalently crosslinked multi-arm precursors comprising a first multi-arm precursor comprising a nucleophile, a second multi-arm precursor comprising an electrophile comprising a first reactive group, and a third multi-arm precursor comprising an electrophile comprising a second reactive group. It also comprises particles comprising a mixture of AAV selected from a group consisting of AAV1, AAV2 such as AAV2.7m8, AAV3, AAV4, AAV5, AAV6, AAV7, AAV8, AAV9, AAV10, AAV11 and a mutant, hybrid or variant thereof at a total amount in the order from 10⁹ to 10¹⁵ vg, such as in the order from 10⁹ to 10¹³ vg and at least one dehydration stabilizer selected from a carbohydrate such as a sugar, such as sucrose and trehalose, a sugar alcohol such as mannitol, or a combination thereof. The total (w/w) % of said dehydration stabilizer is no greater than 60%, no greater than 55%, no greater than 50%, no greater than 45%, or no greater than 40%, or no greater than 30%. The molecular weight between crosslinks in the xerogel is from 7 to 25 kDa, 9 to 20 kDa, or 10 to 15 kDa. The (w/w) % of the total particles comprising a mixture of said AAV and said dehydration stabilizer is no greater than 80%, or no greater than 70%, or no greater than 60%, or no greater than 50% such as from 20% to 40%, from 20% to 30%. The (w/w) % of the total number of multi-arm precursors is from 20% to 80% such as from 35% to 75%, such as from 35% to 65%, from 35% to 55%, from 35% to 45%. The ratio of (1) the (w/w) % of the total particles comprising a mixture of said AAV and said dehydration stabilizer and (2) the (w/w) % of the total number of multi-arm precursors is from 0.3 to 4.0, such as 0.3 to 3.5, from 0.3 to 3.0, from 0.3 to 2.5, from 0.3 to 2.0, from 0.3 to 1.5, from 0.3 to 1.0 or from 0.5 to 4.0, such as 0.5 to 3.5, from 0.5 to 3.0, from 0.5 to 2.5, from 0.5 to 2.0, from 0.5 to 1.5, from 0.5 to 1.0, or from 0.6 to 4.0, 0.6 to 3.5, from 0.6 to 3.0, from 0.6 to 2.5, from 0.6 to 2.0, from 0.6 to 1.5, or from 0.6 to 1.0. In one embodiment, the third multi-arm precursor has a longer hydrolysis half-life as compared to the second multi-arm precursor. In this embodiment, the molar ratio of the first reactive group comprised in the second multi-arm precursor, and the second reactive group comprised in the third multi-arm precursor is 30-90:70-10, 40-80:60-20, 50-70:50-30, or 40-60:60-40. In certain embodiments, the Dv₉₀ particle size is from 10 μm to 200 μm such as 35 μm to 75 μm such as 35 μm to 100 μm, or 35 μm to 150 μm, wherein particles comprise the mixture of the AAV and said dehydration stabilizer such as a carbohydrate, a sugar alcohol, or a combination thereof. In these embodiments, the AAV comprises at least one heterologous nucleic acid sequence. In these embodiments, the heterologous nucleic acid sequence can be either a coding nucleic acid sequence or non-coding nucleic acid sequence. When the heterologous nucleic acid sequence is a coding nucleic acid sequence, it may code for a therapeutic protein as described in the section under the heading of biologic. In some embodiments, the total amount of the AAV is in the order from 10¹⁰ to 10¹⁵ vg. In some embodiments, the total amount of the AAV is in the order from 10¹⁰ to 10¹⁴ vg. In some embodiments, the total amount of the AAV is in the order from 10¹⁰ to 10¹³ vg. In some embodiments, the total amount of the AAV is in the order from 10¹⁰ to 10¹² vg. In some embodiments, the total amount of the AAV is in the order from 10¹⁰ to 10¹¹ vg.

A pharmaceutically acceptable implant is provided according to the invention that comprises a xerogel comprising a matrix comprising covalently crosslinked multi-arm precursors comprising a first multi-arm precursor comprising a comprising a primary amine, a second multi-arm precursor comprising succinimidyl succinate, and a third multiarm precursor comprising succinimidyl glutarate. It also comprises particles comprising a mixture of AAV selected from a group consisting of AAV1, AAV2 such as AAV2.7m8, AAV3, AAV4, AAV5, AAV6, AAV7, AAV8, AAV9, AAV10, AAV11 and a mutant, hybrid or variant thereof at a total amount in the order from 10⁹ to 10¹⁵ vg, such as in the order from 10⁹ to 10¹³ vg at least one dehydration stabilizer selected from a carbohydrate such as a sugar, such as sucrose and trehalose, a sugar alcohol such as mannitol, or a combination thereof. The total (w/w) % of said dehydration stabilizer is no greater than 60%, no greater than 55%, no greater than 50%, no greater than 45%, or no greater than 40%, or no greater than 30%. The molecular weight between crosslinks in the xerogel is from 7 to 25 kDa, 9 to 20 kDa, or 10 to 15 kDa. The (w/w) % of the total particles comprising a mixture of said AAV and said dehydration stabilizer is no greater than 80%, or no greater than 70%, or no greater than 60%, or no greater than 50% such as from 20% to 40%, from 20% to 30%. The (w/w) % of the total number of multi-arm precursors is from 20% to 80% such as from 35% to 75%, such as from 35% to 65%, from 35% to 55%, from 35% to 45%. The ratio of (1) the (w/w) % of the total particles comprising a mixture of said AAV and said dehydration stabilizer and (2) the (w/w) % of the total number of multi-arm precursors is from 0.3 to 4.0, such as 0.3 to 3.5, from 0.3 to 3.0, from 0.3 to 2.5, from 0.3 to 2.0, from 0.3 to 1.5, from 0.3 to 1.0 or from 0.5 to 4.0, such as 0.5 to 3.5, from 0.5 to 3.0, from 0.5 to 2.5, from 0.5 to 2.0, from 0.5 to 1.5, from 0.5 to 1.0, or from 0.6 to 4.0, 0.6 to 3.5, from 0.6 to 3.0, from 0.6 to 2.5, from 0.6 to 2.0, from 0.6 to 1.5, or from 0.6 to 1.0. In one embodiment, the third multi-arm precursor has a longer hydrolysis half-life as compared to the second multi-arm precursor. In this embodiment, molar ratio of the succinimidyl succinate group comprised in the second multi-arm precursor, and the succinimidyl glutarate group comprised in the third multi-arm precursor is 30-90:70-10, 40-80:60-20, 50-70:50-30, or 40-60:60-40. In certain embodiments, the

Dv₉₀ particle size is from 10 μm to 200 μm such as 35 μm to 75 μm such as 35 μm to 100 μm, or 35 μm to 150 μm, wherein particles comprise the mixture of the AAV and said dehydration stabilizer such as a carbohydrate, a sugar alcohol, or a combination thereof. In these embodiments, the AAV comprises at least one heterologous nucleic acid sequence. In these embodiments, the heterologous nucleic acid sequence can be either a coding nucleic acid sequence or non-coding nucleic acid sequence. When the heterologous nucleic acid sequence is a coding nucleic acid sequence, it may code for a therapeutic protein as described in the section under the heading of biologic. In some embodiments, the total amount of the AAV is in the order from 10¹⁰ to 10¹⁵ vg. In some embodiments, the total amount of the AAV is in the order from 10¹⁰ to 10¹⁴ vg. In some embodiments, the total amount of the AAV is in the order from 10¹⁰ to 10¹³ vg. In some embodiments, the total amount of the AAV is in the order from 10¹⁰ to 10¹² vg. In some embodiments, the total amount of the AAV is in the order from 10¹⁰ to 10¹¹ vg.

A pharmaceutically acceptable implant is provided according to the invention that comprises; a xerogel comprising a matrix comprising covalently crosslinked multiarm precursors comprising a first multi-arm precursor comprising a nucleophile, a second multi-arm precursor comprising an electrophile, and optionally, a third multi-arm precursor comprising an electrophile. It also comprises particles comprising a mixture of a recombinant protein and at least one dehydration stabilizer selected from a carbohydrate such as a sugar, such as sucrose and trehalose, a sugar alcohol such as mannitol, or a combination thereof, wherein the particles are dispersed within the xerogel. The total (w/w) % of said dehydration stabilizer is no greater than 60%, no greater than 55%, no greater than 50%, no greater than 45%, or no greater than 40%, or no greater than 30%. In this context, the recombinant protein can be an antibody, an antigen binding fragment, a fusion protein, a cytokine, a transcription factor, an enzyme or a hormone.

A pharmaceutically acceptable implant is provided according to the invention that comprises; a xerogel comprising a matrix comprising covalently crosslinked multiarm precursors comprising a first multi-arm precursor comprising a nucleophile, a second multi-arm precursor comprising an electrophile, and optionally, a third multi-arm precursor comprising an electrophile. It also comprises particles comprising a mixture of a recombinant protein and at least one dehydration stabilizer selected from a carbohydrate such as a sugar, such as sucrose and trehalose, a sugar alcohol such as mannitol, or a combination thereof, wherein the particles are dispersed within the xerogel. In certain embodiments, the Dv₉₀ particle size is from 10 μm to 200 μm such as 35 μm to 75 μm such as 35 μm to 100 μm, or 35 μm to 150 μm, wherein particles comprise the mixture of the recombinant protein and said dehydration stabilizer such as a carbohydrate, a sugar alcohol, or a combination thereof. In this context, the recombinant protein can be an antibody, an antigen binding fragment, a fusion protein, a cytokine, a transcription factor, an enzyme or a hormone.

A pharmaceutically acceptable implant is provided according to the invention that comprises; a xerogel comprising a matrix comprising covalently crosslinked multiarm precursors comprising a first multi-arm precursor comprising a nucleophile, a second multi-arm precursor comprising an electrophile, and optionally, a third multi-arm precursor comprising an electrophile. The molecular weight between crosslinks in the xerogel is from 7 to 25 kDa, 9 to 20 kDa, or 10 to 15 kDa. It also comprises particles comprising a mixture of a recombinant protein and at least one dehydration stabilizer selected from a carbohydrate such as a sugar, such as sucrose and trehalose, a sugar alcohol such as mannitol, or a combination thereof, wherein the particles are dispersed within the xerogel. In this context, the recombinant protein can be an antibody, an antigen binding fragment, a fusion protein, a cytokine, a transcription factor, an enzyme or a hormone, or a hormone.

A pharmaceutically acceptable implant is provided according to the invention that comprises; a xerogel comprising a matrix comprising covalently crosslinked multiarm precursors comprising a first multi-arm precursor comprising a nucleophile, a second multi-arm precursor comprising an electrophile, and optionally, a third multi-arm precursor comprising an electrophile. It also comprises particles comprising a mixture of a recombinant protein and at least one dehydration stabilizer selected from a carbohydrate such as a sugar, such as sucrose and trehalose, a sugar alcohol such as mannitol, or a combination thereof, wherein the particles are dispersed within the xerogel. The (w/w) % of the total particles comprising a mixture of the recombinant protein and said dehydration stabilizer is no greater than 80%, or no greater than 70%, or no greater than 60%, or no greater than 50% such as from 20% to 40%, from 20% to 30%. In this context, the recombinant protein can be an antibody, an antigen binding fragment, a fusion protein, a cytokine, a transcription factor, an enzyme or a hormone.

A pharmaceutically acceptable implant is provided according to the invention that comprises; a xerogel comprising a matrix comprising covalently crosslinked multiarm precursors comprising a first multi-arm precursor comprising a nucleophile, a second multi-arm precursor comprising an electrophile, and optionally, a third multi-arm precursor comprising an electrophile. It also comprises particles comprising a mixture of a recombinant protein and at least one dehydration stabilizer selected from a carbohydrate such as a sugar, such as sucrose and trehalose, a sugar alcohol such as mannitol, or a combination thereof, wherein the particles are dispersed within the xerogel. The (w/w) % of the total number of multi-arm precursors is from 20% to 80% such as from 35% to 75%, such as from 35% to 65%, from 35% to 55%, from 35% to 45%. In this context, the recombinant protein can be an antibody, an antigen binding fragment, a fusion protein, a cytokine, a transcription factor, an enzyme or a hormone.

A pharmaceutically acceptable implant is provided according to the invention that comprises; a xerogel comprising a matrix comprising covalently crosslinked multiarm precursors comprising a first multi-arm precursor comprising a nucleophile, a second multi-arm precursor comprising an electrophile, and optionally, a third multi-arm precursor comprising an electrophile. It also comprises particles comprising a mixture of a biologic and at least one dehydration stabilizer selected from a carbohydrate such as a sugar, such as sucrose and trehalose, a sugar alcohol such as mannitol, or a combination thereof, wherein the particles are dispersed within the xerogel. The ratio of (1) the (w/w) % of the total particles comprising a mixture of the recombinant protein and said dehydration stabilizer and (2) the (w/w) % of the total number of multi-arm precursors is from 0.3 to 4.0, such as 0.3 to 3.5, from 0.3 to 3.0, from 0.3 to 2.5, from 0.3 to 2.0, from 0.3 to 1.5, from 0.3 to 1.0 or from 0.5 to 4.0, such as 0.5 to 3.5, from 0.5 to 3.0, from 0.5 to 2.5, from 0.5 to 2.0, from 0.5 to 1.5, from 0.5 to 1.0, or from 0.6 to 4.0, 0.6 to 3.5, from 0.6 to 3.0, from 0.6 to 2.5, from 0.6 to 2.0, from 0.6 to 1.5, or from 0.6 to 1.0. In this context, the (w/w) % is based on the weight of the pharmaceutically acceptable implant. In this context, the recombinant protein can be an antibody, an antigen binding fragment, a fusion protein, a cytokine, a transcription factor, an enzyme or a hormone.

A pharmaceutically acceptable implant is provided according to the invention that comprises a xerogel comprising a matrix comprising covalently crosslinked multi-arm precursors comprising a first multi-arm precursor comprising a nucleophile, a second multi-arm precursor comprising an electrophile comprising a first reactive group, and a third multi-arm precursor comprising an electrophile comprising a second reactive group. It also comprises particles comprising a mixture of a recombinant protein and at least one dehydration stabilizer selected from a carbohydrate such as a sugar, such as sucrose and trehalose, a sugar alcohol such as mannitol, or a combination thereof. In one embodiment, the third multi-arm precursor has a longer hydrolysis half-life as compared to the second multi-arm precursor. In this embodiment, the molar ratio of the first reactive group comprised in the second multi-arm precursor, and the second reactive group comprised in the third multi-arm precursor is 30-90:70-10, 40-80:60-20, 50-70:50-30, or 40-60:60-40. In this context, the recombinant protein can be an antibody, an antigen binding fragment, a fusion protein, a cytokine, a transcription factor, an enzyme or a hormone.

A pharmaceutically acceptable implant is provided according to the invention that comprises a xerogel comprising a matrix comprising covalently crosslinked multi-arm precursors comprising a first multi-arm precursor comprising a comprising a primary amine, a second multi-arm precursor comprising succinimidyl succinate, and a third multiarm precursor comprising succinimidyl glutarate. It also comprises particles comprising a mixture of a recombinant protein and at least one dehydration stabilizer selected from a carbohydrate such as a sugar, such as sucrose and trehalose, a sugar alcohol such as mannitol, or a combination thereof. In this embodiment, molar ratio of the succinimidyl succinate group comprised in the second multi-arm precursor, and the succinimidyl glutarate group comprised in the third multi-arm precursor is 30-90:70-10, 40-80:60-20, 50-70:50-30, or 40-60:60-40. In this context, the recombinant protein can be an antibody, an antigen binding fragment, a fusion protein, a cytokine, a transcription factor, an enzyme or a hormone.

A pharmaceutically acceptable implant is provided according to the invention that comprises a xerogel comprising a matrix comprising covalently crosslinked multi-arm precursors comprising a first multi-arm precursor comprising a nucleophile, a second multi-arm precursor comprising an electrophile comprising a first reactive group, and a third multi-arm precursor comprising an electrophile comprising a second reactive group. It also comprises particles comprising a mixture of a recombinant protein and at least one dehydration stabilizer selected from a carbohydrate such as a sugar, such as sucrose and trehalose, a sugar alcohol such as mannitol, or a combination thereof. The total (w/w) % of said dehydration stabilizer is no greater than 60%, no greater than 55%, no greater than 50%, no greater than 45%, or no greater than 40%, or no greater than 30%. The molecular weight between crosslinks in the xerogel is from 7 to 25 kDa, 9 to 20 kDa, or 10 to 15 kDa. The (w/w) % of the total particles comprising a mixture of the recombinant protein and said dehydration stabilizer is no greater than 80%, or no greater than 70%, or no greater than 60%, or no greater than 50% such as from 20% to 40%, from 20% to 30%. The (w/w) % of the total number of multi-arm precursors is from 20% to 80% such as from 35% to 75%, such as from 35% to 65%, from 35% to 55%, from 35% to 45%. The ratio of (1) the (w/w) % of the total particles comprising a mixture of the recombinant protein and said dehydration stabilizer and (2) the (w/w) % of the total number of multi-arm precursors is from 0.3 to 4.0, such as 0.3 to 3.5, from 0.3 to 3.0, from 0.3 to 2.5, from 0.3 to 2.0, from 0.3 to 1.5, from 0.3 to 1.0 or from 0.5 to 4.0, such as 0.5 to 3.5, from 0.5 to 3.0, from 0.5 to 2.5, from 0.5 to 2.0, from 0.5 to 1.5, from 0.5 to 1.0, or from 0.6 to 4.0, 0.6 to 3.5, from 0.6 to 3.0, from 0.6 to 2.5, from 0.6 to 2.0, from 0.6 to 1.5, or from 0.6 to 1.0. In one embodiment, the third multi-arm precursor has a longer hydrolysis half-life as compared to the second multi-arm precursor. In this embodiment, the molar ratio of the first reactive group comprised in the second multiarm precursor, and the second reactive group comprised in the third multi-arm precursor is 30-90:70-10, 40-80:60-20, 50-70:50-30, or 40-60:60-40. In certain embodiments, the Dv₉₀ particle size is from 10 μm to 200 μm such as 35 μm to 75 μm such as 35 μm to 100 μm, or 35 μm to 150 μm, wherein particles comprise the mixture of the recombinant protein and said dehydration stabilizer such as a carbohydrate, a sugar alcohol, or a combination thereof. In this context, the recombinant protein can be an antibody, an antigen binding fragment, a fusion protein, a cytokine, a transcription factor, an enzyme or a hormone.

A pharmaceutically acceptable implant is provided according to the invention that comprises a xerogel comprising a matrix comprising covalently crosslinked multi-arm precursors comprising a first multi-arm precursor comprising a comprising a primary amine, a second multi-arm precursor comprising succinimidyl succinate, and a third multiarm precursor comprising succinimidyl glutarate. It also comprises particles comprising a mixture of a recombinant protein and at least one dehydration stabilizer selected from a carbohydrate such as a sugar, such as sucrose and trehalose, a sugar alcohol such as mannitol, or a combination thereof. The total (w/w) % of said dehydration stabilizer is no greater than 60%, no greater than 55%, no greater than 50%, no greater than 45%, or no greater than 40%, or no greater than 30%. The molecular weight between crosslinks in the xerogel is from 7 to 25 kDa, 9 to 20 kDa, or 10 to 15 kDa. The (w/w) % of the total particles comprising a mixture of the recombinant protein and said dehydration stabilizer is no greater than 80%, or no greater than 70%, or no greater than 60%, or no greater than 50% such as from 20% to 40%, from 20% to 30%. The (w/w) % of the total number of multi-arm precursors is from 20% to 80% such as from 35% to 75%, such as from 35% to 65%, from 35% to 55%, from 35% to 45%. The ratio of (1) the (w/w) % of the total particles comprising a mixture of the recombinant protein and said dehydration stabilizer and (2) the (w/w) % of the total number of multi-arm precursors is from 0.3 to 4.0, such as 0.3 to 3.5, from 0.3 to 3.0, from 0.3 to 2.5, from 0.3 to 2.0, from 0.3 to 1.5, from 0.3 to 1.0 or from 0.5 to 4.0, such as 0.5 to 3.5, from 0.5 to 3.0, from 0.5 to 2.5, from 0.5 to 2.0, from 0.5 to 1.5, from 0.5 to 1.0, or from 0.6 to 4.0, 0.6 to 3.5, from 0.6 to 3.0, from 0.6 to 2.5, from 0.6 to 2.0, from 0.6 to 1.5, or from 0.6 to 1.0. In one embodiment, the third multi-arm precursor has a longer hydrolysis half-life as compared to the second multi-arm precursor. In this embodiment, molar ratio of the succinimidyl succinate group comprised in the second multi-arm precursor, and the succinimidyl glutarate group comprised in the third multi-arm precursor is 30-90:70-10, 40-80:60-20, 50-70:50-30, or 40-60:60-40. In certain embodiments, the Dv₉₀ particle size is from 10 μm to 200 μm such as 35 μm to 75 μm such as 35 μm to 100 μm, or 35 μm to 150 μm, wherein particles comprise the mixture of the recombinant protein and said dehydration stabilizer such as a carbohydrate, a sugar alcohol, or a combination thereof. In this context, the recombinant protein can be an antibody, an antigen binding fragment, a fusion protein, a cytokine, a transcription factor, an enzyme or a hormone.

### Other Agents in the Pharmaceutically Acceptable Implant

The pharmaceutically acceptable implant according to the disclosure in this section may or may not comprise a “non-functional polymer”. The term “non-functional polymer” has been defined previously and refers to any polymer that does not participate in the cross-linking reaction between the precursors. This polymer can be further defined as any polymer that is soluble in both organic solvent and water. In one embodiment, the further polymer is a non-functional polymer used as a bulking agent in the pharmaceutically acceptable implant. The MW of this polymer can be from 1,000 to 35,000 Da, for example from 5,000 to 35,000 Da such as from 5,000 to 10,000 Da, from 7,000 to 10,000 Da, from 8,000 to 15,000 Da, from 8,000 to 25,000 Da, or 5,000 Da or more.

This polymer may be selected from poly(ethylene) oxide, polyethylene glycol, polyvinyl pyrrolidinone, polyvinyl alcohol polyalkylene oxide, methacrylic acid or other vinylic monomers, an acyl chloride, for example methacryloyl chloride, an isocyanate, or 2-isocyanatoethyl methacrylate, an electrophilic poly(ethylene glycol) methacrylate (PEGMA).

The further polymer can be branched (multi-arm) or linear. In the case of a branched polymer, a core refers to a contiguous portion of a molecule joined to arms that extend from the core, with the arms having a functional group, which is often at the terminus of the branch. These polymers may have, e.g., 2-100 arms, with each arm having a terminus, bearing in mind that some precursors may be dendrimers or other highly branched materials. An arm refers to a linear chain of chemical groups that connect a cross linkable functional group to a polymer core. In some embodiments, these further polymers can comprise between 3 and 300 arms; artisans will immediately appreciate that all the ranges and values within the explicitly stated ranges are contemplated, e.g., 4, 6, 8, 10, 12, 4 to 16, 8 to 100, 6, 8, 10, 12, or at least 4 arms.

In various embodiments, this further polymer is selected from a group consisting of polyalkylene oxide such as polyethylene glycol, polyvinyl pyrrolidinone, and polyvinyl alcohol.

### Form of the Implant

The pharmaceutically acceptable implant according to the disclosure in this section can be in the form of a microparticle slurry, in situ-gel, sheet, film, rod, or fiber. Each of these forms represents an embodiment of the invention that can be combined with the disclosure in this section.

In various embodiments, the pharmaceutically acceptable implant is in the form of a fiber. The fiber can be characterized by its diameter and/or length. Furthermore, each of the diameter and the length of the fiber can be further characterized as proximal, mid or distal. The proximal, mid and distal diameters can be same or different. The proximal, mid and distal lengths can be same or different. Thus, the term “diameter of the fiber” refers to the proximal diameter, mid diameter, distal diameter or average of these three diameters. In some embodiments, the term “diameter of the fiber” is an average of all these three diameters. Thus, the term “length of the fiber” refers to the proximal length, mid length, distal length or average of these three lengths. In some embodiments, the term “length of the fiber” is an average of all these three lengths.

In various embodiments, the fiber is characterized by a diameter of about 0.1 mm or more and/or a length of about 2.0 mm or more. In some embodiments, the fiber is characterized by a diameter of about 0.15 mm or more and/or length of about 3.0 mm or more.

### The Total Amount or Concentration of the Biologic

The total amount or concentration of the biologic comprised in the pharmaceutically acceptable implant would depend on the type of biologic.

In one embodiment, the biologic is a virus and is comprised in the pharmaceutically acceptable implant at a total amount of at least 10⁹ vg. In certain embodiments, the virus is comprised in the pharmaceutically acceptable implant at a total amount from 10⁹ to 10¹⁵ vg. In other preferred embodiments, the virus is comprised in the pharmaceutically acceptable implant at a total amount from 10⁹ to 10¹⁵ vg, from 10⁹ to 10¹³ vg, or from 10⁹ to 10¹² vg. In some embodiments, the virus is comprised in the pharmaceutically acceptable implant at a total concentration of at least 10¹³ vg/cm³, such as at least 10¹⁴ vg/cm³. In these embodiments, the virus comprises at least one heterologous nucleic acid sequence. In these embodiments, the heterologous nucleic acid sequence can be either a coding nucleic acid sequence or non-coding nucleic acid sequence. When the heterologous nucleic acid sequence is a coding nucleic acid sequence, it may code for a therapeutic protein as described in the section under the heading of biologic.

In one embodiment, the biologic is an adeno-associated virus (AAV) selected from a group consisting of AAV1, AAV2 such as AAV2.7m8, AAV3, AAV4, AAV5, AAV6, AAV7, AAV8, AAV9, AAV10, AAV11 and a mutant, hybrid or variant thereof and is comprised in the pharmaceutically acceptable implant at a total amount of at least 10⁹ vg. In certain embodiments, the AAV is comprised in the pharmaceutically acceptable implant at a total amount from 10⁹ to 10¹⁵ vg. In other preferred embodiments, the AAV is comprised in the pharmaceutically acceptable implant at a total amount from 10⁹ to 10¹⁵ vg, from 10⁹ to 10¹³ vg, or from 10⁹ to 10¹² vg. In some embodiments, the AAV is comprised in the pharmaceutically acceptable implant at a total concentration of at least 10¹³ vg/cm³, such as at least 10¹⁴ vg/cm³. In these embodiments, the AAV comprises at least one heterologous nucleic acid sequence. In these embodiments, the heterologous nucleic acid sequence can be either a coding nucleic acid sequence or non-coding nucleic acid sequence. When the heterologous nucleic acid sequence is a coding nucleic acid sequence, it may code for a therapeutic protein as described in the section under the heading of biologic. In some embodiments, the total amount of the AAV is in the order from 10¹⁰ to 10¹⁵ vg. In some embodiments, the total amount of the AAV is in the order from 10¹⁰ to 10¹⁴ vg. In some embodiments, the total amount of the AAV is in the order from 10¹⁰ to 10¹³ vg. In some embodiments, the total amount of the AAV is in the order from 10¹⁰ to 10¹² vg. In some embodiments, the total amount of the AAV is in the order from 10¹⁰ to 10¹¹ vg.

In one embodiment, the biologic is a recombinant protein and is comprised in the pharmaceutically acceptable implant at a total amount of at least 10 μg. In certain embodiments, the recombinant protein is comprised in the pharmaceutically acceptable implant at a total amount from 10 to 3000 μg. In other preferred embodiments, the recombinant protein is comprised in the pharmaceutically acceptable implant at a total amount from 10 to 2,500 μg, or from 10 to 2000 μg.

In one embodiment, the biologic is an antibody and is comprised in the pharmaceutically acceptable implant at a total amount of at least 100 μg. In certain embodiments, the antibody is comprised in the pharmaceutically acceptable implant at a total amount from 100 to 3000 μg. In other preferred embodiments, the antibody is comprised in the pharmaceutically acceptable implant at a total amount from 300 to 3000 μg.

In one embodiment, the biologic is an anti-VEGF antibody such as ranibizumab and is comprised in the pharmaceutically acceptable implant at a total amount of at least 500 μg. In certain embodiments, ranibizumab is comprised in the pharmaceutically acceptable implant at a total amount from 500 to 1000 μg. In other preferred embodiments, ranibizumab is comprised in the pharmaceutically acceptable implant at a total amount from 300 to 1000 μg.

In one embodiment, the biologic is an anti-VEGF antibody such as bevacizumab and is comprised in the pharmaceutically acceptable implant at a total amount of at least 1,500 μg. In certain embodiments, bevacizumab is comprised in the pharmaceutically acceptable implant at a total amount from 1,500 to 3000 μg. In other preferred embodiments, bevacizumab is comprised in the pharmaceutically acceptable implant at a total amount from 1,500 to 2,000 μg, or 1,250 μg.

In one embodiment, the biologic is a fusion protein such as aflibercept and is comprised in the pharmaceutically acceptable implant at a total amount of at least 2000 μg. In certain embodiments, aflibercept is comprised in the pharmaceutically acceptable implant at a total amount from 2,000 to 3,000 μg. In other preferred embodiments, aflibercept is comprised in the pharmaceutically acceptable implant at a total amount of 2,000 μg.

In one embodiment, a pharmaceutically acceptable implant of the invention is provided that is characterized in that the implant induces an immune response such as an adaptive immune response such as a humoral immune response as measured by detectable serum ADA titer in a rabbit against the biologic comprised in the implant that is no greater than 20,000, or 15,000, or 10,000, or 8,000, or 7,000, or 5,000, or 2,000, or 1,000 or below detection limit as compared to the serum titer of the ADA at baseline in the rabbit. In one embodiment, the implant is administered to the eye of the rabbit such as an intravitreal administration. In one embodiment, the serum titer of ADA pertains to any time point from week 8 to week 13 post-administration such as at week 8 or at week 13 post-administration. In one embodiment, the serum titer of ADA pertains to a corresponding time point when compared to week 8 or week 13 with the biologic being AAV2.7m8. In one embodiment, the biologic is an adeno-associated virus (AAV) selected from a group consisting of AAV1, AAV2 such as AAV2.7m8, AAV3, AAV4, AAV5, AAV6, AAV7, AAV8, AAV9, AAV10, AAV11 and a mutant, hybrid or variant thereof. In one embodiment, it is comprised in the pharmaceutically acceptable implant at a total amount of at least 10⁹ vg. In certain embodiments, the AAV is comprised in the pharmaceutically acceptable implant at a total amount from 10⁹ to 10¹⁵ vg. In other embodiments, the AAV is comprised in the pharmaceutically acceptable implant at a total amount from 10⁹ to 10¹⁵ vg, from 10⁹ to 10¹³ vg, or from 10⁹ to 10¹² vg. In some embodiments, the AAV is comprised in the pharmaceutically acceptable implant at a total concentration of at least 10¹³ vg/cm³, such as at least 10¹⁴ vg/cm³. In on embodiment, the AAV comprises at least one heterologous nucleic acid sequence. In these embodiments, the heterologous nucleic acid sequence can be either a coding nucleic acid sequence or non-coding nucleic acid sequence. When the heterologous nucleic acid sequence is a coding nucleic acid sequence, it may code for a therapeutic protein as described in the section under the heading of biologic. In some embodiments, the total amount of the AAV is in the order from 10¹⁰ to 10¹⁵ vg. In some embodiments, the total amount of the AAV is in the order from 10¹⁰ to 10¹⁴ vg. In some embodiments, the total amount of the AAV is in the order from 10¹⁰ to 10¹³ vg. In some embodiments, the total amount of the AAV is in the order from 10¹⁰ to 10¹² vg. In some embodiments, the total amount of the AAV is in the order from 10¹⁰ to 10¹¹ vg.

In one embodiment, a pharmaceutically acceptable implant of the invention is provided that is characterized in that it provides a total detectable amount of the biologic in the plasma per mL of a rabbit that is at least four log-less, five log-less, six log-less, seven log-less, eight log-less, nine log less or below detection limit as compared to the total amount of the biologic comprised in the implant. In one embodiment, the implant is administered to the eye of the rabbit such as an intravitreal administration. In one embodiment, the total detectable concentration of the biologic pertains to any time point from day 1 to day 3 post-administration such as at day 2 post-administration. In one embodiment, the total detectable concentration of the biologic pertains to a corresponding time point when compared to any time point from day 1 to day 3 post-administration such as at day 2 post-administration with the biologic being AAV2.7m8. In one embodiment, the biologic is an adeno-associated virus (AAV) selected from a group consisting of AAV1, AAV2 such as AAV2.7m8, AAV3, AAV4, AAV5, AAV6, AAV7, AAV8, AAV9, AAV10, AAV11 and a mutant, hybrid or variant thereof. In one embodiment, it is comprised in the pharmaceutically acceptable implant at a total amount of at least 10⁹ vg. In certain embodiments, the AAV is comprised in the pharmaceutically acceptable implant at a total amount from 10⁹ to 10¹⁵ vg. In other embodiments, the AAV is comprised in the pharmaceutically acceptable implant at a total amount from 10⁹ to 10¹⁵ vg, from 10⁹ to 10¹³ vg, or from 10⁹ to 10¹² vg. In some embodiments, the AAV is comprised in the pharmaceutically acceptable implant at a total concentration of at least 10¹³ vg/cm³, such as at least 10¹⁴ vg/cm³. In on embodiment, the AAV comprises at least one heterologous nucleic acid sequence. In these embodiments, the heterologous nucleic acid sequence can be either a coding nucleic acid sequence or non-coding nucleic acid sequence. When the heterologous nucleic acid sequence is a coding nucleic acid sequence, it may code for a therapeutic protein as described in the section under the heading of biologic. In some embodiments, the total amount of the AAV is in the order from 10¹⁰ to 10¹⁵ vg. In some embodiments, the total amount of the AAV is in the order from 10¹⁰ to 10¹⁴ vg. In some embodiments, the total amount of the AAV is in the order from 10¹⁰ to 10¹³ vg. In some embodiments, the total amount of the AAV is in the order from 10¹⁰ to 10¹² vg. In some embodiments, the total amount of the AAV is in the order from 10¹⁰ to 10¹¹ vg. In one embodiment, a pharmaceutically acceptable implant comprising AAV that comprises a heterologous nucleic acid sequence is provided that is characterized in that it provides a total detectable amount of the heterologous nucleic acid sequence in the plasma per mL of a rabbit that is at least four log-less, five log-less, six log-less, seven log-less, eight log-less, nine log less or below detection limit as compared to the total amount of the AAV in the implant. In one embodiment, the implant is administered to the eye of the rabbit such as an intravitreal administration. In one embodiment, the total detectable concentration of the heterologous nucleic acid sequence pertains to any time point from day 1 to day 3 post-administration such as at day 2 post-administration. In one embodiment, the total detectable concentration of the heterologous nucleic acid sequence pertains to a corresponding time point when compared to any time point from day 1 to day 3 post-administration such as at day 2 post-administration with the biologic being AAV2.7m8. In one embodiment, the biologic is an adeno-associated virus (AAV) selected from a group consisting of AAV1, AAV2 such as AAV2.7m8, AAV3, AAV4, AAV5, AAV6, AAV7, AAV8, AAV9, AAV10, AAV11 and a mutant, hybrid or variant thereof. In one embodiment, it is comprised in the pharmaceutically acceptable implant at a total amount of at least 10⁹ vg. In certain embodiments, the AAV is comprised in the pharmaceutically acceptable implant at a total amount from 10⁹ to 10¹⁵ vg. In other embodiments, the AAV is comprised in the pharmaceutically acceptable implant at a total amount from 10⁹ to 10¹⁵ vg, from 10⁹ to 10¹³ vg, or from 10⁹ to 10¹² vg. In some embodiments, the AAV is comprised in the pharmaceutically acceptable implant at a total concentration of at least 10¹³ vg/cm³, such as at least 10¹⁴ vg/cm³. In on embodiment, the AAV comprises at least one heterologous nucleic acid sequence. In these embodiments, the heterologous nucleic acid sequence can be either a coding nucleic acid sequence or non-coding nucleic acid sequence. When the heterologous nucleic acid sequence is a coding nucleic acid sequence, it may code for a therapeutic protein as described in the section under the heading of biologic. In some embodiments, the total amount of the AAV is in the order from 10¹⁰ to 10¹⁵ vg. In some embodiments, the total amount of the AAV is in the order from 10¹⁰ to 10¹⁴ vg. In some embodiments, the total amount of the AAV is in the order from 10¹⁰ to 10¹³ vg. In some embodiments, the total amount of the AAV is in the order from 10¹⁰ to 10¹² vg. In some embodiments, the total amount of the AAV is in the order from 10¹⁰ to 10¹¹ vg.

In one embodiment, the implant such as a pharmaceutically acceptable implant of the present invention does not comprise a biologic adsorbed to silica particles such as mesoporous silica particles or equivalent thereof.

In one embodiment, the implant such as a pharmaceutically acceptable implant of the present invention does not comprise a biologic adsorbed to a fatty acid component or equivalent thereof.

In one embodiment, the implant such as a pharmaceutically acceptable implant of the present invention does not comprise AAV adsorbed to silica particles such as mesoporous silica particles or equivalent thereof.

In one embodiment, the implant such as a pharmaceutically acceptable implant of the present invention does not comprise AAV adsorbed to a fatty acid component or equivalent thereof.

### a Pharmaceutically Acceptable Implant for a Controlled Release of a Biologic

In one aspect of the present invention, a pharmaceutically acceptable implant is provided for a controlled release of the total amount of the biologic comprised therein. Throughout this section, controlled release is to be considered as the controlled release measured from the time and under conditions wherein the implant is first immersed in an aqueous solution under physiological conditions such as at pH 7.2-7.4 and temperature 37° C. After exposure to physiological conditions, the xerogel comprised in the pharmaceutically acceptable implant forms a hydrogel.

Throughout this section, reference to (w/w) % is to be construed as based on the weight of the pharmaceutically acceptable implant.

Throughout this section, in all of the embodiments including all of the parameters disclosed in this section, “particles” or “total particles” or “Dv₉₀ particle size” is each described as pertaining to particles comprising a mixture of the biologic and at least one dehydration stabilizer such as a carbohydrate, sugar alcohol or combination thereof.

Throughout this section, in all of the embodiments including all of the parameters disclosed in this section, “particles”, or “total particles” or “Dv₉₀ particle size” can also refer to a mixture according to the following paragraphs.

In some embodiments, particles, or total particles in the implant of the invention such as a pharmaceutically acceptable implant of the invention comprise a mixture of the biologic as discussed above and at least one dehydration stabilizer as discussed above.

In some embodiments, particles, or total particles in the implant of the invention such as a pharmaceutically acceptable implant of the invention comprise a mixture of the biologic, at least one dehydration stabilizer, and at least one other stabilizer such as a buffer and a surfactant.

In some embodiments, particles, or total particles in the implant of the invention such as a pharmaceutically acceptable implant of the invention comprise a mixture of the biologic, at least one dehydration stabilizer, and at least one other stabilizer such as a buffer such as PBS comprising at least two or at least three salts, and a non-ionic surfactant such as for example F-68, F-127 or F123.

In some embodiments, particles, or total particles in the implant of the invention such as a pharmaceutically acceptable implant of the invention consist of a mixture of the biologic, at least one dehydration stabilizer, and at least one other stabilizer such as a buffer and a surfactant.

In some embodiments, particles, or total particles in the implant of the invention such as a pharmaceutically acceptable implant of the invention consist of a mixture of the biologic, at least one dehydration stabilizer, and at least one other stabilizer such as a buffer such as PBS comprising at least two or at least three salts, and a non-ionic surfactant such as for example F-68, F-127 or F123.

In some embodiments, particle size such as Dv₉₀ particle size refers to particles in the implant of the invention such as a pharmaceutically acceptable implant of the invention comprising a mixture of the biologic as discussed above and at least one dehydration stabilizer as discussed above.

In some embodiments, particle size such as Dv₉₀ particle size refers to particles in the implant of the invention such as a pharmaceutically acceptable implant of the invention comprising a mixture of the biologic, at least one dehydration stabilizer, and at least one other stabilizer such as a buffer and a surfactant.

In some embodiments, particle size such as Dv₉₀ particle size refers to particles in the implant of the invention such as a pharmaceutically acceptable implant of the invention comprising a mixture of the biologic, at least one dehydration stabilizer, and at least one other stabilizer such as a buffer such as PBS comprising at least two or at least three salts, and a non-ionic surfactant such as for example F-68, F-127 or F123.

In some embodiments, particle size such as Dv₉₀ particle size refers to particles in the implant of the invention such as a pharmaceutically acceptable implant of the invention consisting of a mixture of the biologic, at least one dehydration stabilizer, and at least one other stabilizer such as a buffer and a surfactant.

In some embodiments, particle size such as Dv₉₀ particle size refers to particles in the implant of the invention such as a pharmaceutically acceptable implant of the invention consisting of a mixture of the biologic, at least one dehydration stabilizer, and at least one other stabilizer such as a buffer such as PBS comprising at least two or at least three salts, and a non-ionic surfactant such as for example F-68, F-127 or F123.

According to the invention, a pharmaceutically acceptable implant is provided for a controlled release of the total amount of the biologic comprised therein. The controlled release can be characterized as the amount of the biologic released on day 1 is from 0 to 50% of the total amount of the biologic, the amount of the biologic released per day from day 2 until the last day of release is from 0 to 50% of the total amount of the biologic, and/or the number of days required for 100% release of the total amount of the biologic is at least 2 days

According to the invention, a pharmaceutically acceptable implant is provided for a controlled release of the total amount of the biologic comprised therein. The controlled release can be characterized as the amount of the biologic released on day 1 is from 0 to 50% of the total amount of the biologic, the amount of the biologic released per day from day 2 until the last day of release is from 0 to 50% of the total amount of the biologic, and/or the number of days required for 100% release of the total amount of the biologic is at least 3 days.

According to the invention, a pharmaceutically acceptable implant is provided for a controlled release of the total amount of the biologic comprised therein. The controlled release can be characterized as the amount of the biologic released on day 1 is from 0 to 50% of the total amount of the biologic, the amount of the biologic released per day from day 2 until the last day of release is from 0 to 50% of the total amount of the biologic, and/or the number of days required for 100% release of the total amount of the biologic is at least 4-7 days.

According to the invention, a pharmaceutically acceptable implant is provided for a controlled release of the total amount of the biologic comprised therein The controlled release can be characterized as the amount of the biologic released on day 1 is from 0 to 50% of the total amount of the biologic, the amount of the biologic released per day from day 2 until the last day of release is from 0 to 50% of the total amount of the biologic, and/or, the number of days required for 100% release of the total amount of the biologic is at least 10-15 days.

According to the invention, a pharmaceutically acceptable implant is provided for a controlled release of the total amount of the biologic comprised therein. The controlled release can be characterized as the amount of the biologic released on day 1 is from 0 to 50% of the total amount of the biologic, the amount of the biologic released per day from day 2 until the last day of release is from 0 to 50% of the total amount of the biologic, and/or the number of days required for 100% release of the total amount of the biologic is at least 10-30 days.

According to the invention, a pharmaceutically acceptable implant is provided for a controlled release of the total amount of the biologic comprised therein. The controlled release can be characterized as the amount of the biologic released on day 1 is from 0 to 50% of the total amount of the biologic, the amount of the biologic released per day from day 2 until the last day of release is from 0 to 50% of the total amount of the biologic, and/or the number of days required for 100% release of the total amount of the biologic is greater than 30 days.

According to the invention, a pharmaceutically acceptable implant is provided for a controlled release of the total amount of the biologic comprised therein. The controlled release can be characterized as the amount of the biologic released on day 1 is from 0 to 50% of the total amount of the biologic, the amount of the biologic released per day from day 2 until the last day of release is from 0 to 50% of the total amount of the biologic, and/or the number of days required for 100% release of the total amount of the biologic is greater than or equal to 5 weeks.

According to the invention, a pharmaceutically acceptable implant is provided for a controlled release of the total amount of the biologic comprised therein. The controlled release can be characterized as the amount of the biologic released on day 1 is from 0 to 50% of the total amount of the biologic, the amount of the biologic released per day from day 2 until the last day of release is from 0 to 50% of the total amount of the biologic, and/or the number of days required for 100% release of the total amount of the biologic is about 6 weeks.

According to the invention, the controlled release is characterized by: the amount of the biologic released on day 1 is from 0 to 25%, 0 to 20% 0 to 10%, 0 to 5%, or about 0% of the total amount of the biologic, the amount of the biologic released per day from day 2 until the last day of release is from 0 to 50% of the total amount of the biologic, and/or the number of days required for 100% release of the total amount of the biologic is at least 3 days but no greater than 30 days, 25 days, or no greater than 16 days.

According to the invention, a pharmaceutically acceptable implant is provided for a controlled release of the total amount of the biologic comprised therein. The controlled release can be characterized as the amount of the biologic released on day 1 is from 0 to 50% of the total amount of the biologic, the amount of the biologic released per day from day 2 until the last day of release is from 0 to 50% of the total amount of the biologic, and/or the number of days required for 100% release of the total amount of the biologic is at least 2 days. The controlled release can be characterized as the amount of the biologic released on day 1 is from 0 to 25% of the total amount of the biologic, the amount of the biologic released per day from day 2 until the last day of release is from 0 to 50% of the total amount of the biologic, and/or the number of days required for 100% release of the total amount of the biologic is at least 2 days. The controlled release can be characterized as the amount of the biologic released on day 1 is from 0 to 20% of the total amount of the biologic, the amount of the biologic released per day from day 2 until the last day of release is from 0 to 50% of the total amount of the biologic, and/or the number of days required for 100% release of the total amount of the biologic is at least 2 days. The controlled release can be characterized as the amount of the biologic released on day 1 is from 0 to 10% of the total amount of the biologic, the amount of the biologic released per day from day 2 until the last day of release is from 0 to 50% of the total amount of the biologic, and/or the number of days required for 100% release of the total amount of the biologic is at least 2 days. In this context, the biologic is AAV selected from a group consisting of AAV1, AAV2 such as AAV2.7m8, AAV3, AAV4, AAV5, AAV6, AAV7, AAV8, AAV9, AAV10, AAV11 and a mutant, hybrid or variant thereof at a total amount in the order from 10⁹ to 10¹⁵ vg. In these embodiments, the AAV comprises at least one heterologous nucleic acid sequence. In these embodiments, the heterologous nucleic acid sequence can be either a coding nucleic acid sequence or non-coding nucleic acid sequence. When the heterologous nucleic acid sequence is a coding nucleic acid sequence, it may code for a therapeutic protein as described in the section under the heading of biologic. In some embodiments, the total amount of the AAV is in the order from 10¹⁰ to 10¹⁵ vg. In some embodiments, the total amount of the AAV is in the order from 10¹⁰ to 10¹⁴ vg. In some embodiments, the total amount of the AAV is in the order from 10¹⁰ to 10¹³ vg. In some embodiments, the total amount of the AAV is in the order from 10¹⁰ to 10¹² vg. In some embodiments, the total amount of the AAV is in the order from 10¹⁰ to 10¹¹ vg.

According to the invention, a pharmaceutically acceptable implant is provided for a controlled release of the total amount of the biologic comprised therein. The controlled release can be characterized as the amount of the biologic released on day 1 is from 0 to 50% of the total amount of the biologic, the amount of the biologic released per day from day 2 until the last day of release is from 0 to 50% of the total amount of the biologic, and/or the number of days required for 100% release of the total amount of the biologic is at least 3 days. The controlled release can be characterized as the amount of the biologic released on day 1 is from 0 to 25% of the total amount of the biologic, the amount of the biologic released per day from day 2 until the last day of release is from 0 to 50% of the total amount of the biologic, and/or the number of days required for 100% release of the total amount of the biologic is at least 3 days. The controlled release can be characterized as the amount of the biologic released on day 1 is from 0 to 20% of the total amount of the biologic, the amount of the biologic released per day from day 2 until the last day of release is from 0 to 50% of the total amount of the biologic, and/or the number of days required for 100% release of the total amount of the biologic is at least 3 days. The controlled release can be characterized as the amount of the biologic released on day 1 is from 0 to 10% of the total amount of the biologic, the amount of the biologic released per day from day 2 until the last day of release is from 0 to 50% of the total amount of the biologic, and/or the number of days required for 100% release of the total amount of the biologic is at least 3 days. In this context, the biologic is AAV selected from a group consisting of AAV1, AAV2 such as AAV2.7m8, AAV3, AAV4, AAV5, AAV6, AAV7, AAV8, AAV9, AAV10, AAV11 and a mutant, hybrid or variant thereof at a total amount in the order from 10⁹ to 10¹⁵ vg. In these embodiments, the AAV comprises at least one heterologous nucleic acid sequence. In these embodiments, the heterologous nucleic acid sequence can be either a coding nucleic acid sequence or non-coding nucleic acid sequence. When the heterologous nucleic acid sequence is a coding nucleic acid sequence, it may code for a therapeutic protein as described in the section under the heading of biologic. In some embodiments, the total amount of the AAV is in the order from 10¹⁰ to 10¹⁵ vg. In some embodiments, the total amount of the AAV is in the order from 10¹⁰ to 10¹⁴ vg. In some embodiments, the total amount of the AAV is in the order from 10¹⁰ to 10¹³ vg. In some embodiments, the total amount of the AAV is in the order from 10¹⁰ to 10¹² vg. In some embodiments, the total amount of the AAV is in the order from 10¹⁰ to 10¹¹ vg.

According to the invention, a pharmaceutically acceptable implant is provided for a controlled release of the total amount of the biologic comprised therein. The controlled release can be characterized as the amount of the biologic released on day 1 is from 0 to 50% of the total amount of the biologic, the amount of the biologic released per day from day 2 until the last day of release is from 0 to 50% of the total amount of the biologic, and/or the number of days required for 100% release of the total amount of the biologic is at least 5-7 days. The controlled release can be characterized as the amount of the biologic released on day 1 is from 0 to 25% of the total amount of the biologic, the amount of the biologic released per day from day 2 until the last day of release is from 0 to 50% of the total amount of the biologic, and/or the number of days required for 100% release of the total amount of the biologic is at least 5-7 days. The controlled release can be characterized as the amount of the biologic released on day 1 is from 0 to 20% of the total amount of the biologic, the amount of the biologic released per day from day 2 until the last day of release is from 0 to 50% of the total amount of the biologic, and/or the number of days required for 100% release of the total amount of the biologic is at least 5-7 days. The controlled release can be characterized as the amount of the biologic released on day 1 is from 0 to 10% of the total amount of the biologic, the amount of the biologic released per day from day 2 until the last day of release is from 0 to 50% of the total amount of the biologic, and/or the number of days required for 100% release of the total amount of the biologic is at least 5-7 days. In this context, the biologic is AAV selected from a group consisting of AAV1, AAV2 such as AAV2.7m8, AAV3, AAV4, AAV5, AAV6, AAV7, AAV8, AAV9, AAV10, AAV11 and a mutant, hybrid or variant thereof at a total amount in the order from 10⁹ to 10¹⁵ vg. In these embodiments, the AAV comprises at least one heterologous nucleic acid sequence. In these embodiments, the heterologous nucleic acid sequence can be either a coding nucleic acid sequence or non-coding nucleic acid sequence. When the heterologous nucleic acid sequence is a coding nucleic acid sequence, it may code for a therapeutic protein as described in the section under the heading of biologic. In some embodiments, the total amount of the AAV is in the order from 10¹⁰ to 10¹⁵ vg. In some embodiments, the total amount of the AAV is in the order from 10¹⁰ to 10¹⁴ vg. In some embodiments, the total amount of the AAV is in the order from 10¹⁰ to 10¹³ vg. In some embodiments, the total amount of the AAV is in the order from 10¹⁰ to 10¹² vg. In some embodiments, the total amount of the AAV is in the order from 10¹⁰ to 10¹¹ vg.

According to the invention, a pharmaceutically acceptable implant is provided for a controlled release of the total amount of the biologic comprised therein The controlled release can be characterized as the amount of the biologic released on day 1 is from 0 to 50% of the total amount of the biologic, the amount of the biologic released per day from day 2 until the last day of release is from 0 to 50% of the total amount of the biologic, and/or, the number of days required for 100% release of the total amount of the biologic is at least 10-15 days. The controlled release can be characterized as the amount of the biologic released on day 1 is from 0 to 25% of the total amount of the biologic, the amount of the biologic released per day from day 2 until the last day of release is from 0 to 50% of the total amount of the biologic, and/or the number of days required for 100% release of the total amount of the biologic is at least 10-15 days. The controlled release can be characterized as the amount of the biologic released on day 1 is from 0 to 20% of the total amount of the biologic, the amount of the biologic released per day from day 2 until the last day of release is from 0 to 50% of the total amount of the biologic, and/or the number of days required for 100% release of the total amount of the biologic is at least 10-15 days. The controlled release can be characterized as the amount of the biologic released on day 1 is from 0 to 10% of the total amount of the biologic, the amount of the biologic released per day from day 2 until the last day of release is from 0 to 50% of the total amount of the biologic, and/or the number of days required for 100% release of the total amount of the biologic is at least 10-15 days. In this context, the biologic is AAV selected from a group consisting of AAV1, AAV2 such as AAV2.7m8, AAV3, AAV4, AAV5, AAV6, AAV7, AAV8, AAV9, AAV10, AAV11 and a mutant, hybrid or variant thereof at a total amount in the order from 10⁹ to 10¹⁵ vg. In these embodiments, the AAV comprises at least one heterologous nucleic acid sequence. In these embodiments, the heterologous nucleic acid sequence can be either a coding nucleic acid sequence or non-coding nucleic acid sequence. When the heterologous nucleic acid sequence is a coding nucleic acid sequence, it may code for a therapeutic protein as described in the section under the heading of biologic. In some embodiments, the total amount of the AAV is in the order from 10¹⁰ to 10¹⁵ vg. In some embodiments, the total amount of the AAV is in the order from 10¹⁰ to 10¹⁴ vg. In some embodiments, the total amount of the AAV is in the order from 10¹⁰ to 10¹³ vg. In some embodiments, the total amount of the AAV is in the order from 10¹⁰ to 10¹² vg. In some embodiments, the total amount of the AAV is in the order from 10¹⁰ to 10¹¹ vg.

According to the invention, a pharmaceutically acceptable implant is provided for a controlled release of the total amount of the biologic comprised therein. The controlled release can be characterized as the amount of the biologic released on day 1 is from 0 to 50% of the total amount of the biologic, the amount of the biologic released per day from day 2 until the last day of release is from 0 to 50% of the total amount of the biologic, and/or the number of days required for 100% release of the total amount of the biologic is at least 10-30 days. The controlled release can be characterized as the amount of the biologic released on day 1 is from 0 to 25% of the total amount of the biologic, the amount of the biologic released per day from day 2 until the last day of release is from 0 to 50% of the total amount of the biologic, and/or the number of days required for 100% release of the total amount of the biologic is at least 10-30 days. The controlled release can be characterized as the amount of the biologic released on day 1 is from 0 to 20% of the total amount of the biologic, the amount of the biologic released per day from day 2 until the last day of release is from 0 to 50% of the total amount of the biologic, and/or the number of days required for 100% release of the total amount of the biologic is at least 10-30 days. The controlled release can be characterized as the amount of the biologic released on day 1 is from 0 to 10% of the total amount of the biologic, the amount of the biologic released per day from day 2 until the last day of release is from 0 to 50% of the total amount of the biologic, and/or the number of days required for 100% release of the total amount of the biologic is at least 10-30 days. In this context, the biologic is AAV selected from a group consisting of AAV1, AAV2 such as AAV2.7m8, AAV3, AAV4, AAV5, AAV6, AAV7, AAV8, AAV9, AAV10, AAV11 and a mutant, hybrid or variant thereof at a total amount in the order from 10⁹ to 10¹⁵ vg. In these embodiments, the AAV comprises at least one heterologous nucleic acid sequence. In these embodiments, the heterologous nucleic acid sequence can be either a coding nucleic acid sequence or non-coding nucleic acid sequence. When the heterologous nucleic acid sequence is a coding nucleic acid sequence, it may code for a therapeutic protein as described in the section under the heading of biologic. In some embodiments, the total amount of the AAV is in the order from 10¹⁰ to 10¹⁵ vg. In some embodiments, the total amount of the AAV is in the order from 10¹⁰ to 10¹⁴ vg. In some embodiments, the total amount of the AAV is in the order from 10¹⁰ to 10¹³ vg. In some embodiments, the total amount of the AAV is in the order from 10¹⁰ to 10¹² vg. In some embodiments, the total amount of the AAV is in the order from 10¹⁰ to 10¹¹ vg.

According to the invention, a pharmaceutically acceptable implant is provided for a controlled release of the total amount of the biologic comprised therein. The controlled release can be characterized as the amount of the biologic released on day 1 is from 0 to 50% of the total amount of the biologic, the amount of the biologic released per day from day 2 until the last day of release is from 0 to 50% of the total amount of the biologic, and/or the number of days required for 100% release of the total amount of the biologic is greater than 30 days. The controlled release can be characterized as the amount of the biologic released on day 1 is from 0 to 25% of the total amount of the biologic, the amount of the biologic released per day from day 2 until the last day of release is from 0 to 50% of the total amount of the biologic, and/or the number of days required for 100% release of the total amount of the biologic is greater than 30 days. The controlled release can be characterized as the amount of the biologic released on day 1 is from 0 to 20% of the total amount of the biologic, the amount of the biologic released per day from day 2 until the last day of release is from 0 to 50% of the total amount of the biologic, and/or the number of days required for 100% release of the total amount of the biologic is greater than 30 days. The controlled release can be characterized as the amount of the biologic released on day 1 is from 0 to 10% of the total amount of the biologic, the amount of the biologic released per day from day 2 until the last day of release is from 0 to 50% of the total amount of the biologic, and/or the number of days required for 100% release of the total amount of the biologic is greater than 30 days. In this context, the biologic is AAV selected from a group consisting of AAV1, AAV2 such as AAV2.7m8, AAV3, AAV4, AAV5, AAV6, AAV7, AAV8, AAV9, AAV10, AAV11 and a mutant, hybrid or variant thereof at a total amount in the order from 10⁹ to 10¹⁵ vg. In these embodiments, the AAV comprises at least one heterologous nucleic acid sequence. In these embodiments, the heterologous nucleic acid sequence can be either a coding nucleic acid sequence or non-coding nucleic acid sequence. When the heterologous nucleic acid sequence is a coding nucleic acid sequence, it may code for a therapeutic protein as described in the section under the heading of biologic. In some embodiments, the total amount of the AAV is in the order from 10¹⁰ to 10¹⁵ vg. In some embodiments, the total amount of the AAV is in the order from 10¹⁰ to 10¹⁴ vg. In some embodiments, the total amount of the AAV is in the order from 10¹⁰ to 10¹³ vg. In some embodiments, the total amount of the AAV is in the order from 10¹⁰ to 10¹² vg. In some embodiments, the total amount of the AAV is in the order from 10¹⁰ to 10¹¹ vg.

According to the invention, a pharmaceutically acceptable implant is provided for a controlled release of the total amount of the biologic comprised therein. The controlled release can be characterized as the amount of the biologic released on day 1 is from 0 to 50% of the total amount of the biologic, the amount of the biologic released per day from day 2 until the last day of release is from 0 to 50% of the total amount of the biologic, and/or the number of days required for 100% release of the total amount of the biologic is 5 weeks or greater. The controlled release can be characterized as the amount of the biologic released on day 1 is from 0 to 25% of the total amount of the biologic, the amount of the biologic released per day from day 2 until the last day of release is from 0 to 50% of the total amount of the biologic, and/or the number of days required for 100% release of the total amount of the biologic is 5 weeks or greater. The controlled release can be characterized as the amount of the biologic released on day 1 is from 0 to 20% of the total amount of the biologic, the amount of the biologic released per day from day 2 until the last day of release is from 0 to 50% of the total amount of the biologic, and/or the number of days required for 100% release of the total amount of the biologic is 5 weeks or greater. The controlled release can be characterized as the amount of the biologic released on day 1 is from 0 to 10% of the total amount of the biologic, the amount of the biologic released per day from day 2 until the last day of release is from 0 to 50% of the total amount of the biologic, and/or the number of days required for 100% release of the total amount of the biologic is 5 weeks or greater. In this context, the biologic is AAV selected from a group consisting of AAV1, AAV2 such as AAV2.7m8, AAV3, AAV4, AAV5, AAV6, AAV7, AAV8, AAV9, AAV10, AAV11 and a mutant, hybrid or variant thereof at a total amount in the order from 10⁹ to 10¹⁵ vg. In these embodiments, the AAV comprises at least one heterologous nucleic acid sequence. In these embodiments, the heterologous nucleic acid sequence can be either a coding nucleic acid sequence or non-coding nucleic acid sequence. When the heterologous nucleic acid sequence is a coding nucleic acid sequence, it may code for a therapeutic protein as described in the section under the heading of biologic. In some embodiments, the total amount of the AAV is in the order from 10¹⁰ to 10¹⁵ vg. In some embodiments, the total amount of the AAV is in the order from 10¹⁰ to 10¹⁴ vg. In some embodiments, the total amount of the AAV is in the order from 10¹⁰ to 10¹³ vg. In some embodiments, the total amount of the AAV is in the order from 10¹⁰ to 10¹² vg. In some embodiments, the total amount of the AAV is in the order from 10¹⁰ to 10¹¹ vg.

According to the invention, a pharmaceutically acceptable implant is provided for a controlled release of the total amount of the biologic comprised therein. The controlled release can be characterized as the amount of the biologic released on day 1 is from 0 to 50% of the total amount of the biologic, the amount of the biologic released per day from day 2 until the last day of release is from 0 to 50% of the total amount of the biologic, and/or the number of days required for 100% release of the total amount of the biologic is about 6 weeks. The controlled release can be characterized as the amount of the biologic released on day 1 is from 0 to 25% of the total amount of the biologic, the amount of the biologic released per day from day 2 until the last day of release is from 0 to 50% of the total amount of the biologic, and/or the number of days required for 100% release of the total amount of the biologic is about 6 weeks. The controlled release can be characterized as the amount of the biologic released on day 1 is from 0 to 20% of the total amount of the biologic, the amount of the biologic released per day from day 2 until the last day of release is from 0 to 50% of the total amount of the biologic, and/or the number of days required for 100% release of the total amount of the biologic is about 6 weeks. The controlled release can be characterized as the amount of the biologic released on day 1 is from 0 to 10% of the total amount of the biologic, the amount of the biologic released per day from day 2 until the last day of release is from 0 to 50% of the total amount of the biologic, and/or the number of days required for 100% release of the total amount of the biologic is about 6 weeks. In this context, the biologic is AAV selected from a group consisting of AAV1, AAV2 such as AAV2.7m8, AAV3, AAV4, AAV5, AAV6, AAV7, AAV8, AAV9, AAV10, AAV11 and a mutant, hybrid or variant thereof at a total amount in the order from 10⁹ to 10¹⁵ vg. In these embodiments, the AAV comprises at least one heterologous nucleic acid sequence. In these embodiments, the heterologous nucleic acid sequence can be either a coding nucleic acid sequence or non-coding nucleic acid sequence. When the heterologous nucleic acid sequence is a coding nucleic acid sequence, it may code for a therapeutic protein as described in the section under the heading of biologic. In some embodiments, the total amount of the AAV is in the order from 10¹⁰ to 10¹⁵ vg. In some embodiments, the total amount of the AAV is in the order from 10¹⁰ to 10¹⁴ vg. In some embodiments, the total amount of the AAV is in the order from 10¹⁰ to 10¹³ vg. In some embodiments, the total amount of the AAV is in the order from 10¹⁰ to 10¹² vg. In some embodiments, the total amount of the AAV is in the order from 10¹⁰ to 10¹¹ vg.

According to the invention, the controlled release is characterized by: the amount of the biologic released on day 1 is from 0 to 25%, 0 to 20% 0 to 10%, 0 to 5%, or about 0% of the total amount of the biologic, the amount of the biologic released per day from day 2 until the last day of release is from 0 to 50% of the total amount of the biologic, and/or the number of days required for 100% release of the total amount of the biologic is at least 3 days but no greater than 6 weeks, 5 weeks, 30 days, 25 days, or no greater than 16 days. In this context, the biologic is AAV selected from a group consisting of AAV1, AAV2 such as AAV2.7m8, AAV3, AAV4, AAV5, AAV6, AAV7, AAV8, AAV9, AAV10, AAV11 and a mutant, hybrid or variant thereof at a total amount in the order from 10⁹ to 10¹⁵ vg. In these embodiments, the AAV comprises at least one heterologous nucleic acid sequence. In these embodiments, the heterologous nucleic acid sequence can be either a coding nucleic acid sequence or non-coding nucleic acid sequence. When the heterologous nucleic acid sequence is a coding nucleic acid sequence, it may code for a therapeutic protein as described in the section under the heading of biologic. In some embodiments, the total amount of the AAV is in the order from 10¹⁰ to 10¹⁵ vg. In some embodiments, the total amount of the AAV is in the order from 10¹⁰ to 10¹⁴ vg. In some embodiments, the total amount of the AAV is in the order from 10¹⁰ to 10¹³ vg. In some embodiments, the total amount of the AAV is in the order from 10¹⁰ to 10¹² vg. In some embodiments, the total amount of the AAV is in the order from 10¹⁰ to 10¹¹ vg.

According to the invention, a pharmaceutically acceptable implant is provided for a controlled release of the total amount of the biologic comprised therein. The controlled release can be characterized as the amount of the biologic released on day 1 is from 0 to 50% of the total amount of the biologic, the amount of the biologic released per day from day 2 until the last day of release is from 0 to 50% of the total amount of the biologic, and/or the number of days required for 100% release of the total amount of the biologic is at least 2 days. In this context, the recombinant protein can be an antibody, an antigen binding fragment, a fusion protein, a cytokine, a transcription factor, an enzyme or a hormone.

According to the invention, a pharmaceutically acceptable implant is provided for a controlled release of the total amount of the biologic comprised therein. The controlled release can be characterized as the amount of the biologic released on day 1 is from 0 to 50% of the total amount of the biologic, the amount of the biologic released per day from day 2 until the last day of release is from 0 to 50% of the total amount of the biologic, and/or the number of days required for 100% release of the total amount of the biologic is at least 3 days. In this context, the recombinant protein can be an antibody, an antigen binding fragment, a fusion protein, a cytokine, a transcription factor, an enzyme or a hormone.

According to the invention, a pharmaceutically acceptable implant is provided for a controlled release of the total amount of the biologic comprised therein. The controlled release can be characterized as the amount of the biologic released on day 1 is from 0 to 50% of the total amount of the biologic, the amount of the biologic released per day from day 2 until the last day of release is from 0 to 50% of the total amount of the biologic, and/or the number of days required for 100% release of the total amount of the biologic is at least 5-7 days. In this context, the recombinant protein can be an antibody, an antigen binding fragment, a fusion protein, a cytokine, a transcription factor, an enzyme or a hormone.

According to the invention, a pharmaceutically acceptable implant is provided for a controlled release of the total amount of the biologic comprised therein The controlled release can be characterized as the amount of the biologic released on day 1 is from 0 to 50% of the total amount of the biologic, the amount of the biologic released per day from day 2 until the last day of release is from 0 to 50% of the total amount of the biologic, and/or, the number of days required for 100% release of the total amount of the biologic is at least 10-15 days. In this context, the recombinant protein can be an antibody, an antigen binding fragment, a fusion protein, a cytokine, a transcription factor, an enzyme or a hormone.

According to the invention, a pharmaceutically acceptable implant is provided for a controlled release of the total amount of the biologic comprised therein. The controlled release can be characterized as the amount of the biologic released on day 1 is from 0 to 50% of the total amount of the biologic, the amount of the biologic released per day from day 2 until the last day of release is from 0 to 50% of the total amount of the biologic, and/or the number of days required for 100% release of the total amount of the biologic is at least 10-30 days. In this context, the recombinant protein can be an antibody, an antigen binding fragment, a fusion protein, a cytokine, a transcription factor, an enzyme or a hormone.

According to the invention, a pharmaceutically acceptable implant is provided for a controlled release of the total amount of the biologic comprised therein. The controlled release can be characterized as the amount of the biologic released on day 1 is from 0 to 50% of the total amount of the biologic, the amount of the biologic released per day from day 2 until the last day of release is from 0 to 50% of the total amount of the biologic, and/or the number of days required for 100% release of the total amount of the biologic is greater than 30 days. In this context, the recombinant protein can be an antibody, an antigen binding fragment, a fusion protein, a cytokine, a transcription factor, an enzyme or a hormone.

According to the invention, a pharmaceutically acceptable implant is provided for a controlled release of the total amount of the biologic comprised therein. The controlled release can be characterized as the amount of the biologic released on day 1 is from 0 to 50% of the total amount of the biologic, the amount of the biologic released per day from day 2 until the last day of release is from 0 to 50% of the total amount of the biologic, and/or the number of days required for 100% release of the total amount of the biologic is 5 weeks or greater. In this context, the recombinant protein can be an antibody, an antigen binding fragment, a fusion protein, a cytokine, a transcription factor, an enzyme or a hormone.

According to the invention, a pharmaceutically acceptable implant is provided for a controlled release of the total amount of the biologic comprised therein. The controlled release can be characterized as the amount of the biologic released on day 1 is from 0 to 50% of the total amount of the biologic, the amount of the biologic released per day from day 2 until the last day of release is from 0 to 50% of the total amount of the biologic, and/or the number of days required for 100% release of the total amount of the biologic is about 6 weeks. In this context, the recombinant protein can be an antibody, an antigen binding fragment, a fusion protein, a cytokine, a transcription factor, an enzyme or a hormone.

According to the invention, a pharmaceutically acceptable implant is provided for a controlled release of the total amount of the biologic comprised therein. The controlled release is characterized by: the amount of the biologic released on day 1 is from 0 to 25%, 0 to 20% 0 to 10%, 0 to 5%, or about 0% of the total amount of the biologic, the amount of the biologic released per day from day 2 until the last day of release is from 0 to 50% of the total amount of the biologic, and/or the number of days required for 100% release of the total amount of the biologic is at least 3 days but no greater than 6 weeks, 5 weeks, 30 days, 25 days, or no greater than 16 days. In this context, the recombinant protein can be an antibody, an antigen binding fragment, a fusion protein, a cytokine, a transcription factor, an enzyme or a hormone.

In one embodiment, the controlled release characterized above comprises a zero-order release, such as near zero order release, or substantially zero order release. In one embodiment, the zero-order release or near zero order release or substantially zero order release begins at least 1 day after the pharmaceutically acceptable implant has been immersed under physiological conditions such as pH 7.2-7.4 and 37° C.

A dosage form exhibiting zero order release rate would exhibit a relatively straight line in a graphical representation of percent biologic released versus time. In certain embodiments of the present invention, the zero-order release is accomplished over the entire period of release. In certain embodiments of the present invention, the zero-order release is accomplished over a part of the period of release. In certain such embodiments the zero-order release is accomplished from the end of day 1, i.e., from 24 hours after the start of the release, to the end of the release. If less or no release is accomplished before the end of day 1 such release would be considered to have a lag time for one day or 24 hours. Such a lag time could also be longer. If a high release is accomplished before the end of day 1 such release would be considered to have a burst during the first day or 24 hours. Such a burst time could also be longer. Zero order release can also be accomplished during the entire period of release. The entire period of release is, in this context, defined until 95% of the release is accomplished.

Zero order release is defined to be accomplished, within the meaning of the present invention, if during the respective time the release is proportional to elapsed time. Proportional to elapsed time means that the proportional release is calculated based on the entire time of the zero order release defining a straight line (release in % cumulative release during the entire period of time during which zero order is accomplished divided by said entire period of time defining a straight line) and the release at any time point in between, i.e., start of zero order release and end of zero order release is within 20% points of the % cumulative release of said proportional release defined by said straight line.

According to the invention, a pharmaceutically acceptable implant is provided for a controlled release of the total amount of the biologic comprised therein. This pharmaceutically acceptable implant comprises a xerogel comprising a matrix comprising covalently crosslinked multi-arm precursors comprising a first multi-arm precursor comprising a nucleophile, a second multi-arm precursor comprising an electrophile comprising a first reactive group, and optionally a third multi-arm precursor comprising an electrophile comprising a second reactive group. It also comprises particles comprising a mixture of a biologic and at least one dehydration stabilizer selected from a carbohydrate such as a sugar, such as sucrose and trehalose, a sugar alcohol such as mannitol, or a combination thereof. The inventors have found that the total (w/w) % of the carbohydrate, sugar alcohol or combination thereof in the pharmaceutically acceptable implant provides for the controlled release. In this context, the total (w/w) % of said dehydration stabilizer is no greater than 60%, no greater than 55%, no greater than 50%, no greater than 45%, or no greater than 40%, or no greater than 30%.

According to the invention, a pharmaceutically acceptable implant is provided for a controlled release of the total amount of the biologic comprised therein. This pharmaceutically acceptable implant comprises a xerogel comprising a matrix comprising covalently crosslinked multi-arm precursors comprising a first multi-arm precursor comprising a nucleophile, a second multi-arm precursor comprising an electrophile comprising a first reactive group, and optionally a third multi-arm precursor comprising an electrophile comprising a second reactive group. It also comprises particles comprising a mixture of a biologic and at least one dehydration stabilizer selected from a carbohydrate such as a sugar, such as sucrose and trehalose, a sugar alcohol such as mannitol, or a combination thereof. The inventors have found that the D₉₀ particle size such as Dv₉₀ particle size or Dn₉₀ particle size provides for the controlled release. In this context, the Dv₉₀ particle size is from 10 μm to 200 μm such as 35 μm to 75 μm such as 35 μm to 100 μm, or 35 μm to 150 μm, wherein particles comprise the mixture of the biologic and said dehydration stabilizer such as a carbohydrate, a sugar alcohol, or a combination thereof.

According to the invention, a pharmaceutically acceptable implant is provided for a controlled release of the total amount of the biologic comprised therein. This pharmaceutically acceptable implant comprises a xerogel comprising a matrix comprising covalently crosslinked multi-arm precursors comprising a first multi-arm precursor comprising a nucleophile, a second multi-arm precursor comprising an electrophile comprising a first reactive group, and optionally a third multi-arm precursor comprising an electrophile comprising a second reactive group. It also comprises particles comprising a mixture of a biologic and at least one dehydration stabilizer selected from a carbohydrate such as a sugar, such as sucrose and trehalose, a sugar alcohol such as mannitol, or a combination thereof. The inventors have found that the molecular weight between crosslinks in the xerogel comprised in the pharmaceutically acceptable implant provides for the controlled release. The controlled release provided by this feature is as described previously in this section and pertains to day 1 release, per day release from day 2 onwards, and total number of days required for 100% release of the biologic. In this context, the molecular weight between crosslinks in the xerogel is from 7 to 25 kDa, 9 to 20 kDa, or 10 to 15 kDa.

According to the invention, a pharmaceutically acceptable implant is provided for a controlled release of the total amount of the biologic comprised therein. This pharmaceutically acceptable implant comprises a xerogel comprising a matrix comprising covalently crosslinked multi-arm precursors comprising a first multi-arm precursor comprising a nucleophile, a second multi-arm precursor comprising an electrophile comprising a first reactive group, and optionally a third multi-arm precursor comprising an electrophile comprising a second reactive group. It also comprises particles comprising a mixture of a biologic and at least one dehydration stabilizer selected from a carbohydrate such as a sugar, such as sucrose and trehalose, a sugar alcohol such as mannitol, or a combination thereof. The inventors have found that the (w/w) % of the total particles comprising the mixture of the biologic and the carbohydrate, sugar alcohol or combination thereof in the pharmaceutically acceptable implant provides for the controlled release. The controlled release provided by this feature is as described previously in this section and pertains to day 1 release, per day release from day 2 onwards, and total number of days required for 100% release of the biologic. In this context, the (w/w) % of the total particles comprising a mixture of the biologic and said dehydration stabilizer is no greater than 80%, or no greater than 70%, or no greater than 60%, or no greater than 50% such as from 20% to 40%, from 20% to 30%

According to the invention, a pharmaceutically acceptable implant is provided for a controlled release of the total amount of the biologic comprised therein. This pharmaceutically acceptable implant comprises a xerogel comprising a matrix comprising covalently crosslinked multi-arm precursors comprising a first multi-arm precursor comprising a nucleophile, a second multi-arm precursor comprising an electrophile comprising a first reactive group, and optionally a third multi-arm precursor comprising an electrophile comprising a second reactive group. It also comprises particles comprising a mixture of a biologic and at least one dehydration stabilizer selected from a carbohydrate such as a sugar, such as sucrose and trehalose, a sugar alcohol such as mannitol, or a combination thereof. The inventors have found that the (w/w) % of the total number of multi-arm precursors in the pharmaceutically acceptable implant provides for the controlled release. The controlled release provided by this feature is as described previously in this section and pertains to day 1 release, per day release from day 2 onwards, and total number of days required for 100% release of the biologic. In this context, the (w/w) % of the total number of multi-arm precursors is from 20% to 80% such as from 35% to 75%, such as from 35% to 65%, from 35% to 55%, from 35% to 45%.

According to the invention, a pharmaceutically acceptable implant is provided for a controlled release of the total amount of the biologic comprised therein. This pharmaceutically acceptable implant comprises a xerogel comprising a matrix comprising covalently crosslinked multi-arm precursors comprising a first multi-arm precursor comprising a nucleophile, a second multi-arm precursor comprising an electrophile comprising a first reactive group, and optionally a third multi-arm precursor comprising an electrophile comprising a second reactive group. It also comprises particles comprising a mixture of a biologic and at least one dehydration stabilizer selected from a carbohydrate such as a sugar, such as sucrose and trehalose, a sugar alcohol such as mannitol, or a combination thereof. The inventors have found that the ratio of the (w/w) % of the total particles comprising the mixture of the biologic and the carbohydrate, sugar alcohol or combination thereof in the pharmaceutically acceptable implant and the (w/w) % of the total number of multi-arm precursors in the pharmaceutically acceptable implant provides for the controlled release. The controlled release provided by this feature is as described previously in this section and pertains to day 1 release, per day release from day 2 onwards, and total number of days required for 100% release of the biologic. In this context, the ratio of (1) the (w/w) % of the total particles comprising a mixture of the biologic and said dehydration stabilizer and (2) the (w/w) % of the total number of multi-arm precursors is from 0.3 to 4.0, such as 0.3 to 3.5, from 0.3 to 3.0, from 0.3 to 2.5, from 0.3 to 2.0, from 0.3 to 1.5, from 0.3 to 1.0 or from 0.5 to 4.0, such as 0.5 to 3.5, from 0.5 to 3.0, from 0.5 to 2.5, from 0.5 to 2.0, from 0.5 to 1.5, from 0.5 to 1.0, or from 0.6 to 4.0, 0.6 to 3.5, from 0.6 to 3.0, from 0.6 to 2.5, from 0.6 to 2.0, from 0.6 to 1.5, or from 0.6 to 1.0.

According to the invention, a pharmaceutically acceptable implant is provided for a controlled release of the total amount of the biologic comprised therein. This pharmaceutically acceptable implant comprises a xerogel comprising a matrix comprising covalently crosslinked multi-arm precursors comprising a first multi-arm precursor comprising a nucleophile, a second multi-arm precursor comprising an electrophile comprising a first reactive group, and a third multi-arm precursor comprising an electrophile comprising a second reactive group. It also comprises particles comprising a mixture of a biologic and at least one dehydration stabilizer selected from a carbohydrate such as a sugar, such as sucrose and trehalose, a sugar alcohol such as mannitol, or a combination thereof. The molecular weight between crosslinks in the xerogel comprised in the pharmaceutically acceptable implant provides for the controlled release. The inventors have found that the molar ratio of the first reactive group comprised in the second multi-arm precursor in the pharmaceutically acceptable implant, and the second reactive group comprised in the third multi-arm precursor in the pharmaceutically acceptable implant provides for the controlled release. The controlled release provided by this feature is as described previously in this section and pertains to day 1 release, per day release from day 2 onwards, and total number of days required for 100% release of the biologic. In this context, the third multi-arm precursor has a longer hydrolysis half-life as compared to the second multi-arm precursor. In this context, the molar ratio of the first reactive group comprised in the second multi-arm precursor, and the second reactive group comprised in the third multi-arm precursor is 30-90:70-10, 40-80:60-20, 50-70:50-30, or 40-60:60-40.

According to the invention, a pharmaceutically acceptable implant is provided for a controlled release of the total amount of the biologic comprised therein. This pharmaceutically acceptable implant comprises a xerogel comprising a matrix comprising covalently crosslinked multi-arm precursors comprising a first multi-arm precursor comprising a nucleophile, a second multi-arm precursor comprising an electrophile comprising a first reactive group, and optionally a third multi-arm precursor comprising an electrophile comprising a second reactive group. It also comprises particles comprising a mixture of a biologic and at least one dehydration stabilizer selected from a carbohydrate such as a sugar, such as sucrose and trehalose, a sugar alcohol such as mannitol, or a combination thereof. The inventors have found that if the desired controlled release is characterized such that the number of days required for 100% release of the total amount of the biologic is about 2 days or greater, the total (w/w) % of said dehydration stabilizer should be no greater than 60%, no greater than 55%, no greater than 50%, no greater than 45%, or no greater than 40%, or no greater than 30%. According to the invention, a pharmaceutically acceptable implant is provided for a controlled release of the total amount of the biologic comprised therein. This pharmaceutically acceptable implant comprises a xerogel comprising a matrix comprising covalently crosslinked multi-arm precursors comprising a first multi-arm precursor comprising a nucleophile, a second multi-arm precursor comprising an electrophile comprising a first reactive group, and optionally a third multi-arm precursor comprising an electrophile comprising a second reactive group. It also comprises particles comprising a mixture of a biologic and at least one dehydration stabilizer selected from a carbohydrate such as a sugar, such as sucrose and trehalose, a sugar alcohol such as mannitol, or a combination thereof. The inventors have found that if the desired controlled release is characterized such that the number of days required for 100% release of the total amount of the biologic is about 2 days or greater, the Dv₉₀ particle size is from 10 μm to 200 μm such as 35 μm to 75 μm such as 35 μm to 100 μm, or 35 μm to 150 μm, wherein particles comprise the mixture of the biologic and said dehydration stabilizer such as a carbohydrate, a sugar alcohol, or a combination thereof.

According to the invention, a pharmaceutically acceptable implant is provided for a controlled release of the total amount of the biologic comprised therein. This pharmaceutically acceptable implant comprises a xerogel comprising a matrix comprising covalently crosslinked multi-arm precursors comprising a first multi-arm precursor comprising a nucleophile, a second multi-arm precursor comprising an electrophile comprising a first reactive group, and optionally a third multi-arm precursor comprising an electrophile comprising a second reactive group. It also comprises particles comprising a mixture of a biologic and at least one dehydration stabilizer selected from a carbohydrate such as a sugar, such as sucrose and trehalose, a sugar alcohol such as mannitol, or a combination thereof. The inventors have found that if the desired controlled release is characterized such that the number of days required for 100% release of the total amount of the biologic is about 3 days or greater, the total (w/w) % of said dehydration stabilizer should be no greater than 60%, no greater than 55%, no greater than 50%, no greater than 45%, or no greater than 40%, or no greater than 30%.

According to the invention, a pharmaceutically acceptable implant is provided for a controlled release of the total amount of the biologic comprised therein. This pharmaceutically acceptable implant comprises a xerogel comprising a matrix comprising covalently crosslinked multi-arm precursors comprising a first multi-arm precursor comprising a nucleophile, a second multi-arm precursor comprising an electrophile comprising a first reactive group, and optionally a third multi-arm precursor comprising an electrophile comprising a second reactive group. It also comprises particles comprising a mixture of a biologic and at least one dehydration stabilizer selected from a carbohydrate such as a sugar, such as sucrose and trehalose, a sugar alcohol such as mannitol, or a combination thereof. The inventors have found that if the desired controlled release is characterized such that the number of days required for 100% release of the total amount of the biologic is about 3 days or greater, the Dv₉₀ particle size is from 10 μm to 200 μm such as 35 μm to 75 μm such as 35 μm to 100 μm, or 35 μm to 150 μm, wherein particles comprise the mixture of the biologic and said dehydration stabilizer such as a carbohydrate, a sugar alcohol, or a combination thereof.

According to the invention, a pharmaceutically acceptable implant is provided for a controlled release of the total amount of the biologic comprised therein. This pharmaceutically acceptable implant comprises a xerogel comprising a matrix comprising covalently crosslinked multi-arm precursors comprising a first multi-arm precursor comprising a nucleophile, a second multi-arm precursor comprising an electrophile comprising a first reactive group, and optionally a third multi-arm precursor comprising an electrophile comprising a second reactive group. It also comprises particles comprising a mixture of a biologic and at least one dehydration stabilizer selected from a carbohydrate such as a sugar, such as sucrose and trehalose, a sugar alcohol such as mannitol, or a combination thereof. The inventors have found that if the desired controlled release is characterized such that the number of days required for 100% release of the total amount of the biologic is about 4 days or greater, the total (w/w) % of said dehydration stabilizer should be no greater than 60%, no greater than 55%, no greater than 50%, no greater than 45%, or no greater than 40%, or no greater than 30%.

According to the invention, a pharmaceutically acceptable implant is provided for a controlled release of the total amount of the biologic comprised therein. This pharmaceutically acceptable implant comprises a xerogel comprising a matrix comprising covalently crosslinked multi-arm precursors comprising a first multi-arm precursor comprising a nucleophile, a second multi-arm precursor comprising an electrophile comprising a first reactive group, and optionally a third multi-arm precursor comprising an electrophile comprising a second reactive group. It also comprises particles comprising a mixture of a biologic and at least one dehydration stabilizer selected from a carbohydrate such as a sugar, such as sucrose and trehalose, a sugar alcohol such as mannitol, or a combination thereof. The inventors have found that if the desired controlled release is characterized such that the number of days required for 100% release of the total amount of the biologic is about 4 days or greater, the Dv₉₀ particle size is from 10 μm to 200 μm such as 35 μm to 75 μm such as 35 μm to 100 μm, or 35 μm to 150 μm, wherein particles comprise the mixture of the biologic and said dehydration stabilizer such as a carbohydrate, a sugar alcohol, or a combination thereof.

According to the invention, a pharmaceutically acceptable implant is provided for a controlled release of the total amount of the biologic comprised therein. This pharmaceutically acceptable implant comprises a xerogel comprising a matrix comprising covalently crosslinked multi-arm precursors comprising a first multi-arm precursor comprising a nucleophile, a second multi-arm precursor comprising an electrophile comprising a first reactive group, and optionally a third multi-arm precursor comprising an electrophile comprising a second reactive group. It also comprises particles comprising a mixture of a biologic and at least one dehydration stabilizer selected from a carbohydrate such as a sugar, such as sucrose and trehalose, a sugar alcohol such as mannitol, or a combination thereof. The inventors have found that if the desired controlled release is characterized such that the number of days required for 100% release of the total amount of the biologic is about 2 days or greater, the total (w/w) % of said dehydration stabilizer should be no greater than 60%.

According to the invention, a pharmaceutically acceptable implant is provided for a controlled release of the total amount of the biologic comprised therein. This pharmaceutically acceptable implant comprises a xerogel comprising a matrix comprising covalently crosslinked multi-arm precursors comprising a first multi-arm precursor comprising a nucleophile, a second multi-arm precursor comprising an electrophile comprising a first reactive group, and optionally a third multi-arm precursor comprising an electrophile comprising a second reactive group. It also comprises particles comprising a mixture of a biologic and at least one dehydration stabilizer selected from a carbohydrate such as a sugar, such as sucrose and trehalose, a sugar alcohol such as mannitol, or a combination thereof. The inventors have found that if the desired controlled release is characterized such that the number of days required for 100% release of the total amount of the biologic is about 3 days or greater, the total (w/w) % of said dehydration stabilizer should be no greater than 60%.

According to the invention, a pharmaceutically acceptable implant is provided for a controlled release of the total amount of the biologic comprised therein. This pharmaceutically acceptable implant comprises a xerogel comprising a matrix comprising covalently crosslinked multi-arm precursors comprising a first multi-arm precursor comprising a nucleophile, a second multi-arm precursor comprising an electrophile comprising a first reactive group, and optionally a third multi-arm precursor comprising an electrophile comprising a second reactive group. It also comprises particles comprising a mixture of a biologic and at least one dehydration stabilizer selected from a carbohydrate such as a sugar, such as sucrose and trehalose, a sugar alcohol such as mannitol, or a combination thereof. The inventors have found that if the desired controlled release is characterized such that the number of days required for 100% release of the total amount of the biologic is about 4 days or greater, the total (w/w) % of said dehydration stabilizer should be no greater than 60%.

According to the invention, a pharmaceutically acceptable implant is provided for a controlled release of the total amount of the biologic comprised therein. This pharmaceutically acceptable implant comprises a xerogel comprising a matrix comprising covalently crosslinked multi-arm precursors comprising a first multi-arm precursor comprising a nucleophile, a second multi-arm precursor comprising an electrophile comprising a first reactive group, and optionally a third multi-arm precursor comprising an electrophile comprising a second reactive group. It also comprises particles comprising a mixture of a biologic and at least one dehydration stabilizer selected from a carbohydrate such as a sugar, such as sucrose and trehalose, a sugar alcohol such as mannitol, or a combination thereof. The inventors have found that if the desired controlled release is characterized such that the number of days required for 100% release of the total amount of the biologic is about 2 days or greater, the total (w/w) % of said dehydration stabilizer should be no greater than 50%.

According to the invention, a pharmaceutically acceptable implant is provided for a controlled release of the total amount of the biologic comprised therein. This pharmaceutically acceptable implant comprises a xerogel comprising a matrix comprising covalently crosslinked multi-arm precursors comprising a first multi-arm precursor comprising a nucleophile, a second multi-arm precursor comprising an electrophile comprising a first reactive group, and optionally a third multi-arm precursor comprising an electrophile comprising a second reactive group. It also comprises particles comprising a mixture of a biologic and at least one dehydration stabilizer selected from a carbohydrate such as a sugar, such as sucrose and trehalose, a sugar alcohol such as mannitol, or a combination thereof. The inventors have found that if the desired controlled release is characterized such that the number of days required for 100% release of the total amount of the biologic is about 3 days or greater, the total (w/w) % of said dehydration stabilizer should be no greater than 50%.

According to the invention, a pharmaceutically acceptable implant is provided for a controlled release of the total amount of the biologic comprised therein. This pharmaceutically acceptable implant comprises a xerogel comprising a matrix comprising covalently crosslinked multi-arm precursors comprising a first multi-arm precursor comprising a nucleophile, a second multi-arm precursor comprising an electrophile comprising a first reactive group, and optionally a third multi-arm precursor comprising an electrophile comprising a second reactive group. It also comprises particles comprising a mixture of a biologic and at least one dehydration stabilizer selected from a carbohydrate such as a sugar, such as sucrose and trehalose, a sugar alcohol such as mannitol, or a combination thereof. The inventors have found that if the desired controlled release is characterized such that the number of days required for 100% release of the total amount of the biologic is about 4 days or greater, the total (w/w) % of said dehydration stabilizer should be no greater than 50%.

According to the invention, a pharmaceutically acceptable implant is provided for a controlled release of the total amount of the biologic comprised therein. This pharmaceutically acceptable implant comprises a xerogel comprising a matrix comprising covalently crosslinked multi-arm precursors comprising a first multi-arm precursor comprising a nucleophile, a second multi-arm precursor comprising an electrophile comprising a first reactive group, and optionally a third multi-arm precursor comprising an electrophile comprising a second reactive group. It also comprises particles comprising a mixture of a biologic and at least one dehydration stabilizer selected from a carbohydrate such as a sugar, such as sucrose and trehalose, a sugar alcohol such as mannitol, or a combination thereof. The inventors have found that if the desired controlled release is characterized such that the number of days required for 100% release of the total amount of the biologic is about 2 days or greater, the total (w/w) % of said dehydration stabilizer should be no greater than 40%.

According to the invention, a pharmaceutically acceptable implant is provided for a controlled release of the total amount of the biologic comprised therein. This pharmaceutically acceptable implant comprises a xerogel comprising a matrix comprising covalently crosslinked multi-arm precursors comprising a first multi-arm precursor comprising a nucleophile, a second multi-arm precursor comprising an electrophile comprising a first reactive group, and optionally a third multi-arm precursor comprising an electrophile comprising a second reactive group. It also comprises particles comprising a mixture of a biologic and at least one dehydration stabilizer selected from a carbohydrate such as a sugar, such as sucrose and trehalose, a sugar alcohol such as mannitol, or a combination thereof. The inventors have found that if the desired controlled release is characterized such that the number of days required for 100% release of the total amount of the biologic is about 3 days or greater, the total (w/w) % of said dehydration stabilizer should be no greater than 40%.

According to the invention, a pharmaceutically acceptable implant is provided for a controlled release of the total amount of the biologic comprised therein. This pharmaceutically acceptable implant comprises a xerogel comprising a matrix comprising covalently crosslinked multi-arm precursors comprising a first multi-arm precursor comprising a nucleophile, a second multi-arm precursor comprising an electrophile comprising a first reactive group, and optionally a third multi-arm precursor comprising an electrophile comprising a second reactive group. It also comprises particles comprising a mixture of a biologic and at least one dehydration stabilizer selected from a carbohydrate such as a sugar, such as sucrose and trehalose, a sugar alcohol such as mannitol, or a combination thereof. The inventors have found that if the desired controlled release is characterized such that the number of days required for 100% release of the total amount of the biologic is about 4 days or greater, the total (w/w) % of said dehydration stabilizer should be no greater than 40%.

According to the invention, a pharmaceutically acceptable implant is provided for a controlled release of the total amount of an AAV comprised therein selected from AAV1, AAV2 such as AAV2.7m8, AAV3, AAV4, AAV5, AAV6, AAV7, AAV8, AAV9, AAV10, AAV11 and a mutant, hybrid or variant thereof at a total amount in the order from 10⁹ to 10¹⁵ vg, such as in the order from 10⁹ to 10¹³ vg. In some embodiments, the total amount of the AAV is in the order from 10¹⁰ to 10¹⁵ vg. In some embodiments, the total amount of the AAV is in the order from 10¹⁰ to 10¹⁴ vg. In some embodiments, the total amount of the AAV is in the order from 10¹⁰ to 10¹³ vg. In some embodiments, the total amount of the AAV is in the order from 10¹⁰ to 10¹² vg. In some embodiments, the total amount of the AAV is in the order from 10¹⁰ to 10¹¹ vg. In these embodiments, the AAV comprises at least one heterologous nucleic acid sequence. In these embodiments, the heterologous nucleic acid sequence can be either a coding nucleic acid sequence or non-coding nucleic acid sequence. When the heterologous nucleic acid sequence is a coding nucleic acid sequence, it may code for a therapeutic protein as described in the section under the heading of biologic. The controlled release can be characterized as the amount of the AAV released on day 1 is no greater than 9.0×10⁹ to 1.5×10¹⁰ AAV vg released on day 1, no greater than in the order 10¹¹ vg AAV per day such as in the order 10⁸, or 10⁹ or 10¹⁰ such as 5.0×10⁹ to 1.5×10¹⁰ AAV vg released per day from day 2 until the last day of the controlled release, and/or number of days required for 100% release of the AAV is not less than 4 days.

According to the invention, a pharmaceutically acceptable implant is provided for a controlled release of the total amount of an AAV comprised therein selected from AAV1, AAV2 such as AAV2.7m8, AAV3, AAV4, AAV5, AAV6, AAV7, AAV8, AAV9, AAV10, AAV11 and a mutant, hybrid or variant thereof at a total amount in the order from 10⁹ to 10¹⁵ vg, such as in the order from 10⁹ to 10¹³ vg. In some embodiments, the total amount of the AAV is in the order from 10¹⁰ to 10¹⁵ vg. In some embodiments, the total amount of the AAV is in the order from 10¹⁰ to 10¹⁴ vg. In some embodiments, the total amount of the AAV is in the order from 10¹⁰ to 10¹³ vg. In some embodiments, the total amount of the AAV is in the order from 10¹⁰ to 10¹² vg. In some embodiments, the total amount of the AAV is in the order from 10¹⁰ to 10¹¹ vg. In these embodiments, the AAV comprises at least one heterologous nucleic acid sequence. In these embodiments, the heterologous nucleic acid sequence can be either a coding nucleic acid sequence or non-coding nucleic acid sequence. When the heterologous nucleic acid sequence is a coding nucleic acid sequence, it may code for a therapeutic protein as described in the section under the heading of biologic. The controlled release can be characterized as the amount of the AAV released on day 1 is no greater than 9.0×10⁹ to 1.5×10¹⁰ AAV vg released on day 1, no greater than in the order 10¹¹ vg AAV per day such as in the order 10⁸, or 10⁹ or 10¹⁰ such as 5.0×10⁹ to 1.5×10¹⁰ AAV vg released per day from day 2 until the last day of the controlled release, and/or number of days required for 100% release of the AAV is greater than 7 days.

According to the invention, a pharmaceutically acceptable implant is provided for a controlled release of the total amount of an AAV comprised therein selected from AAV1, AAV2 such as AAV2.7m8, AAV3, AAV4, AAV5, AAV6, AAV7, AAV8, AAV9, AAV10, AAV11 and a mutant, hybrid or variant thereof at a total amount in the order from 10⁹ to 10¹⁵ vg, such as in the order from 10⁹ to 10¹³ vg. In some embodiments, the total amount of the AAV is in the order from 10¹⁰ to 10¹⁵ vg. In some embodiments, the total amount of the AAV is in the order from 10¹⁰ to 10¹⁴ vg. In some embodiments, the total amount of the AAV is in the order from 10¹⁰ to 10¹³ vg. In some embodiments, the total amount of the AAV is in the order from 10¹⁰ to 10¹² vg. In some embodiments, the total amount of the AAV is in the order from 10¹⁰ to 10¹¹ vg. In these embodiments, the AAV comprises at least one heterologous nucleic acid sequence. In these embodiments, the heterologous nucleic acid sequence can be either a coding nucleic acid sequence or non-coding nucleic acid sequence. When the heterologous nucleic acid sequence is a coding nucleic acid sequence, it may code for a therapeutic protein as described in the section under the heading of biologic. The controlled release can be characterized as the amount of the AAV released on day 1 is no greater than 9.0×10⁹ to 1.5×10¹⁰ AAV vg released on day 1, no greater than in the order 10¹¹ vg AAV per day such as in the order 10⁸, or 10⁹ or 10¹⁰ such as 5.0×10⁹ to 1.5×10¹⁰ AAV vg released per day from day 2 until the last day of the controlled release, and/or number of days required for 100% release of the AAV is greater than 14 days.

According to the invention, a pharmaceutically acceptable implant is provided for a controlled release of the total amount of an AAV comprised therein selected from AAV1, AAV2 such as AAV2.7m8, AAV3, AAV4, AAV5, AAV6, AAV7, AAV8, AAV9, AAV10, AAV11 and a mutant, hybrid or variant thereof at a total amount in the order from 10⁹ to 10¹⁵ vg, such as in the order from 10⁹ to 10¹³ vg. In some embodiments, the total amount of the AAV is in the order from 10¹⁰ to 10¹⁵ vg. In some embodiments, the total amount of the AAV is in the order from 10¹⁰ to 10¹⁴ vg. In some embodiments, the total amount of the AAV is in the order from 10¹⁰ to 10¹³ vg. In some embodiments, the total amount of the AAV is in the order from 10¹⁰ to 10¹² vg. In some embodiments, the total amount of the AAV is in the order from 10¹⁰ to 10¹¹ vg. In these embodiments, the AAV comprises at least one heterologous nucleic acid sequence. In these embodiments, the heterologous nucleic acid sequence can be either a coding nucleic acid sequence or non-coding nucleic acid sequence. When the heterologous nucleic acid sequence is a coding nucleic acid sequence, it may code for a therapeutic protein as described in the section under the heading of biologic. The controlled release can be characterized as the amount of the AAV released on day 1 is no greater than 9.0×10⁹ to 1.5×10¹⁰ AAV vg released on day 1, no greater than in the order 10¹¹ vg AAV per day such as in the order 10⁸, or 10⁹ or 10¹⁰ such as 5.0×10⁹ to 1.5×10¹⁰ AAV vg released per day from day 2 until the last day of the controlled release, and/or number of days required for 100% release of the AAV is greater than 25 days.

According to the invention, a pharmaceutically acceptable implant is provided for a controlled release of the total amount of an AAV comprised therein selected from AAV1, AAV2 such as AAV2.7m8, AAV3, AAV4, AAV5, AAV6, AAV7, AAV8, AAV9, AAV10, AAV11 and a mutant, hybrid or variant thereof at a total amount in the order from 10⁹ to 10¹⁵ vg, such as in the order from 10⁹ to 10¹³ vg. In some embodiments, the total amount of the AAV is in the order from 10¹⁰ to 10¹⁵ vg. In some embodiments, the total amount of the AAV is in the order from 10¹⁰ to 10¹⁴ vg. In some embodiments, the total amount of the AAV is in the order from 10¹⁰ to 10¹³ vg. In some embodiments, the total amount of the AAV is in the order from 10¹⁰ to 10¹² vg. In some embodiments, the total amount of the AAV is in the order from 10¹⁰ to 10¹¹ vg. In these embodiments, the AAV comprises at least one heterologous nucleic acid sequence. In these embodiments, the heterologous nucleic acid sequence can be either a coding nucleic acid sequence or non-coding nucleic acid sequence. When the heterologous nucleic acid sequence is a coding nucleic acid sequence, it may code for a therapeutic protein as described in the section under the heading of biologic. The controlled release can be characterized as the amount of the AAV released on day 1 is no greater than 9.0×10⁹ to 1.5×10¹⁰ AAV vg released on day 1, no greater than in the order 10¹¹ vg AAV per day such as in the order 10⁸, or 10⁹ or 10¹⁰ such as 5.0×10⁹ to 1.5×10¹⁰ AAV vg released per day from day 2 until the last day of the controlled release, and/or number of days required for 100% release of the AAV is greater than 30 days.

According to the invention, a pharmaceutically acceptable implant is provided for a controlled release of the total amount of an AAV comprised therein selected from AAV1, AAV2 such as AAV2.7m8, AAV3, AAV4, AAV5, AAV6, AAV7, AAV8, AAV9, AAV10, AAV11 and a mutant, hybrid or variant thereof at a total amount in the order from 10⁹ to 10¹⁵ vg, such as in the order from 10⁹ to 10¹³ vg. In some embodiments, the total amount of the AAV is in the order from 10¹⁰ to 10¹⁵ vg. In some embodiments, the total amount of the AAV is in the order from 10¹⁰ to 10¹⁴ vg. In some embodiments, the total amount of the AAV is in the order from 10¹⁰ to 10¹³ vg. In some embodiments, the total amount of the AAV is in the order from 10¹⁰ to 10¹² vg. In some embodiments, the total amount of the AAV is in the order from 10¹⁰ to 10¹¹ vg. In these embodiments, the AAV comprises at least one heterologous nucleic acid sequence. In these embodiments, the heterologous nucleic acid sequence can be either a coding nucleic acid sequence or non-coding nucleic acid sequence. When the heterologous nucleic acid sequence is a coding nucleic acid sequence, it may code for a therapeutic protein as described in the section under the heading of biologic. The controlled release can be characterized as the amount of the AAV released on day 1 is no greater than 9.0×10⁹ to 1.5×10¹⁰ AAV vg released on day 1, no greater than in the order 10¹¹ vg AAV per day such as in the order 10⁸, or 10⁹ or 10¹⁰ such as 5.0×10⁹ to 1.5×10¹⁰ AAV vg released per day from day 2 until the last day of the controlled release, and/or number of days required for 100% release of the AAV is 5 weeks or greater.

According to the invention, a pharmaceutically acceptable implant is provided for a controlled release of the total amount of an AAV comprised therein selected from AAV1, AAV2 such as AAV2.7m8, AAV3, AAV4, AAV5, AAV6, AAV7, AAV8, AAV9, AAV10, AAV11 and a mutant, hybrid or variant thereof at a total amount in the order from 10⁹ to 10¹⁵ vg, such as in the order from 10⁹ to 10¹³ vg. In some embodiments, the total amount of the AAV is in the order from 10¹⁰ to 10¹⁵ vg. In some embodiments, the total amount of the AAV is in the order from 10¹⁰ to 10¹⁴ vg. In some embodiments, the total amount of the AAV is in the order from 10¹⁰ to 10¹³ vg. In some embodiments, the total amount of the AAV is in the order from 10¹⁰ to 10¹² vg. In some embodiments, the total amount of the AAV is in the order from 10¹⁰ to 10¹¹ vg. In these embodiments, the AAV comprises at least one heterologous nucleic acid sequence. In these embodiments, the heterologous nucleic acid sequence can be either a coding nucleic acid sequence or non-coding nucleic acid sequence. When the heterologous nucleic acid sequence is a coding nucleic acid sequence, it may code for a therapeutic protein as described in the section under the heading of biologic. The controlled release can be characterized as the amount of the AAV released on day 1 is no greater than 9.0×10⁹ to 1.5×10¹⁰ AAV vg released on day 1, no greater than in the order 10¹¹ vg AAV per day such as in the order 10⁸, or 10⁹ or 10¹⁰ such as 5.0×10⁹ to 1.5×10¹⁰ AAV vg released per day from day 2 until the last day of the controlled release, and/or number of days required for 100% release of the AAV is about 6 weeks.

According to the invention, a pharmaceutically acceptable implant is provided for a controlled release of the total amount of an AAV comprised therein selected from AAV1, AAV2 such as AAV2.7m8, AAV3, AAV4, AAV5, AAV6, AAV7, AAV8, AAV9, AAV10, AAV11 and a mutant, hybrid or variant thereof at a total amount in the order from 10⁹ to 10¹⁵ vg, such as in the order from 10⁹ to 10¹³ vg. In some embodiments, the total amount of the AAV is in the order from 10¹⁰ to 10¹⁵ vg. In some embodiments, the total amount of the AAV is in the order from 10¹⁰ to 10¹⁴ vg. In some embodiments, the total amount of the AAV is in the order from 10¹⁰ to 10¹³ vg. In some embodiments, the total amount of the AAV is in the order from 10¹⁰ to 10¹² vg. In some embodiments, the total amount of the AAV is in the order from 10¹⁰ to 10¹¹ vg. In these embodiments, the AAV comprises at least one heterologous nucleic acid sequence. In these embodiments, the heterologous nucleic acid sequence can be either a coding nucleic acid sequence or non-coding nucleic acid sequence. When the heterologous nucleic acid sequence is a coding nucleic acid sequence, it may code for a therapeutic protein as described in the section under the heading of biologic. The controlled release can be characterized as the amount of the AAV released on day 1 is no greater than 9.0×10⁹ to 1.5×10¹⁰ AAV vg released on day 1, no greater than in the order 10¹¹ vg AAV per day such as in the order 10⁸, or 10⁹ or 10¹⁰ such as 5.0×10⁹ to 1.5×10¹⁰ AAV vg released per day from day 2 until the last day of the controlled release, and/or number of days required for 100% release of the AAV is greater than 30 days. This pharmaceutically acceptable implant discussed in the previous paragraphs comprises a xerogel comprising a matrix comprising covalently crosslinked multi-arm precursors comprising a first multi-arm precursor comprising a nucleophile, a second multi-arm precursor comprising an electrophile comprising a first reactive group, and optionally a third multi-arm precursor comprising an electrophile comprising a second reactive group. It also comprises particles comprising a mixture of AAV and at least one dehydration stabilizer selected from a carbohydrate such as a sugar, such as sucrose and trehalose, a sugar alcohol such as mannitol, or a combination thereof. This pharmaceutically acceptable implant comprises the total (w/w) % of said dehydration stabilizer no greater than 60%, no greater than 55%, no greater than 50%, no greater than 45%, or no greater than 40%, or no greater than 30%. Further, this pharmaceutically acceptable implant also comprises the molecular weight between crosslinks in the xerogel from 7 to 25 kDa, 9 to 20 kDa, or 10 to 15 kDa.

This pharmaceutically acceptable implant discussed in the previous paragraphs comprises a xerogel comprising a matrix comprising covalently crosslinked multi-arm precursors comprising a first multi-arm precursor comprising a nucleophile, a second multiarm precursor comprising an electrophile comprising a first reactive group, and optionally a third multi-arm precursor comprising an electrophile comprising a second reactive group. It also comprises particles comprising a mixture of AAV and at least one dehydration stabilizer selected from a carbohydrate such as a sugar, such as sucrose and trehalose, a sugar alcohol such as mannitol, or a combination thereof. This pharmaceutically acceptable implant comprises the total (w/w) % of said dehydration stabilizer no greater than 60%, no greater than 55%, no greater than 50%, no greater than 45%, or no greater than 40%, or no greater than 30%. Further, this pharmaceutically acceptable implant also comprises the (w/w) % of the total particles comprising a mixture of said AAV and said dehydration stabilizer at no greater than 80%, or no greater than 70%, or no greater than 60%, or no greater than 50% such as from 20% to 40%, from 20% to 30%. This pharmaceutically acceptable implant discussed in the previous paragraphs comprises a xerogel comprising a matrix comprising covalently crosslinked multi-arm precursors comprising a first multi-arm precursor comprising a nucleophile, a second multiarm precursor comprising an electrophile comprising a first reactive group, and optionally a third multi-arm precursor comprising an electrophile comprising a second reactive group. It also comprises particles comprising a mixture of AAV and at least one dehydration stabilizer selected from a carbohydrate such as a sugar, such as sucrose and trehalose, a sugar alcohol such as mannitol, or a combination thereof. This pharmaceutically acceptable implant comprises the total (w/w) % of said dehydration stabilizer no greater than 60%, no greater than 55%, no greater than 50%, no greater than 45%, or no greater than 40%, or no greater than 30%. Further, this pharmaceutically acceptable implant also comprises the (w/w) % of the total number of multi-arm precursors at 20% to 80% such as from 35% to 75%, such as from 35% to 65%, from 35% to 55%, from 35% to 45%. This pharmaceutically acceptable implant discussed in the previous paragraphs comprises a xerogel comprising a matrix comprising covalently crosslinked multiarm precursors comprising a first multi-arm precursor comprising a nucleophile, a second multi-arm precursor comprising an electrophile comprising a first reactive group, and optionally a third multi-arm precursor comprising an electrophile comprising a second reactive group. It also comprises particles comprising a mixture of AAV and at least one dehydration stabilizer selected from a carbohydrate such as a sugar, such as sucrose and trehalose, a sugar alcohol such as mannitol, or a combination thereof. This pharmaceutically acceptable implant comprises the total (w/w) % of said dehydration stabilizer no greater than 60%, no greater than 55%, no greater than 50%, no greater than 45%, or no greater than 40%, or no greater than 30%. Further, this pharmaceutically acceptable implant also comprises the ratio of (1) the (w/w) % of the total particles comprising a mixture of the biologic and said dehydration stabilizer and (2) the (w/w) % of the total number of multi-arm precursors is from 0.3 to 4.0, such as 0.3 to 3.5, from 0.3 to 3.0, from 0.3 to 2.5, from 0.3 to 2.0, from 0.3 to 1.5, from 0.3 to 1.0 or from 0.5 to 4.0, such as 0.5 to 3.5, from 0.5 to 3.0, from 0.5 to 2.5, from 0.5 to 2.0, from 0.5 to 1.5, from 0.5 to 1.0, or from 0.6 to 4.0, 0.6 to 3.5, from 0.6 to 3.0, from 0.6 to 2.5, from 0.6 to 2.0, from 0.6 to 1.5, or from 0.6 to 1.0. This pharmaceutically acceptable implant discussed in the previous paragraphs comprises a xerogel comprising a matrix comprising covalently crosslinked multi-arm precursors comprising a first multi-arm precursor comprising a nucleophile, a second multi-arm precursor comprising an electrophile comprising a first reactive group, and a third multi-arm precursor comprising an electrophile comprising a second reactive group. It also comprises particles comprising a mixture of AAV and at least one dehydration stabilizer selected from a carbohydrate such as a sugar, such as sucrose and trehalose, a sugar alcohol such as mannitol, or a combination thereof. This pharmaceutically acceptable implant comprises the total (w/w) % of said dehydration stabilizer no greater than 60%, no greater than 55%, no greater than 50%, no greater than 45%, or no greater than 40%, or no greater than 30%. Further, this pharmaceutically acceptable implant also comprises the molar ratio of the first reactive group comprised in the second multi-arm precursor, and the second reactive group comprised in the third multi-arm precursor is 30-90:70-10, 40-80:60-20, 50-70:50-30, or 40-60:60-40. This pharmaceutically acceptable implant discussed in the previous paragraphs comprises a xerogel comprising a matrix comprising covalently crosslinked multi-arm precursors comprising a first multi-arm precursor comprising a nucleophile, a second multi-arm precursor comprising an electrophile comprising a first reactive group, and a third multiarm precursor comprising an electrophile comprising a second reactive group. It also comprises particles comprising a mixture of AAV and at least one dehydration stabilizer selected from a carbohydrate such as a sugar, such as sucrose and trehalose, a sugar alcohol such as mannitol, or a combination thereof. The Dv₉₀ particle size is from 10 μm to 200 μm such as 35 μm to 75 μm such as 35 μm to 100 μm, or 35 μm to 150 μm, wherein particles comprise the mixture of the AAV and said dehydration stabilizer such as a carbohydrate, a sugar alcohol, or a combination thereof.

In one embodiment, a pharmaceutically acceptable implant for controlled release of a total amount of a biologic is provided that is characterized in that the implant induces an immune response such as an adaptive immune response such as a humoral immune response as measured by detectable serum ADA titer in a rabbit against the biologic comprised in the implant that is no greater than 20,000, or 15,000, or 10,000, or 8,000, or 7,000, or 5,000, or 2,000, or 1,000 or below detection limit as compared to the serum titer of the ADA at baseline in the rabbit. In one embodiment, the implant is administered to the eye of the rabbit such as an intravitreal administration. In one embodiment, the serum titer of ADA pertains to any time point from week 8 to week 13 post-administration such as at week 8 or at week 13 post-administration. In one embodiment, the serum titer of ADA pertains to a corresponding time point when compared to week 8 or week 13 with the biologic being AAV2.7m8. In one embodiment, the biologic is an adeno-associated virus (AAV) selected from a group consisting of AAV1, AAV2 such as AAV2.7m8, AAV3, AAV4, AAV5, AAV6, AAV7, AAV8, AAV9, AAV10, AAV11 and a mutant, hybrid or variant thereof. In one embodiment, it is comprised in the pharmaceutically acceptable implant at a total amount of at least 10⁹ vg. In certain embodiments, the AAV is comprised in the pharmaceutically acceptable implant at a total amount from 10⁹ to 10¹⁵ vg. In other embodiments, the AAV is comprised in the pharmaceutically acceptable implant at a total amount from 10⁹ to 10¹⁵ vg, from 10⁹ to 10¹³ vg, or from 10⁹ to 10¹² vg. In some embodiments, the AAV is comprised in the pharmaceutically acceptable implant at a total concentration of at least 10¹³ vg/cm³, such as at least 10¹⁴ vg/cm³. In some embodiments, the AAV is comprised in the pharmaceutically acceptable implant at a total amount from 10¹⁰ to 10¹⁵ vg. In some embodiments, the AAV is comprised in the pharmaceutically acceptable implant at a total amount from 10¹⁰ to 10¹⁴ vg. In some embodiments, the AAV is comprised in the pharmaceutically acceptable implant at a total amount from 10¹⁰ to 10¹³ vg. In some embodiments, the AAV is comprised in the pharmaceutically acceptable implant at a total amount from 10¹⁰ to 10¹² vg. In some embodiments, the AAV is comprised in the pharmaceutically acceptable implant at a total amount from 10¹⁰ to 10¹¹ vg. In on embodiment, the AAV comprises at least one heterologous nucleic acid sequence. In these embodiments, the heterologous nucleic acid sequence can be either a coding nucleic acid sequence or non-coding nucleic acid sequence. When the heterologous nucleic acid sequence is a coding nucleic acid sequence, it may code for a therapeutic protein as described in the section under the heading of biologic.

In one embodiment, a pharmaceutically acceptable implant for controlled release of a total amount of a biologic is provided is provided that is characterized in that it provides a total detectable amount of the biologic in the plasma per mL of a rabbit that is at least four log-less, five log-less, six log-less, seven log-less, eight log-less, nine log less or below detection limit as compared to the total amount of the biologic comprised in the implant. In one embodiment, the implant is administered to the eye of the rabbit such as an intravitreal administration. In one embodiment, the total detectable concentration of the biologic pertains to any time point from day 1 to day 3 post-administration such as at day 2 post-administration. In one embodiment, the total detectable concentration of the biologic pertains to a corresponding time point when compared to any time point from day 1 to day 3 post-administration such as at day 2 post-administration with the biologic being AAV2.7m8. In one embodiment, the biologic is an adeno-associated virus (AAV) selected from a group consisting of AAV1, AAV2 such as AAV2.7m8, AAV3, AAV4, AAV5, AAV6, AAV7, AAV8, AAV9, AAV10, AAV11 and a mutant, hybrid or variant thereof. In one embodiment, it is comprised in the pharmaceutically acceptable implant at a total amount of at least 10⁹ vg. In certain embodiments, the AAV is comprised in the pharmaceutically acceptable implant at a total amount from 10⁹ to 10¹⁵ vg. In other embodiments, the AAV is comprised in the pharmaceutically acceptable implant at a total amount from 10⁹ to 10¹⁵ vg, from 10⁹ to 10¹³ vg, or from 10⁹ to 10¹² vg. In some embodiments, the AAV is comprised in the pharmaceutically acceptable implant at a total concentration of at least 10¹³ vg/cm³, such as at least 10¹⁴ vg/cm³. In some embodiments, the AAV is comprised in the pharmaceutically acceptable implant at a total amount from 10¹⁰ to 10¹⁵ vg. In some embodiments, the AAV is comprised in the pharmaceutically acceptable implant at a total amount from 10¹⁰ to 10¹⁴ vg. In some embodiments, the AAV is comprised in the pharmaceutically acceptable implant at a total amount from 10¹⁰ to 10¹³ vg. In some embodiments, the AAV is comprised in the pharmaceutically acceptable implant at a total amount from 10¹⁰ to 10¹² vg. In some embodiments, the AAV is comprised in the pharmaceutically acceptable implant at a total amount from 10¹⁰ to 10¹¹ vg. In on embodiment, the AAV comprises at least one heterologous nucleic acid sequence. In these embodiments, the heterologous nucleic acid sequence can be either a coding nucleic acid sequence or non-coding nucleic acid sequence. When the heterologous nucleic acid sequence is a coding nucleic acid sequence, it may code for a therapeutic protein as described in the section under the heading of biologic.

In one embodiment, a pharmaceutically acceptable implant comprising AAV that comprises a heterologous nucleic acid sequence for controlled release of a total amount of the AAV is provided that is characterized in that it provides a total detectable amount of the heterologous nucleic acid sequence in the plasma per ml of a rabbit that is at least four log-less, five log-less, six log-less, seven log-less, eight log-less, nine log less or below detection limit as compared to the total amount of the AAV in the implant. In one embodiment, the implant is administered to the eye of the rabbit such as an intravitreal administration. In one embodiment, the total detectable concentration of the heterologous nucleic acid sequence pertains to any time point from day 1 to day 3 post-administration such as at day 2 post-administration. In one embodiment, the total detectable concentration of the heterologous nucleic acid sequence pertains to a corresponding time point when compared to any time point from day 1 to day 3 post-administration such as at day 2 post-administration with the biologic being AAV2.7m8. In one embodiment, the biologic is an adeno-associated virus (AAV) selected from a group consisting of AAV1, AAV2 such as AAV2.7m8, AAV3, AAV4, AAV5, AAV6, AAV7, AAV8, AAV9, AAV10, AAV11 and a mutant, hybrid or variant thereof. In one embodiment, it is comprised in the pharmaceutically acceptable implant at a total amount of at least 10⁹ vg. In certain embodiments, the AAV is comprised in the pharmaceutically acceptable implant at a total amount from 10⁹ to 10¹⁵ vg. In other embodiments, the AAV is comprised in the pharmaceutically acceptable implant at a total amount from 10⁹ to 10¹⁵ vg, from 10⁹ to 10¹³ vg, or from 10⁹ to 10¹² vg. In some embodiments, the AAV is comprised in the pharmaceutically acceptable implant at a total concentration of at least 10¹³ vg/cm³, such as at least 10¹⁴ vg/cm³. In some embodiments, the AAV is comprised in the pharmaceutically acceptable implant at a total amount from 10¹⁰ to 10¹⁵ vg. In some embodiments, the AAV is comprised in the pharmaceutically acceptable implant at a total amount from 10¹⁰ to 10¹⁴ vg. In some embodiments, the AAV is comprised in the pharmaceutically acceptable implant at a total amount from 10¹⁰ to 10¹³ vg. In some embodiments, the AAV is comprised in the pharmaceutically acceptable implant at a total amount from 10¹⁰ to 10¹² vg. In some embodiments, the AAV is comprised in the pharmaceutically acceptable implant at a total amount from 10¹⁰ to 10¹¹ vg. In one embodiment, the AAV comprises at least one heterologous nucleic acid sequence. In these embodiments, the heterologous nucleic acid sequence can be either a coding nucleic acid sequence or non-coding nucleic acid sequence. When the heterologous nucleic acid sequence is a coding nucleic acid sequence, it may code for a therapeutic protein as described in the section under the heading of biologic.

In one embodiment, the pharmaceutically acceptable implant for controlled release of the biologic is in the form of a fiber. In some embodiments, the fiber changes its size during the period of controlled release. The size of the fiber can be characterized by its diameter and/or length. Furthermore, each of the diameter and the length of the fiber can be further characterized as proximal, mid or distal. The proximal, mid and distal diameters can be same or different. The proximal, mid and distal lengths can be same or different. Thus, the term “diameter of the fiber” refers to the proximal diameter, mid diameter, distal diameter or average of these three diameters. In some embodiments, the term “diameter of the fiber” is an average of all these three diameters. Thus, the term “length of the fiber” refers to the proximal length, mid length, distal length or average of these three lengths. In some embodiments, the term “length of the fiber” is an average of all these three lengths.

In various embodiments, the fiber is characterized by a diameter of about 0.1 mm or more and/or a length of about 2.0 mm or more before it is first immersed under physiological conditions (in vitro or in vivo) such as an aqueous solution at pH 7.2-7.4 and 37° C. Once the fiber is immersed under physiological conditions, which is also the time point when the controlled release begins, the fiber may change its diameter and/or length. Thus, in some embodiments, the fiber changes its length and/or diameter at any time point during the controlled release.

In one embodiment, the diameter and/or length of the fiber increases by about 1.5-fold or about 2 folds at any time point during the controlled release as measured from the time and under conditions when it is first immersed under physiological conditions.

In some embodiments, the length of the fibre may not change at all during the controlled release as measured from the time and under conditions when it is first immersed under physiological conditions. In such embodiments, the fiber has been, for example, stretched before it has been immersed under physiological conditions.

In some embodiments, the length and/or diameter of the fiber at one time point during the controlled release decreases as compared to the length and/or diameter of the fiber at an earlier time point during the controlled release.

In one embodiment, the implant such as a pharmaceutically acceptable implant of the present invention for controlled release of a biologic does not comprise a biologic adsorbed to silica particles such as mesoporous silica particles or equivalent thereof.

In one embodiment, the implant such as a pharmaceutically acceptable implant of the present invention for controlled release of a biologic does not comprise a biologic adsorbed to a fatty acid component or equivalent thereof.

In one embodiment, the implant such as a pharmaceutically acceptable implant of the present invention for controlled release of AAV does not comprise AAV adsorbed to silica particles such as mesoporous silica particles or equivalent thereof.

In one embodiment, the implant such as a pharmaceutically acceptable implant of the present invention for controlled release of AAV does not comprise AAV adsorbed to a fatty acid component or equivalent thereof.

### Methods of Treatment

A method of treating an ocular disorder, such as an ocular genetic disorder is also provided comprising administering to a subject a pharmaceutically acceptable implant discussed in a separate section herewith. This pharmaceutically acceptable implant is for controlled release of any biologic that has been discussed in a separate section herewith. The controlled release is also defined herewith in a separate section.

In one embodiment, the biologic is a recombinant protein such as an antibody, an antigen binding fragment, a fusion protein, a cytokine, a transcription factor, an enzyme or a hormone. In this embodiment, the controlled release in the method of treatment is characterized by the amount of the biologic released on day 1 is from 0 to 50% of the total amount of the biologic, the amount of the biologic released per day from day 2 until the last day of release is from 0 to 50% of the total amount of the biologic, and/or the number of days required for 100% release of the total amount of the biologic is at least 2 days. In some embodiments, the number of days required for 100% release of the total amount of the biologic is at least 3 days, or at least 4 days. In this embodiment, the controlled release in the method of treatment is characterized by the amount of the biologic released on day 1 is from 0 to 25% of the total amount of the biologic, the amount of the biologic released per day from day 2 until the last day of release is from 0 to 50% of the total amount of the biologic, and/or the number of days required for 100% release of the total amount of the biologic is at least 2 days. In some embodiments, the number of days required for 100% release of the total amount of the biologic is at least 3 days, or at least 4 days. In this embodiment, the controlled release in the method of treatment is characterized by the amount of the biologic released on day 1 is from 0 to 20% of the total amount of the biologic, the amount of the biologic released per day from day 2 until the last day of release is from 0 to 50% of the total amount of the biologic, and/or the number of days required for 100% release of the total amount of the biologic is at least 2 days. In some embodiments, the number of days required for 100% release of the total amount of the biologic is at least 3 days, or at least 4 days. In this embodiment, the controlled release in the method of treatment is characterized by the amount of the biologic released on day 1 is from 0 to 10% of the total amount of the biologic, the amount of the biologic released per day from day 2 until the last day of release is from 0 to 50% of the total amount of the biologic, and/or the number of days required for 100% release of the total amount of the biologic is at least 2 days. In some embodiments, the number of days required for 100% release of the total amount of the biologic is at least 3 days, or at least 4 days

In another embodiment, the biologic is a virus. In some embodiments, the virus is an adeno-associated virus (AAV) selected from a group consisting of AAV1, AAV2 such as AAV2.7m8, AAV3, AAV4, AAV5, AAV6, AAV7, AAV8, AAV9, AAV10, AAV11 and a mutant, hybrid or variant thereof at a total amount in the order from 10⁹ to 10¹⁵ vg, such as in the order from 10⁹ to 10¹³ vg. In some embodiments, the total amount of the AAV is from 10¹⁰ to 10¹⁵ vg. In some embodiments, the total amount of the AAV is from 10¹⁰ to 10¹⁴ vg. In some embodiments, the total amount of the AAV is from 10¹⁰ to 10¹³ vg. In some embodiments, the total amount of the AAV is from 10¹⁰ to 10¹² vg. In some embodiments, the total amount of the AAV is from 10¹⁰ to 10¹¹ vg. In these embodiments, the AAV comprises at least one heterologous nucleic acid sequence. In these embodiments, the heterologous nucleic acid sequence can be either a coding nucleic acid sequence or non-coding nucleic acid sequence. When the heterologous nucleic acid sequence is a coding nucleic acid sequence, it may code for a therapeutic protein as described in the section under the heading of biologic. In certain embodiments, the heterologous nucleic acid(s) code(s) for a therapeutic protein, which is absent in the subject, or present at a reduced level in the subject as compared to the same but healthy subject. In one embodiment, the controlled release is characterized by the amount of the AAV released on day 1 is from 0 to 50% of the total amount of the AAV, the amount of the AAV released per day from day 2 until the last day of release is from 0 to 50% of the total amount of the AAV, and/or the number of days required for 100% release of the total amount of the AAV is at least 4 days. In one embodiment, the controlled release is characterized by the amount of the AAV released on day 1 is from 0 to 25% of the total amount of the AAV, the amount of the AAV released per day from day 2 until the last day of release is from 0 to 50% of the total amount of the AAV, and/or the number of days required for 100% release of the total amount of the AAV is at least 4 days. In one embodiment, the controlled release is characterized by the amount of the AAV released on day 1 is from 0 to 20% of the total amount of the AAV, the amount of the AAV released per day from day 2 until the last day of release is from 0 to 50% of the total amount of the AAV, and/or the number of days required for 100% release of the total amount of the AAV is at least 4 days. In one embodiment, the controlled release is characterized by the amount of the AAV released on day 1 is from 0 to 10% of the total amount of the AAV, the amount of the AAV released per day from day 2 until the last day of release is from 0 to 50% of the total amount of the AAV, and/or the number of days required for 100% release of the total amount of the AAV is at least 4 days.

Method of Controlling Inflammation when Treating an Ocular Disorder

A method of controlling inflammation when treating an ocular disorder such as an ocular genetic disorder is also provided comprising administering to a subject a pharmaceutically acceptable implant discussed in a separate section herewith. The pharmaceutically acceptable implant is for controlled release of any biologic that has been discussed in a separate section herewith. The controlled release of the biologic is also defined herewith in a separate section. In one embodiment, the biologic is a virus. In some embodiments, the virus is an adeno-associated virus (AAV) selected from a group consisting of AAV1, AAV2 such as AAV2.7m8, AAV3, AAV4, AAV5, AAV6, AAV7, AAV8, AAV9, AAV10, AAV11 and a mutant, hybrid or variant thereof at a total amount in the order from 10⁹ to 10¹⁵ vg, such as in the order from 10⁹ to 10¹³ vg. In some embodiments, the total amount of the AAV is from 10¹⁰ to 10¹⁵ vg. In some embodiments, the total amount of the AAV is from 10¹⁰ to 10¹⁴ vg. In some embodiments, the total amount of the AAV is from 10¹⁰ to 10¹³ vg. In some embodiments, the total amount of the AAV is from 10¹⁰ to 10¹² vg. In some embodiments, the total amount of the AAV is from 10¹⁰ to 10¹¹ vg. In these embodiments, the AAV comprises at least one heterologous nucleic acid sequence. In these embodiments, the heterologous nucleic acid sequence can be either a coding nucleic acid sequence or non-coding nucleic acid sequence. When the heterologous nucleic acid sequence is a coding nucleic acid sequence, it may code for a therapeutic protein as described in the section under the heading of biologic. In one embodiment, the controlled release is characterized by the amount of the AAV released on day 1 is from 0 to 50% of the total amount of the AAV, the amount of the AAV released per day from day 2 until the last day of release is from 0 to 50% of the total amount of the AAV, and/or the number of days required for 100% release of the total amount of the AAV is at least 4 days. In one embodiment, the controlled release is characterized by the amount of the AAV released on day 1 is from 0 to 25% of the total amount of the AAV, the amount of the AAV released per day from day 2 until the last day of release is from 0 to 50% of the total amount of the AAV, and/or the number of days required for 100% release of the total amount of the AAV is at least 4 days. In one embodiment, the controlled release is characterized by the amount of the AAV released on day 1 is from 0 to 20% of the total amount of the AAV, the amount of the AAV released per day from day 2 until the last day of release is from 0 to 50% of the total amount of the AAV, and/or the number of days required for 100% release of the total amount of the AAV is at least 4 days. In one embodiment, the controlled release is characterized by the amount of the AAV released on day 1 is from 0 to 10% of the total amount of the AAV, the amount of the AAV released per day from day 2 until the last day of release is from 0 to 50% of the total amount of the AAV, and/or the number of days required for 100% release of the total amount of the AAV is at least 4 days. In certain embodiments, the heterologous nucleic acid(s) code(s) for a therapeutic protein, which is absent in the subject, or present at a reduced level in the subject as compared to the same but healthy subject.

In one embodiment “controlling inflammation” refers to obtaining a lower inflammation score at the inflammation peak after administering the pharmaceutically acceptable implant of the present invention as compared to the inflammation score obtained at the inflammation peak after administering another composition that comprises the same biologic at the same dose as that of the pharmaceutically acceptable implant of the invention. In some embodiments, the “another composition” comprises a different release profile of the biologic (such as administration of a bolus) as compared to that of the pharmaceutically acceptable implant. Preferably, the inflammation scores have been assessed using the same method.

A skilled artisan is aware of methods that can be used to assess inflammation, for example, an ocular scoring system, such as the Standardization of Uveitis Nomenclature (SUN), semiquantitative preclinical ocular toxicology scoring (SPOTS), McDonald-Shadduck, the Hackett-McDonald systems at the inflammation peak. In some embodiments, the method is the semiquantitative preclinical ocular toxicology scoring (SPOTS).

In various embodiments, the inflammation peak is characterized as the period between the second week and two months, or three months, or four months following administration of the pharmaceutically acceptable implant to a subject, such as between the second week and one month, such as between two to five weeks following administration of the pharmaceutically acceptable implant to a subject. In some embodiments, the inflammation peak is characterized as the period between third- and fourth week following administration of the pharmaceutically acceptable implant to a rabbit. In some embodiments, the inflammation peak is characterized as the period between eight- and sixteen-weeks following administration of the pharmaceutically acceptable implant to a non-human primate. In some embodiments, the inflammation peak is characterized as the period between four weeks to five weeks, four weeks to six weeks, four weeks to seven weeks, four weeks to eight weeks, four weeks to nine weeks, four weeks to ten weeks, four weeks to eleven weeks, four weeks to twelve weeks, four weeks to thirteen weeks, four weeks to fourteen weeks, four weeks to fifteen weeks, four weeks to sixteen weeks or four weeks following administration of the pharmaceutically acceptable implant to a human.

In some embodiments, the inflammation peak is characterized as the period between five weeks to six weeks, five weeks to seven weeks, five weeks to eight weeks, five weeks to nine weeks, five weeks to ten weeks, five weeks to eleven weeks, five weeks to twelve weeks, five weeks to thirteen weeks, five weeks to fourteen weeks, five weeks to fifteen weeks, five weeks to sixteen weeks or five weeks following administration of the pharmaceutically acceptable implant to a human.

In some embodiments, the inflammation peak is characterized as the period between six weeks to seven weeks, six weeks to eight weeks, six weeks to nine weeks, six weeks to ten weeks, six weeks to eleven weeks, six weeks to twelve weeks, six weeks to thirteen weeks, six weeks to fourteen weeks, six weeks to fifteen weeks, six weeks to sixteen weeks or six weeks following administration of the pharmaceutically acceptable implant to a human.

In some embodiments, the inflammation peak is characterized as the period between seven weeks to eight weeks, seven weeks to nine weeks, seven weeks to ten weeks, seven weeks to eleven weeks, seven weeks to twelve weeks, seven weeks to thirteen weeks, seven weeks to fourteen weeks, seven weeks to fifteen weeks, seven weeks to sixteen weeks or seven weeks following administration of the pharmaceutically acceptable implant to a human.

In some embodiments, the inflammation peak is characterized as the period between eight weeks to nine weeks, eight weeks to ten weeks, eight weeks to eleven weeks, eight weeks to twelve weeks, eight weeks to thirteen weeks, eight weeks to fourteen weeks, eight weeks to fifteen weeks, eight weeks to sixteen weeks or eight weeks following administration of the pharmaceutically acceptable implant to a human.

In some embodiments, the inflammation peak is characterized as the period between nine weeks to ten weeks, nine weeks to eleven weeks, nine weeks to twelve weeks, nine weeks to thirteen weeks, nine weeks to fourteen weeks, nine weeks to fifteen weeks, nine weeks to sixteen weeks or nine weeks following administration of the pharmaceutically acceptable implant to a human.

In some embodiments, the inflammation peak is characterized as the period between ten weeks to eleven weeks, ten weeks to twelve weeks, ten weeks to thirteen weeks, ten weeks to fourteen weeks, ten weeks to fifteen weeks, ten weeks to sixteen weeks or ten weeks following administration of the pharmaceutically acceptable implant to a human.

In some embodiments, the inflammation peak is characterized as the period between eleven weeks to twelve weeks, eleven weeks to thirteen weeks, eleven weeks to fourteen weeks, eleven weeks to fifteen weeks, eleven weeks to sixteen weeks or eleven weeks following administration of the pharmaceutically acceptable implant to a human.

In some embodiments, the inflammation peak is characterized as the period between twelve weeks to thirteen weeks, twelve weeks to fourteen weeks, twelve weeks to fifteen weeks, twelve weeks to sixteen weeks or twelve weeks following administration of the pharmaceutically acceptable implant to a human.

In some embodiments, the inflammation peak is characterized as the period between thirteen weeks to fourteen weeks, thirteen weeks to fifteen weeks, thirteen weeks to sixteen weeks or thirteen weeks following administration of the pharmaceutically acceptable implant to a human.

In some embodiments, the inflammation peak is characterized as the period between fourteen weeks to fifteen weeks, fourteen weeks to sixteen weeks, or fourteen weeks following administration of the pharmaceutically acceptable implant to a human.

In some embodiments, the inflammation peak is characterized as the period between fifteen weeks to sixteen weeks, or sixteen weeks following administration of the pharmaceutically acceptable implant to a human.

In another embodiment, “controlling inflammation” refers to obtaining a lower inflammation score at least 4 weeks after administering the pharmaceutically acceptable implant of the present invention as compared to the inflammation score obtained at least 4 weeks after administering another composition that comprises the same biologic at the same dose as that of the pharmaceutically acceptable implant of the invention. Preferably, the inflammation scores have been assessed using the same method. In some embodiments, the “another composition” comprises a different release profile of the biologic (such as administration of a bolus) as compared to that of the pharmaceutically acceptable implant.

In another embodiment, “controlling inflammation” refers to obtaining a lower inflammation score from at the inflammation peak described above such as any period between 4 weeks up to 12 weeks, or 4 weeks up to 16 weeks after administering the pharmaceutically acceptable implant of the present invention as compared to the inflammation score obtained from 4 weeks up to 12 weeks after administering another composition that comprises the same biologic at the same dose as that of the pharmaceutically acceptable implant of the invention. Preferably, the inflammation scores have been assessed using the same method. In some embodiments, the “another composition” comprises a different release profile of the biologic (such as administration of a bolus) as compared to that of the pharmaceutically acceptable implant.

In another embodiment, “controlling inflammation” refers to obtaining a lower inflammation score at the inflammation peak described above such as any period from day 30 to day 120, or day 30 to day 90, or day 30 up to day 72 after administering the pharmaceutically acceptable implant of the present invention as compared to the inflammation score obtained from day 30 up to day 72 after administering another composition that comprises the same biologic at the same dose as that of the pharmaceutically acceptable implant of the invention. In some embodiments, the “another composition” comprises a different release profile of the biologic (such as administration of a bolus) as compared to that of the pharmaceutically acceptable implant. Preferably, the inflammation scores have been assessed using the same method.

The term “lower inflammation score” refers to an inflammation score obtained after administering the pharmaceutically acceptable implant of the present invention that is at least 25%, such as 50% less than the inflammation score obtained with another composition that comprises the same biologic at the same dose as that of the pharmaceutically acceptable implant of the invention. In some embodiments, the “another composition” comprises a different release profile of the biologic (such as administration of a bolus) as compared to that of the pharmaceutically acceptable implant. Preferably, the inflammation scores have been assessed using the same method.

In some embodiments, “lower inflammation score” or “acceptable inflammation level” is correlated with “higher expression” of the therapeutic protein coded by the heterologous nucleic acid sequence in the AAV comprised in the pharmaceutically acceptable implant. In one embodiment, the biologic is an AAV selected from a group consisting of AAV1, AAV2 such as AAV2.7m8, AAV3, AAV4, AAV5, AAV6, AAV7, AAV8, AAV9, AAV10, AAV11 and a mutant, hybrid or variant thereof at a total amount in the order from 10⁹ to 10¹⁵ vg, such as in the order from 10⁹ to 10¹³ vg. In some embodiments, the total amount of the AAV is from 10¹⁰ to 10¹⁵ vg. In some embodiments, the total amount of the AAV is from 10¹⁰ to 10¹⁴ vg. In some embodiments, the total amount of the AAV is from 10¹⁰ to 10¹³ vg. In some embodiments, the total amount of the AAV is from 10¹⁰ to 10¹² vg. In some embodiments, the total amount of the AAV is from 10¹⁰ to 10¹¹ vg. In these embodiments, the AAV comprises at least one heterologous nucleic acid sequence. In these embodiments, the heterologous nucleic acid sequence can be either a coding nucleic acid sequence or non-coding nucleic acid sequence. When the heterologous nucleic acid sequence is a coding nucleic acid sequence, it may code for a therapeutic protein as described in the section under the heading of biologic. In this embodiment, the inventors have found that the inflammation can be controlled by the controlled release characterized by no greater than 9.0×10⁹ to 1.5×10¹⁰ vg AAV released on day 1, no greater than in the order 10¹¹ vg AAV per day such as in the order 10⁸, or 10⁹ or 10¹⁰ such as 5.0×10⁹ to 1.5×10¹⁰ vg AAV released per day from day 2 until the last day of the controlled release, and/or the number of days required for 100% release of the AAV is not less than 4 days. In certain embodiments, the heterologous nucleic acid(s) code(s) for a therapeutic protein, which is absent in the subject, or present at a reduced level in the subject as compared to the same but healthy subject.

In one embodiment, the method of controlling inflammation when treating an ocular disorder such as an ocular genetic disorder comprises intravitreal injection of the pharmaceutically acceptable implant comprising a biologic to the subject in need thereof. In one embodiment, the biologic is an AAV selected from a group consisting of AAV1, AAV2 such as AAV2.7m8, AAV3, AAV4, AAV5, AAV6, AAV7, AAV8, AAV9, AAV10, AAV11 and a mutant, hybrid or variant thereof at a total amount in the order from 10⁹ to 10¹⁵ vg, such as in the order from 10⁹ to 10¹³ vg. In some embodiments, the total amount of the AAV is from 10¹⁰ to 10¹⁵ vg. In some embodiments, the total amount of the AAV is from 10¹⁰ to 10¹⁴ vg. In some embodiments, the total amount of the AAV is from 10¹⁰ to 10¹³ vg. In some embodiments, the total amount of the AAV is from 10¹⁰ to 10¹² vg. In some embodiments, the total amount of the AAV is from 10¹⁰ to 10¹¹ vg. In these embodiments, the AAV comprises at least one heterologous nucleic acid sequence. In these embodiments, the heterologous nucleic acid sequence can be either a coding nucleic acid sequence or non-coding nucleic acid sequence. When the heterologous nucleic acid sequence is a coding nucleic acid sequence, it may code for a therapeutic protein as described in the section under the heading of biologic. In this embodiment, the inventors have found that the inflammation is controlled by a controlled release characterized by no greater than 6×10⁹ to 1.0×10¹⁰ AAV vg per mL of the vitreous volume of the subject released on day 1, no greater than 3.5×10⁹ to 1.0×10¹⁰ AAV vg per mL of the vitreous volume of the subject released per day from day 2 until the last day of the controlled release, and/or the number of days required for 100% release of the AAV is not less than 4 days. In certain embodiments, the heterologous nucleic acid(s) code(s) for a therapeutic protein, which is absent in the subject, or present at a reduced level in the subject as compared to the same but healthy subject.

In another embodiment, the method of controlling inflammation when treating an ocular disorder such as an ocular genetic disorder comprises administering the pharmaceutically acceptable implant that comprises AAV in the order less than 2.0×10¹⁰ vg and the controlled release is characterized in that the number of days required for 100% release of the AAV is at least 4 days.

In another embodiment, the method of controlling inflammation when treating an ocular disorder such as an ocular genetic disorder comprises administering the pharmaceutically acceptable implant that comprises AAV order greater than 2.0×10¹⁰ vg and up to the order of 10¹⁵ vg and the controlled release is characterized in that the number of days required for 100% release of the AAV is greater than 10 days or more.

In another embodiment, the method of controlling inflammation when treating an ocular disorder such as an ocular genetic disorder comprises administering the pharmaceutically acceptable implant that comprises AAV order greater than 2.0×10¹⁰ vg and up to the order of 10¹⁵ vg and the controlled release is characterized in that the number of days required for 100% release of the AAV is greater than 15 days or more.

In another embodiment, the method of controlling inflammation when treating an ocular disorder such as an ocular genetic disorder comprises administering the pharmaceutically acceptable implant that comprises AAV order greater than 2.0×10¹⁰ vg and up to the order of 10¹³ vg and the controlled release is characterized in that the number of days required for 100% release of the AAV is greater than 10 days or more.

In another embodiment, the method of controlling inflammation when treating an ocular disorder such as an ocular genetic disorder comprises administering the pharmaceutically acceptable implant that comprises AAV order greater than 2.0×10¹⁰ vg and up to the order of 10¹³ vg and the controlled release is characterized in that the number of days required for 100% release of the AAV is greater than 15 days or more.

In another embodiment, the method of controlling inflammation when treating an ocular disorder such as an ocular genetic disorder comprises administering the pharmaceutically acceptable implant that comprises AAV order greater than 2.0×10¹⁰ vg and up to the order of 10¹² vg and the controlled release is characterized in that the number of days required for 100% release of the AAV is greater than 10 days or more.

In another embodiment, the method of controlling inflammation when treating an ocular disorder such as an ocular genetic disorder comprises administering the pharmaceutically acceptable implant that comprises AAV order greater than 2.0×10¹⁰ vg and up to the order of 10¹² vg and the controlled release is characterized in that the number of days required for 100% release of the AAV is greater than 15 days or more.

In another embodiment, the method of controlling inflammation when treating an ocular disorder such as an ocular genetic disorder comprises administering the pharmaceutically acceptable implant that comprises AAV order greater than 2.0×10¹⁰ vg and up to the order of 10¹¹ vg and the controlled release is characterized in that the number of days required for 100% release of the AAV is greater than 10 days or more.

In another embodiment, the method of controlling inflammation when treating an ocular disorder such as an ocular genetic disorder comprises administering the pharmaceutically acceptable implant that comprises AAV order greater than 2.0×10¹⁰ vg and up to the order of 10¹¹ vg and the controlled release is characterized in that the number of days required for 100% release of the AAV is greater than 15 days or more. In some embodiments, the implant that comprises the AAV is capable of sustained release of the AAV in vivo for at least about 4 weeks, at least about 5 weeks, at least about 6 weeks, at least about 7 weeks, or at least about 8 weeks. In some embodiments, the implant that comprises the AAV is capable of providing sustained release of the AAV in vivo for more than 8 weeks. As such, in some aspects, the method comprises controlling inflammation when treating an ocular disorder such as an ocular genetic disorder comprises administering the pharmaceutically acceptable implant that comprises the AAV, wherein the implant is capable of sustained release of the AAV in vivo for at least 8 weeks.

### Combination Treatment

The methods described in this section can also comprise administration of the pharmaceutically acceptable implant in combination with another agent also termed combination therapy.

In one embodiment, the combination therapy comprises administering a pharmaceutically acceptable implant of the invention comprising an AAV selected from a group consisting of AAV1, AAV2 such as AAV2.7m8, AAV3, AAV4, AAV5, AAV6, AAV7, AAV8, AAV9, AAV10, AAV11 and a mutant, hybrid or variant thereof at a total amount in the order from 10⁹ to 10¹⁵ vg, such as in the order from 10⁹ to 10¹³ vg. In some embodiments, the total amount of the AAV is from 10¹⁰ to 10¹⁵ vg. In some embodiments, the total amount of the AAV is from 10¹⁰ to 10¹⁴ vg. In some embodiments, the total amount of the AAV is from 10¹⁰ to 10¹³ vg. In some embodiments, the total amount of the AAV is from 10¹⁰ to 10¹² vg. In some embodiments, the total amount of the AAV is from 10¹⁰ to 10¹¹ vg. In some embodiments, the AAV comprises more than one heterologous nucleotide sequences, wherein each of the more than one heterologous nucleotide sequences codes for a different therapeutic protein. A description of therapeutic proteins is described in the section under the heading of biologic. In certain embodiments, the heterologous nucleic acid(s) code(s) for a therapeutic protein, which is absent in the subject, or present at a reduced level in the subject as compared to the same but healthy subject.

In another embodiment, combination therapy comprises administering the pharmaceutically acceptable implant of the invention in combination with one or more additional agents either on the same or different day. In one embodiment, the additional agent to be administered in a combination therapy can be a liquid formulation of the agent, and or it may be comprised in a vector. Thus, the additional agent can be any small molecule, large molecule, a protein, a nanoparticle, or another virus. In one embodiment, the additional agent to be administered in a combination therapy with the pharmaceutically acceptable implant of the present invention can be an immunosuppressant such as triamcinolone, prednisolone. In another embodiment, the additional agent to be administered in a combination therapy with the pharmaceutically acceptable implant of the present invention is cyclosporin, cyclophsphamide, sirolimus, or tacrolimus. In another embodiment, the additional agent can be an anti-NAb, an anti-T-cell antibody such as anti-CD40L, or a viral transduction enhancer. In another embodiment, the additional agent is a tyrosine kinase inhibitor (TKI), such as axitinib, sunitinib, sorafenib, paxopanib, or tivozanib. In this embodiment, the TKI may be administered in a liquid formulation, in the form of a separate implant or the same implant. In one embodiment, the additional agents to be administered in a combination therapy can be an anti-inflammatory agents, anti-vaso-proliferative agents, corticosteroids, non-steroidal anti-inflammatory drugs (NSAIDs), interocular pressure (IOP) lowering agents, anti-infective drugs, antibiotics, anti-mitotic agents, antivirals, antifungals, antimetabolites, antifibrotic agents, glaucoma medications, anti-neovascular agents, integrins, integrin antagonists, complement antagonists, cytokines, cytokine inhibitors, antibody-blocking agents, angiogenesis inhibitors, vaccines, immunomodulatory agents, anticoagulants, anti-neoplastic agents, anaesthetics, analgesics, adrenergic agonists or antagonists, cholinergic agonists or antagonists, enzymes, enzyme inhibitors, neuroprotective agents, cytoprotective agents, regenerative agents, antisense oligonucleotides, aptamers, antibodies, or combinations thereof. These agents may be comprised of proteins, peptides, lipids, carbohydrates, hormones, metals, radioactive elements and compounds, or combinations thereof.

In some embodiments, corticosteroids can comprise hydrocortisone, loteprednol, cortisol, cortisone, prednisolone, methylprednisolone, dexamethasone, betamethasone, triamcinolone, aldosterone or fludrocortisone.

In some embodiments, NSAIDs can comprise diclofenac (e.g., diclofenac sodium), flubiprofen (e.g., flubiprofen sodium), ketorolac (e.g., ketorolac tromethamine), bromfenac, or nepafenac.

In some embodiments, IOP lowering agents and/or glaucoma medications can comprise prostaglandin analogs (e.g., bimatoprost, latanoprost, travoprost, or latanoprostene bunod), rho kinase inhibitor (e.g., netarsudil), adrenergic agonists (epinephrine or dipivefrin), beta-adrenergic antagonists also known as beta blockers (e.g., timolol, levobunolol, metipranolol, carteolol, or betaxolol), alpha2-adrenergic agonists (e.g., apraclonidine, brimonidine, or brimonidine tartrate), carbonic anhydrase inhibitors (e.g., brinzolamide, dichlorphenamide, methazolamide acetazolamide, acetazolamide, or dorzolamide), pilocarpine, echothiophate, demercarium, physostigmine, and/or isofluorophate.

In some embodiments, anti-infective can comprise antibiotics comprising ciprofloxacin, tobramycin, erythromycin, ofloxacin, gentamicin, fluoroquinolone antibiotics, moxifloxacin, and/or gatifloxacin; antivirals comprising ganciclovir, idoxuridine, vidarabine, and/or trifluridine; and/or antifungals comprising amphotericin B, natamycin, voriconazole, fluconazole, miconazole, clotrimazole, ketoconazole, posaconazole, echinocandin, caspofungin, and/or micafungin.

In some embodiments, antimetabolites can comprise methotrexate, mycophenolate, or azathioprine.

In some embodiments, antifibrotic agents can comprise maitomycin C or 5-fluorouracil.

In some embodiments, angiogenesis inhibitors can comprise anti-VEGF agents (e.g., aflibercept, ranibizumab, bevacizumab), PDGF-B inhibitors (e.g., Fovista®), complement antagonists (e.g., eculizumab), tyrosine kinase inhibitors (e.g. sunitinib, axitinib), and/or integrin antagonists (e.g., natalizumab and vedolizumab).

In some embodiments, cytoprotective agents can comprise ebselen, sulforaphane, oltipraz or dimethyl fumarate.

In some embodiments, neuroprotective agents can comprise ursodiol, memantine or acetylcysteine.

In some embodiments, anesthetic agents can comprise lidocaine, proparacaine or bupivacaine.

In some embodiments, the agent can be dexamethasone, ketorolac, diclofenac, vancomycin, moxifloxacin, gatifloxicin, besifloxacin, travoprost, 5-fluorouracil, methotrexate, mitomycin C, prednisolone, bevacizumab (Avastin®), ranibizumab (Lucentis®), sunitinib, pegaptanib (Macugen®), timolol, latanoprost, brimonidine, nepafenac, bromfenac, triamcinolone, difluprednate, fluocinolide, aflibercept, or combinations thereof. In some embodiments, the agent may be dexamethasone, ketorolac, diclofenac, moxifloxacin, travoprost, 5-fluorouracil, or methotrexate. In some embodiments, the agent is dexamethasone. In some embodiments, the agent is ketorolac. In some embodiments, the agent is dexamethasone.

The method of treatment comprising administering the pharmaceutically acceptable implant as described in this section may comprise any one of intravitreal, intracameral, subconjunctival, retrobulbar, sub-tenon, subretinal, and suprachoroidal injections. The method of administration may also be topical.

The additional agent to be administered in a combination therapy may also be a diagnostic agent. Diagnostic agents may be substances used to examine the body in order to detect impairment of its normal functions. In some cases, diagnostic agents may be agents with a functional purpose, such as for use in the detection of ocular deformities, ailments, and pathophysiological aspects. For example, the diagnostic agent may be an important and effective diagnostic adjuvant, such as a dye (e.g., fluorescein dye, indocyanine green, trypan blue, a dark quencher such as a cyanine dye, an azo dye, an acridine, a fluorone, an oxazine, a phenanthridine, a naphthalimide, a rhodamine, a benzopyrone, a perylene, a benzanthrone, pra benzoxanthrone), to aid in visualization of ocular tissues. The diagnostic agent may comprise paramagnetic molecules, fluorescent compounds, magnetic molecules, radionuclides, x-ray imaging agents, and/or contrast media. In some embodiments, a diagnostic agent may include radiopharmaceuticals, contrast agents for use in imaging techniques, allergen extracts, activated charcoal, different testing strips (e.g., cholesterol, ethanol, and glucose), pregnancy test, breath test with urea 13C, and various stains/markers. In some embodiments, the labelling moiety is a fluorescent dye or a dark quencher, selected from the group consisting of a coumarin, a cyanine dye, an azo dye, an acridine, a fluorone, an oxazine, a phenanthridine, a naphthalimide, a rhodamine, a benzopyrone, a perylene, a benzanthrone, and a benzoxanthrone. In particular non-limiting embodiments, the fluorescent dye is or is the residue of a compound selected from the group consisting of Coumarin, Fluorescein, Cyanine 3 (Cy3), Cyanine 5 (Cy5), Cyanine 7 (Cy7), Alexa dyes, bodipy derivatives, (E)-2-(4-(phenyldiazenyl) phenoxy) acetic acid, 3-(3′,3′-dimethyl-6-nitrospiro[chromene-2,2′-indolin]-1′-yl) propanoate (Spiropyran), 3,5-dihydroxybenzoate and (E)-2-(4-(phenyldiazenyl) phenoxy) acetic acid, or combinations thereof.

### Ocular Disease

In one embodiment the ocular disease is selected from retinal neovascularisation, choroidal neovascularisation, Wet AMD, Dry AMD, retinal vein occlusion, diabetic macular edema, retinal degeneration, corneal graft rejection, retinoblastoma, melanoma, glaucoma, autoimmune uveitis, uveitis, proliferative vitreoretinopathy, and corneal degeneration, acute and chronic macular neuroretinopathy, central serous chorioretinopathy, macular edema, acute multifocal placoid pigment epitheliopathy, Behcet's disease, birdshot retinochoroidopathy, posterior uveitis, posterior scleritis, serpignous choroiditis, subretinal fibrosis, uveitis syndrome, Vogt-Koyanagi-Harada syndrome, retinal arterial occlusive disease, central retinal vein occlusion, disseminated intravascular coagulopathy, branch retinal vein occlusion, hypertensive fundus changes, ocular ischemic syndrome, retinal arterial microaneurysms, Coat's disease, parafoveal telangiectasis, hemi-retinal vein occlusion, papillophlebitis, carotid artery disease (CAD), frosted branch angitis, sickle cell retinopathy, angioid streaks, familial exudative vitreoretinopathy, Eales disease, proliferative vitreal retinopathy, diabetic retinopathy, retinal disease associated with tumors, congenital hypertrophy of the retinal pigment epithelium (RPE), posterior uveal melanoma, choroidal hemangioma, choroidal osteoma, choroidal metastasis, combined hamartoma of the retina and retinal pigmented epithelium, retinoblastoma, vasoproliferative tumors of the ocular fundus, retinal astrocytoma, intraocular lymphoid tumors, myopic retinal degeneration, acute retinal pigment epithelitis, glaucoma, endophthalmitis, cytomegalovirus retinitis, retinal cancers, retinitis pigmentosa, Leber's Congenital Amaurosis, Choroideremia, X-linked retinitis pigmentosa, best vitelliform macular dystrophy, x-linked retinoschisis, achromatopsia CNGA3, achromotopsia CNGB3, LHON, Stargardt disease, Usher syndrome, Norrie disease, Bardet-Biedl syndrome, Goldmann Favre, rod-cone dystrophy, Best disease and red-green colour blindness.

In some embodiments, the ocular disease is an ocular genetic disease and is selected from a group consisting of retinitis pigmentosa, Leber's Congenital Amaurosis, Choroideremia, X-linked retinitis pigmentosa, best vitelliform macular dystrophy, x-linked retinoschisis, achromatopsia CNGA3, achromotopsia CNGB3, LHON, Stargardt disease, Usher syndrome, Norrie disease, Bardet-Biedl syndrome, and red-green colour blindness.

Method of Controlling an Immune Response when Treating

In one embodiment, a method of controlling an immune response such as an adaptive immune response or humoral immune response when treating an ocular disorder such as an ocular genetic disorder is provided comprising administering to a subject a pharmaceutically acceptable implant as discussed before. In one embodiment, “controlling an immune response such as an adaptive immune response or humoral immune response” is characterized by inducing an anti-drug antibody (ADA) titer against a biologic comprised in the implant after administration of the implant to the subject that is no greater than 20,000, or 15,000, or 10,000, or 8,000, or 7,000, or 5,000, or 2,000, or 1,000 or below detection limit as compared to the ADA titer at baseline. In one embodiment, the ADA titer refers to serum ADA titer.

In one embodiment, a method of controlling an immune response such as an adaptive immune response or humoral immune response when treating an ocular disorder such as an ocular genetic disorder is provided comprising administering to a subject a pharmaceutically acceptable implant comprising a total amount of biologic as discussed before. In one embodiment, “controlling an immune response such as an adaptive immune response or humoral immune response” is characterized by inducing an anti-drug antibody (ADA) titer against the biologic after administration of the implant to the subject that is lower than the ADA titer against the same biologic obtained after administering another composition such as a bolus comprising the same total amount of the biologic and administered to a comparison subject at the same time. In one embodiment, the ADA titer refers to serum ADA titer.

In one embodiment, “controlling an immune response such as an adaptive immune response or humoral immune response” is characterized by inducing an anti-drug antibody (ADA) titer against the biologic after administration of the implant to the subject that is lower by at least 10%, or at least 20%, or at least, 30%, or at least 40%, or at least 50%, or at least 60%, or at least 70%, or at least 80%, or at least 90% or 100% than the ADA titer against the same biologic obtained after administering another composition such as a bolus comprising the same total amount of the biologic and administered to a comparison subject at the same time. In one embodiment, the ADA titer refers to serum ADA titer.

In one embodiment, “controlling an immune response such as an adaptive immune response or humoral immune response” is characterized by inducing an anti-drug antibody (ADA) titer against the biologic comprised in the implant after administration of the implant to the subject that is lower by at least 20,000, or 15,000, or 10,000, or 8,000, or 7,000, or 5,000, or 2,000, or 1,000 than the ADA titer against the same biologic obtained after administering another composition such as a bolus comprising the same total amount of the biologic and administered to a comparison subject at the same time. In one embodiment, the ADA titer refers to serum ADA titer.

In one embodiment, “another composition” is a bolus comprising the same biologic at the same total amount.

In another embodiment, “another composition” is a pharmaceutically acceptable implant comprising the same biologic at the same total amount characterized in that the total number of days required for 100% release of the total amount of the biologic is fewer, wherein the release is measured from the time and under conditions wherein the implant is first immersed in an aqueous solution under physiological conditions such as at pH 7.2-7.4 and temperature 37° C.

In another embodiment, “another composition” is a pharmaceutically acceptable implant comprising a higher total amount of the same biologic characterized in that the total number of days required for 100% release of the total amount of the biologic is fewer, wherein the release is measured from the time and under conditions wherein the implant is first immersed in an aqueous solution under physiological conditions such as at pH 7.2-7.4 and temperature 37° C.

In one embodiment, “fewer” refers to at least 5 days fewer, such as 12 days fewer or 10 days fewer.

In another embodiment, “another composition” is a pharmaceutically acceptable implant comprising a higher total amount of the same biologic. In one embodiment, the higher total amount refers to at least 3 times higher, such as 4 times higher, 5 times higher, 6 times higher, 7 times higher, 8 times higher, 9 times higher, one log higher, two log higher, three log higher, or four log higher.

In one embodiment, the ADA titer pertains to ADA titer at any time from week 8 to week 13 post-administration such as at week 8 or at week 13 post-administration. This time point may depend on the biologic. If the biologic is AAV, this time point may depend on the AAV serotype.

In one embodiment, the ADA titer pertains to any time from week 4 to week 24 post administration. In one embodiment, the ADA titer pertains to any time from week 4 to week 23, from week 4 to week 22, from week 4 to week 21, from week 4 to week 20, from week 4 to week 19, from week 4 to week 18, from week 4 to week 17, from week 4 to week 16, from week 4 to week 15, from week 4 to week 14, from week 4 to week 13, from week 4 to week 12, from week 4 to week 11, from week 4 to week 10, from week 4 to week 9, from week 4 to week 8, from week 4 to week 7, from week 4 to week 6, from week 4 to week 5, or at week 4 post-administration.

In one embodiment, the ADA titer pertains to any time from week 4 to week 24 post administration. In one embodiment, the ADA titer pertains to any time from week 5 to week 23, from week 5 to week 22, from week 5 to week 21, from week 5 to week 20, from week 5 to week 19, from week 5 to week 18, from week 5 to week 17, from week 5 to week 16, from week 5 to week 15, from week 5 to week 14, from week 5 to week 13, from week 5 to week 12, from week 5 to week 11, from week 5 to week 10, from week 5 to week 9, from week 5 to week 8, from week 5 to week 7, from week 5 to week 6, or at week 5 post-administration.

In one embodiment, the ADA titer pertains to any time from week 6 to week 24 post administration. In one embodiment, the ADA titer pertains to any time from week 6 to week 23, from week 6 to week 22, from week 6 to week 21, from week 6 to week 20, from week 6 to week 19, from week 6 to week 18, from week 6 to week 17, from week 6 to week 16, from week 6 to week 15, from week 6 to week 14, from week 6 to week 13, from week 6 to week 12, from week 6 to week 11, from week 6 to week 10, from week 6 to week 9, from week 6 to week 8, from week 6 to week 7, or at week 6 post-administration.

In one embodiment, the ADA titer pertains to any time from week 7 to week 24 post administration. In one embodiment, the ADA titer pertains to any time from week 7 to week 23, from week 7 to week 22, from week 7 to week 21, from week 7 to week 20, from week 7 to week 19, from week 7 to week 18, from week 7 to week 17, from week 7 to week 16, from week 7 to week 15, from week 7 to week 14, from week 7 to week 13, from week 7 to week 12, from week 7 to week 11, from week 7 to week 10, from week 7 to week 9, from week 7 to week 8, or at week 7 post-administration.

In one embodiment, the ADA titer pertains to any time from week 8 to week 24 post administration. In one embodiment, the ADA titer pertains to any time from week 8 to week 23, from week 8 to week 22, from week 8 to week 21, from week 8 to week 20, from week 8 to week 19, from week 8 to week 18, from week 8 to week 17, from week 8 to week 16, from week 8 to week 15, from week 8 to week 14, from week 8 to week 13, from week 8 to week 12, from week 8 to week 11, from week 8 to week 10, from week 8 to week 9, or at week 8 post-administration.

In one embodiment, the ADA titer pertains to any time from week 9 to week 24 post administration. In one embodiment, the ADA titer pertains to any time from week 9 to week 23, from week 9 to week 22, from week 9 to week 21, from week 9 to week 20, from week 9 to week 19, from week 9 to week 18, from week 9 to week 17, from week 9 to week 16, from week 9 to week 15, from week 9 to week 14, from week 9 to week 13, from week 9 to week 12, from week 9 to week 11, from week 9 to week 10, or at week 9 post-administration.

In one embodiment, the ADA titer pertains to any time from week 10 to week 24 post administration. In one embodiment, the ADA titer pertains to any time from week 4 to week 23, from week 10 to week 22, from week 10 to week 21, from week 10 to week 20, from week 10 to week 19, from week 10 to week 18, from week 10 to week 17, from week 10 to week 16, from week 10 to week 15, from week 10 to week 14, from week 10 to week 13, from week 10 to week 12, from week 10 to week 11, or at week 10 post-administration.

In one embodiment, the ADA titer pertains to any time from week 11 to week 24 post administration. In one embodiment, the ADA titer pertains to any time from week 11 to week 23, from week 11 to week 22, from week 11 to week 21, from week 11 to week 20, from week 11 to week 19, from week 11 to week 18, from week 11 to week 17, from week 11 to week 16, from week 11 to week 15, from week 11 to week 14, from week 11 to week 13, from week 11 to week 12, or at week 11 post-administration.

In one embodiment, the ADA titer pertains to any time from week 12 to week 24 post administration. In one embodiment, the ADA titer pertains to any time from week 12 to week 23, from week 12 to week 22, from week 12 to week 21, from week 12 to week 20, from week 12 to week 19, from week 12 to week 18, from week 12 to week 17, from week 12 to week 16, from week 12 to week 15, from week 12 to week 14, from week 12 to week 13, or at week 12 post-administration.

In one embodiment, the ADA titer pertains to any time from week 13 to week 24 post administration. In one embodiment, the ADA titer pertains to any time from week 13 to week 23, from week 13 to week 22, from week 13 to week 21, from week 13 to week 20, from week 13 to week 19, from week 13 to week 18, from week 13 to week 17, from week 13 to week 16, from week 13 to week 15, from week 13 to week 14, or at week 13 post-administration.

In one embodiment, the ADA titer pertains to any time from week 14 to week 24 post administration. In one embodiment, the ADA titer pertains to any time from week 14 to week 23, from week 14 to week 22, from week 14 to week 21, from week 14 to week 20, from week 14 to week 19, from week 14 to week 18, from week 14 to week 17, from week 14 to week 16, from week 14 to week 15, or at week 14 post-administration.

In one embodiment, the ADA titer pertains to any time from week 15 to week 24 post administration. In one embodiment, the ADA titer pertains to any time from week 15 to week 23, from week 15 to week 22, from week 15 to week 21, from week 15 to week 20, from week 15 to week 19, from week 15 to week 18, from week 15 to week 17, from week 15 to week 16, or at week 15 post-administration.

In one embodiment, the ADA titer pertains to any time from week 16 to week 24 post administration. In one embodiment, the ADA titer pertains to any time from week 16 to week 23, from week 16 to week 22, from week 16 to week 21, from week 16 to week 20, from week 16 to week 19, from week 16 to week 18, from week 16 to week 17, or at week 16 post-administration.

In one embodiment, the ADA titer pertains to any time from week 17 to week 24 post administration. In one embodiment, the ADA titer pertains to any time from week 17 to week 23, from week 17 to week 22, from week 17 to week 21, from week 17 to week 20, from week 17 to week 19, from week 17 to week 18, or at week 17 post-administration.

In one embodiment, the ADA titer pertains to any time from week 18 to week 24 post administration. In one embodiment, the ADA titer pertains to any time from week 18 to week 23, from week 18 to week 22, from week 18 to week 21, from week 18 to week 20, from week 18 to week 19, or at week 18 post-administration.

In one embodiment, the ADA titer pertains to any time from week 19 to week 24 post administration. In one embodiment, the ADA titer pertains to any time from week 19 to week 23, from week 19 to week 22, from week 19 to week 21, from week 19 to week 20, or at week 19 post-administration.

In one embodiment, the ADA titer pertains to any time from week 20 to week 24 post administration. In one embodiment, the ADA titer pertains to any time from week 20 to week 23, from week 20 to week 22, from week 20 to week 21, or at week 20 post-administration.

In one embodiment, the ADA titer pertains to any time from week 21 to week 24 post administration. In one embodiment, the ADA titer pertains to any time from week 21 to week 23, from week 21 to week 22, or at week 21 post-administration.

In one embodiment, the ADA titer pertains to any time from week 22 to week 24 post administration. In one embodiment, the ADA titer pertains to any time from week 22 to week 23, or at week 22 post-administration.

In one embodiment, the ADA titer pertains to any time from week 23 to week 24 post administration or at week 23 post-administration or at week 24 post administration.

In one embodiment, the ADA titer is measured at a time point post administration in which the ADA titer due to the bolus is at least 5,000, or at least 10,000 or at least 15,000 or at least 20,000. In one embodiment, the ADA titer refers to serum ADA titer.

In one embodiment, the implant is administered to the eye of the subject such as by an intravitreal injection.

In one embodiment, the biologic is an AAV selected from a group consisting of AAV1, AAV2 such as AAV2.7m8, AAV3, AAV4, AAV5, AAV6, AAV7, AAV8, AAV9, AAV10, AAV11 and a mutant, hybrid or variant thereof at a total amount in the order from 10⁹ to 10¹⁵ vg, such as in the order from 10⁹ to 10¹³ vg. In some embodiments, the total amount of the AAV is from 10¹⁰ to 10¹⁵ vg. In some embodiments, the total amount of the AAV is from 10¹⁰ to 10¹⁴ vg. In some embodiments, the total amount of the AAV is from 10¹⁰ to 10¹³ vg. In some embodiments, the total amount of the AAV is from 10¹⁰ to 10¹² vg. In some embodiments, the total amount of the AAV is from 10¹⁰ to 10¹¹ vg. In these embodiments, the AAV comprises at least one heterologous nucleic acid sequence. In these embodiments, the heterologous nucleic acid sequence can be either a coding nucleic acid sequence or non-coding nucleic acid sequence. When the heterologous nucleic acid sequence is a coding nucleic acid sequence, it may code for a therapeutic protein as described in the section under the heading of biologic. In this embodiment, the inventors have found that the inflammation can be controlled by the controlled release characterized by no greater than 9.0×10⁹ to 1.5×10¹⁰ vg AAV released on day 1, no greater than in the order 10¹¹ vg AAV per day such as in the order 10⁸, or 10⁹ or 10¹⁰ such as 5.0×10⁹ to 1.5×10¹⁰ vg AAV released per day from day 2 until the last day of the controlled release, and/or the number of days required for 100% release of the AAV is not less than 4 days. In certain embodiments, the heterologous nucleic acid(s) code(s) for a therapeutic protein, which is absent in the subject, or present at a reduced level in the subject as compared to the same but healthy subject.

In one embodiment, the method of controlling an immune response such as an adaptive immune response or humoral immune response when treating an ocular disorder such as an ocular genetic disorder comprises intravitreal injection of the pharmaceutically acceptable implant comprising a biologic to the subject in need thereof. In one embodiment, the biologic is an AAV selected from a group consisting of AAV1, AAV2 such as AAV2.7m8, AAV3, AAV4, AAV5, AAV6, AAV7, AAV8, AAV9, AAV10, AAV11 and a mutant, hybrid or variant thereof at a total amount in the order from 10⁹ to 10¹⁵ vg, such as in the order from 10⁹ to 10¹³ vg. In some embodiments, the total amount of the AAV is from 10¹⁰ to 10¹⁵ vg. In some embodiments, the total amount of the AAV is from 10¹⁰ to 10¹⁴ vg. In some embodiments, the total amount of the AAV is from 10¹⁰ to 10¹³ vg. In some embodiments, the total amount of the AAV is from 10¹⁰ to 10¹² vg. In some embodiments, the total amount of the AAV is from 10¹⁰ to 10¹¹ vg. In these embodiments, the AAV comprises at least one heterologous nucleic acid sequence. In these embodiments, the heterologous nucleic acid sequence can be either a coding nucleic acid sequence or noncoding nucleic acid sequence. When the heterologous nucleic acid sequence is a coding nucleic acid sequence, it may code for a therapeutic protein as described in the section under the heading of biologic. In this embodiment, the inventors have found that the inflammation is controlled by a controlled release characterized by no greater than 6×10⁹ to 1.0×10¹⁰ AAV vg per mL of the vitreous volume of the subject released on day 1, no greater than 3.5×10⁹ to 1.0×10¹⁰ AAV vg per mL of the vitreous volume of the subject released per day from day 2 until the last day of the controlled release, and/or the number of days required for 100% release of the AAV is not less than 4 days. In certain embodiments, the heterologous nucleic acid(s) code(s) for a therapeutic protein, which is absent in the subject, or present at a reduced level in the subject as compared to the same but healthy subject.

In another embodiment, the method of controlling an immune response such as an adaptive immune response or humoral immune response when treating an ocular disorder such as an ocular genetic disorder comprises administering the pharmaceutically acceptable implant that comprises AAV in the order less than 2.0×10¹⁰ vg and the controlled release is characterized in that the number of days required for 100% release of the AAV is at least 4 days.

In another embodiment, the method of controlling an immune response such as an adaptive immune response or humoral immune response when treating an ocular disorder such as an ocular genetic disorder comprises administering the pharmaceutically acceptable implant that comprises AAV order greater than 2.0×10¹⁰ vg and up to the order of 10¹⁵ vg and the controlled release is characterized in that the number of days required for 100% release of the AAV is greater than 10 days or more.

In another embodiment, the method of controlling an immune response such as an adaptive immune response or humoral immune response when treating an ocular disorder such as an ocular genetic disorder comprises administering the pharmaceutically acceptable implant that comprises AAV order greater than 2.0×10¹⁰ vg and up to the order of 10¹⁵ vg and the controlled release is characterized in that the number of days required for 100% release of the AAV is greater than 15 days or more.

In another embodiment, the method of controlling an immune response such as an adaptive immune response or humoral immune response when treating an ocular disorder such as an ocular genetic disorder comprises administering the pharmaceutically acceptable implant that comprises AAV order greater than 2.0×10¹⁰ vg and up to the order of 10¹³ vg and the controlled release is characterized in that the number of days required for 100% release of the AAV is greater than 10 days or more.

In another embodiment, the method of controlling an immune response such as an adaptive immune response or humoral immune response when treating an ocular disorder such as an ocular genetic disorder comprises administering the pharmaceutically acceptable implant that comprises AAV order greater than 2.0×10¹⁰ vg and up to the order of 10¹³ vg and the controlled release is characterized in that the number of days required for 100% release of the AAV is greater than 15 days or more.

In another embodiment, the method of controlling an immune response such as an adaptive immune response or humoral immune response when treating an ocular disorder such as an ocular genetic disorder comprises administering the pharmaceutically acceptable implant that comprises AAV order greater than 2.0×10¹⁰ vg and up to the order of 10¹² vg and the controlled release is characterized in that the number of days required for 100% release of the AAV is greater than 10 days or more.

In another embodiment, the method of controlling an immune response such as an adaptive immune response or humoral immune response when treating an ocular disorder such as an ocular genetic disorder comprises administering the pharmaceutically acceptable implant that comprises AAV order greater than 2.0×10¹⁰ vg and up to the order of 10¹² vg and the controlled release is characterized in that the number of days required for 100% release of the AAV is greater than 15 days or more.

In another embodiment, the method of controlling an immune response such as an adaptive immune response or humoral immune response when treating an ocular disorder such as an ocular genetic disorder comprises administering the pharmaceutically acceptable implant that comprises AAV order greater than 2.0×10¹⁰ vg and up to the order of 10¹¹ vg and the controlled release is characterized in that the number of days required for 100% release of the AAV is greater than 10 days or more.

In another embodiment, the method of controlling an immune response such as an adaptive immune response or humoral immune response when treating an ocular disorder such as an ocular genetic disorder comprises administering the pharmaceutically acceptable implant that comprises AAV order greater than 2.0×10¹⁰ vg and up to the order of 10¹¹ vg and the controlled release is characterized in that the number of days required for 100% release of the AAV is greater than 15 days or more.

### Method of Effective Treatment of an Ocular Disorder

In one embodiment, a method of effective treatment of an ocular disorder such as an ocular genetic disorder is provided comprising administering to a subject a pharmaceutically acceptable implant as discussed before. In one embodiment, effective treatment is characterized by the total detectable amount of the biologic in the systemic circulation of the subject that is lower than the total amount of the biologic comprised in the implant. In one embodiment. The total detectable amount of the biologic pertains to the amount between day 1 to day 10 post administration such as day 1 to day 4, such as day 1 to day 3, or day 2 post administration. In one embodiment, systemic circulation refers to blood, plasma, serum or lymph.

In one embodiment, the biologic is AAV and the effective treatment is characterized by the total detectable amount of an endogeneous nucleic acid sequence of said AAV in the systemic circulation of the subject that is at least four log-less, five log-less, six log-less, seven log-less, eight log-less, nine log less or below detection limit as compared to the total amount of the AAV in gc comprised in the implant. In one embodiment. The total detectable amount of the endogenous nucleic acid sequence of the AAV pertains to the amount between day 1 to day 10 post administration such as day 1 to day 4, such as day 1 to day 3, or day 2 post administration. In one embodiment, systemic circulation refers to blood, plasma, serum or lymph.

In one embodiment, the biologic is AAV comprising a heterologous nucleic acid sequence, and the effective treatment is characterized by the total detectable amount of the heterologous nucleic acid sequence in the systemic circulation of the subject that is at least four log-less, five log-less, six log-less, seven log-less, eight log-less, nine log less or below detection limit as compared to the total amount of the AAV in gc comprised in the implant. In one embodiment. The total detectable amount of the heterologous nucleic acid sequence pertains to the amount between day 1 to day 10 post administration such as day 1 to day 4, such as day 1 to day 3, or day 2 post administration. In one embodiment, systemic circulation refers to blood, plasma, serum or lymph.

In one embodiment, a method of effective treatment of an ocular disorder such as an ocular genetic disorder is provided comprising administering to a subject a pharmaceutically acceptable implant as discussed before. In one embodiment, the implant comprises a total amount of a biologic and the effective treatment is characterized by the total detectable amount of the biologic in the systemic circulation of the subject that is lower as compared to the total detectable amount of the same biologic in the systemic circulation of the subject obtained after administering another composition such as a bolus comprising the same total amount of the biologic and administered to a comparison subject at the same time. In one embodiment. The total detectable amount of the biologic pertains to the amount between day 1 to day 10 post administration such as day 1 to day 4, such as day 1 to day 3, or day 2 post administration. In one embodiment, systemic circulation refers to blood, plasma, serum or lymph.

In one embodiment, the implant comprises a total amount of a biologic and the effective treatment is characterized by a lower Cₘₐₓ in the subject as compared to the Cₘₐₓ obtained in a subject after administering another composition such as a bolus comprising the same total amount of the biologic and administered to a subject at the same time. In one embodiment, Cmax pertains to the Cmax at any time during the entire period of treatment.

In one embodiment, the implant comprises a total amount of AAV and the effective treatment is characterized by the total detectable amount of the endogenous nucleic acid sequence of said AAV in the systemic circulation of the subject that is at least one log-less, two log-less, three log-less, four log-less, five log-less post administration as compared to the total detectable amount of the same endogenous nucleic acid sequence of said AAV in the systemic circulation of a subject obtained after administering another composition such as a bolus comprising the same total amount of said AAV and administered to a comparison subject at the same time. In one embodiment. The total detectable amount of the endogenous nucleic acid sequence of the AAV pertains to the amount between day 1 to day 10 post administration such as day 1 to day 4, such as day 1 to day 3, or day 2 post administration. In one embodiment, systemic circulation refers to blood, plasma, serum or lymph.

In one embodiment, the implant comprises a total amount of AAV comprised in the implant and the effective treatment is characterized by the Cₘₐₓ of the total endogenous nucleic acid sequence of said AAV in the subject that is at least one log-less, two log-less, three log-less, four log-less, five log-less as compared to the Cₘₐₓ of the same endogenous nucleic acid sequence of said AAV obtained after administering another composition such as a bolus comprising the same total amount of said AAV and administered to a subject at the same time. In one embodiment, Cmax pertains to the Cmax at any time during the entire period of treatment.

In one embodiment, the implant comprises a total amount of AAV that comprises a heterologous nucleic acid sequence and the effective treatment is characterized by the total detectable amount of the heterologous nucleic acid sequence in the systemic circulation of the subject that is at least one log-less, two log-less, three log-less, four log-less, five log-less post administration as compared to the total detectable amount of the same heterologous nucleic acid sequence in the systemic circulation of a subject obtained after administering another composition such as a bolus comprising the same total amount of said AAV and administered to a subject at the same time. In one embodiment. The total detectable amount of the heterologous nucleic acid sequence pertains to the amount between day 1 to day 10 post administration such as day 1 to day 4, such as day 1 to day 3, or day 2 post administration. In one embodiment, systemic circulation refers to blood, plasma, serum or lymph.

In one embodiment, the implant comprises a total amount of AAV that comprises a heterologous nucleic acid sequence and the effective treatment is characterized by the Cₘₐₓ of the heterologous nucleic acid sequence in the subject that is at least one log-less, two log-less, three log-less, four log-less, five log-less as compared to the Cₘₐₓ of the same heterologous nucleic acid sequence obtained after administering another composition such as a bolus comprising the same total amount of said AAV and administered to a subject at the same time. In one embodiment, Cmax pertains to the Cmax at any time during the entire period of treatment. In one embodiment, systemic circulation refers to blood, plasma, serum or lymph.

In one embodiment, “another composition” is a bolus comprising the same biologic at the same total amount.

In another embodiment, “another composition” is a pharmaceutically acceptable implant comprising the same biologic at the same total amount characterized in that the total number of days required for 100% release of the total amount of the biologic is fewer, wherein the release is measured from the time and under conditions wherein the implant is first immersed in an aqueous solution under physiological conditions such as at pH 7.2-7.4 and temperature 37° C.

In another embodiment, “another composition” is a pharmaceutically acceptable implant comprising a higher total amount of the same biologic characterized in that the total number of days required for 100% release of the total amount of the biologic is fewer, wherein the release is measured from the time and under conditions wherein the implant is first immersed in an aqueous solution under physiological conditions such as at pH 7.2-7.4 and temperature 37° C.

In one embodiment, “fewer” refers to at least 5 days fewer, such as 12 days fewer or 10 days fewer.

In another embodiment, “another composition” is a pharmaceutically acceptable implant comprising a higher total amount of the same biologic. In one embodiment, the higher total amount refers to at least 3 times higher, such as 4 times higher, 5 times higher, 6 times higher, 7 times higher, 8 times higher, 9 times higher, one log higher, two log higher, three log higher, or four log higher.

In one embodiment, “fewer” refers to at least 5 days fewer, such as 12 days fewer or 10 days fewer.

In certain embodiments, the method of “effective” treatment of an ocular disorder such as an ocular genetic disorder is defined as any of the embodiments above.

In certain embodiments, the method of “effective” treatment of an ocular disorder such as an occur genetic disorder in addition comprises at least one of the following
(A) controlling inflammation as descried in the previous sections,
(B) controlling an immune response as described in the previous sections.

In one embodiment, the biologic is an AAV selected from a group consisting of AAV1, AAV2 such as AAV2.7m8, AAV3, AAV4, AAV5, AAV6, AAV7, AAV8, AAV9, AAV10, AAV11 and a mutant, hybrid or variant thereof at a total amount in the order from 10⁹ to 10¹⁵ vg, such as in the order from 10⁹ to 10¹³ vg. In some embodiments, the total amount of the AAV is from 10¹⁰ to 10¹⁵ vg. In some embodiments, the total amount of the AAV is from 10¹⁰ to 10¹⁴ vg. In some embodiments, the total amount of the AAV is from 10¹⁰ to 10¹³ vg. In some embodiments, the total amount of the AAV is from 10¹⁰ to 10¹² vg. In some embodiments, the total amount of the AAV is from 10¹⁰ to 10¹¹ vg. In these embodiments, the AAV comprises at least one heterologous nucleic acid sequence. In these embodiments, the heterologous nucleic acid sequence can be either a coding nucleic acid sequence or non-coding nucleic acid sequence. When the heterologous nucleic acid sequence is a coding nucleic acid sequence, it may code for a therapeutic protein as described in the section under the heading of biologic. In this embodiment, the inventors have found that the inflammation can be controlled by the controlled release characterized by no greater than 9.0×10⁹ to 1.5×10¹⁰ vg AAV released on day 1, no greater than in the order 10¹¹ vg AAV per day such as in the order 10⁸, or 10⁹ or 10¹⁰ such as 5.0×10⁹ to 1.5×10¹⁰ vg AAV released per day from day 2 until the last day of the controlled release, and/or the number of days required for 100% release of the AAV is not less than 4 days.

In one embodiment, the method of effective treatment of ocular disorder such as an ocular genetic disorder comprises intravitreal injection of the pharmaceutically acceptable implant comprising a biologic to the subject in need thereof. In one embodiment, the biologic is an AAV selected from a group consisting of AAV1, AAV2 such as AAV2.7m8, AAV3, AAV4, AAV5, AAV6, AAV7, AAV8, AAV9, AAV10, AAV11 and a mutant, hybrid or variant thereof at a total amount in the order from 10⁹ to 10¹⁵ vg, such as in the order from 10⁹ to 10¹³ vg. In some embodiments, the total amount of the AAV is from 10¹⁰ to 10¹⁵ vg. In some embodiments, the total amount of the AAV is from 10¹⁰ to 10¹⁴ vg. In some embodiments, the total amount of the AAV is from 10¹⁰ to 10¹³ vg. In some embodiments, the total amount of the AAV is from 10¹⁰ to 10¹² vg. In some embodiments, the total amount of the AAV is from 10¹⁰ to 10¹¹ vg. In these embodiments, the AAV comprises at least one heterologous nucleic acid sequence. In these embodiments, the heterologous nucleic acid sequence can be either a coding nucleic acid sequence or noncoding nucleic acid sequence. When the heterologous nucleic acid sequence is a coding nucleic acid sequence, it may code for a therapeutic protein as described in the section under the heading of biologic. In this embodiment, the inventors have found that the inflammation is controlled by a controlled release characterized by no greater than 6×10⁹ to 1.0×10¹⁰ AAV vg per mL of the vitreous volume of the subject released on day 1, no greater than 3.5×10⁹ to 1.0×10¹⁰ AAV vg per mL of the vitreous volume of the subject released per day from day 2 until the last day of the controlled release, and/or the number of days required for 100% release of the AAV is not less than 4 days.

In another embodiment, the method of effective treatment of ocular disorder such as an ocular genetic disorder comprises administering the pharmaceutically acceptable implant that comprises AAV in the order less than 2.0×10¹⁰ vg and the controlled release is characterized in that the number of days required for 100% release of the AAV is at least 4 days.

In another embodiment, the method of effective treatment of ocular disorder such as an ocular genetic disorder comprises administering the pharmaceutically acceptable implant that comprises AAV order greater than 2.0×10¹⁰ vg and up to the order of 10¹⁵ vg and the controlled release is characterized in that the number of days required for 100% release of the AAV is greater than 10 days or more.

In another embodiment, the method of effective treatment of ocular disorder such as an ocular genetic disorder comprises administering the pharmaceutically acceptable implant that comprises AAV order greater than 2.0×10¹⁰ vg and up to the order of 10¹⁵ vg and the controlled release is characterized in that the number of days required for 100% release of the AAV is greater than 15 days or more.

In another embodiment, the method of effective treatment of ocular disorder such as an ocular genetic disorder comprises administering the pharmaceutically acceptable implant that comprises AAV order greater than 2.0×10¹⁰ vg and up to the order of 10¹³ vg and the controlled release is characterized in that the number of days required for 100% release of the AAV is greater than 10 days or more.

In another embodiment, the method of effective treatment of ocular disorder such as an ocular genetic disorder comprises administering the pharmaceutically acceptable implant that comprises AAV order greater than 2.0×10¹⁰ vg and up to the order of 10¹³ vg and the controlled release is characterized in that the number of days required for 100% release of the AAV is greater than 15 days or more.

In another embodiment, the method of effective treatment of ocular disorder such as an ocular genetic disorder comprises administering the pharmaceutically acceptable implant that comprises AAV order greater than 2.0×10¹⁰ vg and up to the order of 10¹² vg and the controlled release is characterized in that the number of days required for 100% release of the AAV is greater than 10 days or more.

In another embodiment, the method of effective treatment of ocular disorder such as an ocular genetic disorder comprises administering the pharmaceutically acceptable implant that comprises AAV order greater than 2.0×10¹⁰ vg and up to the order of 10¹² vg and the controlled release is characterized in that the number of days required for 100% release of the AAV is greater than 15 days or more.

In another embodiment, the method of effective treatment of ocular disorder such as an ocular genetic disorder comprises administering the pharmaceutically acceptable implant that comprises AAV order greater than 2.0×10¹⁰ vg and up to the order of 10¹¹ vg and the controlled release is characterized in that the number of days required for 100% release of the AAV is greater than 10 days or more.

In another embodiment, the method of effective treatment of ocular disorder such as an ocular genetic disorder comprises administering the pharmaceutically acceptable implant that comprises AAV order greater than 2.0×10¹⁰ vg and up to the order of 10¹¹ vg and the controlled release is characterized in that the number of days required for 100% release of the AAV is greater than 15 days or more.

The methods of treatment described in this section are also to be construed as describing a pharmaceutically acceptable implant for use in the methods of treatment. Further, the methods of treatment described in this section are also to be construed as describing use of the pharmaceutically acceptable implant for the manufacture of a medicament for the treatment.

### Method of Prophylactic Inflammation Treatment

A method of prophylactic inflammation treatment in a subject when treating an ocular disorder or an ocular genetic disorder comprising the following sequential steps:
(A) administering a composition to the eye of the subject comprising a total amount of a virus such as AAV comprising at least one heterologous nucleic acid sequence,
(B) assessing the total detectable amount of an endogenous nucleic acid sequence of said virus such as AAV or the heterologous nucleic acid sequence comprised in said virus such as AAV in the systemic circulation of the subject such as on day 1, day 2 or day 3 post administration,
(D) providing to the subject a prophylactic inflammation treatment if the detectable amount is higher than five log-less such as four log-less, three log-less, two log-less or one log-less as compared to the total amount of the AAV in the composition, wherein the prophylactic inflammation treatment comprises administering an anti-inflammatory agent to the eye of the subject.

In one embodiment, systemic circulation refers to blood, plasma, serum or lymph. In one embodiment, the anti-inflammatory agent is selected from hydrocortisone, loteprednol, cortisol, cortisone, prednisolone, methylprednisolone, dexamethasone, betamethasone, triamcinolone, aldosterone or fludrocortisone.

In one embodiment, the composition may be the pharmaceutically acceptable implant described herein. In another embodiment, the composition may be any composition comprising a virus comprising at least one heterologous nucleic acid sequence that is administered to the eye of the subject for treatment of an ocular disorder such as an ocular genetic disorder.

In one embodiment, the virus is an AAV selected from a group consisting of AAV1, AAV2 such as AAV2.7m8, AAV3, AAV4, AAV5, AAV6, AAV7, AAV8, AAV9, AAV10, AAV11 and a mutant, hybrid or variant thereof at a total amount in the order from 10⁹ to 10¹⁵ vg, such as in the order from 10⁹ to 10¹³ vg.

### Method of Treating Inflammation

The present invention also provides methods of treating inflammation in the eye of a subject in need thereof.

The invention provides a method of treating inflammation in the eye of a subject by administering a therapeutically effective amount of a tyrosine kinase inhibitor to the eye of the subject in need thereof. In one embodiment, the inflammation is caused by an innate immune response.

In certain embodiments, the administration of the tyrosine kinase inhibitor is conducted within 24 hours of the inflammation causing event. In some embodiments, the tyrosine kinase inhibitor is co-administered with the inflammation causing event.

In various embodiments, the tyrosine kinase inhibitor is selected from a group consisting of axitinib, sunitinib, sorafenib, paxopanib, or tivozanib. In some embodiments, the tyrosine kinase inhibitor is axitinib.

The methods of treatment described in this section are also to be construed as describing a therapeutically effective amount of a tyrosine kinase inhibitor for use in the methods of treatment. Further, the methods of treatment described in this section are also to be construed as describing use of a therapeutically effective amount of a tyrosine kinase inhibitor for the manufacture of a medicament for the treatment.

### Methods of Treatment Other than Ocular

### Localized Environment

The methods of treatment for ocular disorders described in the previous sections can also be applied to other areas such as areas with a localized environment of the subject in need thereof.

In certain embodiments, a method of treating a disorder or disease of central nervous system, ear including the cochlea, skin, ovaries, uterus, or joints such as articular joints, cartilage, ligaments, tendons, and surrounding tissues comprising administering the pharmaceutically acceptable implant of the invention to a subject in need thereof is provided.

In certain embodiments, the route of administration of the pharmaceutically acceptable implant of the invention to a subject includes intra-articular, intrathecal, epidural, intramuscular, subcutaneous, transdermal, intratumoral, intracochlear, intranasal, or intrauterine.

In certain embodiments, the pharmaceutically acceptable implant comprises a xerogel, at least one dehydration stabilizer and a biologic all of which are described in the previous sections.

In certain embodiments, the pharmaceutically acceptable implant is for a controlled release of the biologic.

In one embodiment, the biologic is a recombinant protein such as an antibody, an antigen binding fragment, a fusion protein, a cytokine, a transcription factor, an enzyme or a hormone.

In this embodiment, the controlled release in the method of treatment is characterized by the amount of the biologic released on day 1 is from 0 to 50% of the total amount of the biologic, the amount of the biologic released per day from day 2 until the last day of release is from 0 to 50% of the total amount of the biologic, and/or the number of days required for 100% release of the total amount of the biologic is at least 2 days. In one embodiment, the controlled release in the method of treatment is characterized by the amount of the biologic released on day 1 is from 0 to 25% of the total amount of the biologic, the amount of the biologic released per day from day 2 until the last day of release is from 0 to 50% of the total amount of the biologic, and/or the number of days required for 100% release of the total amount of the biologic is at least 2 days. In one embodiment, the controlled release in the method of treatment is characterized by the amount of the biologic released on day 1 is from 0 to 20% of the total amount of the biologic, the amount of the biologic released per day from day 2 until the last day of release is from 0 to 50% of the total amount of the biologic, and/or the number of days required for 100% release of the total amount of the biologic is at least 2 days. In one embodiment, the controlled release in the method of treatment is characterized by the amount of the biologic released on day 1 is from 0 to 10% of the total amount of the biologic, the amount of the biologic released per day from day 2 until the last day of release is from 0 to 50% of the total amount of the biologic, and/or the number of days required for 100% release of the total amount of the biologic is at least 2 days. In some embodiments, the number of days required for 100% release of the total amount of the biologic is at least 3 days, or at least 4 days.

In certain embodiments, the pharmaceutically acceptable implant comprises a biologic, which is a viral vector selected from a group consisting of retrovirus, adenovirus, adeno-associated virus (AAV), lentivirus and herpes simplex virus.

In certain embodiments, the pharmaceutically acceptable implant comprises adeno-associated virus (AAV).

In certain embodiments, the pharmaceutically acceptable implant comprises the adeno-associated virus (AAV) selected from a group consisting of AAV1, AAV2 such as AAV2.7m8, AAV3, AAV4, AAV5, AAV6, AAV7, AAV8, AAV9, AAV10, AAV11 and a mutant, hybrid or variant thereof.

In some embodiments, the AAV in the pharmaceutically acceptable implant is at a total amount in the order from 10⁹ to 10¹⁷ vg such as 10⁹ to 10¹⁵ vg, such as in the order from 10¹⁰ to 10¹³ vg.

In these embodiments, the AAV comprises at least one heterologous nucleic acid sequence(s). In these embodiments, the heterologous nucleic acid sequence can be either a coding nucleic acid sequence or non-coding nucleic acid sequence. When the heterologous nucleic acid sequence is a coding nucleic acid sequence, it may code for a therapeutic protein as described in the section under the heading of biologic.

In certain embodiments, the heterologous nucleic acid(s) code(s) for a therapeutic protein, which is absent in the subject, or present at a reduced level in the subject as compared to the same but healthy subject.

In one embodiment, the controlled release is characterized by the amount of the AAV released on day 1 is from 0 to 50% of the total amount of the AAV, the amount of the AAV released per day from day 2 until the last day of release is from 0 to 50% of the total amount of the AAV, and/or the number of days required for 100% release of the total amount of the AAV is at least 4 days. In one embodiment, the controlled release is characterized by the amount of the AAV released on day 1 is from 0 to 25% of the total amount of the AAV, the amount of the AAV released per day from day 2 until the last day of release is from 0 to 50% of the total amount of the AAV, and/or the number of days required for 100% release of the total amount of the AAV is at least 4 days. In one embodiment, the controlled release is characterized by the amount of the AAV released on day 1 is from 0 to 20% of the total amount of the AAV, the amount of the AAV released per day from day 2 until the last day of release is from 0 to 50% of the total amount of the AAV, and/or the number of days required for 100% release of the total amount of the AAV is at least 4 days. In one embodiment, the controlled release is characterized by the amount of the AAV released on day 1 is from 0 to 10% of the total amount of the AAV, the amount of the AAV released per day from day 2 until the last day of release is from 0 to 50% of the total amount of the AAV, and/or the number of days required for 100% release of the total amount of the AAV is at least 4 days.

### Central Nervous System

In certain embodiments, a method of treating a central nervous system disorder such as a central nervous system genetic disorder comprising administering to a subject the pharmaceutically acceptable implant of the invention is provided.

In some embodiments, the disorder of the central nervous system is selected from a group consisting of huntington's disease, epilepsy, parkinson's disease, Alzheimer's disease, stroke, corticobasal degeneration, corticogasal ganglionic degeneration, frontotemporal dementia, multiple system atrophy, progressive supranuclear palsay, Adrenoleukodystrophy, Alzheimer's disease, Amyotropic lateral sclerosis, Angelman syndrome, Ataxia telangiectasia, Charco-Marie-Tooth Syndrome, Cockayne syndrome, Deafness, Duchenne Muscular Dystrophy, Epilepsy, essential tremor, fragile X syndrome, Friedrich's ataxia, Gaucher disease, Huntington disease, Lesch-Nyhan syndrome, Maple syrup urine disease, Menkes syndrome, Myotonic dystrophy, narcolepsy, neurofibromatosis, niemann-pick disease Parkinson disease, phenylketonuria, prader-willi syndrome, refsum disease, Rett syndrome, spinal muscular atrophy, spinocerebellar ataxia, Tangier disease, Taysachs disease, Tuberous sclerosis, Von Hippel-Lindau syndrome, Williams syndrome, Wilson's disease, Zellwegger syndrome, and brain cancer.

In certain embodiments, a method of treating a central nervous system disorder such as a central nervous system genetic disorder comprises administering to a subject the pharmaceutically acceptable implant of the invention intrathecally, intracranially, intracerebroventricularly, lateral cerebro ventricularly, intranasally, endovascularly, or intraparenchymally.

In certain embodiments, the pharmaceutically acceptable implant comprises a xerogel, at least one dehydration stabilizer and a biologic all of which are described in the previous sections.

In certain embodiments, the pharmaceutically acceptable implant is for a controlled release of the biologic.

In one embodiment, the biologic is a recombinant protein such as an antibody, an antigen binding fragment, a fusion protein, a cytokine, a transcription factor, an enzyme or a hormone.

In this embodiment, the controlled release in the method of treatment is characterized by the amount of the biologic released on day 1 is from 0 to 50% of the total amount of the biologic, the amount of the biologic released per day from day 2 until the last day of release is from 0 to 50% of the total amount of the biologic, and/or the number of days required for 100% release of the total amount of the biologic is at least 2 days. In some embodiments, the number of days required for 100% release of the total amount of the biologic is at least 3 days, or at least 4 days.

In certain embodiments, the pharmaceutically acceptable implant comprises a biologic, which is a viral vector selected from a group consisting of retrovirus, adenovirus, adeno-associated virus (AAV), lentivirus and herpes simplex virus.

In certain embodiments, the pharmaceutically acceptable implant comprises adeno-associated virus (AAV).

In certain embodiments, the pharmaceutically acceptable implant comprises the adeno-associated virus (AAV) selected from a group consisting of AAV1, AAV2 such as AAV2.7m8, AAV3, AAV4, AAV5, AAV6, AAV7, AAV8, AAV9, AAV10, AAV11 and a mutant, hybrid or variant thereof.

In some embodiments, the AAV in the pharmaceutically acceptable implant is at a total amount in the order from 10⁹ to 10¹⁷ such as 10⁹ to 10¹⁵ vg, such as in the order from 10¹⁰ to 10¹³ vg.

In these embodiments, the AAV comprises at least one heterologous nucleic acid sequence(s). In these embodiments, the heterologous nucleic acid sequence can be either a coding nucleic acid sequence or non-coding nucleic acid sequence. When the heterologous nucleic acid sequence is a coding nucleic acid sequence, it may code for a therapeutic protein as described in the section under the heading of biologic.

In certain embodiments, the heterologous nucleic acid(s) code(s) for a therapeutic protein, which is absent in the subject, or present at a reduced level in the subject as compared to the same but healthy subject.

In one embodiment, the controlled release is characterized by the amount of the AAV released on day 1 is from 0 to 50% of the total amount of the AAV, the amount of the AAV released per day from day 2 until the last day of release is from 0 to 50% of the total amount of the AAV, and/or the number of days required for 100% release of the total amount of the AAV is at least 4 days. In one embodiment, the controlled release is characterized by the amount of the AAV released on day 1 is from 0 to 25% of the total amount of the AAV, the amount of the AAV released per day from day 2 until the last day of release is from 0 to 50% of the total amount of the AAV, and/or the number of days required for 100% release of the total amount of the AAV is at least 4 days. In one embodiment, the controlled release is characterized by the amount of the AAV released on day 1 is from 0 to 20% of the total amount of the AAV, the amount of the AAV released per day from day 2 until the last day of release is from 0 to 50% of the total amount of the AAV, and/or the number of days required for 100% release of the total amount of the AAV is at least 4 days. In one embodiment, the controlled release is characterized by the amount of the AAV released on day 1 is from 0 to 10% of the total amount of the AAV, the amount of the AAV released per day from day 2 until the last day of release is from 0 to 50% of the total amount of the AAV, and/or the number of days required for 100% release of the total amount of the AAV is at least 4 days.

### Articular Disease

In certain embodiments, a method of treating an articular disease comprising administering to a subject the pharmaceutically acceptable implant of the invention is provided.

In some embodiments, the articular disease includes osteoarthritis, rheumatoid arthritis and related disorders, neuromuscular disease, autoimmune disorder or a joint injury or defect.

In certain embodiments, a method of treating an articular disease comprises administering to a subject the pharmaceutically acceptable implant of the invention intramuscularly, subcutaneously, intra-articularly, or periaritcularly.

In certain embodiments, the pharmaceutically acceptable implant comprises a xerogel, at least one dehydration stabilizer and a biologic all of which are described in the previous sections.

In certain embodiments, the pharmaceutically acceptable implant is for a controlled release of the biologic.

In one embodiment, the biologic is a recombinant protein such as an antibody, an antigen binding fragment, a fusion protein, a cytokine, a transcription factor, an enzyme or a hormone.

In this embodiment, the controlled release in the method of treatment is characterized by the amount of the biologic released on day 1 is from 0 to 50% of the total amount of the biologic, the amount of the biologic released per day from day 2 until the last day of release is from 0 to 50% of the total amount of the biologic, and/or the number of days required for 100% release of the total amount of the biologic is at least 2 days. In some embodiments, the number of days required for 100% release of the total amount of the biologic is at least 3 days, or at least 4 days.

In certain embodiments, the pharmaceutically acceptable implant comprises a biologic, which is a viral vector selected from a group consisting of retrovirus, adenovirus, adeno-associated virus (AAV), lentivirus and herpes simplex virus.

In certain embodiments, the pharmaceutically acceptable implant comprises adeno-associated virus (AAV).

In certain embodiments, the pharmaceutically acceptable implant comprises the adeno-associated virus (AAV) selected from a group consisting of AAV1, AAV2 such as AAV2.7m8, AAV3, AAV4, AAV5, AAV6, AAV7, AAV8, AAV9, AAV10, AAV11 and a mutant, hybrid or variant thereof.

In some embodiments, the AAV in the pharmaceutically acceptable implant is at a total amount in the order from 10⁹ to 10¹⁷ such as 10⁹ to 10¹⁵ vg, such as in the order from 10¹⁰ to 10¹³ vg.

In these embodiments, the AAV comprises at least one heterologous nucleic acid sequence(s). In these embodiments, the heterologous nucleic acid sequence can be either a coding nucleic acid sequence or non-coding nucleic acid sequence. When the heterologous nucleic acid sequence is a coding nucleic acid sequence, it may code for a therapeutic protein as described in the section under the heading of biologic.

In certain embodiments, the heterologous nucleic acid(s) code(s) for a therapeutic protein, which is absent in the subject, or present at a reduced level in the subject as compared to the same but healthy subject.

In one embodiment, the controlled release is characterized by the amount of the AAV released on day 1 is from 0 to 50% of the total amount of the AAV, the amount of the AAV released per day from day 2 until the last day of release is from 0 to 50% of the total amount of the AAV, and/or the number of days required for 100% release of the total amount of the AAV is at least 4 days. In one embodiment, the controlled release is characterized by the amount of the AAV released on day 1 is from 0 to 25% of the total amount of the AAV, the amount of the AAV released per day from day 2 until the last day of release is from 0 to 50% of the total amount of the AAV, and/or the number of days required for 100% release of the total amount of the AAV is at least 4 days. In one embodiment, the controlled release is characterized by the amount of the AAV released on day 1 is from 0 to 20% of the total amount of the AAV, the amount of the AAV released per day from day 2 until the last day of release is from 0 to 50% of the total amount of the AAV, and/or the number of days required for 100% release of the total amount of the AAV is at least 4 days. In one embodiment, the controlled release is characterized by the amount of the AAV released on day 1 is from 0 to 10% of the total amount of the AAV, the amount of the AAV released per day from day 2 until the last day of release is from 0 to 50% of the total amount of the AAV, and/or the number of days required for 100% release of the total amount of the AAV is at least 4 days.

### Regeneration of Chondrocytes or Cartilage

In certain embodiments, a method of regeneration of chondrocytes or cartilage type cells such as articular cartilage repair including regeneration of chondrocytes or other cartilage-type cells and/or the generation and/or repair of cartilage tissue comprising administering to a subject in need thereof the pharmaceutically acceptable implant of the invention is provided.

In some embodiments, the subject in need thereof incudes a subject having intervertebral disc disease, chondrodystrophies including osteoarthritis, achondroplasia, costochondritis, or spinal disc herniation.

In certain embodiments, a method of regeneration of chondrocytes or cartilage type cells such as articular cartilage repair including regeneration of chondrocytes or other cartilage-type cells and/or the generation and/or repair of cartilage tissue comprises administering to a subject the pharmaceutically acceptable implant of the invention intramuscularly, subcutaneously, intra-articularly, or peri-aritcularly.

In certain embodiments, the pharmaceutically acceptable implant comprises a xerogel, at least one dehydration stabilizer and a biologic all of which are described in the previous sections.

In certain embodiments, the pharmaceutically acceptable implant is for a controlled release of the biologic.

In one embodiment, the biologic is a recombinant protein such as an antibody, an antigen binding fragment, a fusion protein, a cytokine, a transcription factor, an enzyme or a hormone.

In this embodiment, the controlled release in the method of treatment is characterized by the amount of the biologic released on day 1 is from 0 to 50% of the total amount of the biologic, the amount of the biologic released per day from day 2 until the last day of release is from 0 to 50% of the total amount of the biologic, and/or the number of days required for 100% release of the total amount of the biologic is at least 2 days. In some embodiments, the number of days required for 100% release of the total amount of the biologic is at least 3 days, or at least 4 days.

In certain embodiments, the pharmaceutically acceptable implant comprises a biologic, which is a viral vector selected from a group consisting of retrovirus, adenovirus, adeno-associated virus (AAV), lentivirus and herpes simplex virus.

In certain embodiments, the pharmaceutically acceptable implant comprises adeno-associated virus (AAV).

In certain embodiments, the pharmaceutically acceptable implant comprises the adeno-associated virus (AAV) selected from a group consisting of AAV1, AAV2 such as AAV2.7m8, AAV3, AAV4, AAV5, AAV6, AAV7, AAV8, AAV9, AAV10, AAV11 and a mutant, hybrid or variant thereof.

In some embodiments, the AAV in the pharmaceutically acceptable implant is at a total amount in the order from 10⁹ to 10¹⁷ vg such as 10⁹ to 10¹⁵ vg, such as in the order from 10¹⁰ to 10¹³ vg.

In these embodiments, the AAV comprises at least one heterologous nucleic acid sequence(s). In these embodiments, the heterologous nucleic acid sequence can be either a coding nucleic acid sequence or non-coding nucleic acid sequence. When the heterologous nucleic acid sequence is a coding nucleic acid sequence, it may code for a therapeutic protein as described in the section under the heading of biologic.

In certain embodiments, the heterologous nucleic acid(s) code(s) for a therapeutic protein, which is absent in the subject, or present at a reduced level in the subject as compared to the same but healthy subject.

In one embodiment, the controlled release is characterized by the amount of the AAV released on day 1 is from 0 to 50% of the total amount of the AAV, the amount of the AAV released per day from day 2 until the last day of release is from 0 to 50% of the total amount of the AAV, and/or the number of days required for 100% release of the total amount of the AAV is at least 4 days. In one embodiment, the controlled release is characterized by the amount of the AAV released on day 1 is from 0 to 25% of the total amount of the AAV, the amount of the AAV released per day from day 2 until the last day of release is from 0 to 50% of the total amount of the AAV, and/or the number of days required for 100% release of the total amount of the AAV is at least 4 days. In one embodiment, the controlled release is characterized by the amount of the AAV released on day 1 is from 0 to 20% of the total amount of the AAV, the amount of the AAV released per day from day 2 until the last day of release is from 0 to 50% of the total amount of the AAV, and/or the number of days required for 100% release of the total amount of the AAV is at least 4 days. In one embodiment, the controlled release is characterized by the amount of the AAV released on day 1 is from 0 to 10% of the total amount of the AAV, the amount of the AAV released per day from day 2 until the last day of release is from 0 to 50% of the total amount of the AAV, and/or the number of days required for 100% release of the total amount of the AAV is at least 4 days.

### Benign Tumors

In certain embodiments, a method of treating a tumor such as a benign tumor comprising administering to a subject the pharmaceutically acceptable implant of the invention is provided.

In certain embodiments, a method of treating a tumor such as a benign tumor comprises administering to a subject the pharmaceutically acceptable implant of the invention intratumorally.

In certain embodiments, the pharmaceutically acceptable implant comprises a xerogel, at least one dehydration stabilizer and a biologic all of which are described in the previous sections.

In certain embodiments, the pharmaceutically acceptable implant is for a controlled release of the biologic.

In one embodiment, the biologic is a recombinant protein such as an antibody, an antigen binding fragment, a fusion protein, a cytokine, a transcription factor, an enzyme or a hormone.

In this embodiment, the controlled release in the method of treatment is characterized by the amount of the biologic released on day 1 is from 0 to 50% of the total amount of the biologic, the amount of the biologic released per day from day 2 until the last day of release is from 0 to 50% of the total amount of the biologic, and/or the number of days required for 100% release of the total amount of the biologic is at least 2 days. In some embodiments, the number of days required for 100% release of the total amount of the biologic is at least 3 days, or at least 4 days.

In certain embodiments, the pharmaceutically acceptable implant comprises a biologic, which is a viral vector selected from a group consisting of retrovirus, adenovirus, adeno-associated virus (AAV), lentivirus and herpes simplex virus.

In certain embodiments, the pharmaceutically acceptable implant comprises adeno-associated virus (AAV).

In certain embodiments, the pharmaceutically acceptable implant comprises the adeno-associated virus (AAV) selected from a group consisting of AAV1, AAV2 such as AAV2.7m8, AAV3, AAV4, AAV5, AAV6, AAV7, AAV8, AAV9, AAV10, AAV11 and a mutant, hybrid or variant thereof.

In some embodiments, the AAV in the pharmaceutically acceptable implant is at a total amount in the order from 10⁹ to 10¹⁷ such as 10⁹ to 10¹⁵ vg, such as in the order from 10¹⁰ to 10¹³ vg.

In these embodiments, the AAV comprises at least one heterologous nucleic acid sequence(s). In these embodiments, the heterologous nucleic acid sequence can be either a coding nucleic acid sequence or non-coding nucleic acid sequence. When the heterologous nucleic acid sequence is a coding nucleic acid sequence, it may code for a therapeutic protein as described in the section under the heading of biologic.

In one embodiment, the controlled release is characterized by the amount of the AAV released on day 1 is from 0 to 50% of the total amount of the AAV, the amount of the AAV released per day from day 2 until the last day of release is from 0 to 50% of the total amount of the AAV, and/or the number of days required for 100% release of the total amount of the AAV is at least 4 days. In one embodiment, the controlled release is characterized by the amount of the AAV released on day 1 is from 0 to 25% of the total amount of the AAV, the amount of the AAV released per day from day 2 until the last day of release is from 0 to 50% of the total amount of the AAV, and/or the number of days required for 100% release of the total amount of the AAV is at least 4 days. In one embodiment, the controlled release is characterized by the amount of the AAV released on day 1 is from 0 to 20% of the total amount of the AAV, the amount of the AAV released per day from day 2 until the last day of release is from 0 to 50% of the total amount of the AAV, and/or the number of days required for 100% release of the total amount of the AAV is at least 4 days. In one embodiment, the controlled release is characterized by the amount of the AAV released on day 1 is from 0 to 10% of the total amount of the AAV, the amount of the AAV released per day from day 2 until the last day of release is from 0 to 50% of the total amount of the AAV, and/or the number of days required for 100% release of the total amount of the AAV is at least 4 days.

In certain embodiments, the heterologous nucleic acid sequences codes for a protein pertaining to the apoptosis, cell lysis, anti-tumor immunity or angio-genesis inhibition pathway.

### EXAMPLES

The following Examples are included to demonstrate certain aspects and embodiments of the invention as described in the claims. It should be appreciated by those of skill in the art, however, that the following description is illustrative only and should not be taken in any way as a restriction of the invention.

### Example 1—Formulation: Proof-of-Concept that AAV can Maintain Infectivity after Organogel Processing

### Example 1.1: AAV2—Effects of Lyophilization, Organic Solvent Exposure and PEG Crosslinking Chemistry

This study evaluated whether adeno-associated viruses (AAVs) could maintain their infectivity after organogel processing. The first 3 steps, freeze-drying, DMC exposure, and PEG crosslinking were evaluated. The 3rd step was modified in this study to expose the freeze-dried AAVs to PEG crosslinking chemistry, but without organogel formation.

Infectivity testing was performed through a cell assay. AAVs with a GFP reporter were cultured with HEK293 cells and were assessed for % transduction efficiency (GFP+ cells/Total cells) and mean fluorescent intensity (MFI) of GFP+ cells.

AAV2 serotype was evaluated in this study.

Three sterile DMC solutions were made 1) anhydrous DMC, 2) 2% 4a20k PEG SS in DMC, and 3) 8% 20k methoxy PEG NH2 in DMC. Solutions were sterile filtered through an acrodisc syringe filter, 0.2 μm Nylon (PALL, PN4433) and frozen at −20° C. prior to use.

Groups and formulations prepared for AAV2 cell transduction efficiency testing are as outlined in Table 1 below. Sample preparation for each group was conducted as described in Table 2 below.

TABLE 1
Outline of groups and formulations prepared for AAV2 cell transduction efficiency testing

| | | | | | 2^{nd} Lyophilization | | |
|---|---|---|---|---|---|---|---|
| | | | | | | 2% 20k | |
| | | | | 1^{st} | | Methoxy | 8% |
| | | | | Lyophilization | | PEG NH2 | 4a20kSS |
| | | | Config- | AAV Stock | DMC | in DMC | in DMC |
| Virus | Group | Description | uration | (uL) | (uL) | (uL) | (uL) |
| AAV2- | G1_AAV2 | (+) Control - | Liquid | 30 | | | |
| CMV- | | Virus in PBS | | | | | |
| GFP | G2_AAV2 | Lyophilized | Powder | 30 | | | |
| | G3_AAV2 | Lyo → +DMC | Powder | 30 | 100 | | |
| | | → Lyo | | | | | |
| | G4_AAV2 | Lyo → +DMC | Powder | 30 | | 50 | 50 |
| | | +PEG → Lyo | | | | | |

TABLE 2
Sample preparation for each group

| Group 1-(+) Control: *No prep work needed | Group 2-Lyophilized: Freeze-dry virus |
|---|---|
| Group 3-Lyo → + DMC -> Lyo | Group 4-Lyo → + DMC + PEG -> Lyo* |
| Freeze-dry virus | Freeze-dry virus |
| Let DMC thaw to room temperature | Let DMC + PEG_A and DMC + PEG_E |
| Add 100 uL of DMC to powder | thaw to room temperature |
| Mix (eg, pipetting, vortexing) | Add 50 uL of DMC + PEG_A solution to |
| Allow mix to stand for 10 minutes | powder |
| Freeze-dry | Add 50 uL of DMC + PEG_E solution |
| | Mix (eg, pipetting, vortexing) |
| | Allow mix to stand for 10 minutes |
| | Freeze-Dry |
| | *PEG is dissolved in DMC solvent |

Control group (G1) was kept frozen at −80° C. G2-G4 were freeze-dried in a manifold lyophilizer (Labconco FreeZone 2.5L). 30 μL of AAV2 aliquots were removed from the −80° C. freezer and caps were loosened. AAV2 aliquots were transferred to the lyophilizer while still frozen, and the first lyophilization was done overnight (no set time) at a vacuum of 60 mTorr and the condenser set at −50° C. No additional excipients were added to the AAVs prior to freeze-drying.

Prior to the second lyophilization run, DMC solutions were added to G3 and G4 samples. The amounts added are summarized in Tables 1 and 2. For G4 samples, the equal volumetric additions of 2% 4a20k SS and 8% 20k methoxy PEG NH2 resulted in a 5% PEG solution. Additionally, the use of a monofunctional methoxy PEG NH2 would prevent network formation, even as chemical reactions took place between the SS and NH2 end groups.

After DMC solutions were added to the dried powder, the mixture was briefly vortexed and sat for 10 minutes at ambient lab conditions. Samples were then frozen in the −80° C. freezer for 2 h and lyophilized overnight with a similar procedure to the first lyophilization run to remove the DMC. All samples were stored at −80° C. prior to use.

For cell transduction efficiency assessment, lyophilized samples (G2-G4) were reconstituted with 30 μL of sterile ddH2O. All groups were then diluted with culture media to the appropriate titer before adding to the cells. The well plate layout is shown in Table 3.

TABLE 3
96 well plate layout (HEK293 for AAV2)

| | | | | | MOI 1 | | | MOI 2 | | | MOI 3 | | |
|---|---|---|---|---|---|---|---|---|---|---|---|---|---|
| | | No Virus | | | (1 × 10³) | | | (1 × 10⁴) | | | (1 × 10⁵) | | |
| Serotype | Group | 1 | 2 | 3 | 4 | 5 | 6 | 7 | 8 | 9 | 10 | 11 | 12 |

| | | |
|---|---|---|
| AAV2 in | 1 | A |
| HEK293 | 2 | B |
| | 3 | C |
| | 4 | D |

48 h post-infection, the plate was analyzed for % transduction efficiency and MFI of GFP+ cells via flow cytometry.

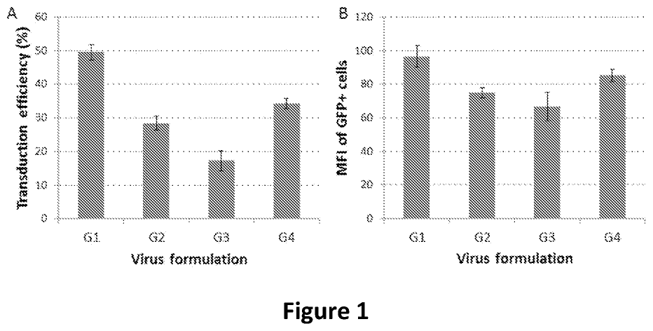

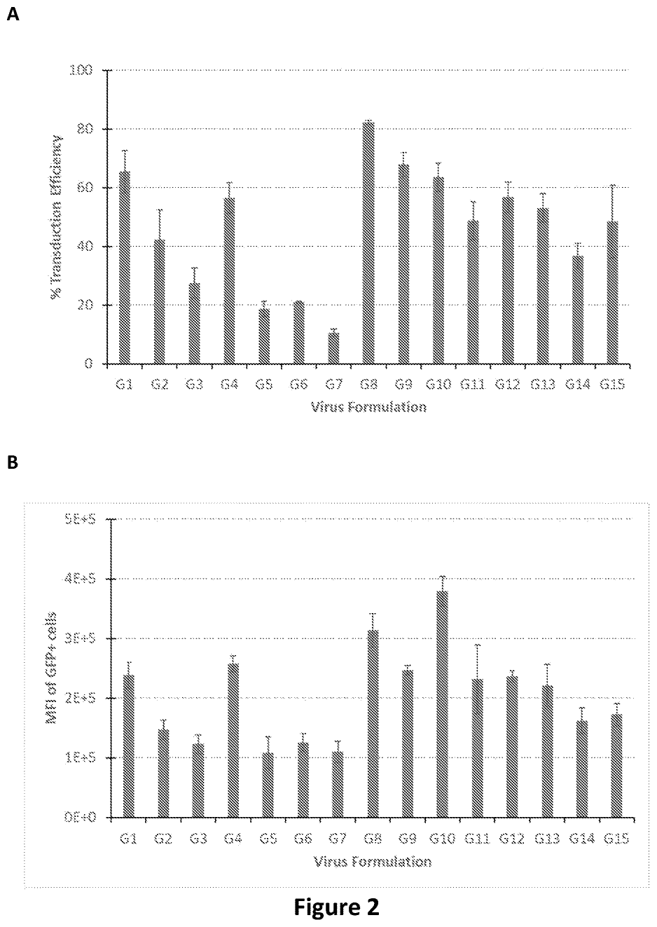

For cells infected with AAV2, a drop in transduction efficiency and MFI was seen following lyophilization (FIG. 1). An additional drop in infectivity was seen with DMC exposure.

### Example 1.2: AAV Infectivity can be Protected During Lyophilization, Organic Solvent Exposure, Organogel and Xerogel Processing

The primary goals of this study were:
Improve infectivity of lyophilized AAVs through the addition of lyoprotectant excipients.
Evaluate infectivity of encapsulated and released AAVs from the xerogel that is a hydrogel after exposure to an aqueous solution.

An outline of the 15 groups evaluated in this study are highlighted in Table 4 below.

TABLE 4
Outline of groups evaluated for a cell transduction assay

| | | | Overnight |
|---|---|---|---|
| | | | PBS |
| Group | Description | Configuration | Incubation |
| G1 | (+) Control-Virus in PBS | Liquid | — |
| G2 | Lyo | Powder | — |
| G3 | Lyo → + DMC → Lyo | Powder | — |
| G4 | Lyo → + DMC + PEG → Lyo | Powder | — |
| G5 | Lyo | Powder | Yes |
| G6 | Lyo → + DMC + PEG → Lyo | Powder | Yes |
| G7 | Lyo → Organogel → Lyo | Gel | Yes |
| G8 | +Sucrose → Lyo | Powder | — |
| G9 | +Sucrose → Lyo → + DMC → Lyo | Powder | — |
| G10 | +Sucrose → Lyo → + DMC + | Powder | — |
| | PEG → Lyo | | |
| G11 | +Sucrose → Lyo → + DMC + | Powder | Yes |
| | PEG → Lyo | | |
| G12 | +Sucrose → Lyo → Organogel → Lyo | Gel | Yes |
| G13 | +Sucrose-4a40k NH2 → Lyo → | Gel | Yes |
| | Organogel → Lyo | | |
| G14 | +4a40k NH2 → Lyo → Organogel → | Gel | Yes |
| | Lyo | | |
| G15 | +Trehalose → Lyo → Organogel → | Gel | Yes |
| | Lyo | | |

G1-G4 are the same as in the previous section to assess repeatability of results. G5-G7 assessed the impact of overnight PBS, PH=7.4 incubation at 37° C. on AAV infectivity. G7 additionally encapsulated AAVs in an organogel (5% PEG, 5:1 molar ratio of 4a40k NH2: 4a40k SS) and assessed the released AAVs after overnight PBS incubation.

G8-G15 evaluated similar steps, but with the incorporation of lyoprotectants (sucrose, trehalose, 4a40k PEG NH2, or a combination of sucrose and 4a40k NH2) prior to the first freeze-dry step.

4 sterile aqueous and 4 sterile organic solutions were made. Table 5 outlines the solutions prepared:

TABLE 5
Aqueous and organic solutions prepared for AAV sample preparation

| Aqueous/Organic | Solution | Excipient | Concentration |
|---|---|---|---|
| Aqueous | Sugar | 1.6M Sucrose | 548 mg/mL |
| | Sugar-PEG | 0.4M Sucrose \| | 137 mg/mL \| |
| | | 4a40k NH2 | 83 mg/mL |
| | PEG_A | 4a40k NH2 | 83 mg/mL |
| | Sugar_2 | 1.6M Trehalose | 605 mg/mL |
| | | Dihydrate | |
| Organic | DMC | N/A | N/A |
| | 1a-A_DMC | 20k Methoxy PEG | 5% |
| | | Amine | |
| | E_DMC | 4a40k SS | 5% |
| | 4a-A_DMC | 4a40k NH2 | 5% |

Aqueous solutions were prepared in 1×PBS, pH=7.4, diluted from premixed 10×PBS (Sigma, 11666789001), and filtered with an Acrodisc, 0.2 μm Supor membrane (PALL, PN 4652). Organic solutions were prepared in DMC and filtered with an Acrodisc syringe filter, 0.2 μm Nylon (PALL, PM4433). After sterile filtering in the BSC, samples were frozen at −20° C. prior to use.

An outline of the groups prepared, and solution volumes used is shown in Table 6 below. Table 7 outlines the final formulations amounts in each vial for cell analysis.

TABLE 6
AAV2 groups and formulations prepared for cell transduction efficiency assessment

| | | 1st Lyophilization | | | | | |
|---|---|---|---|---|---|---|---|
| | | AAV | 1x | | Sucrose + | | |
| | | Stock | PBS | Sucrose | 4a40kNH2 | 4a40kNH2 | Trehelose |
| Group | Description | (uL) | (uL) | (uL) | (uL) | (uL) | (uL) |
| G1 | (+) Control - | 30 | | | | | |
| | Virus in PBS | | | | | | |
| G2 | Lyo | 30 | | | | | |
| G3 | Lyo → +DMC | 30 | | | | | |
| | → Lyo | | | | | | |
| G4 | Lyo → +1a-A_DMC | 30 | | | | | |
| | +E_DMC → Lyo | | | | | | |
| G5 | Lyo | 30 | | | | | |
| G6 | Lyo → +1a-A_DMC | 30 | | | | | |
| | +E_DMC → Lyo | | | | | | |
| G7 | Lyo → +4a-A_DMC | 30 | | | | | |
| | +E_DMC → Lyo | | | | | | |
| G8 | +Sugar →Lyo | 30 | 37.5 | 12.5 | | | |
| G9 | +Sugar → | 30 | 37.5 | 12.5 | | | |
| | Lyo → +DMC | | | | | | |
| | → Lyo | | | | | | |
| G10 | +Sugar → | 30 | 37.5 | 12.5 | | | |
| | Lyo → +1a-A_DMC | | | | | | |
| | +E_DMC → Lyo | | | | | | |
| G11 | +Sugar → | 30 | 37.5 | 12.5 | | | |
| | Lyo → +1a-A_DMC | | | | | | |
| | +E_DMC → Lyo | | | | | | |
| G12 | +Sugar → | 30 | 37.5 | 12.5 | | | |
| | Lyo → +4a-A_DMC | | | | | | |
| | +E_DMC → Lyo | | | | | | |
| G13 | +Sugar-PEG | 30 | | | 50 | | |
| | →Lyo | | | | | | |
| | →+DMC | | | | | | |
| | +E_DMC | | | | | | |
| | →Lyo | | | | | | |
| G14 | +PEG_A | 30 | | | | 50 | |
| | →Lyo | | | | | | |
| | →+DMC | | | | | | |
| | +E_DMC | | | | | | |
| | →Lyo | | | | | | |
| G15 | +Sugar_2 | 30 | 37.5 | | | | 12.5 |
| | →Lyo →+4a- | | | | | | |
| | A_DMC +E_DMC | | | | | | |
| | →Lyo | | | | | | |

| | 1st Lyophilization | | | | | | |
|---|---|---|---|---|---|---|---|
| | Total | | | 2nd Lyophilization | | | |
| | Lyo | Sugar | 4a40kNH2 | | Methoxy | | |
| | Volume | Conc | Conc | DMC | PEG NH2 | 4a40kSS | 4a40kNH2 |
| Group | (uL) | (mg/mL) | (mg/mL) | (uL) | (uL) | (uL) | (uL) |
| G1 | 30 | | | | | | |
| G2 | 30 | | | | | | |
| G3 | 30 | | | 100 | | | |
| G4 | 30 | | | | 67 | 33 | |
| G5 | 30 | | | | | | |
| G6 | 30 | | | | 67 | 33 | |
| G7 | 30 | | | | | 17 | 83 |
| G8 | 80 | 86 | | | | | |
| G9 | 80 | 86 | | 100 | | | |
| G10 | 80 | 86 | | | 67 | 33 | |
| G11 | 80 | 86 | | | 67 | 33 | |
| G12 | 80 | 86 | | | | 17 | 83 |
| G13 | 80 | 86 | 52 | 83 | | 17 | |
| G14 | 80 | | 52 | 83 | | 17 | |
| G15 | 80 | 86 | | | | 17 | 83 |

TABLE 7
Final formulation amounts per vial for cell analysis

| | | | | | 20k | | |
|---|---|---|---|---|---|---|---|
| | | | | 4a40k | Methoxy | | |
| | | PBS | Su- | PEG | PEG | 4a40k | Total |
| | AAV | Salts | crose | NH2 | NH2 | PEG SS | PEG |
| Group | (GC) | (mg) | (mg) | (mg) | (mg) | (mg) | (mg) |
| G1 | 3.30E+11 | 0.30 | 0 | 0 | 0 | 0 | 0 |
| G2 | 3.30E+11 | 0.30 | 0 | 0 | 0 | 0 | 0 |
| G3 | 3.30E+11 | 0.30 | 0 | 0 | 0 | 0 | 0 |
| G4 | 3.30E+11 | 0.30 | 0 | 0 | 3.35 | 1.65 | 5.00 |
| G5 | 3.30E+11 | 0.30 | 0 | 0 | 0 | 0 | 0 |
| G6 | 3.30E+11 | 0.30 | 0 | 0 | 3.35 | 1.65 | 5.00 |
| G7 | 3.30E+11 | 0.30 | 0 | 4.15 | 0 | 0.85 | 5.00 |
| G8 | 3.30E+11 | 0.80 | 6.85 | 0 | 0 | 0 | 0 |
| G9 | 3.30E+11 | 0.80 | 6.85 | 0 | 0 | 0 | 0 |
| G10 | 3.30E+11 | 0.80 | 6.85 | 0 | 3.35 | 1.65 | 5.00 |
| G11 | 3.30E+11 | 0.80 | 6.85 | 0 | 3.35 | 1.65 | 5.00 |
| G12 | 3.30E+11 | 0.80 | 6.85 | 4.15 | 0 | 0.85 | 5.00 |
| G13 | 3.30E+11 | 0.80 | 6.85 | 4.15 | 0 | 0.85 | 5.00 |
| G14 | 3.30E+11 | 0.80 | 0 | 4.15 | 0 | 0.85 | 5.00 |
| G15 | 3.30E+11 | 0.80 | 6.85 | 4.15 | 0 | 0.85 | 5.00 |

Briefly, G1-G7 were kept frozen at −80° C. For G8-G15, 50 μL of sugar and/or PEG NH2 solution were added to the 30 μL of AAV stock. The final sucrose and trehalose concentration was 86 mg/ml, and the final 4a40k PEG NH2 concentration was 52 mg/mL prior to lyophilization.

In a prior experimental design, the final sucrose concentration for lyophilization was 342 mg/mL, or 1M sucrose. This concentration was shown to be protective for AAVs during freeze-drying. However, a solution of 1.6M sucrose with 83 mg/mL 4a40k NH2 was too viscous to handle and syringe filter. The concentration was reduced to 0.4M sucrose with 83 mg/mL 4a40k NH2. As a result, the amount of sucrose added was reduced to give a final sugar concentration of 86 mg/ml, or 0.25M.

After mixing, samples were re-frozen at −80° C. for 2 h. G2-G15 AAV samples were then transferred to a manifold lyophilizer (Labconco FreeZone 2.5L) while still frozen, and the first lyophilization was done overnight (no set time) at a vacuum of 60 mTorr and the condenser set at −50° C.

Prior to the second lyophilization cycle, the appropriate DMC solutions and amounts were added to the freeze-dried samples (see Table 6). All PEG containing groups had a final 5% concentration in DMC. 20k Methoxy PEG Amine and 4a40k SS were combined in a 1:1 molar ratio. 4a40k NH2 and 4a40k SS were combined in a 5:1 molar ratio. This ratio was chosen to create a hydrogel that could degrade overnight in physiological pH at 37° C. Groups 7, 12, 14, and 15 formed an organogel within 15 min in a tube.

After combining the DMC solutions with the lyophilized product, vials were briefly vortexed and allowed to sit for 15-30 min at ambient lab conditions. Samples were then frozen in the −80° C. freezer for 2 h and lyophilized overnight with a similar procedure to the first lyophilization to remove the DMC. All samples were stored at −80° C. prior to use.

For cell transduction assay, 300 μL DPBS, pH=7.4 were added to groups undergoing overnight incubation at 37° C.: G5-G7 and G12-G15. Lyophilized samples (G2-G4 and G8-G10) were reconstituted with 300 μL DPBS, pH=7.4. All groups were then diluted with culture media to the appropriate titer prior before adding to the cells. The well plate layout is shown in Table 8.

TABLE 8
Two 96-well plate layouts to assess 16 AAV2 groups

| | | | | | MOI 1 | | | MOI 2 | | | MOI 3 | | |
|---|---|---|---|---|---|---|---|---|---|---|---|---|---|
| | | No Virus | | | (1,000) | | | (10,000) | | | (100,000) | | |
| Serotype | Group | 1 | 2 | 3 | 4 | 5 | 6 | 7 | 8 | 9 | 10 | 11 | 12 |

Plate 1

| | | |
|---|---|---|
| AAV2 in | 1 | A |
| HEK293 | 2 | B |
| | 3 | C |
| | 4 | D |
| | 5 | E |
| | 6 | F |
| | 7 | G |
| | 8 | H |

Plate 2

| | | |
|---|---|---|
| AAV2 in | 9 | A |
| HEK293 | 10 | B |
| | 11 | C |
| | 12 | D |
| | 13 | E |
| | 14 | F |
| | 15 | G |
| | 16 | H |

48 h post-infection, the plate was analyzed for Transduction efficiency (% GFP+ cells) and Mean Fluorescent Intensity (MFI) of GFP+ cells by flow cytometry.

Results for G1-G4 were similar to the study in the previous section, demonstrating repeatability of the cell assay. FIG. 2 shows % transduction efficiency and MFI results for all groups.

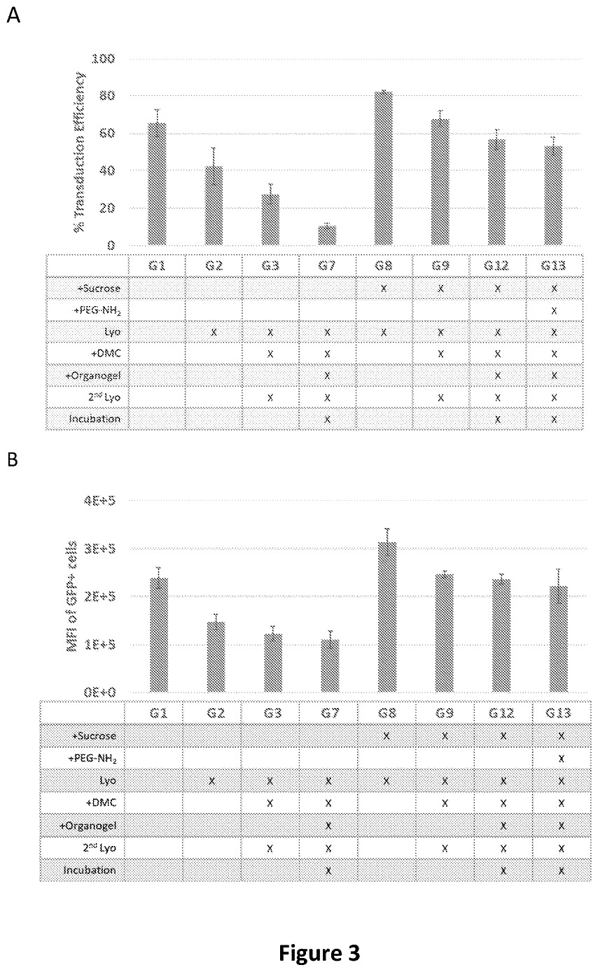

The first goal of this study was to assess the addition of lyoprotectant excipients into the buffer to improve infectivity of AAV post-lyophilization. In a direct comparison between G2 and G8, the addition of sucrose as a lyoprotectant had a marked improvement on % transduction efficiency and MFI. In a subgroup analysis (FIGS. 3A and 3B), sucrose and sucrose+4a40k NH2 also had a beneficial effect on AAV infectivity against DMC exposure and organogel formation. % Transduction efficiency and MFI of GFP+ cells were comparable to the positive control.

The results of this study demonstrate that sucrose and the combination of sucrose+4a40k NH2 were effective excipients for maintaining AAV2 infectivity through the 4 steps of organogel processing steps: 1) lyophilization of AAV, 2) DMC exposure, 3) PEG crosslinking and organogel formation, and 4) drying of organogel to form a xerogel.

### Example 1.3: AAV2.7m8 and AAV8 Infectivity can be Protected During Lyophilization, Organic Solvent Exposure, Organogel and Xerogel Processing

The purpose of this study was to evaluate the impact of lyoprotectants on the transduction efficiency of AAV2.7m8 and AAV8 in a cell assay.

AAV8 and AAV2.7m8 are potential viral vectors for ocular gene therapies.

In previous cell studies, organogel platform compatibility with AAV2 was evaluated. In the current study, the impact of lyoprotectants on the transduction efficiency of AAV2.7m8 and AAV8 after lyophilization was assessed. Crosslinking chemistry was used for this cell study to investigate the effect of crosslinking on AAV infectivity. However, full crosslinking and thus, organogel formation was not done. When they are fully crosslinked, samples may need to be kept at elevated pH (8.0-8.4), and temperature (37° C.) for extended time (2-10 days) to fully degrade the hydrogel and release AAVs.

According to literature and previous experiments, storing samples at pH=8.4, 37° C., and extended time may lower AAV infectivity. Because of this reason, a 10:1 PEG-NH2: SS molar ratio was used that did not form a gel. In this way, the effect of crosslinking chemistry without encapsulation of lyophilized AAV particles could be assessed.

Table 9 lists the AAV serotype, vendor, lot #, stock concentration, and buffer formulation of each AAV used in this study.

TABLE 9
AAV Sources used

| | | Buffer |
|---|---|---|
| AAV Serotype | Stock Conc. | Formulation |
| AAV8-CMV- | 2E+13 GC/mL | 1× PBS + |
| GFP | | 0.001% Pluronic |
| AAV2.7m8- | 2E+13 GC/mL | 1× PBS + |
| CMV-GFP | | 0.001% Pluronic + |
| | | 100 mM |
| | | sodium citrate |
| AAV2.7m8- | 4.2E+13 | 1× PBS + |
| CMV-EGFP | GC/mL | 0.001% Pluronic + |
| | | 200 mM NaCl |

Table 10 outlines AAV groups and formulations prepared for cell transduction efficiency assessment.

TABLE 10
AAV groups and formulations prepared for cell transduction efficiency assessment

| | | | 1st Lyophilization | | | | | | | | | |
|---|---|---|---|---|---|---|---|---|---|---|---|---|
| Group Description | | | | | Sucrose | Sucrose | Mannitol | Trehalose | Final | 2nd Lyophilization | | Dilution/ |
| | | Prepa- | AAV | | 200 | 20 | 200 | 200 | Concen- | 4a20kNH2 | 4a40kNH2 | Recons. |
| | | ration | Stock | PBS* | mg/mL | mg/mL | mg/mL | mg/mL | tration | (uL) | (uL) | PBS* |
| Group | Name | Steps | (uL) | (uL) | (uL) | (uL) | (uL) | (uL) | in Lyo** | in DMC | in DMC | (uL) |
| G1 | AAV8 | (+) Control - | 35 | | | | | | | | | 365 |
| | | Virus in PBS | | | | | | | | | | |
| G2 | AAV2.7m8 | (+) Control - | 35 | | | | | | | | | 365 |
| | Vendor 1 | Virus in PBS | | | | | | | | | | |
| G3 | AAV2.7m8 | (+) Control - | 10 | | | | | | | | | 390 |
| | Vendor 2 | Virus in PBS | | | | | | | | | | |
| G4 | AAV8 50S- | Lyo → DMC | 35 | | 17.5 | | 17.5 | | 50 mg/mL | 100 | 10 | 400 |
| | 50M | +PEG → Lyo | | | | | | | Sucrose + | | | |
| | | | | | | | | | 50 mg/mL | | | |
| | | | | | | | | | Mannitol | | | |
| G5 | AAV8 50T- | Lyo → DMC | 35 | | | | 17.5 | 17.5 | 50 mg/mL | 100 | 10 | 400 |
| | 50M | +PEG → Lyo | | | | | | | Trehalose + | | | |
| | | | | | | | | | 50 mg/mL | | | |
| | | | | | | | | | Mannitol | | | |
| G6 | AAV8 10S | Lyo → DMC | 35 | | | 35 | | | 10 mg/mL | 100 | 10 | 400 |
| | | +PEG → Lyo | | | | | | | Sucrose | | | |
| G7 | Lyo AAV8 | Lyo | 35 | | | 35 | | | 10 mg/mL | | | 400 |
| | 10S | | | | | | | | Sucrose | | | |
| G8 | AAV2.7m8 | Lyo → DMC | 35 | | 17.5 | | 17.5 | | 50 mg/mL | 100 | 10 | 400 |
| | Vendor 1 | +PEG → Lyo | | | | | | | Sucrose + | | | |
| | 50S-50M | | | | | | | | 50 mg/mL | | | |
| | | | | | | | | | Mannitol | | | |
| | | | | | | | | | (+12.9 mg/mL | | | |
| | | | | | | | | | sodium citrate) | | | |
| G9 | AAV2.7m8 | Lyo → DMC | 35 | | | | 17.5 | 17.5 | 50 mg/mL | 100 | 10 | 400 |
| | Vendor 1 | +PEG → Lyo | | | | | | | Trehalose + | | | |
| | 50T-50M | | | | | | | | 50 mg/mL | | | |
| | | | | | | | | | Mannitol | | | |
| | | | | | | | | | (+12.9 mg/mL | | | |
| | | | | | | | | | sodium citrate) | | | |
| G10 | AAV2.7m8 | Lyo → DMC | 20 | 15 | | | 17.5 | 17.5 | 50 mg/mL | 100 | 10 | 400 |
| | Vendor 2 | +PEG → Lyo | | | | | | | Trehalose + | | | |
| | 50T-50M | | | | | | | | 50 mg/mL | | | |
| | | | | | | | | | Mannitol | | | |
| | | | | | | | | | (+3.3 mg/mL | | | |
| | | | | | | | | | NaCl) | | | |
| G11 | AAV2.7m8 | Lyo → DMC | 20 | 15 | | 35 | | | 10 mg/mL | 100 | 10 | 400 |
| | Vendor 2 | +PEG → Lyo | | | | | | | Sucrose | | | |
| | 10S | | | | | | | | (+3.3 mg/mL | | | |
| | | | | | | | | | NaCl) | | | |
| G12 | AAV8 30T- | Lyo → DMC | 35 | 14 | | | 10.5 | 10.5 | 30 mg/mL | 100 | 10 | 400 |
| | 30M | +PEG → Lyo | | | | | | | Trehalose + | | | |
| | | | | | | | | | 30 mg/mL | | | |
| | | | | | | | | | Mannitol | | | |

*1x PBS + 0.001% Pluronic
**Total lyo volume was kept at 70 μL for all lyophilized samples (G4-G12)

AAV Lyophilization was conducted as follows:
1. 5 mL volumetric flasks were used for solution preparation. Volumetric flasks were labelled accordingly. Sugar concentrations listed below were prepared in PBS+0.001% Pluronic at room temperature.
   200 mg/mL Sucrose
   20 mg/mL Sucrose
   200 mg/mL Mannitol
   200 mg/mL Trehalose
2. Prepared sugar solutions were filtered into autoclaved 5 mL protein lo-bind Eppendorf tubes using Mustang E filters (PALL MSTG25E3). 5 mL protein lo-bind Eppendorf tubes were labelled accordingly.
3. Autoclaved 1.5 mL protein lo-bind Eppendorf tubes were used for sample preparation and labelled accordingly.
4. AAVs were removed from −80° C. freezer and thawed at room temperature.
5. First, sugar solutions were added into appropriate 1.5 mL protein lo-bind Eppendorf tubes. Then, AAV stock solutions were added into appropriate tubes. 35 μL stock solution used for AAV8 and AAV2.7m8 Vendor 1 samples. 20 μL stock solution used for AAV2.7m8 Vendor 2 samples. Solutions were pipette mixed before lyophilization. Table 10 shows the constituents and volumes of each sample.
6. After pipette mixing, the frozen aluminum block removed from −80° C. freezer. Samples were placed in the block and double-pouched using sterile 7.5″×13″ autoclave pouches.

Samples were freeze-dried in a shelf lyophilizer for 3 days. Table 11 shows the lyophilization cycle.

TABLE 11
Lyophilization cycle for AAVs

| | | Temp | Time | | Pressure |
|---|---|---|---|---|---|
| | Steps | (° C.) | (min) | Ramp/Hold | (mTorr) |
| Thermal Treatment | 1 | −40 | 60 | Hold | — |
| | 2 | −20 | 40 | Ramp | — |
| | 3 | −20 | 180 | Hold | — |
| | 4 | −40 | 40 | Ramp | — |
| Freeze | — | −40 | 60 | — | — |
| Primary Drying* | 1 | −38 | 90 | Hold | 100 |
| | 2 | −35 | 25 | Ramp | 100 |
| | 3 | −35 | 1000 | Hold | 100 |
| | 4 | −35 | 1000 | Hold | 100 |
| | 5 | −10 | 200 | Ramp | 100 |
| | 6 | −10 | 240 | Hold | 100 |
| | 7 | 0 | 80 | Ramp | 100 |
| | 8 | 0 | 240 | Hold | 100 |
| | 9 | 25 | 200 | Ramp | 100 |
| | 10 | 25 | 480 | Hold | 100 |

*Condenser set at −70° C.

PEG-NH₂+PEG-SS Preparation in DMC was conducted as follows.
1. 5 mL volumetric flasks were used for PEG solution preparation. 6% 4a20k PEG-NH₂ and 6% 4a20k PEG-SS solutions in DMC were prepared and sterile filtered through an acrodisc syringe filter, 0.2 μm Nylon (PALL, PN4433).
2. 100 μL PEG-NH₂+10 μL PEG-SS in DMC were mixed with lyophilized AAV powder by gentle pipette tip stirring.
3. Samples were kept in BSC for 2 hours for full DMC exposure. This is the approximate time fibers are kept in DMC chamber during fiber preparation.
4. After 2 hours, samples were placed in the lyophilizer for 2 days to remove the DMC. The shelf temperature was set at 25° C. and the condenser at −70° C.

Sample preparation before use was conducted as follows.
1. After samples were dried, they were removed from lyophilizer.
2. All dry samples (G4-G12) were reconstituted with sterile 400 μL 1×PBS+0.001% Pluronic at pH 7.2.
3. AAVs were removed from −80° C. freezer and thawed at room temperature to prepare controls (G1-G3).
4. Control samples (G1-G3) were prepared in autoclaved 1.5 mL protein lo-bind Eppendorf tubes. 1.5 mL protein lo-bind Eppendorf tubes were labelled appropriately. After 1×PBS+0.001% at pH=7.2 addition, AAVs were added and pipette-mixed.
5. After dilution/reconstitution, all samples were flash frozen in liquid nitrogen. Samples were shipped on dry ice and kept in −80° C. freezer until they were used for infectivity assay testing.

Table 12 lists lot #s, MOI #s, concentrations, and volumes of all groups.

TABLE 12
Groups descriptions for cell assay

| GN | Name | MOI# | Concentration | Volume |
|---|---|---|---|---|
| G1 | AAV8 Control | 5e5 & 1e6 | 1.75 e12 GC/mL | 400 μL |
| G2 | AAV2.7m8 Vendor | 1e5 & 5e5 | 1.75 e12 GC/mL | 400 μL |
| | 1 control | | | |
| G3 | AAV2.7m8 Vendor | 1e5 & 5e5 | 1.05 e12 GC/mL | 400 μL |
| | 2 control | | | |
| G4 | AAV8 50S-50M | 5e5 & 1e6 | 1.75 e12 GC/mL | 400 μL |
| G5 | AAV8 50T-50M | 5e5 & 1e6 | 1.75 e12 GC/mL | 400 μL |
| G6 | AAV8 10S | 5e5 & 1e6 | 1.75 e12 GC/mL | 400 μL |
| G7 | Lyo AAV8 10S | 5e5 & 1e6 | 1.75 e12 GC/mL | 400 μL |
| G8 | AAV2.7m8 Vendor | 1e5 & 5e5 | 1.75 e12 GC/mL | 400 μL |
| | 1 50S-50M | | | |
| G9 | AAV2.7m8 Vendor | 1e5 & 5e5 | 1.75 e12 GC/mL | 400 μL |
| | 1 50T-50M | | | |
| G10 | AAV2.7m8 Vendor | 1e5 & 5e5 | 2.1e12 GC/mL | 400 μL |
| | 2 50T-50M | | | |
| G11 | AAV2.7m8 Vendor | 1e5 & 5e5 | 2.1e12 GC/mL | 400 μL |
| | 2 10S | | | |
| G12 | AAV8 30T-30M | 5e5 & 1e6 | 1.75 e12 GC/mL | 400 μL |

For cell transduction assay, all groups were diluted with culture media to the appropriate titer prior before adding to the cells. The well plate layout is shown in Table 13.

TABLE 13
96 well plate design for the cell assay

| | | Treatment Groups | | | | | |
|---|---|---|---|---|---|---|---|
| | | G1 | G2 | G3 | G4 | G5 | G6 |
| | | 1 | 2 | 3 | 4 | 5 | 6 |
| No Virus | A | | | | | | |
| | B | | | | | | |
| Low MOI | C | 5.00E+05 | 1.00E+05 | 1.00E+05 | 5.00E+05 | 5.00E+05 | 5.00E+05 |
| | D | | | | | | |
| | E | | | | | | |
| High MOI | F | 1.00E+06 | 5.00E+05 | 5.00E+05 | 1.00E+06 | 1.00E+06 | 1.00E+06 |
| | G | | | | | | |
| | H | | | | | | |
| | | Treatment Groups | | | | | |
| | | G7 | G8 | G9 | G10 | G11 | G12 |
| | | 7 | 8 | 9 | 10 | 11 | 12 |
| No Virus | A | | | | | | |
| | B | | | | | | |
| Low MOI | C | 5.00E+05 | 1.00E+05 | 1.00E+05 | 1.00E+05 | 1.00E+05 | 5.00E+05 |
| | D | | | | | | |
| | E | | | | | | |
| High MOI | F | 1.00E+06 | 5.00E+05 | 5.00E+05 | 5.00E+05 | 5.00E+05 | 1.00E+06 |
| | G | | | | | | |
| | H | | | | | | |

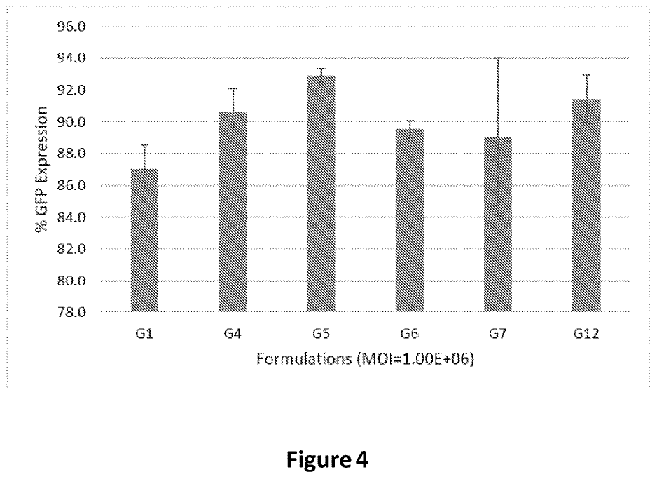

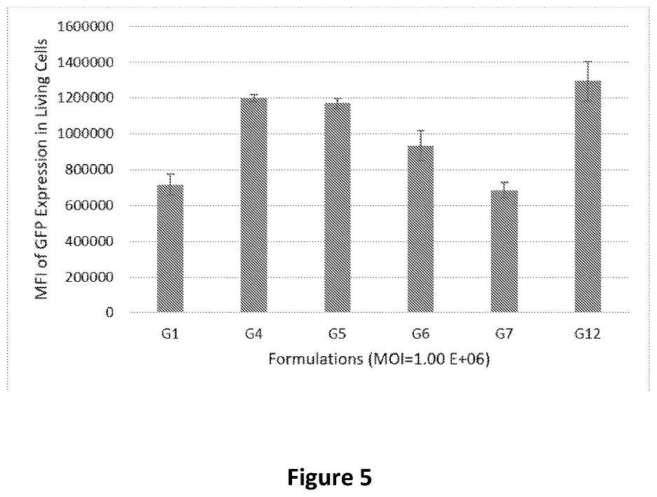

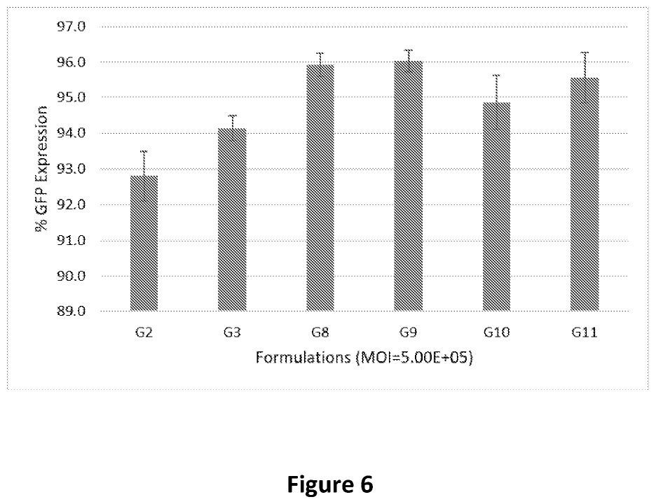

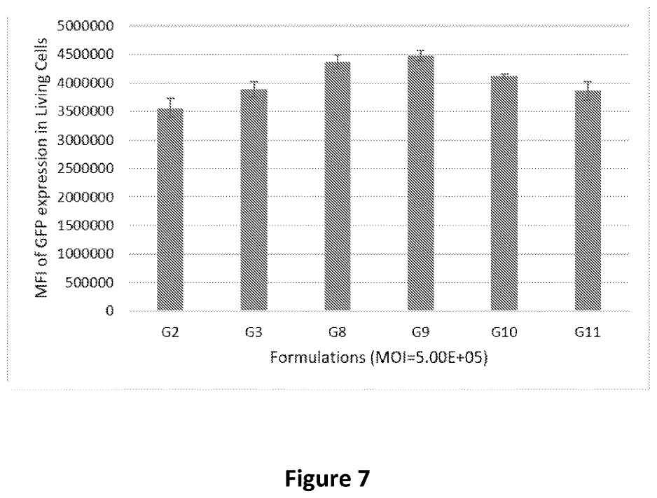

There were overall high levels of GFP expression in all the 12 groups tested in this assay (FIGS. 4, 5, 6 and 7). It was found that lyophilization, DMC addition, PEG crosslinking chemistry did not have a negative effect on the infectivity of AAV2.7m8 and AAV8. All lyoprotectants tested in this study showed higher MFI than control groups. Infectivity assay results demonstrate the compatibility of organogel platform with AAV2.7m8 and AAV8.

### Example 2—Formulation: Fiber Fabrication Process

In this example, a complete process encompassing all organogel and xerogel processing steps culminating in the formation of an implant in the form of a fiber was carried out in two different ways namely Process A and Process B.

AAVs are icosahedral particles approximately 25 nm in diameter. To screen multiple formulations, AuNPs of similar size were chosen as a surrogate due their lower expense, and ease of analysis via UV/Vis. Specifically, spherical AuNPs that were conjugated with methoxy-PEG (28-36 nm in diameter) were used.

### Example 2.1: Process A-AAV/Functional PEG Syringe (all Contents Lyophilized Together)+PEG Ester Syringe (all Contents Lyophilized Together)

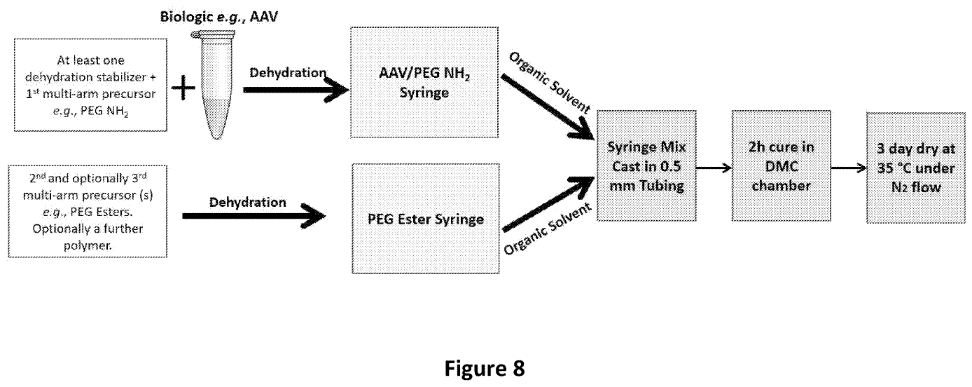

The fiber fabrication according to Process A was carried out as shown in FIG. 8.

The fiber fabrication with AuNP/AAV according to Process A was done as follows: Briefly, two syringes were lyophilized for each group: 1) AuNP+PEG NH2 syringe and 2) Ester syringe. AuNP solution was mixed with a concentrated cryo solution that consisted of PEG NH2 and sucrose dissolved in 1×PBS+0.001% Pluronic F-68. The appropriate volumes and concentrations were combined to achieve the target PEG NH2 and sucrose concentrations for the AuNP+PEG NH2 syringe. For the Ester syringe, PEG ester was dissolved in WFI, pH=3.8, kept on ice. Solutions were then loaded into 1 mL syringes and lyophilized as outlined in Table 14. All formulations had a 1:1 NH2: Ester molar ratio.

1 mL Soft-Ject luer-lock syringes (HSW) with the plunger removed were loaded with the solution to be freeze-dried. Filled syringes were placed in a 1 ml aluminum block holder cooled to −20° C. and loaded in the shelf freeze-dryer. The recipe used is outlined in Table 14 below:

TABLE 14
Lyophilization Recipe for AuNPs

| | | Temp | Time | | Pressure |
|---|---|---|---|---|---|
| | Steps | (° C.) | (min) | Ramp/Hold | (mTorr) |
| Thermal | — | — | — | — | — |
| Treatment | | | | | |
| Freeze | — | −48 | 60 | — | — |
| Primary | 1 | −48 | 30 | Ramp | 50 |
| Drying* | 2 | −45 | 25 | Ramp | 50 |
| | 3 | −45 | 480 | Hold | 50 |
| | 4 | −10 | 285 | Ramp | 50 |
| | 5 | −10 | 120 | Hold | 50 |
| | 6 | 0 | 80 | Ramp | 50 |
| | 7 | 0 | 120 | Hold | 50 |
| | 8 | 25 | 200 | Ramp | 50 |
| | 9 | 25 | 1255 | Hold | 50 |

*Condenser set at −70° C.

The glass transition temperature of the freeze-concentrated solution (Tg′) was evaluated on a DSC 6000 (PerkinElmer). Samples were cooled to −75° C. at 1° C./min, held for 10 min at −75° C., then heated to 30° C. at 10° C./min. A baseline shift in the heating curve was evaluated for Tg′.

After completing the freeze-dry cycle, an appropriate amount of DMC was pipetted into the AuNP+PEG NH2 and Ester syringes and the mixture was allowed to sit for 30-60 min to dissolve the PEGs. The AuNP+PEG NH2 syringe was then connected to the PEG Ester syringe via a luer connector and the contents of both syringes were mixed together for 30 sec. After syringe mixing, the mixture was drawn into one syringe. The plunger was pulled to form a slight vacuum in the syringe. With a slight vacuum present, the syringe was flicked 3-5 times to promote degassing of the mixture.

The empty syringe and luer connector were then detached. The syringe with the mixture was connected to a needle inserted into tubing and the plunger was depressed to cast. The mixing, degassing, and casting steps all occurred prior to gelation of the formulation (typically within 90 s). After casting, the distal and proximal ends of the mixture were clamped and then placed in an enclosed chamber to cure for 2 h in the presence of DMC vapor. The DMC vapor was present to prevent premature drying of the casted gel prior to full curing. After curing, the casted tubing was placed in a 35-37° C. oven to dry for 3 days.

AAV fibers were fabricated in a similar process by replacing the AuNP solution with the AAV stock solution.

### Example 2.2: Process B—AAV/PEG Syringe (AAV Solution Separately Lyophilized+Functional PEG Separately Lyophilized)+PEG Ester Syringe (all Contents Lyophilized Together)

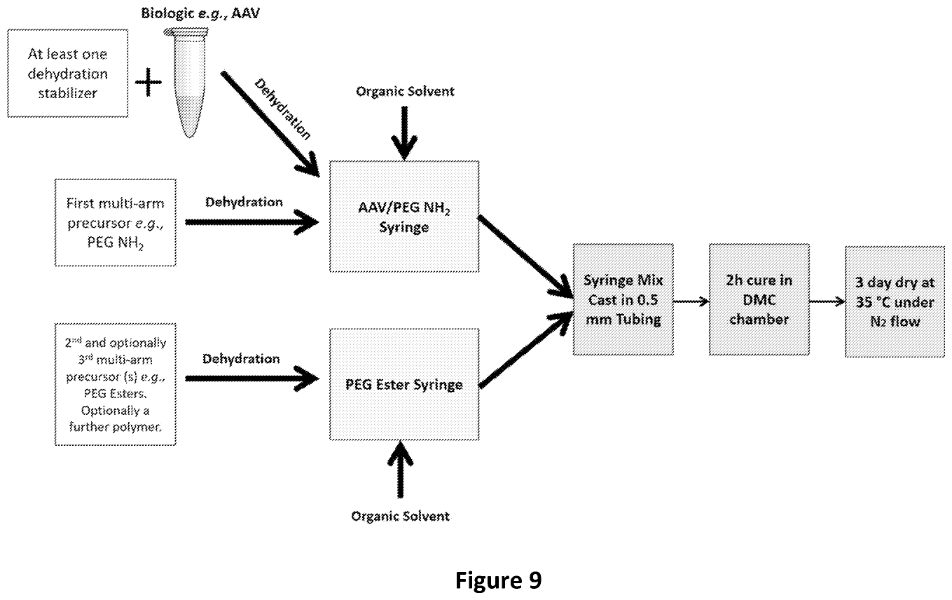

Alternatively, the fiber fabrication process was carried out according to process B as shown in FIG. 9.

1 mL stock AuNP solution was diluted with 4 ml of 1.25×PBS+0.00125% Pluronic F-68. AuNP solution was mixed with a concentrated cryo solution that consisted of sucrose dissolved in 1×PBS+0.001% Pluronic F-68. Solutions were then loaded into 1 mL syringes and lyophilized as outlined in Table 15. The appropriate volumes and concentrations were combined to achieve the target sucrose concentration (5 mg/mL) for the AuNP+sucrose syringe (Table 16). Sucrose concentration, AuNP and cryo volumes were kept constant.

TABLE 16
Formulation and Composition of Lyophilized AuNPs

| | | Lyo Volume | | | % Composition of | | |
|---|---|---|---|---|---|---|---|
| Cryo soln | Lyo soln | AuNP | Cryo | Total Lyo | Lyophilized Product | | |
| Sucrose | Sucrose | Solution | Solution | Solution | | PBS | Pluronic |
| (mg/mL) | (mg/mL) | (uL) | (uL) | (uL) | Sucrose | Salts | F-68 |
| 55 | 5 | 500 | 50 | 550 | 33.6% | 66.4% | 0.1% |

10% 4a20k PEG-NH2, 10% 4a20k PEG-SS, 10% 4a40k PEG-SG, and 10% 8k linear PEG solutions in DMC were prepared. After dissolving the polymers in DMC at room temperature, the appropriate volumes of SS, SG, and 8k linear PEG were combined into a 1.5 mL Eppendorf tube to achieve the target SS/SG ratios for the Ester syringe (Table 17 in the next Example).

1 mL HSW Soft-Ject syringes with the plunger removed were loaded with the solution to be freeze-dried. Filled syringes were placed in a 1 mL aluminum block holder cooled to −80° C. and loaded in the shelf freeze-dryer. The recipe used is outlined in Table 15 below:

TABLE 15
Lyophilization Recipe for AuNPs

| | Temp | Time | | Pressure |
|---|---|---|---|---|
| Steps | (° C.) | (min) | Ramp/Hold | (mTorr) |
| Prefreeze | −40 | 60 | — | — |
| 1 | −38 | 90 | Hold | 100 |
| 2 | −35 | 25 | Ramp | 100 |
| 3 | −35 | 1000 | Hold | 100 |
| 4 | −35 | 1000 | Hold | 100 |
| 5 | −10 | 200 | Ramp | 100 |
| 6 | −10 | 240 | Hold | 100 |
| 7 | 0 | 80 | Ramp | 100 |
| 8 | 0 | 240 | Hold | 100 |
| 9 | 25 | 200 | Ramp | 100 |
| 10 | 25 | 480 | Hold | 100 |

After completing the freeze-dry cycle, the appropriate volume of PEG-NH2-DMC solution was loaded from front side to the AuNP+sucrose loaded syringes while pulling plunger down. All powder was wetted with PEG-NH2 DMC solution by degassing 3× and slowly pushing the mixed solution to the syringe tip. Then, the syringe was capped with a female luer cap.

The appropriate amount of combined PEG-ester solution was loaded from front side to a new syringe. The AuNP+PEG NH2 syringe was then connected to the PEG Ester syringe via a luer connector, and the contents of both syringes were mixed for 30 sec. After syringe mixing, the mixture was drawn into one syringe. The plunger was pulled to form a slight vacuum in the syringe. With a slight vacuum present, the syringe was flicked 3-5 times to promote degassing of the mixture. The empty syringe and luer connector were then detached. The syringe with the mixture was connected to a 19G, 0.5″ blunt needle inserted into 0.93 mm Pellethane tubing (Nordson Medical, Lot #201005-11, wall thickness=0.007″±0.001″), and the plunger was depressed to cast. The mixing, degassing, and casting steps all occurred prior to gelation of the formulation (typically within 90 s). After casting, the distal and proximal ends of the mixture were clamped and then placed in an enclosed chamber to cure for 2 h in the presence of DMC vapor. The DMC vapor was present to prevent premature drying of the casted gel prior to full curing. Gelation time was noted. After curing, the casted tubing was placed in a 35-37° C. oven to dry.

### Example 3—Release Profile: AuNP

As discussed previously, AAVs are icosahedral particles approximately 25 nm in diameter. To screen multiple formulations, spherical AuNPs of similar size (28-36 nm in diameter) were chosen as a surrogate due to their lower expense, and ease of analysis via UV/Vis.

### Example 3.1: AuNP IV Release—SS:SG Ratios can be Selected to Control the Release Profile

In this study, different variables were investigated to see their influence on release kinetics of encapsulated AuNPs according to Process A or Process B described in the previous section. PEG NH2 (4a40k, 4a20k, and 8a20k) were combined with different PEG esters (4a40k and 4a20k) at varying SS:SG ratios. (Table 17).

Groups fabricated with Process A included 3.1A to 3.1W. The lyophilization formulation for AuNP+PEG NH2 syringe is provided in Table 17A below.

Groups fabricated with Process B included 3.1× to 3.1AC. The lyophilization formulation for the AuNP syringe is provided in Table 17B below.

For the samples shown in Table 17 below, 2-3 cm sections of AuNP fibers (n=2) were cut from the dried strand and immersed in 1-1.75 ml of PBS+0.001% Pluronic F-68, pH=7.2-7.4 at 37° C. Samples with lower SS % (≤50) were kept at pH=8.0 & 37° C. to observe accelerated release profiles. These samples were 3.1X, 3.1Y, 3.1Z, 3.1AA, 3.1AB and 3.1AC.

At each timepoint, 250 μL-1 mL of PBS was removed for measurement. Solutions were evaluated for AuNP absorbance via UV/Vis at 519 nm.

TABLE 17
Formulation and Composition of AuNP Fibers for controlled release with SS:SG ratios

| | PEG Formulation | | | | | | % Composition of Dried Implant | | | | | | |
|---|---|---|---|---|---|---|---|---|---|---|---|---|---|
| | % | | PEG Ester | PEG Ester | Ester | | | | | | Multi- | | |
| | maPEG | PEG | 1 | 2 | SS:SG | MWc | | PBS | Pluronic | Linear | arm | LDP:PEG | LDP:maPEG |
| Group | in DMC** | Amine | (SS) | (SG) | Ratio | (kDa) | Sucrose | Salts | F-68 | PEG | PEG | Ratio*** | Ratio |
| 3.1A | 5% | 4a40k | 4a40k | 4a40k | 80:20 | 20 | 14.3% | 28.4% | 0.02% | 0.0% | 57.3% | 0.75 | 0.75 |
| 3.1B | 5% | 4a40k | 4a40k | 4a40k | 70:30 | 20 | 14.3% | 28.4% | 0.02% | 0.0% | 57.3% | 0.75 | 0.75 |
| 3.1C | 5% | 4a40k | 4a20k | 4a20k | 90:10 | 15 | 14.3% | 28.4% | 0.02% | 0.0% | 57.3% | 0.75 | 0.75 |
| 3.1D | 5% | 4a40k | 4a20k | 4a20k | 80:20 | 15 | 14.3% | 28.4% | 0.02% | 0.0% | 57.3% | 0.75 | 0.75 |
| 3.1E | 5% | 4a20k | 4a40k | 4a40k | 90:10 | 15 | 14.3% | 28.4% | 0.02% | 0.0% | 57.3% | 0.75 | 0.75 |
| 3.1F | 5% | 4a20k | 4a40k | 4a40k | 80:20 | 15 | 14.3% | 28.4% | 0.02% | 0.0% | 57.3% | 0.75 | 0.75 |
| 3.1G | 5% | 8a20k | 4a40k | 4a40k | 100:0 | 12.5 | 14.3% | 28.4% | 0.02% | 0.0% | 57.3% | 0.75 | 0.75 |
| 3.1H | 5% | 8a20k | 4a40k | 4a40k | 90:10 | 12.5 | 14.3% | 28.4% | 0.02% | 0.0% | 57.3% | 0.75 | 0.75 |
| 3.1I | 6% | 4a20k | 4a20k | 4a20k | 85:15 | 10 | 12.9% | 25.4% | 0.02% | 0.0% | 61.7% | 0.62 | 0.62 |
| 3.1J | 6% | 4a20k | 4a20k | 4a20k | 90:10 | 10 | 12.9% | 25.4% | 0.02% | 0.0% | 61.7% | 0.62 | 0.62 |
| 3.1K | 6% | 4a40k | 4a40k | 4a40k | 100:0 | 20 | 12.9% | 25.4% | 0.02% | 0.0% | 61.7% | 0.62 | 0.62 |
| 3.1L | 6% | 4a40k | 4a40k | 4a40k | 95:5 | 20 | 12.9% | 25.4% | 0.02% | 0.0% | 61.7% | 0.62 | 0.62 |
| 3.1M | 6% | 4a40k | 4a40k | 4a40k | 90:10 | 20 | 12.9% | 25.4% | 0.02% | 0.0% | 61.7% | 0.62 | 0.62 |
| 3.1N | 6% | 4a20k | 4a40k | 4a40k | 100:0 | 15 | 12.9% | 25.4% | 0.02% | 0.0% | 61.7% | 0.62 | 0.62 |
| 3.1O | 6% | 4a20k | 4a40k | 4a40k | 95:5 | 15 | 12.9% | 25.4% | 0.02% | 0.0% | 61.7% | 0.62 | 0.62 |
| 3.1P | 6% | 4a20k | 4a40k | 4a40k | 90:10 | 15 | 12.9% | 25.4% | 0.02% | 0.0% | 61.7% | 0.62 | 0.62 |
| 3.1Q | 6% | 4a20k | 4a20k | 4a40k | 100:0 | 10 | 12.9% | 25.4% | 0.02% | 0.0% | 61.7% | 0.62 | 0.62 |
| 3.1R | 6% | 4a20k | 4a20k | 4a40k | 95:5 | 10.25 | 12.9% | 25.4% | 0.02% | 0.0% | 61.7% | 0.62 | 0.62 |
| 3.1S | 6% | 4a20k | 4a20k | 4a40k | 90:10 | 10.5 | 12.9% | 25.4% | 0.02% | 0.0% | 61.7% | 0.62 | 0.62 |
| 3.1T | 5% | 4a20k | 4a20k | 4a40k | 90:10 | 10.5 | 10.2% | 20.3% | 0.02% | 26.1% | 43.4% | 0.44 | 0.70 |
| 3.1U | 5% | 4a20k | 4a20k | 4a40k | 85:15 | 10.75 | 10.2% | 20.3% | 0.02% | 26.1% | 43.4% | 0.44 | 0.70 |
| 3.1V | 5% | 4a20k | 4a20k | 4a40k | 85:15 | 10.75 | 10.2% | 20.3% | 0.02% | 26.1% | 43.4% | 0.44 | 0.70 |
| 3.1W | 5% | 4a20k | 4a20k | 4a40k | 80:20 | 11 | 10.2% | 20.3% | 0.02% | 26.1% | 43.4% | 0.44 | 0.70 |
| 3.1X | 8% | 4a20k | 4a20k | 4a40k | 0:100 | 10 | 9.5% | 18.8% | 0.02% | 14.3% | 57.4% | 0.39 | 0.49 |
| 3.1Y | 8% | 4a20k | 4a20k | 4a40k | 15:85 | 14.25 | 9.5% | 18.8% | 0.02% | 14.3% | 57.4% | 0.39 | 0.49 |
| 3.1Z | 6% | 4a20k | 4a20k | 4a40k | 40:60 | 13 | 9.5% | 18.8% | 0.02% | 28.7% | 43% | 0.39 | 0.66 |
| 3.1AA | 6% | 4a20k | 4a20k | 4a40k | 50:50 | 12.5 | 9.5% | 18.8% | 0.02% | 28.7% | 43% | 0.39 | 0.66 |
| 3.1AB | 8% | 4a20k | 4a20k | 4a40k | 30:70 | 13.5 | 9.5% | 18.8% | 0.02% | 14.3% | 57.4% | 0.39 | 0.49 |
| 3.1AC | 8% | 4a20k | 4a20k | 4a40k | 40:60 | 13 | 9.5% | 18.8% | 0.02% | 14.3% | 57.4% | 0.39 | 0.49 |
| 3.1AD | 6% | 4a20k | 4a20k | 4a40k | 70:30 | 11.5 | 9.5% | 18.8% | 0.02% | 28.7% | 43% | 0.39 | 0.66 |
| 3.1AE | 6% | 4a20k | 4a20k | 4a40k | 65:35 | 11.75 | 9.5% | 18.8% | 0.02% | 28.7% | 43% | 0.39 | 0.66 |
| 3.1AF | 6% | 4a20k | 4a20k | 4a40k | 60:40 | 12 | 9.5% | 18.8% | 0.02% | 28.7% | 43% | 0.39 | 0.66 |

**maPEG: multi-arm PEG
***LDP:PEG ratio: Lyophilized Dry Powder:PEG mass ratio

TABLE 17A
Formulation and Composition of Lyophilized AuNPs-Fiber Fabrication of the groups according to Process A

| | | | | | | Lyo Volume | | | | | | |
|---|---|---|---|---|---|---|---|---|---|---|---|---|
| | | | | | | | | Total | | | | |
| | Cryo soln | | | Lyo soln | | AuNP | Cryo | Lyo | % Composition of Lyophilized Product | | | |
| | | Sucrose | PEG NH2 | Sucrose | PEG NH2 | Solution | Solution | Solution | | PBS | Pluronic | PEG |
| Group | PEG NH2 | (mg/mL) | (mg/mL) | (mg/mL) | (mg/mL) | (uL) | (uL) | (uL) | Sucrose | Salts | F-68 | NH2 |
| 3.1A | 4a40k | 34.2 | 68.4 | 5 | 10.0 | 300 | 51.3 | 351.3 | 20.1% | 39.7% | 0.03% | 40.1% |
| 3.1B | 4a40k | 34.2 | 68.4 | 5 | 10.0 | 300 | 51.3 | 351.3 | 20.1% | 39.7% | 0.03% | 40.1% |
| 3.1C | 4a40k | 34.2 | 91.2 | 5 | 13.3 | 300 | 51.3 | 351.3 | 17.7% | 35.1% | 0.03% | 47.2% |
| 3.1D | 4a40k | 34.2 | 91.2 | 5 | 13.3 | 300 | 51.3 | 351.3 | 17.7% | 35.1% | 0.03% | 47.2% |
| 3.1E | 4a20k | 34.2 | 45.6 | 5 | 6.7 | 300 | 51.3 | 351.3 | 23.2% | 45.9% | 0.04% | 30.9% |
| 3.1F | 4a20k | 34.2 | 45.6 | 5 | 6.7 | 300 | 51.3 | 351.3 | 23.2% | 45.9% | 0.04% | 30.9% |
| 3.1G | 8a20k | 34.2 | 27.4 | 5 | 4.0 | 300 | 51.3 | 351.3 | 26.5% | 52.3% | 0.05% | 21.1% |
| 3.1H | 8a20k | 34.2 | 27.4 | 5 | 4.0 | 300 | 51.3 | 351.3 | 26.5% | 52.3% | 0.05% | 21.1% |
| 3.1I | 4a20k | 34.2 | 82.0 | 5 | 12.0 | 300 | 51.3 | 351.3 | 18.6% | 36.8% | 0.03% | 44.6% |
| 3.1J | 4a20k | 34.2 | 82.0 | 5 | 12.0 | 300 | 51.3 | 351.3 | 18.6% | 36.8% | 0.03% | 44.6% |
| 3.1K | 4a40k | 34.2 | 82.0 | 5 | 12.0 | 300 | 51.3 | 351.3 | 18.6% | 36.8% | 0.03% | 44.6% |
| 3.1L | 4a40k | 34.2 | 82.0 | 5 | 12.0 | 300 | 51.3 | 351.3 | 18.6% | 36.8% | 0.03% | 44.6% |
| 3.1M | 4a40k | 34.2 | 82.0 | 5 | 12.0 | 300 | 51.3 | 351.3 | 18.6% | 36.8% | 0.03% | 44.6% |
| 3.1N | 4a20k | 34.2 | 54.7 | 5 | 8.0 | 300 | 51.3 | 351.3 | 21.8% | 43.2% | 0.04% | 34.9% |
| 3.1O | 4a20k | 34.2 | 54.7 | 5 | 8.0 | 300 | 51.3 | 351.3 | 21.8% | 43.2% | 0.04% | 34.9% |
| 3.1P | 4a20k | 34.2 | 54.7 | 5 | 8.0 | 300 | 51.3 | 351.3 | 21.8% | 43.2% | 0.04% | 34.9% |
| 3.1Q | 4a20k | 34.2 | 82.0 | 5 | 12.0 | 300 | 51.3 | 351.3 | 18.6% | 36.8% | 0.03% | 44.6% |
| 3.1R | 4a20k | 34.2 | 80.0 | 5 | 11.7 | 300 | 51.3 | 351.3 | 18.8% | 37.2% | 0.03% | 44.0% |
| 3.1S | 4a20k | 34.2 | 78.1 | 5 | 11.4 | 300 | 51.3 | 351.3 | 19.0% | 37.6% | 0.03% | 43.4% |
| 3.1T | 4a20k | 24.0 | 48.4 | 5 | 10.1 | 240 | 63.3 | 303.3 | 20.0% | 39.6% | 0.04% | 40.4% |
| 3.1U | 4a20k | 24.0 | 47.2 | 5 | 9.9 | 240 | 63.3 | 303.3 | 20.2% | 40.0% | 0.04% | 39.8% |
| 3.1V | 4a20k | 24.0 | 47.2 | 5 | 9.9 | 240 | 63.3 | 303.3 | 20.2% | 40.0% | 0.04% | 39.8% |
| 3.1W | 4a20k | 24.0 | 46.2 | 5 | 9.6 | 240 | 63.3 | 303.3 | 20.4% | 40.3% | 0.04% | 39.3% |

TABLE 17B
Formulation and Composition of Lyophilized AuNPs-Fiber Fabrication
of the groups according to Process B

| | | | Lyo Volume | | | % Composition of | | |
|---|---|---|---|---|---|---|---|---|
| | Cryo soln | Lyo soln | AuNP | Cryo | Total Lyo | Lyophilized Product | | |
| | Sucrose | Sucrose | Solution | Solution | Solution | | PBS | Pluronic |
| Groups | (mg/mL) | (mg/mL) | (uL) | (uL) | (uL) | Sucrose | Salts | F-68 |
| 3.1X to | 55 | 5 | 500 | 50 | 550 | 33.6% | 66.4% | 0.1% |
| 3.1AF | | | | | | | | |

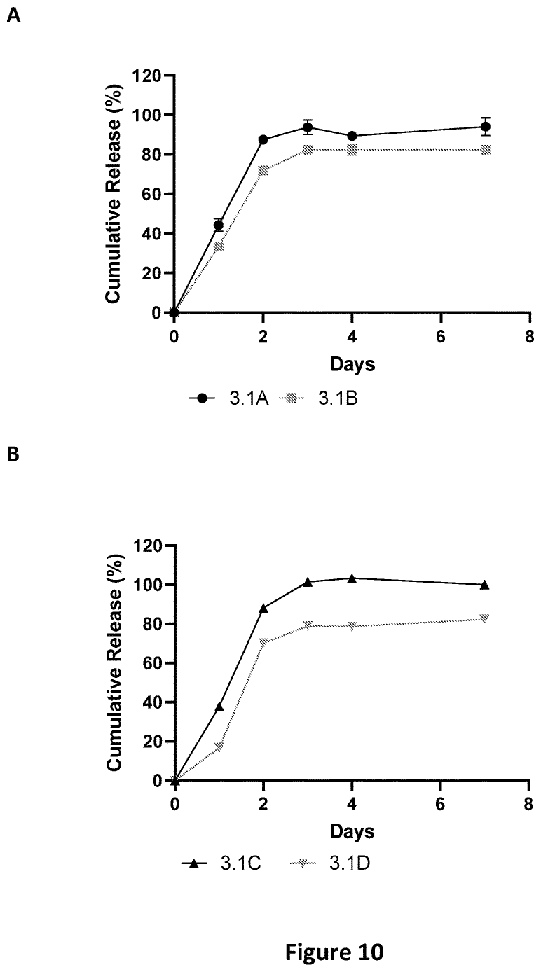

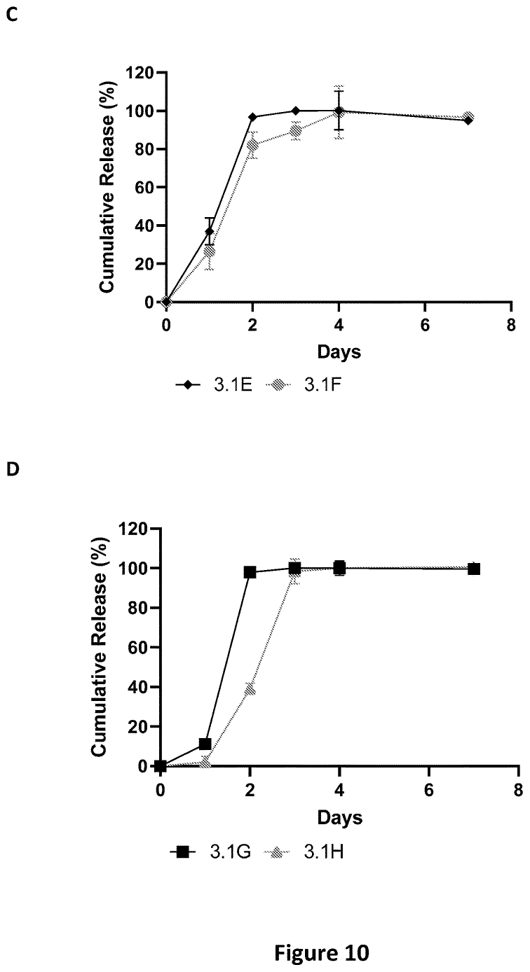

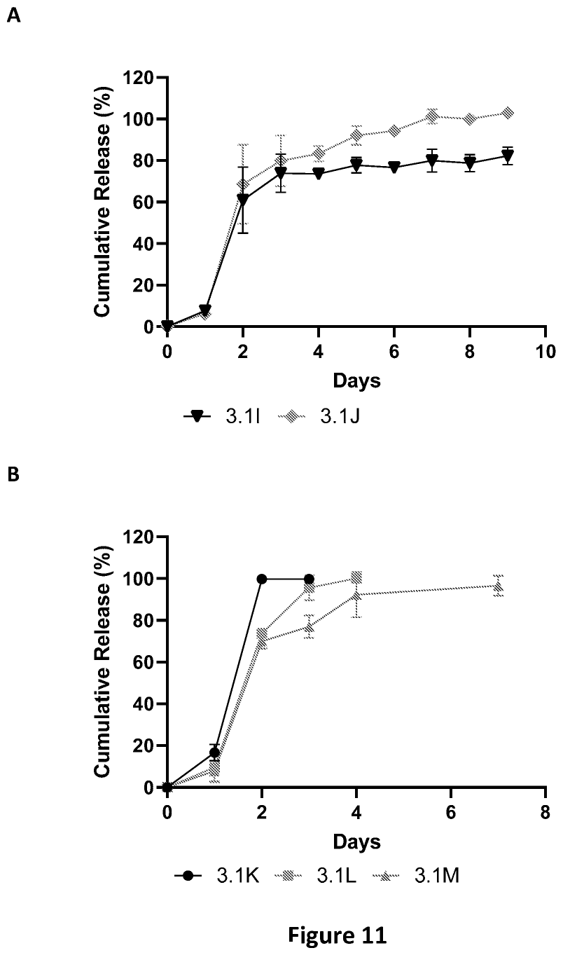

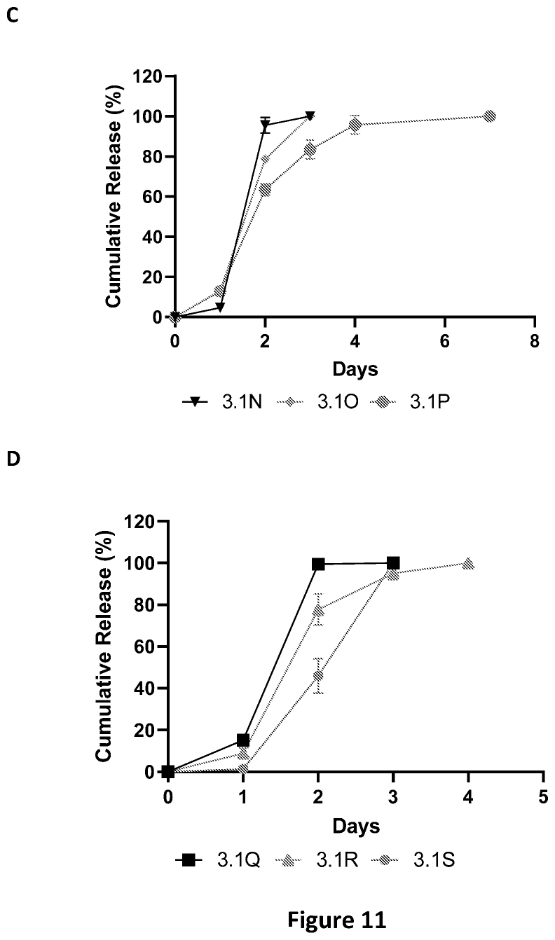

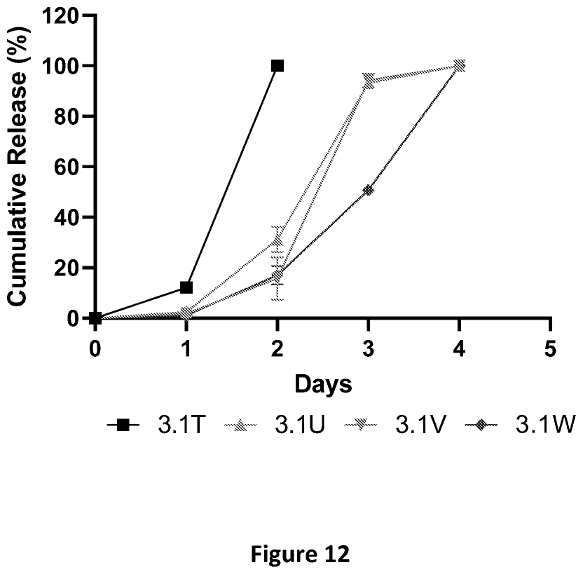

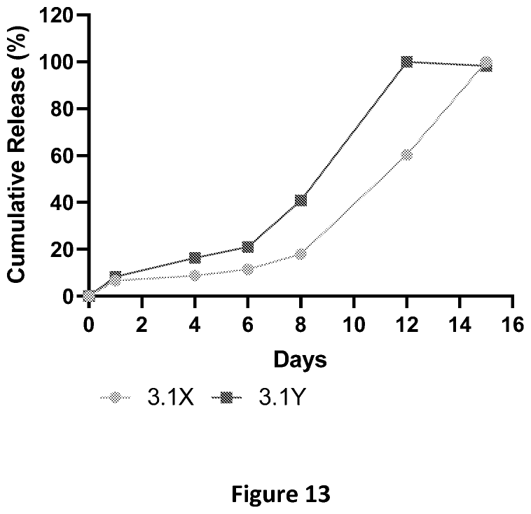

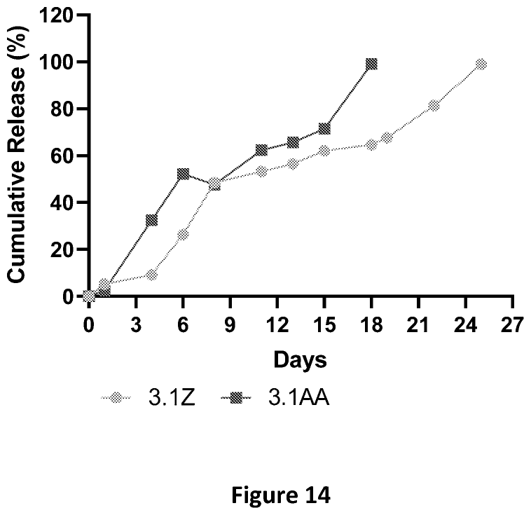

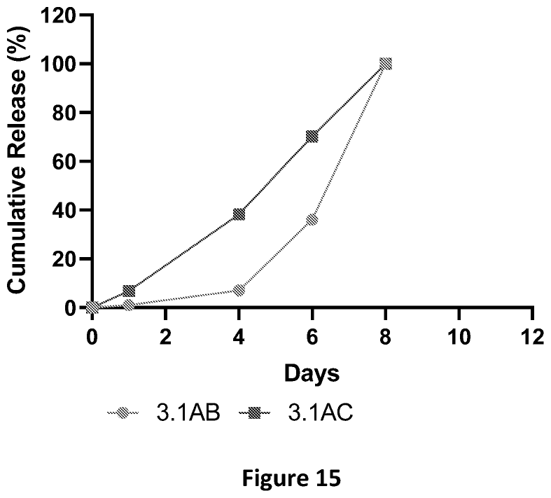

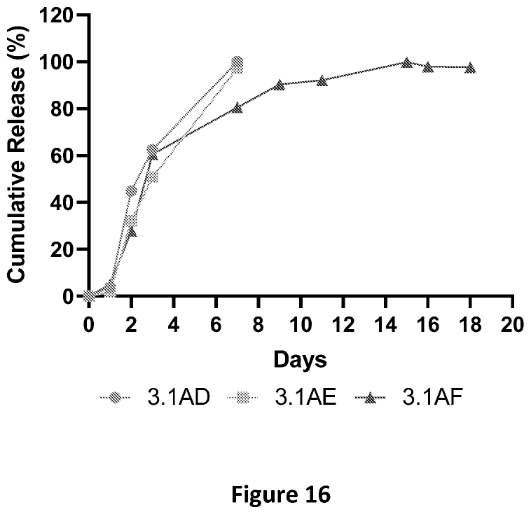

FIGS. 10-16 depict the release profile of different groups. These results clearly highlight the difference in the release profile by changing the SS:SG ratio. As the amount of SG is increased e.g., from 10% to 20%, the degradation of the fiber takes longer and the release is slower. This effect can be seen at day 1 release, per day release throughout the release profile, as well as number of days required for 100% release. Thus, it can be concluded that SS:SG ratio has a big impact on release kinetics and fiber degradation. Succinimidyl succinate groups typically degrade in the order of a few days, while succinimidyl glutarate groups degrade in the order of weeks. As the SS groups degrade, the hydrogel network mesh size increases rapidly in the first 2-4 days, allowing for the release of AuNPs. The remaining SG groups take longer to degrade, resulting in slower release of AuNPs.

### Example 3.2: AuNP IV Release—MW Between Crosslinks can be Selected to Control the Release Profile

To test the effect of MW between crosslinks, PEG NH2 (4a40k, 4a20k) were combined with different PEG esters (4a40k and 4a20k) to result in varying Mw between crosslinks. (Table 18).

The following Groups were fabricated with Process A. The lyophilization formulation for AuNP+PEG NH2 syringe is provided in Table 18A below.

TABLE 18
Formulation and Composition of AuNP Fibers for controlled release with Mw between crosslinks

| | PEG Formulation | | | | | | % Composition of Dried Implant | | | | | | |
|---|---|---|---|---|---|---|---|---|---|---|---|---|---|
| | % | | PEG | PEG | Ester | | | | | | Multi- | | LDP: |
| | maPEG | PEG | Ester 1 | Ester 2 | SS:SG | MWc | | PBS | Pluronic | Linear | arm | LDP:PEG | maPEG |
| Group | in DMC** | Amine | (SS) | (SG) | Ratio | (kDa) | Sucrose | Salts | F-68 | PEG | PEG | Ratio*** | Ratio |
| 3.2A | 5% | 4a40k | 4a40k | 4a40k | 80:20 | 20 | 14.3% | 28.4% | 0.02% | 0.0% | 57.3% | 0.75 | 0.75 |
| 3.2B | 5% | 4a20k | 4a20k | 4a20k | 80:20 | 10 | 14.3% | 28.4% | 0.02% | 0.0% | 57.3% | 0.75 | 0.75 |
| 3.2C | 6% | 4a20k | 4a20k | 4a20k | 90:10 | 10 | 12.9% | 25.4% | 0.02% | 0.0% | 61.7% | 0.62 | 0.62 |
| 3.2D | 6% | 4a40k | 4a40k | 4a40k | 90:10 | 20 | 12.9% | 25.4% | 0.02% | 0.0% | 61.7% | 0.62 | 0.62 |

**maPEG: multi-arm PEG
***LDP: PEG ratio: Lyophilized Dry Powder:PEG mass ratio

TABLE 18A
Formulation and Composition of Lyophilized AuNPs-Fiber Fabrication of the groups according to Process A

| | | | | | | Lyo Volume | | | | | | |
|---|---|---|---|---|---|---|---|---|---|---|---|---|
| | | | | | | | | Total | % Composition of | | | |
| | Cryo soln | | | Lyo soln | | AuNP | Cryo | Lyo | Lyophilized Product* | | | |
| | | Sucrose | PEG NH2 | Sucrose | PEG NH2 | Solution | Solution | Solution | | PBS | Pluronic | PEG |
| Group | PEG NH2 | (mg/mL) | (mg/mL) | (mg/mL) | (mg/mL) | (uL) | (uL) | (uL) | Sucrose | Salts | F-68 | NH2 |
| 3.2A | 4a40k | 34.2 | 68.4 | 5 | 10.0 | 300 | 51.3 | 351.3 | 20.1% | 39.7% | 0.03% | 40.1% |
| 3.2B | 4a20k | 34.2 | 68.4 | 5 | 10.0 | 300 | 51.3 | 351.3 | 20.1% | 39.7% | 0.03% | 40.1% |
| 3.2C | 4a20k | 34.2 | 82.0 | 5 | 12.0 | 300 | 51.3 | 351.3 | 18.6% | 36.8% | 0.03% | 44.6% |
| 3.2D | 4a40k | 34.2 | 82.0 | 5 | 12.0 | 300 | 51.3 | 351.3 | 18.6% | 36.8% | 0.03% | 44.6% |

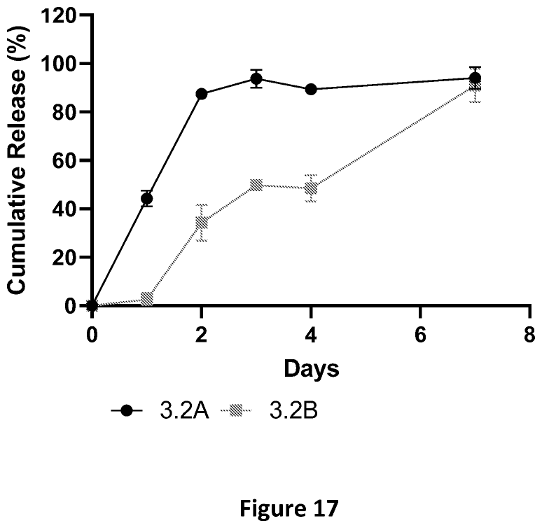

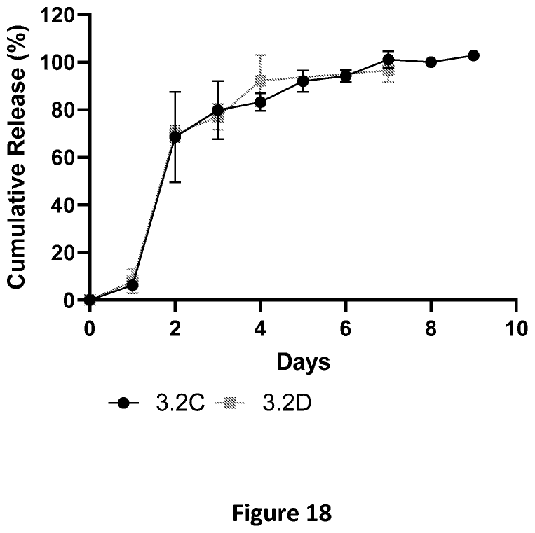

FIGS. 17 and 18 depict the release profiles of these groups. In a comparison of Day 1 release, 3.2A has a greater release at 44% as well as the larger starting molecular weight between crosslinks (MWc) at 20 kDa. In contrast, 3.2B has a Day 1 release of 2.5% and a MWc of 10 kDa. Similar trends can be seen when comparing per day release.

Furthermore, comparing groups 3.2C and 3.2D, it can be seen that as the MW between cross links is smaller the number of days required for 100% release is higher.

### Example 3.3: AuNP IV Release—LDP:maPEG Ratio can be Selected to Control the Release Profile

To test the effect of LDP:maPEG ratios, PEG NH2 (4a40k, 4a20k) were combined with different PEG esters (4a40k and 4a20k) at varying LDP:maPEG ratios. (Table 19).

Groups fabricated with Process A included 3.1E to 3.1J. The lyophilization formulation for AuNP+PEG NH2 syringe is provided in Table 19A and Table 19AA below.

Groups fabricated with Process B included 3.3A to 3.3D. The lyophilization formulation for the AuNP and AAV syringes is provided in Table 19B and 19BB below, respectively.

TABLE 19
Formulation and Composition of AuNP and AAV Fibers for controlled release with LDP:maPEG

| | PEG Formulation | | | | | | % Composition of Dried Implant | | | | | | |
|---|---|---|---|---|---|---|---|---|---|---|---|---|---|
| | % | | PEG | PEG | Ester | | | | | | Multi- | LDP: | LDP: |
| | maPEG in | PEG | Ester 1 | Ester 2 | SS:SG | MWc | | PBS | Pluronic | Linear | arm | PEG | maPEG |
| Group | DMC** | Amine | (SS) | (SG) | Ratio | (kDa) | Sucrose | Salts | F-68 | PEG | PEG | Ratio*** | Ratio |
| 3.3A | 6% | 4a20k | 4a20k | 4a40k | 70:30 | 11.5 | 9.5% | 18.8% | 0.02% | 28.7% | 43% | 0.39 | 0.66 |
| 3.3B | 6% | 4a20k | 4a20k | 4a40k | 70:30 | 11.5 | 14.4% | 27.5% | 0.01% | 23.2% | 34.7% | 0.60 | 1.0 |
| 3.3C | 8% | 4a20k | 4a20k | 4a40k | 60:40 | 12 | 9.5% | 18.8% | 0.02% | 14.3% | 57.4% | 0.39 | 0.49 |
| 3.3D | 8% | 4a20k | 4a20k | 4a40k | 60:40 | 12 | 14.4% | 27.5% | 0.01% | 11.6% | 46.3% | 0.60 | 0.75 |
| 3.3E | 5% | 4a40k | 4a20k | 4a20k | 90:10 | 15 | 14.3% | 28.4% | 0.02% | 0.0% | 57.3% | 0.75 | 0.75 |
| 3.3F | 6% | 4a40k | 4a20k | 4a20k | 90:10 | 15 | 12.9% | 25.4% | 0.02% | 0.0% | 61.7% | 0.62 | 0.62 |
| 3.3G | 5% | 4a20k | 4a40k | 4a40k | 80:20 | 15 | 14.3% | 28.4% | 0.02% | 0.0% | 57.3% | 0.75 | 0.75 |
| 3.3H | 6% | 4a20k | 4a40k | 4a40k | 80:20 | 15 | 12.9% | 25.4% | 0.02% | 0.0% | 61.7% | 0.62 | 0.62 |
| 3.3I | 5% | 4a20k | 4a20k | 4a20k | 90:10 | 10 | 14.3% | 28.4% | 0.02% | 0.0% | 57.3% | 0.75 | 0.75 |
| 3.3J | 6% | 4a20k | 4a20k | 4a20k | 90:10 | 10 | 12.9% | 25.4% | 0.02% | 0.0% | 61.7% | 0.62 | 0.62 |

**maPEG: multi-arm PEG
***LDP:PEG ratio: Lyophilized Dry Powder:PEG mass ratio

TABLE 19A
Formulation and Composition of Lyophilized AuNPs-Fiber Fabrication of the groups according to Process A

| | | | | | | Lyo Volume | | | | | | |
|---|---|---|---|---|---|---|---|---|---|---|---|---|
| | | | | | | | | Total | % Composition | | | |
| | Cryo soln | | | Lyo soln | | AuNP | Cryo | Lyo | of Lyophilized Product | | | |
| | PEG | Sucrose | PEG NH2 | Sucrose | PEG NH2 | Solution | Solution | Solution | | PBS | Pluronic | PEG |
| Group | NH2 | (mg/mL) | (mg/mL) | (mg/mL) | (mg/mL) | (uL) | (uL) | (uL) | Sucrose | Salts | F-68 | NH2 |
| 3.3E | 4a40k | 34.2 | 91.2 | 5 | 13.3 | 300 | 51.3 | 351.3 | 17.7% | 35.1% | 0.03% | 47.2% |
| 3.3F | 4a40k | 34.2 | 109.4 | 5 | 16.0 | 300 | 51.3 | 351.3 | 16.2% | 32.0% | 0.03% | 51.7% |
| 3.3G | 4a20k | 34.2 | 45.6 | 5 | 6.7 | 300 | 51.3 | 351.3 | 23.2% | 45.9% | 0.04% | 30.9% |
| 3.3H | 4a20k | 34.2 | 54.7 | 5 | 8.0 | 300 | 51.3 | 351.3 | 21.8% | 43.2% | 0.04% | 34.9% |
| 3.3I | 4a20k | 34.2 | 68.4 | 5 | 10.0 | 300 | 51.3 | 351.3 | 20.1% | 39.7% | 0.03% | 40.1% |
| 3.3J | 4a20k | 34.2 | 82.0 | 5 | 12.0 | 300 | 51.3 | 351.3 | 18.6% | 36.8% | 0.03% | 44.6% |

TABLE 19AA
Formulation and Composition of Lyophilized AuNP fibers with Varying LDP:PEG ratios

| | PEG Formulation | | | | | % Composition of Dried Implant**** | | | | | | |
|---|---|---|---|---|---|---|---|---|---|---|---|---|
| | % | | PEG | PEG | Ester | | | | | Multi- | LDP: | LDP: |
| | maPEG | PEG | Ester 1 | Ester 2 | SS:SG | | PBS | Pluronic | AuN | arm | PEG | maPEG |
| GN* | in DMC** | Amine | (SS) | (SG) | Ratio | Sucrose | Salts | F-68 | P | PEG | Ratio*** | Ratio |
| 131-1 | 6% | 4a20k | 4a20k | 4a40k | 70:30 | 15.4% | 33.3% | 0.02% | 1.2% | 50% | 1.00 | 1.00 |
| 131-2 | 6% | 4a20k | 4a20k | 4a40k | 70:30 | 17.2% | 37.1% | 0.02% | 1.3% | 44.5% | 1.25 | 1.25 |
| 132-1 | 6% | 4a20k | 4a20k | 4a40k | 70:30 | 18.5% | 40% | 0.02% | 1.4% | 40% | 1.50 | 1.50 |
| 132-2 | 6% | 4a20k | 4a20k | 4a40k | 70:30 | 19.7% | 42.5% | 0.02% | 1.5% | 36.3% | 1.75 | 1.75 |
| 133 | 6% | 4a20k | 4a20k | 4a40k | 70:30 | 20.6% | 44.5% | 0.02% | 1.6% | 33.4% | 2.00 | 2.00 |

*Lab notebook page number shortened (131-1--> MP-553-131-1)
**maPEG: multi-arm PEG
***LDP:PEG ratio: Lyophilized Dry Powder:PEG mass ratio
****Linear PEG % was kept at 0.

TABLE 19B
Formulation and Composition of Lyophilized AuNPs-Fiber Fabrication of the groups according to Process B

| | | | Lyo Volume | | | | | |
|---|---|---|---|---|---|---|---|---|
| | Cryo soln | Lyo soln | AuNP | Cryo | Total Lyo | % Composition of Lyophilized Product | | |
| | Sucrose | Sucrose | Solution | Solution | Solution | | PBS | Pluronic |
| Groups | (mg/mL) | (mg/mL) | (uL) | (uL) | (uL) | Sucrose | Salts | F-68 |
| 3.3A and | 55 | 5 | 500 | 50 | 550 | 33.6% | 66.4% | 0.1% |
| 3.3C | | | | | | | | |

TABLE 19BB
Formulation and Composition of Lyophilized AAVs of groups 3.3B and 3.3D-Fiber Fabrication of the groups according
to Process B

| | Cryo | Lyo Solution | | Lyo Volume | | | % Mass Composition of Lyophilized Product | | | |
|---|---|---|---|---|---|---|---|---|---|---|
| | Solution | | Sodium | AAV | Cryo | Total Lyo | | | PBS Salts + | |
| | Sucrose | Sucrose | Chloride | Solution | Solution | Solution | | | Sodium | Pluronic |
| Group | (mg/mL) | (mg/mL) | (mg/mL) | (uL) | (uL) | (uL) | AAV | Sucrose | Chloride | F-68 |
| 3.3B | 15.7 | 10 | 4.2 | 181.3 | 325.5 | 506.8 | 0.6% | 41.3% | 58.1% | 0.04% |
| 3.3D | 15.7 | 10 | 4.2 | 181.3 | 325.5 | 506.8 | 0.6% | 41.3% | 58.1% | 0.04% |

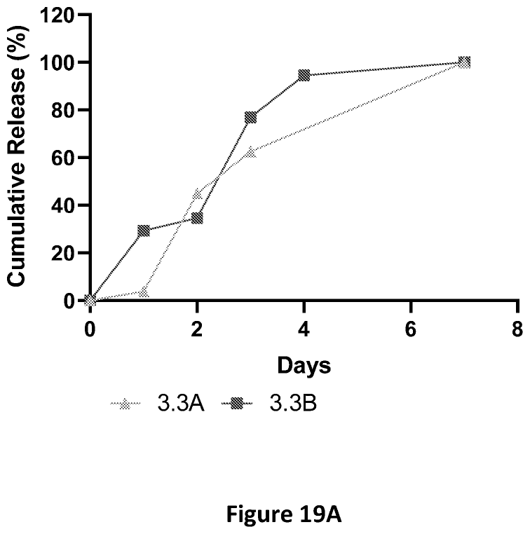

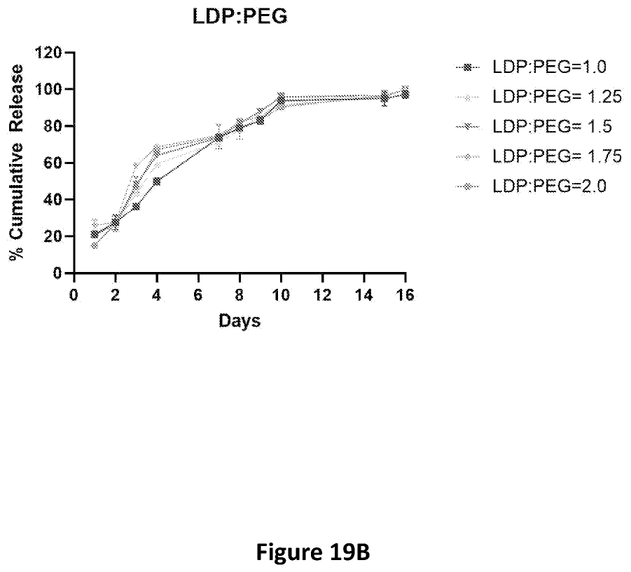

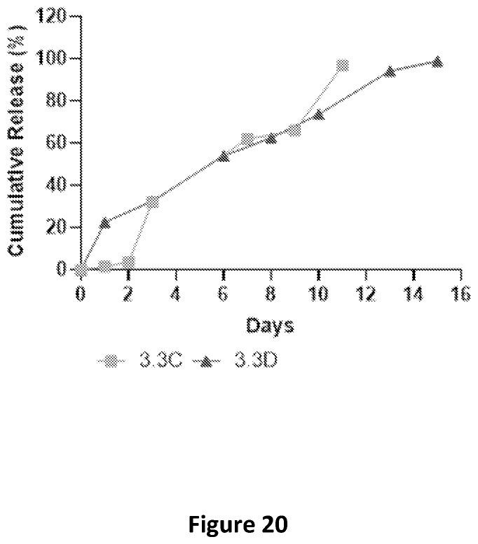

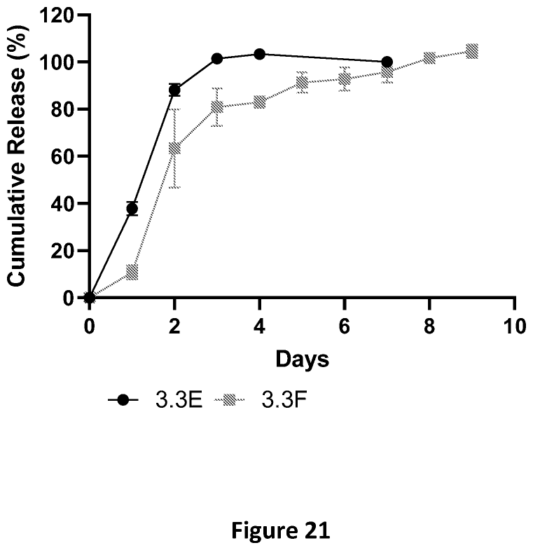

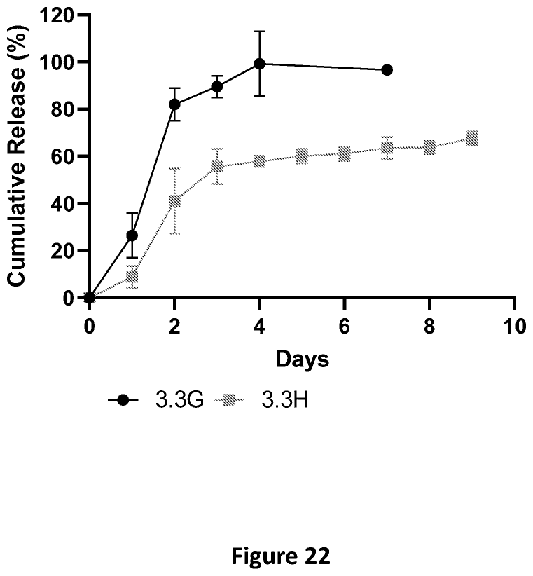

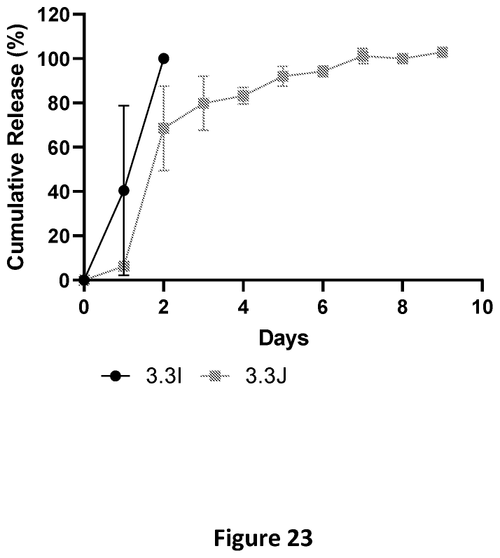

FIGS. 19-23 depict the release profiles of these groups. From these results, it is seen that a greater % maPEG concentration and lower LDP:maPEG ratios results in slower release kinetics, likely due to the increased crosslinking density. The use of 6% maPEG in DMC resulted in lower LDP:maPEG ratios and a more densely crosslinked matrix which slowed degradation, and AuNP release, compared to 5% maPEG and higher LDP:maPEG ratio.

Comparing, for example, groups 3.3A and 3.3B, the effect of LDP:maPEG ratio can be clearly seen on day 1 release. Similar trends can be seen when comparing groups 3.3C and 3.3D. When comparing 3.3E with 3.3F, 3.3G with 3.3H, and 3.3I with 3.3J, the effects of LDP:maPEG ratios on day 1 release, per day release and number of days required for 100% release are clear.

### Overall Conclusion of Examples 3.1 to 3.3

From the results discussed above, it is clear that SS:SG ratios, Mw between cross links, % PEG and (LDP:maPEG) ratios alone and in a complex interplay affect the release profiles. Thus, each of these features alone or in combination with each other can be selected to customize the release profile.

### Example 3.4—AuNP IV Release—Number of Days Required to Achieve 100% Release

In Examples 3.1 to 3.3, the main goal was to assess the major players that affect the release profile. In this Example, it was aimed to develop PEG formulations with fast (<7 days), medium (7-14 days), and slow-release profiles (21-28 days).

Fibers were fabricated according to Process B shown in Example 2.2.

2 cm sections of AuNP fibers were cut from the dried strand and immersed in 1 mL of PBS+0.001% Pluronic F-68, pH=7.2 & pH=8.0 at 37° C. At each timepoint, 250 μL of PBS was removed and replaced with fresh PBS. At pH=8.0, fibers degrade faster so it shows us accelerated release profiles of the PEG formulations.

Accelerated degradation factor=EXP(1.766*(8−7.2))=4.1

IV release samples were stored in the 4° C. fridge. 100 μL of IV release sample was pipetted into a 96 well plate. Standards were prepared using AuNP stock solution. Standard curve checked before testing samples. Solutions were evaluated for AuNP absorbance via UV/Vis at 519 nm.

In this study, different variables were investigated to see their influence on release kinetics of encapsulated AuNPs. PEG NH2 (4a20k) was combined with different PEG esters (4a20k and 4a40k) at varying SS:SG ratios. The % multi-arm PEG concentration in DMC was varied between 6-10%. Sucrose concentration (5 mg/mL) was kept constant in all fibers. Table 20 shows the formulation and composition of the final AuNP-loaded fibers.

TABLE 20
Formulation and Composition of AuNP Fibers

| | PEG Formulation | | | | | % Composition of Dried Implant | | | | | | |
|---|---|---|---|---|---|---|---|---|---|---|---|---|
| | % maPEG | | PEG | PEG | Ester | | | | | Multi- | | LDP: |
| | in | PEG | Ester 1 | Ester 2 | SS:SG | | PBS | Pluronic | Linear | arm | LDP:PEG | maPEG |
| GN* | DMC** | Amine | (SS) | (SG) | Ratio | Sucrose | Salts | PF-68 | PEG | PEG | Ratio*** | Ratio |
| 48-1 | 6% | 4a20k | 4a20k | 4a40k | 85:15 | 9.5% | 18.8% | 0.02% | 28.7% | 43% | 0.39 | 0.66 |
| 48-2 | 6% | 4a20k | 4a20k | 4a40k | 82.5:17.5 | 9.5% | 18.8% | 0.02% | 28.7% | 43% | 0.39 | 0.66 |
| 48-3 | 7% | 4a20k | 4a20k | 4a40k | 87.5:12.5 | 9.5% | 18.8% | 0.02% | 21.5% | 50.2% | 0.39 | 0.56 |
| 48-4 | 7% | 4a20k | 4a20k | 4a40k | 85:15 | 9.5% | 18.8% | 0.02% | 21.5% | 50.2% | 0.39 | 0.56 |
| 49-1 | 8% | 4a20k | 4a20k | 4a40k | 0:100 | 9.5% | 18.8% | 0.02% | 14.3% | 57.4% | 0.39 | 0.49 |
| 49-2 | 8% | 4a20k | 4a20k | 4a40k | 15:85 | 9.5% | 18.8% | 0.02% | 14.3% | 57.4% | 0.39 | 0.49 |
| 49-3 | 10% | 4a20k | 4a20k | 4a40k | 0:100 | 9.5% | 18.8% | 0.02% | 0.0% | 71.7% | 0.39 | 0.39 |
| 49-4 | 10% | 4a20k | 4a20k | 4a40k | 15:85 | 9.5% | 18.8% | 0.02% | 0.0% | 71.7% | 0.39 | 0.39 |
| 54-2 | 6% | 4a20k | 4a20k | 4a40k | 30:70 | 9.5% | 18.8% | 0.02% | 28.7% | 43% | 0.39 | 0.66 |
| 54-3 | 6% | 4a20k | 4a20k | 4a40k | 40:60 | 9.5% | 18.8% | 0.02% | 28.7% | 43% | 0.39 | 0.66 |
| 54-4 | 6% | 4a20k | 4a20k | 4a40k | 50:50 | 9.5% | 18.8% | 0.02% | 28.7% | 43% | 0.39 | 0.66 |
| 54-5 | 8% | 4a20k | 4a20k | 4a40k | 30:70 | 9.5% | 18.8% | 0.02% | 14.3% | 57.4% | 0.39 | 0.49 |
| 54-6 | 8% | 4a20k | 4a20k | 4a40k | 40:60 | 9.5% | 18.8% | 0.02% | 14.3% | 57.4% | 0.39 | 0.49 |
| 56-1 | 6% | 4a20k | 4a20k | 4a40k | 70:30 | 9.5% | 18.8% | 0.02% | 28.7% | 43% | 0.39 | 0.66 |
| 56-2 | 6% | 4a20k | 4a20k | 4a40k | 65:35 | 9.5% | 18.8% | 0.02% | 28.7% | 43% | 0.39 | 0.66 |
| 56-3 | 6% | 4a20k | 4a20k | 4a40k | 60:40 | 9.5% | 18.8% | 0.02% | 28.7% | 43% | 0.39 | 0.66 |
| 56-4 | 8% | 4a20k | 4a20k | 4a40k | 65:35 | 9.5% | 18.8% | 0.02% | 14.3% | 57.4% | 0.39 | 0.49 |
| 56-5 | 8% | 4a20k | 4a20k | 4a40k | 60:40 | 9.5% | 18.8% | 0.02% | 14.3% | 57.4% | 0.39 | 0.49 |
| 56-6 | 8% | 4a20k | 4a20k | 4a40k | 55:45 | 9.5% | 18.8% | 0.02% | 14.3% | 57.4% | 0.39 | 0.49 |

**maPEG: multi-arm PEG
***LDP:PEG ratio: Lyophilized Dry Powder:PEG mass ratio

All groups were fabricated with Process B. The lyophilization formulation for the AuNP syringe is provided in Table 20B below.

TABLE 20B
Formulation and Composition of Lyophilized AuNPs

| | Lyo soln | Lyo Volume | | | | | |
|---|---|---|---|---|---|---|---|
| Cryo soln | Sucrose | AuNP | Cryo | Total Lyo | % Composition of Lyophilized Product | | |
| Sucrose | (mg/ | Solution | Solution | Solution | | PBS | Pluronic |
| (mg/mL) | mL) | (uL) | (uL) | (uL) | Sucrose | Salts | F-68 |
| 55 | 5 | 500 | 50 | 550 | 33.6% | 66.4% | 0.1% |

Table 21 shows the effects of % maPEG and SS:SG ratio on gelation time. Increasing PEG SS resulted in faster gelation. While the 20:80 SS:SG formulation with 6% maPEG gelled in 5 minute 32 seconds, the 70:30 SS:SG formulation with the same % maPEG gelled in 2 minutes 33 seconds. It is observed that a greater % maPEG concentration results in faster gelation since there are more available amine and ester groups in the environment for crosslinking. 8% maPEG formulations with the same SS:SG ratios had shorter gelation time compared to 6% maPEG formulations.

TABLE 21
Effect of % maPEG and SS:SG Ratio on Gelation Time

| | % maPEG | SS:SG | |
|---|---|---|---|
| GN | in DMC** | Ratio | Gelation Time |
| 54-1 | 6% | 20:80 | 5 min 32 sec |
| 54-2 | 6% | 30:70 | 4 min 15 sec |
| 54-3 | 6% | 40:60 | 3 min 43 sec |
| 54-4 | 6% | 50:50 | 3 min 02 sec |
| 56-3 | 6% | 60:40 | 2 min 15 sec |
| 56-2 | 6% | 65:35 | 2 min 25 sec |
| 56-1 | 6% | 70:30 | 2 min 33 sec |
| 54-5 | 8% | 30:70 | 3 min 13 sec |
| 54-6 | 8% | 40:60 | 2 min 30 sec |
| 56-6 | 8% | 55:45 | 1 min 43 sec |
| 56-5 | 8% | 60:40 | 1 min 39 sec |

The samples with high SS % (>82.5) degraded less than 4 days at pH=7.2 & 37° C. On day 4, there wasn't any visible fibers in the buffer solutions. Samples with lower SS % (≤50) were kept at pH=8.0 & 37° C. to observe accelerated release profiles.

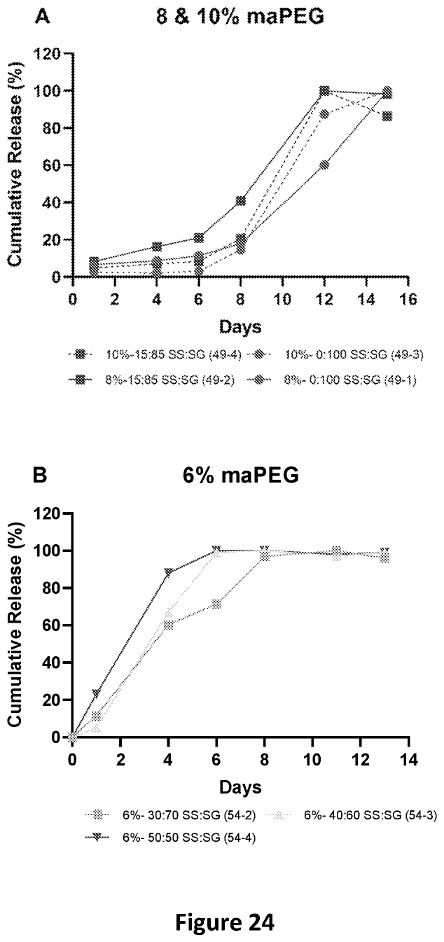

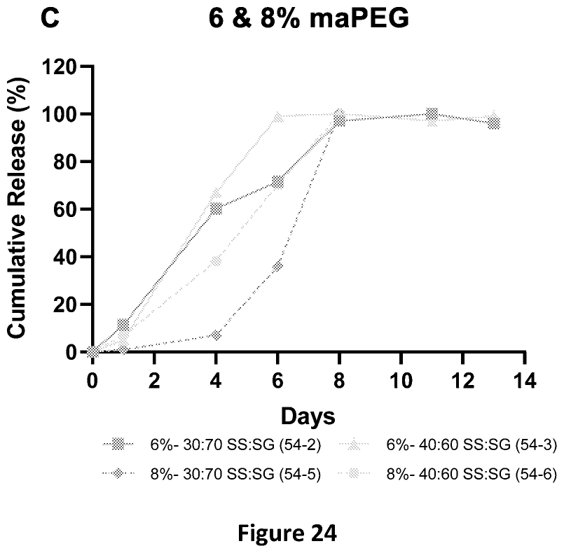

FIG. 24 includes the IV release curves at pH=8.0 & 37° C. It is seen that a greater % maPEG concentration results in slower release kinetics initially, likely due to the increased crosslinking density and smaller mesh size (FIGS. 24A & 24C). While the 6% maPEG formulation with 40:60 SS:SG reached 67% release in 4 days, the 8% maPEG formulation with the same SS:SG ratio reached only 38% release in 4 days. FIG. 24B shows the release curves for 6% maPEG formulations with varying SS:SG ratios. Increasing PEG SG resulted in slower degrading fibers and slower release. While the 50:50 SS:SG formulation reached 100% release in 6 days, the 20:80 SS:SG formulation reached 100% release in 11 days.

Based on IV release results at pH=8.0 & 37° C., 100% cumulative release at pH: 7.2 & 37° C. estimated using accelerated degradation factor (Table 22). Formulations within the desired range (14-28 days) were selected for further IV release study at pH: 7.2 & 37° C.

TABLE 22
Estimated IV Release at pH: 7.2 & 37° C.

| | | SS (%) | SG (%) | Estimated |
|---|---|---|---|---|
| | maPEG | 4arm | 4arm | Release* |
| GN | (%) | 20k | 40k | (days) |
| 54-1 | 6 | 20 | 80 | ~32-45 |
| 54-2 | 6 | 30 | 70 | ~24-33 |
| 54-3 | 6 | 40 | 60 | ~16-25 |
| 54-4 | 6 | 50 | 50 | ~16-25 |
| 49-1 | 8 | 0 | 100 | ~48-61 |
| 49-2 | 8 | 15 | 85 | ~32-48 |
| 54-5 | 8 | 30 | 70 | ~24-33 |
| 54-6 | 8 | 40 | 60 | ~24-33 |
| 49-3 | 10 | 0 | 100 | ~48-61 |
| 49-4 | 10 | 15 | 85 | ~32-48 |

*Estimated release calculated based on Accelerated Degradation Factor = 4.1 x

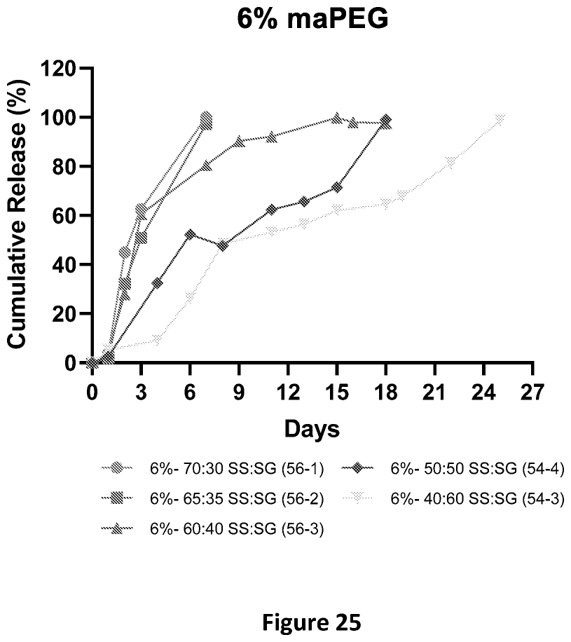

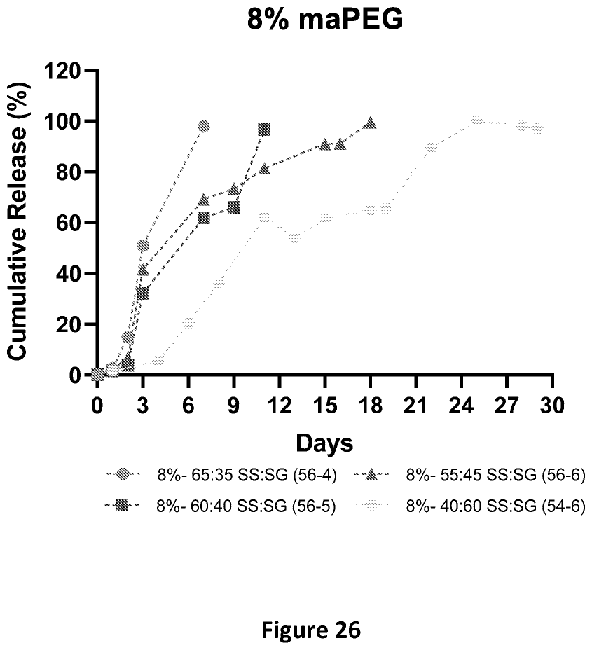

FIGS. 25 & 26 show the release curves for 6% & 8% maPEG formulations with varying SS:SG ratios at pH: 7.2 & 37° C. From these results, it's seen that the SS:SG ratio has a great impact on release kinetics and fiber degradation. Increasing SS content led to a faster degrading fibers and faster release. While the 70:30 SS:SG formulation with 6% maPEG reached 100% release within 4-7 days, the 40:60 SS:SG formulation at the same % maPEG reached 100% release in 25 days. Same phenomena observed with 8% maPEG formulations as well.

From the formulation development work, three formulations listed below (Table 23) were selected for fast, medium, and slow-release kinetics. The impact of release kinetics on inflammation and AAV transduction will be studied further with an ocular in vivo study. The sustained release of AAVs may be beneficial for in vivo ocular applications to mitigate acute inflammatory response.

TABLE 23
Selected Formulations for Further Ocular In Vivo Investigation

| | | | | % 100 |
|---|---|---|---|---|
| Release | maPEG | SS (%) | SG (%) | Release |
| profile | (%) | 4arm 20k | 4arm 40k | (days) |
| Fast | 6 | 70 | 30 | 4-7 |
| Medium | 8 | 60 | 40 | 11 |
| Slow | 6 | 40 | 60 | 25 |

### Example 3.5: Sugar Content and Release Profile

In this release study, the release profile of AuNP was studied using various sugar levels. To test the effect of sugar, PEG NH2 (4a20k) was combined with different PEG esters SS and SG (4a40k and 4a20k). The sugar concentrations tested were 1) 50 mg/ml sucrose+50 mg/ml mannitol and 2) 50 mg/mL trehalose+50 mg/mL mannitol. This resulted in (w/w %) of total sugar in the dried implant of approximately 65-70%.

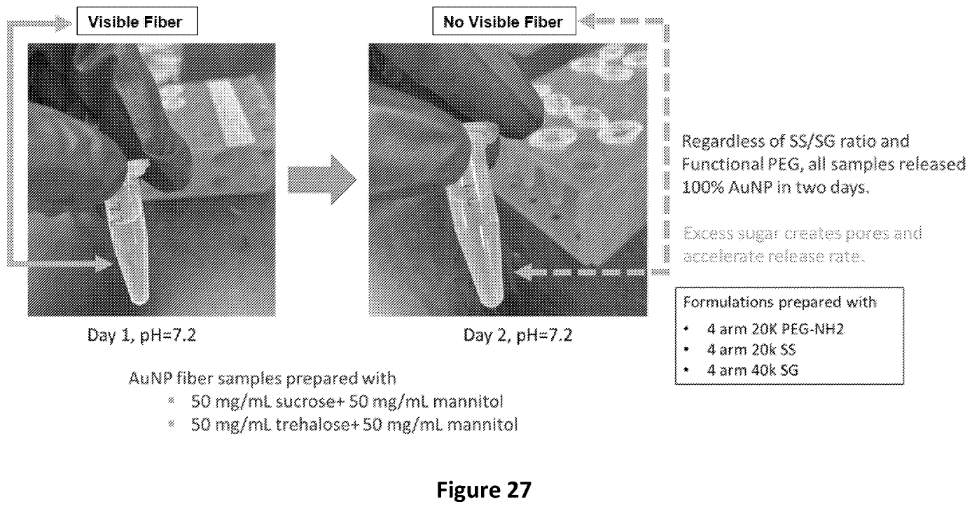

It was surprisingly found that the sugar content must be controlled in the implant in order to control the release profile. Regardless of SS/SG ratio and functional PEG, all samples released 100% AuNP within about two days in PBS solution at 37° C. and pH 7.2 when total sugar concentration is 100 mg/mL (either with 50 mg/ml of sucrose+50 mg/mL mannitol or 50 mg/mL trehalose+50 mg/mL mannitol) (see FIG. 27).

Thus, if the desired 100% release is at least two days or greater than 2 days, sugar (w/w) % in the dried implant should be controlled.

### Example 3.6: AuNP Release Profiles can be Reproduced with AAVs

In this experiment, AAVs were encapsulated in xerogel fibers that is a hydrogel after exposure to an aqueous solution and the release kinetics were evaluated via PCR titration.

It is crucial that the above demonstrated release profiles with AuNPs be reproduced with AAVs. Thus, two AuNP fibers were formulated as shown in Table 24 below namely 448-056B and 448-056D.

The experiments with AAVs were conducted with the same formulations as 448-056B and 448-056BD to test the reproducibility of the release profiles from AuNP fibers to AAV fibers. Furthermore, it is crucial that the release profile of AAVs remains substantially same at different doses. Two AAV doses were tested: a 1× and 4× dose. Thus, the final AAV fibres were namely 549-059A (low dose—similar formulation to 448-056B), 549-059B (high dose dose—similar formulation to 448-056B), 549-059C (low dose—similar formulation to 448-056D), 549-059D (high dose—similar formulation to 448-056D) as shown in Table 25 below.

TABLE 24
AuNP formulations

| | PEG Formulation | | | | | | % Composition of Dried Implant | | | | | | |
|---|---|---|---|---|---|---|---|---|---|---|---|---|---|
| | % | | PEG | PEG | Ester | | | | | | Multi- | | LDP: |
| | maPEG | PEG | Ester 1 | Ester 2 | SS:SG | MWc | | PBS | Pluronic | Linear | arm | LDP:PEG | maPEG |
| Group | in DMC* | Amine | (SS) | (SG) | Ratio | (kDa) | Sucrose | Salts | F-68 | PEG | PEG | Ratio** | Ratio |
| 448-056B | 5% | 4a40k | 4a20k | 4a40k | 85:15 | 15.75 | 10.4% | 20.6% | 0.02% | 25.9% | 43.1% | 0.45 | 0.72 |
| 448-056D | 5% | 4a20k | 4a20k | 4a40k | 85:15 | 10.75 | 10.4% | 20.6% | 0.02% | 25.9% | 43.1% | 0.45 | 0.72 |

TABLE 25
AAV formulations

| | PEG Formulation | | | | | | % Composition of Dried Implant | | | | | | | |
|---|---|---|---|---|---|---|---|---|---|---|---|---|---|---|
| | | | PEG | PEG | Ester | | | | | | | Multi- | | LDP: |
| | % maPEG | PEG | Ester 1 | Ester 2 | SS:SG | MWc | | | PBS | Pluronic | Linear | arm | LDP:PEG | maPEG |
| Group | in DMC* | Amine | (SS) | (SG) | Ratio | (kDa) | AAV | Sucrose | Salts | F-68 | PEG | PEG | Ratio** | Ratio |
| 549-059A | 5% | 4a40k | 4a20k | 4a40k | 85:15 | 15.75 | 0.1% | 10.4% | 20.6% | 0.02% | 25.8% | 43.0% | 0.45 | 0.72 |
| 549-059B | 5% | 4a40k | 4a20k | 4a40k | 85:15 | 15.75 | 0.3% | 10.4% | 20.5% | 0.02% | 25.8% | 43.0% | 0.45 | 0.73 |
| 549-059C | 5% | 4a20k | 4a20k | 4a40k | 85:15 | 10.75 | 0.1% | 10.4% | 20.6% | 0.02% | 25.8% | 43.0% | 0.45 | 0.72 |
| 549-059D | 5% | 4a20k | 4a20k | 4a40k | 85:15 | 10.75 | 0.3% | 10.4% | 20.5% | 0.02% | 25.8% | 43.0% | 0.45 | 0.73 |

*maPEG: multi-arm PEG
**LDP:PEG ratio: Lyophilized Dry Powder:PEG mass ratio

TABLE 26
Formulation and Composition of Lyophilized AuNPs-Fiber Fabrication of the groups according to Process A

| | | | | | | Lyo Volume | | | | | | |
|---|---|---|---|---|---|---|---|---|---|---|---|---|
| | Cryo soln | | | Lyo soln | | AuNP | Cryo | Total Lyo | % Composition of Lyophilized Product* | | | |
| | | Sucrose | PEG NH2 | Sucrose | PEG NH2 | Solution | Solution | Solution | | PBS | Pluronic | PEG |
| Group | PEG NH2 | (mg/mL) | (mg/mL) | (mg/mL) | (mg/mL) | (uL) | (uL) | (uL) | Sucrose | Salts | F-68 | NH2 |
| 448-056B | 4a40k | 16.8 | 44.5 | 5 | 13.3 | 240 | 101.9 | 341.9 | 17.8% | 35.2% | 0.04% | 46.9% |
| 448-056D | 4a20k | 16.8 | 32.6 | 5 | 9.7 | 240 | 101.9 | 341.9 | 20.4% | 40.3% | 0.04% | 39.3% |

TABLE 27
Formulation and Composition of Lyophilized AAVs - Fiber Fabrication of the groups according to Process A

| | | | | Lyo soln | | | | | | | | | |
|---|---|---|---|---|---|---|---|---|---|---|---|---|---|
| | | | | Su- | PEG | Lyo Volume | | | % Composition of | | | | |
| | Cryo soln | | | crose | NH2 | AuNP | Cryo | Total Lyo | Lyophilized Product | | | | |
| | PEG | Sucrose | PEG NH2 | (mg/ | (mg/ | Solution | Solution | Solution | | Su- | PBS | Pluronic | PEG |
| Group | NH2 | (mg/mL) | (mg/mL) | mL) | mL) | (μL) | (μL) | (μL) | AAV | crose | Salts | F-68 | NH2 |
| 549-059A | 4a40k | 6.1 | 16.0 | 5 | 13.2 | 60 | 283.4 | 343.4 | 0.1% | 17.8% | 35.3% | 0.04% | 46.7% |
| 549-059B | 4a40k | 16.8 | 44.5 | 5 | 13.3 | 240 | 101.9 | 341.9 | 0.5% | 17.7% | 35.1% | 0.04% | 46.7% |
| 549-059C | 4a20k | 6.1 | 11.7 | 5 | 9.7 | 60 | 283.4 | 343.4 | 0.1% | 20.4% | 40.3% | 0.04% | 39.1% |
| 549-059D | 4a20k | 16.8 | 32.6 | 5 | 9.7 | 240 | 101.9 | 341.9 | 0.6% | 20.3% | 40.1% | 0.04% | 39.1% |

2 mm AAV implants (n=2) were placed in a 1.5 mL protein LoBind tube (Eppendorf) and immersed in 1 ml of PBS+0.001% Pluronic F-68, pH=7.4, at 37° C. At each timepoint, 300 μL of PBS was removed and replaced with fresh PBS. The IV release sample was then flash frozen in liquid nitrogen and stored at −80° C. prior to analysis. AAV IV release samples were analysed via qPCR with primers to the inverted terminal repeats in the viral genome.

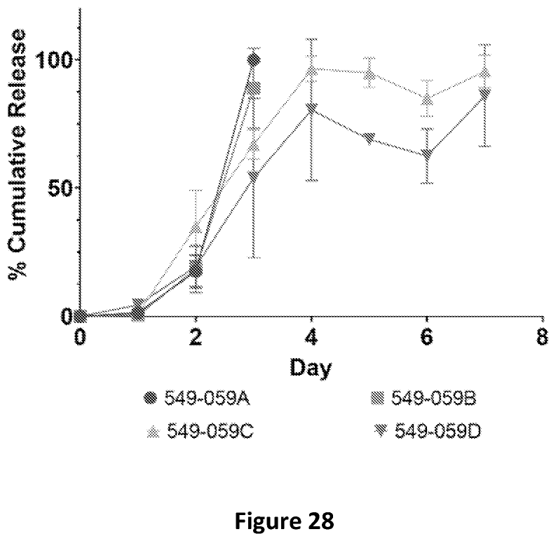

FIG. 28 shows the AAV release curves for 549-059 formulations. Two AAV doses: a 1× and 4× dose. Between 059A (1× dose) and 059B (4× dose), the % cumulative release was similar, regardless of dose. Both had full release of AAVs by day 3. Likewise, 059C and 059D had a similar release curve with full release at approximately day 4.

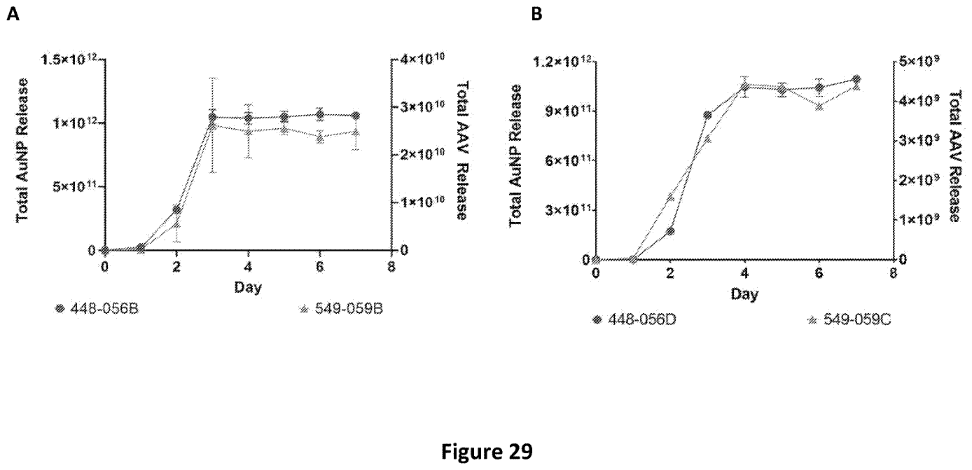

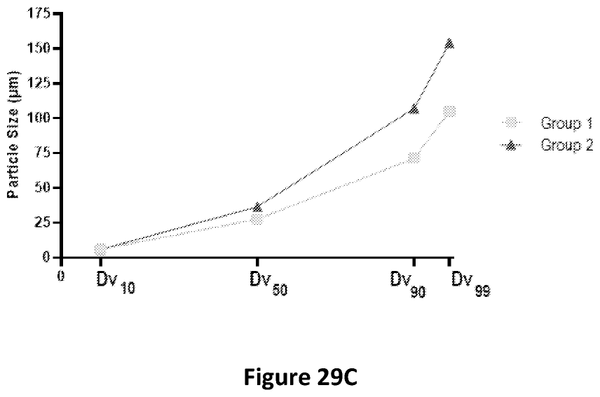

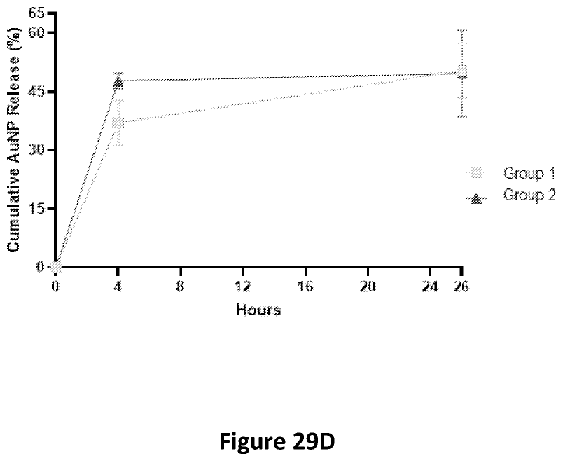

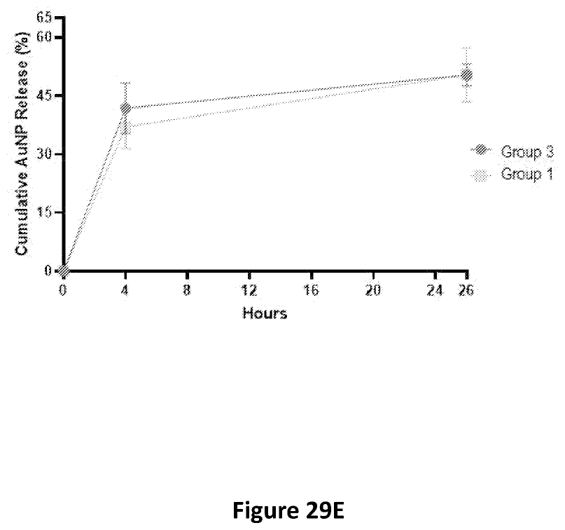

FIG. 29(A) and 29 (B) compares the total release of AuNP and AAV particles from the same formulation. It can be seen that the release profiles were comparable and that AuNP work well as a surrogate for AAV release.

### Example 3.6: Particle Size can be Selected to Control the Release Profile

To test the effect of particle size on the release profile, two implants were fabricated comprising different particle size distribution by volume (Dv₉₀) as shown in Tables 27A and 27B. The desired Dv₉₀ was achieved by using different lyophilization recipes, in particular by altering the annealing parameters (Groups 1 and 2). The lyophilization recipe with or without the annealing parameters is shown in Tables 27C and 27D, respectively.

As shown in FIG. 29(C), two different Dv₉₀ particle sizes (Groups 1 and 2) could be successfully achieved by altering the lyophilization recipe. The in vitro release profile of each of Groups 1 and 2 is shown in FIG. 29(D). As can be seen from FIG. 29(D) the higher the Dv₉₀ particle size, the higher the percentage release. It was observed that the Dv₉₀ particle size seems to specifically influence the day 1 release as evidenced by the burst release within 4 hours.

Another method of achieving the desired particle size was tested comprising the use of a microemulsifier after lyophilization. With this method Dv₉₀ particle size of 33 μm could be achieved (Group 3). However, the method of using a microemulsifer may have caused formation of bubbles because of which the trend in the in vitro release profile was not as expected (FIG. 29(E)).

TABLE 27A
Formulation of Fibres to test the effect of particle size on in vitro
release profile

| | PEG Formulation | | | | | % Composition of Dried Implant*** | | | | | | |
|---|---|---|---|---|---|---|---|---|---|---|---|---|
| | % | | PEG | PEG | Ester | | | | | Multi- | LDP: | LDP: |
| | maPEG | PEG | Ester 1 | Ester 2 | SS:SG | | PBS | Pluronic | | arm | PEG | maPEG |
| Group | in DMC* | Amine | (SS) | (SG) | Ratio | Sucrose | Salts | F-68 | AuNP | PEG | Ratio** | Ratio |
| 1 | 6% | 4a20k | 4a20k | 4a40k | 70:30 | 15.8% | 34% | 0.02% | 1.2% | 49% | 1.04 | 1.04 |
| 2 | 6% | 4a20k | 4a20k | 4a40k | 70:30 | 15.8% | 34% | 0.02% | 1.2% | 49% | 1.04 | 1.04 |

*maPEG: multi-arm PEG
**LDP:PEG ratio: Lyophilized Dry Powder:PEG mass ratio
***Linear PEG % was kept at 0.

TABLE 27B
Dv₉₀ particle size of groups 1 and 2
according to Table 27A

| | Dv₉₀ |
|---|---|
| Group | (μm) |
| 1 | 71.1 |
| 2 | 107.0 |

TABLE 27C
AuNP Lyophilization With Annealing Recipe

| | | Temp | Time | | Pressure |
|---|---|---|---|---|---|
| | Steps | (° C.) | (min) | Ramp/Hold | (mTorr) |
| Thermal Treatment | 1 | −40 | 60 | Hold | — |
| | 2 | −20 | 40 | Ramp | — |
| | 3 | −20 | 180 | Hold | — |
| | 4 | −40 | 40 | Ramp | — |
| Freeze | — | −40 | 60 | — | — |
| Primary Drying* | 1 | −38 | 90 | Hold | 100 |
| | 2 | −35 | 25 | Ramp | 100 |
| | 3 | −35 | 1000 | Hold | 100 |
| | 4 | −35 | 1000 | Hold | 100 |
| | 5 | −10 | 200 | Ramp | 100 |
| | 6 | −10 | 240 | Hold | 100 |
| | 7 | 0 | 80 | Ramp | 100 |
| | 8 | 0 | 240 | Hold | 100 |
| | 9 | 25 | 200 | Ramp | 100 |
| | 10 | 25 | 480 | Hold | 100 |

*Condenser set at −70° C.

TABLE 27D
AuNP Lyophilization Without Annealing Recipe

| | | Temp | Time | | Pressure |
|---|---|---|---|---|---|
| | Steps | (° C.) | (min) | Ramp/Hold | (mTorr) |
| Freeze | — | −40 | 60 | — | — |
| Primary Drying* | 1 | −38 | 90 | Hold | 100 |
| | 2 | −35 | 25 | Ramp | 100 |
| | 3 | −35 | 1000 | Hold | 100 |
| | 4 | −35 | 1000 | Hold | 100 |
| | 5 | −10 | 200 | Ramp | 100 |
| | 6 | −10 | 240 | Hold | 100 |
| | 7 | 0 | 80 | Ramp | 100 |
| | 8 | 0 | 240 | Hold | 100 |
| | 9 | 25 | 200 | Ramp | 100 |
| | 10 | 25 | 480 | Hold | 100 |

*Condenser set at −70° C.

### Example 4—In Vivo AAV Implants—Expression of the Gene Comprised in the AAV

The objective of this study was to evaluate transduction of AAV2-CMV-Luc delivery following a single intravitreal injection in Sprague-Dawley rats. This study consisted of four groups, with 3 animals each in Groups 1, 2, 3, and 4. On Day 0, animals in Group 1 received a single bilateral 10 μL intravitreal dose of AAV-Bolus (1× dose) and Group 2 received a single bilateral 10 UL intravitreal dose of AAV-Bolus (4× dose). Animals in Groups 3 received a single bilateral AAV-Sustained implant (1× dose), and animals in Groups 4 received a single bilateral AAV-Sustained implant (4× dose). Animals in Groups 3 and 4 received a 4-day release formulation. The summary of these groups is depicted in Table 28 below.

TABLE 28
In vivo transduction study

| | No. of | | | Dose | Release |
|---|---|---|---|---|---|
| Group | Animals | Treatment | Test Article | (vg/eye) | Rate |
| 1 | 3 | AAV-Bolus 1x Dose | 10 μL | 2.5E+09 | Intermediate |
| 2 | 3 | AAV-Bolus 4x Dose | | 1.2E+10 | Release |
| 3 | 3 | AAV-Sustained 1x Dose | Fiber Implant | 6.2E+09 | 4-day release |
| 4 | 3 | AAV-Sustained 4x Dose | | 2.5E+10 | |

IVIS imaging was performed on Day 0 (baseline), 4, 7, 10, 14, 17, 21, 24, and 28. All animals were anesthetized with inhaled isoflurane and then dosed with luciferin at 150 mg/kg via intraperitoneal (IP) injection. At 5-15 minutes post luciferin administration, all animals had IVIS imaging performed.

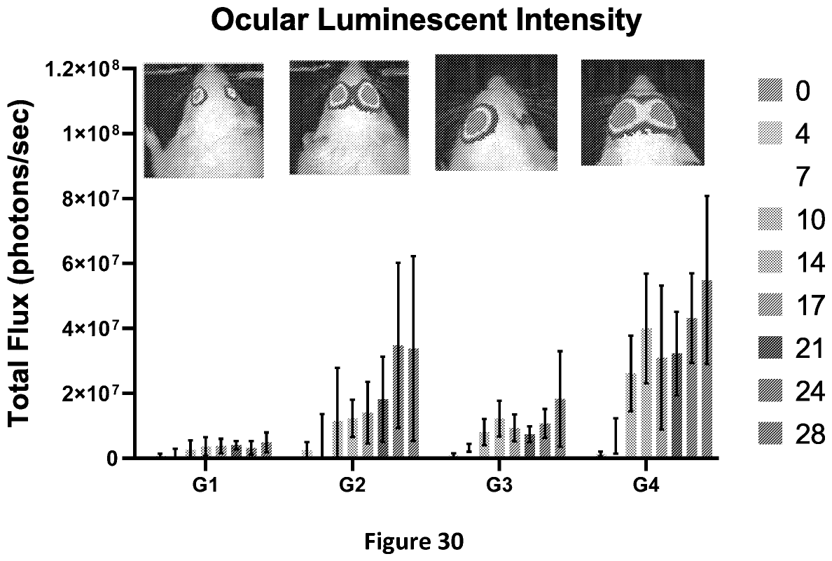

The results of the study showed that the AAV implant was placed in the intravitreal and/or subretinal space. Transduction of ocular tissues was confirmed through detection of luminescent intensity via IVIS imaging. FIG. 30 shows increasing luminescent intensity over time in all groups and a dose response was seen with greater luminescence in higher dose groups. Additionally, AAV2-CMV-Luc releasing hydrogel implants successfully transduced ocular tissues in vivo and demonstrated similar trends to the bolus controls.

### Example 5—In Vivo AAV2 Implants—GFP Expression and Inflammation Scores

In the previous Example, proof-of-concept in vivo transduction of AAV2-CMV-Luc loaded implants in rats via an intravitreal injection was demonstrated. For follow-up animal studies, rabbit models were chosen for their larger ocular dimensions.

In this example, a rabbit model was utilized to compare a single liquid injection of AAV2-CMV-GFP against an AAV2-loaded implant from an intravitreal injection. Primary outcome assessments were FAF imaging to examine GFP expression and ophthalmic examinations for inflammation scores.

Additional groups were evaluated to assess the impact of triamcinolone acetate (4 mg) or OTX-TKI (200 μg) pretreatment on GFP expression and inflammation. Triamcinolone acetate (TA) is a strong anti-inflammatory steroid that is commonly used in ophthalmic applications. OTX-TKI contains 200 ug of axinitib. Table 29 outlines the experimental design for groups and dose administration. Table 30 displays the ocular assessments and study end points.

TABLE 29
Experimental Design - Dose Administration

| | | | Prophylactic anti-inflammatory | | | Experimental Compound | | |
|---|---|---|---|---|---|---|---|---|
| | | | | | Dose | | | Dose |
| Group | No. of | Test | | Dose | Route/ | | Dose | Route/ |
| No. | Animals | Material | Concentration | Volume | Regimen | Concentration | Volume | Regimen |
| 1 | 3 | Vehicle Liquid | NA | | | 1.2 × 10¹⁰ | OD - 50 μL | Bilateral |
| | | and Vehicle | | | | gc/eye | Vehicle Liquid | IVT dose |
| | | Fiber | | | | | OS - 1 | on D0 |
| | | | | | | | Vehicle Fiber | |
| 2 | 3 | AAV-Bolus | | | | | 50 μL | |
| 3 | 3 | AAV- | | | | | 1 Fiber | |
| | | Sustained Fiber | | | | | Implant | |
| 4 | 3 | AAV-Bolus + | Triamcinolone^{a} | 100 μL | Bilateral | | 50 μL | |
| | | Triamcinolone | (4 mg/100 μL) | | IVT dose | | | |
| | | (4 mg/100 μL) | | | on D-3 | | | |
| 5 | 3 | AAV- | Triamcinolone^{a} | | | | 1 Fiber | |
| | | Sustained Fiber + | (4 mg/100 μL) | | | | Implant | |
| | | Triamcinolone | | | | | | |
| | | (4 mg/100μ L) | | | | | | |
| 6 | 3 | AAV-Bolus + | TKI^{a} (200 μg) | 1 Fiber | | | 50 μL | |
| | | TKI | Fiber | implant | | | | |
| | | (200 μg) Fiber | | | | | | |
| 7 | 3 | AAV- | TKIª (200 μg) | | | | 1 Fiber | |
| | | Sustained Fiber + | Fiber | | | | Implants^{b} | |
| | | TKI (200μ g) | | | | | | |
| | | Fiber | | | | | | |

^{a}Groups receiving triamcinolone or TKI will be dosed 3 days prior to D0
^{b}7003 OS received no TKI Fiber and 2 AAV Fiber Implants

TABLE 30
Experimental Design - Assessments and End Points

| Study | Pre- | Post- | | | | | | | |
|---|---|---|---|---|---|---|---|---|---|
| Para- | dose | dose | Day | Day | Day | Week | Week | Week | Week |
| meter | (D-3) | (D0) | 2 | 4 | 7 | 2 | 4 | 6 | 8 |
| Ophthal- | X | | X | X | X | X | X | X | X |
| mic | | | | | | | | | |
| Exam | | | | | | | | | |
| OCT | X | X | X | X | X | X | X | X | X |
| Fundus | X | X | X | X | X | X | X | X | X |
| Auto- | | | | | | | | | |
| Fluores- | | | | | | | | | |
| cent | | | | | | | | | |
| Imaging | | | | | | | | | |
| Serum | X | — | — | — | — | X | X | X | X |
| Antibody | | | | | | | | | |
| and NAb* | | | | | | | | | |
| Vector | X | — | X | X | X | X | — | — | — |
| Shedding* | | | | | | | | | |
| Ocular | — | — | — | — | — | — | — | — | X |
| Biodistri- | | | | | | | | | |
| bution | | | | | | | | | |
| (OD)* | | | | | | | | | |
| Histopath- | — | — | — | — | — | — | — | — | X |
| ology/ | | | | | | | | | |
| IHC (OS)* | | | | | | | | | |

*samples collected and held until decision was made for analysis

### Materials and Methods

### Implant Manufacture

Sterilization: All items were autoclaved or depyrogenated before use.

PEG Endotoxin Removal & AAV Lyophilization: 1) Preparation of PEG-NH2+Sucrose+AAV Syringe: To remove endotoxins, 4a20k NH2+Sucrose was dissolved in 1×PBS+0.001% Pluronic F-68 in 5 mL volumetric flask, syringe filtered through a Mustang E (Pall, MSTG25E3) filter into the BSC using a syringe pump (Harvard Apparatus, Pump 11 Elite) at 2.5 mL/min. The filtered solution was collected into a 5 ml tube.

AAV stock vials were thawed, centrifuged to collect all the solution, and brought into the BSC. AAV stock solutions were combined with a concentrated 4a20k NH2+Sucrose solution in 1.5 mL Protein LoBind tubes. Depyrogenated aluminum syringe block was removed from the −80° C. freezer and aseptically transferred into the BSC. The AAV+4a20k NH2+Sucrose solutions were loaded in 1 mL Soft-Ject syringes (Henke Sass Wolf, 8300018745) and placed in frozen aluminum block. The loaded syringe block was then double bagged into autoclaved pouches and transferred into the lyophilizer. The lyophilization composition of the groups is outlined in Table 31. The lyophilizer (1-Shelf Viritis Advantage) was prepared for use by ensuring that the entire chamber was cleaned using Kimwipes and sterile 70% isopropanol. The shelf was frozen to a target temperature of −40° C. prior to starting the process.

PEG Endotoxin Removal & AAV Lyophilization: 2) Preparation of PEG Ester Syringe: 8k linear PEG, 4a20k SS, and 4a40k SG solutions were made in cold WFI, pH=3.8 in 5 mL volumetric flasks and kept on ice to increase the PEG ester pot life. PEG esters were combined in appropriate volumetric ratios into a 5 ml tube. Combined PEG ester solution was syringe filtered with a Mustang E filter into the BSC using a syringe pump (Harvard Apparatus, Pump 11 Elite) at 2.5 mL/min. The filtered solution was collected into a 5 mL tube. Depyrogenated aluminum syringe block was removed from the −80° C. freezer and aseptically transferred into the BSC. The PEG esters solutions were loaded in 1 mL Soft-Ject syringes (Henke Sass Wolf, 8300018745) and placed in frozen aluminum block. The loaded syringe block was then double bagged into autoclaved pouches and transferred into the lyophilizer. After loading both AAV+PEG-NH2+Sucrose and PEG ester syringes, the lyophilization recipe was then confirmed to have the correct parameters (Table 32) and the cycle was started.

TABLE 31
Formulation and Composition of Lyophilized AAVs

| | | | | | Lyo Volume | | | | | | | | |
|---|---|---|---|---|---|---|---|---|---|---|---|---|---|
| | Cryo Solution | | Lyo Solution | | | | Total | | | | | | |
| | Su- | PEG- | Su- | PEG- | AAV | Cryo | Lyo | | | | | | |
| | crose | NH2 | crose | NH2 | Solu- | Solu- | Solu- | % Lyophilization Composition | | | | | |
| | (mg/ | (mg/ | (mg/ | (mg/ | tion | tion | tion | | | Su- | | Plur- | PEG- |
| Group | mL) | mL) | mL) | mL) | (μL) | (μL) | (μL) | AAV | IDX* | crose | PBS | onic | NH2 |
| Placebo** | 7.8 | 14.1 | 5 | 9.0 | 145 | 256 | 401 | 0% | 0% | 20.9% | 41.3% | 0.04% | 37.7% |
| Implant 1 | 5.3 | 8.5 | 5 | 8.0 | 28 | 440 | 468 | 0.2% | 11.1% | 19.4% | 38.3% | 0.06% | 31.1% |
| Implant 2 | 5.7 | 10.3 | 5 | 9.0 | 52 | 360 | 412 | 0.3% | 20.2% | 16.7% | 32.8% | 0.07% | 30% |

*Iodixanol in the AAV stock from the manufacturer
**The placebo group has no AAV and used PBS + 0.001% Pluronic F68 as a surrogate.

TABLE 32
Lyophilization Recipe for AAV-PEG NH2 and PEG Ester syringes

| | | Temp | Time | | Pressure |
|---|---|---|---|---|---|
| | Steps | (° C.) | (min) | Ramp/Hold | (mTorr) |
| Thermal | — | — | — | — | — |
| Treatment | | | | | |
| Freeze | — | −40 | 60 | — | — |
| Primary | 1 | −38 | 90 | Hold | 100 |
| Drying* | 2 | −35 | 25 | Ramp | 100 |
| | 3 | −35 | 1000 | Hold | 100 |
| | 4 | −35 | 1000 | Hold | 100 |
| | 5 | −10 | 200 | Ramp | 100 |
| | 6 | −10 | 240 | Hold | 100 |
| | 7 | 0 | 80 | Ramp | 100 |
| | 8 | 0 | 240 | Hold | 100 |
| | 9 | 25 | 200 | Ramp | 100 |
| | 10 | 25 | 480 | Hold | 100 |

*Condenser set at −70° C.

Fiber Casting: This section describes the methodology used for fabricating the strands loaded with the AAVs. The previously lyophilized AAV+PEG-NH2+Sucrose and PEG esters were removed from the lyophilizer and aseptically transferred into the BSC.

PEGs were reconstituted with sterile filtered, anhydrous DMC in the syringes. The PEG formulation for each group is listed in Table 33.

TABLE 33
PEG Formulation and Volumes for each cast

| | | | | | | | | | | PEG Volumes | |
|---|---|---|---|---|---|---|---|---|---|---|---|
| | PEG Formulation | | | | | | Ester Blend | | | for Casting | |
| | | | | PEG | PEG | | Volumetric Ratio | | | | PEG |
| | | % | | Ester | Ester | Ester | | | 8k | PEG | Ester |
| | % | Linear | PEG | 1 | 2 | SS:SG | 4a20k | 4a40k | Linear | NH2 | Blend |
| Group | maPEG | PEG | Amine | (SS) | (SG) | Ratio | SS | SG | PEG | (μL) | (μL) |
| Placebo | 5% | 1% | 4a20k | 4a20k | 4a40k | 87.5:12.5 | 3.5 | 1 | 1.7 | 53.0 | 82.1 |
| Implant | 5% | 3% | 4a20k | 4a20k | 4a40k | 87.5:12.5 | 3.5 | 1 | 5.1 | 40.2 | 96.5 |
| 1 | | | | | | | | | | | |
| Implant | 5% | 3% | 4a20k | 4a20k | 4a40k | 87.5:12.5 | 3.5 | 1 | 5.1 | 39.9 | 95.7 |
| 2 | | | | | | | | | | | |

7.

The necessary amount of DMC was combined with the lyophilized AAV+PEG-NH2+Sucrose and PEG ester powders. First, a plunger was reinstalled onto the lyophilized 1 mL HSW syringes, and DMC was loaded using a pipette, dispensing the solution through the narrow front end of the syringe while pulling the plunger down. The syringe was then degassed three times by covering the open end of the syringe while pulling the plunger down. The mixed solution was then carefully pushed to the syringe tip and capped with a female luer cap. The syringe sat at ambient lab conditions for 30 min to allow the PEGs to dissolve.

Casting the AAV/Placebo strands required the use of LDPE tubing of an inner diameter of 0.559 mm and a wall thickness of 0.127 mm cut into 45 cm long segments, micro size paper binder clips, luer connectors, and 22G blunt needles. The tubing was prepared by carefully inserting the blunt 22G needle into one end of the tubing. One clip was placed towards the distal end of the tube and the other was placed towards the proximal end.

The PEG ester and PEG amine syringes were mated with a luer connector and then mixed for 30 seconds. Before removing the luer connector, the syringe was degassed by pulling the plunger down to roughly the 0.3 mL mark to degas and the syringe was flicked to remove any remaining air bubbles. The luer connecter was then removed and the solution was pushed towards the tip of the syringe.

The syringe was then attached to the tube using the inserted 22G blunt needle and the plunger was slowly pushed to cast the solution into the tube. The distal end of the tubing was clipped once the necessary amount of solution was dispensed. The tube was then slightly pressurized, and the proximal end was then clipped. The remaining solution in the syringe was then used to record the gel time, listed below in Table 34.

TABLE 34
Gel Times for Casted Strands

| Fiber | | Gel Time |
|---|---|---|
| Formulation | Lot # | (min:sec) |
| Placebo | MP-553-27C | 3:48 |
| Implant 1 | MP-553-21B | 3:30 |
| Implant 2 | MP-533-27B | 3:09 |

The casted tubes were then placed into a glass curing chamber in the presence of DMC vapor for 2 hours. The DMC vapor was present in the chamber to prevent premature drying of the casted gel prior to full curing. After curing, the tubing/strand was transferred outside of the BSC and into a 37° C. oven with a nitrogen gas flow set to 30 SCFH to dry for 3 days. Final composition of the dried AAV/Placebo implants are listed in Table 35.

TABLE 35
Theoretical % Mass Compositions of Dried Implants

| | | | | PBS | Pluronic | Linea | | LDP:PEG | LDP:maPEG |
|---|---|---|---|---|---|---|---|---|---|
| Group | AAV | IDX* | Sucrose | Salts | F-68 | r PEG | maPEG** | Ratio*** | ratio |
| Placebo | 0% | 0% | 13.2% | 26.1% | 0.03% | 10.1% | 50.6% | 1.0 | 1.2 |
| Implant | 0.1% | 5.4% | 9.4% | 18.6% | 0.03% | 19.3% | 47.2% | 0.5 | 0.7 |
| 1 | | | | | | | | | |
| Implant | 0.1% | 10.0% | 8.3% | 16.2% | 0.04% | 18.9% | 46.4% | 0.5 | 0.7 |
| 2 | | | | | | | | | |

*Iodixanol in the AAV stock from the manufacturer
**maPEG = multi-arm PEG
***LDP:PEG Ratio = Lyophilized Product:PEG mass ratio

Fiber Cutting: The previously casted and dried fibers were removed from the 37° C. oven and aseptically transferred into the BSC. Working with one casted strand at a time, the casted tubing/strand was placed onto the depyrogenated stainless steel cutting apparatus. Microtome blades were used to cut the strands into 4 mm fibers. To prevent dull fiber cuts, the blades were replaced after cutting five times in the beginning, middle, and end areas of the blade for a total of fifteen cuts per blade. After cutting, the fibers were kept in the tubing and transferred into the appropriate LoBind tubes designated for PCR, Bioburden, Endotoxin, IV release, and needle-loading.

Using an 18G needle, four holes were punched into the top of the LoBind tubes containing the cut fibers. The LoBind tubes were then double pouched in autoclaved pouches and transferred into a nitrogen glove box for conditioning for a minimum of 3 days.

Needle Loading and Packaging: The cut fibers were removed from the glove box and aseptically passed inside of the BSC, along with the forceps and 2″ nitinol wires. The 27G 1.25″ needle (NIPRO Hypodermic Needle, 27Gx1.25″, No. AH+2732) were transferred into the BSC one at a time and visually inspected for any damage to the tip of the needle. An implant was then grabbed with forceps and loaded into the needle through the tip.

Once the fiber was loaded into the needle a 2 cm nitinol wire was inserted into the needle tip and a polypropylene hub cap was inserted into the needle hub thus securing the fiber in place for further handling. This assembly was then placed into a small, autoclaved Tyvek pouch and sealed. Once all needles were loaded and sealed in the autoclaved pouches, they were transferred outside of the BSC.

Outside of the BSC, the autoclaved pouches containing the loaded needles were placed into an aluminum pouch (Oliver Healthcare Packaging, Part No. 90-2003-002 REV C), labeled, and transferred into the glove box for conditioning for 3 days. After conditioning the aluminum pouches were sealed using a pouch sealer and the completed assemblies were sterile and ready for use. In addition to AAV implants, 200 ug TKI implants (JI-525-076) were prepared following the same procedure.

### Implant Characterization

Bioburden and Endotoxin Testing: To prepare endotoxin and bioburden samples, 4× implants were placed into a depyrogenated 10 mL serum vial. The groups used to assess manufacturing process were Placebo and Implant 1. For endotoxin samples, vials were crimp sealed after adding the fibers and evaluated using a kinetic turbidimetric method. For bioburden samples, 1 mL of 1×PBS+0.001% Pluronic F-68, pH=8.4) was added to the implants and the vial was crimp sealed. The vials were then transferred into a 37° C. incubator and placed onto a shaker plate for vigorous shaking. After full degradation, the samples were removed from the incubator and kept at room temperature until ready to analyse for aerobic, anaerobic, yeast/mold and heat shock bioburden.

Dimensional Analysis: AuNP fibers with the same formulation were prepared for dimensional analysis. 4 mm fiber implants were cut from proximal, mid, and distal sections of two strands (total=6 fibers). Dry dimensions (length and diameter) were measured via the Starrett Vision Systems. 1× fiber placed in a 1.5 mL Eppendorf Lo-protein binding tube (VWR Cat #80077-232). 1 mL of buffer solution (1×PBS+0.001% Pluronic F-68 at pH=7.2) was added into each tube. Samples were kept at 37° C. Swollen dimensions were measured daily via Starrett Vision Systems.

Dose Measurement: qPCR Titer & ELISA: 4 mm AAV fiber implants were cut from the strand and placed in an autoclaved 1.5 mL Eppendorf Protein LoBind tube (VWR Cat #80077-232) each. 1 mL of sterile filtered buffer solution (1×PBS+0.001% Pluronic F-68 at pH=8.4) were added into each tube. Each sample was then placed in a 37° C. oven under vigorous shaking for two days. Upon full fiber degradation at 37° C., each sample was pipet mixed and aliquoted into four 0.5 mL Protein LoBind tubes (VWR Cat #8007-228) at 250 μL each. Samples were flash frozen in liquid nitrogen and stored at −80° C. Titer was determined via qPCR. ELISA samples were tested using an AAV2 Xpress ELISA kits (Progen Cat #PRAAV2XP).

IV Release Study-ELISA Analysis: 4 mm AAV fiber implants were cut from the strand and placed in a 1.5 mL Eppendorf Lo-protein binding tube (VWR Cat #80077-232). 1 mL of buffer solution (1×PBS+0.001% Pluronic F-68 at pH=7.2) added into each tube. 3 samples were prepared for each group (total=3 fiber implants per group). Samples were kept at 37° C. At each timepoint, 250 μL of PBS was removed and replaced with fresh PBS. The IV release sample was then flash frozen in liquid nitrogen and stored at −80° C. prior to ELISA analysis. AAV2 Xpress ELISA kits (Progen Cat #PRAAV2XP) were used to measure IV release. % Cumulative release was calculated by each sample's maximally released dose.

In Vivo Study Design An 8-week inflammation and GFP expression study of AAV2 was conducted in New Zealand White rabbits. Biofluids and ocular tissues were collected for vector shedding, ocular biodistribution, and anti-AAV2 IgG bioanalytical analysis.

### Results

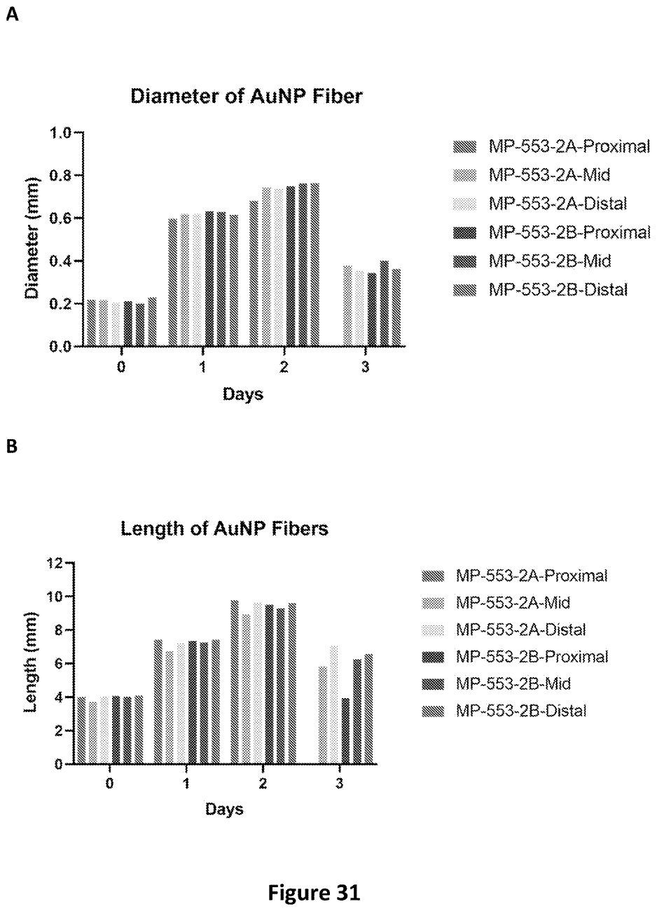

Dimensional Analysis: Since AAV fiber implants became transparent when they are immersed in water, AuNP fibers were used to measure dry and swollen dimensions. AuNP fibers were prepared with the same formulations. FIG. 31A shows the change in diameter over time and FIG. 31B shows the change in length over time. Fibers showed high degree of swelling with longer length and thicker diameter on Day 1 & 2. They were swelling for two days and then started to shrink on Day 3 prior to full degradation. There was not any difference observed in swelling among different sections (proximal, mid, and distal), showing uniform fiber fabrication.

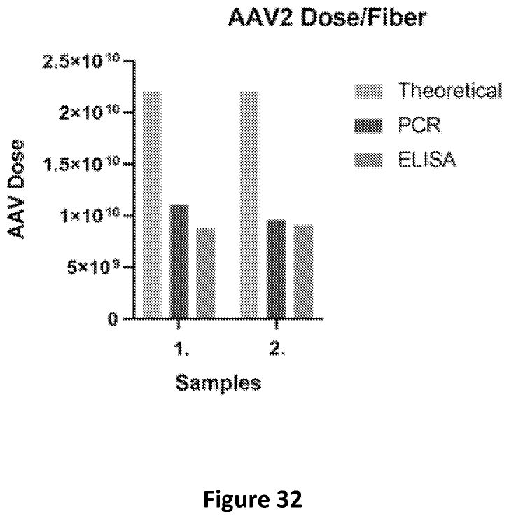

Dose Measurement: qPCR Titer &ELISA: FIG. 32 shows the PCR and ELISA analysis results of AAV2 samples. The PCR and ELISA results were comparable; however they were lower than the theoretical value (2.2 e10/implant). Any of the PCR or ELISA results can be taken to determine the actual dose. In this case, the averaged PCR results were used to determine the AAV2 liquid dose at 1.2E±10 GC/eye.

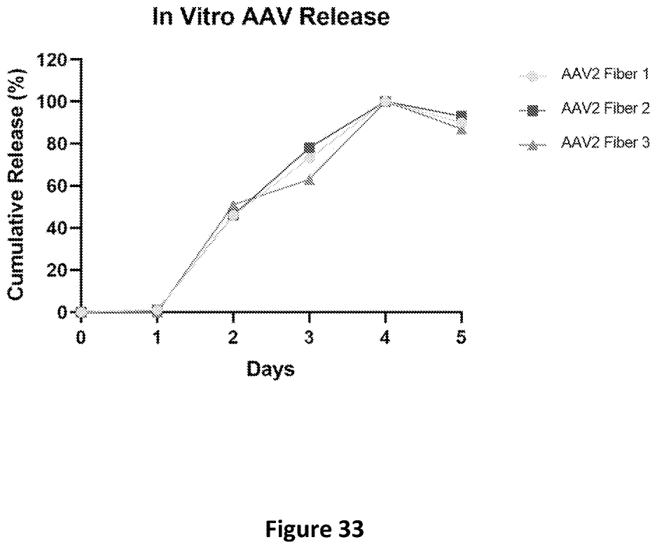

In Vitro AAV Release-ELISA: FIG. 33 shows the IV release curves of AAV2 implant samples. AAV release rate accelerated significantly after day 1 and reached 100% on Day 4. IV release profiles from 3 fibers were comparable, showing uniform implant fabrication.

A summary of AAV Implant characterization, including bioburden and endotoxin results, are shown in Table 36.

TABLE 36
Summary of AAV Implant Characterization

| Characterization | Test | Sample | Specification | Result |
|---|---|---|---|---|
| Dry Dimension | Starrett | AuNP Fiber | Diameter: <0.25 mm | Diameter: 0.2 mm |
| | Measurements | | Length: 3.8-4.2 mm | Length: 4.0 mm |
| Max Swelling | Starrett | AuNP Fiber | Max Diameter: | Day 2 Diameter: |
| | Measurements | | N/A | 0.7 mm |
| | | | Max Length: | Day 2 Length: |
| | | | <14 mm | 9.5 mm |
| Degradation | Visual | AuNP Fiber | 4-7 days | 4-7 days |
| | Observation | | | |
| Assay | qPCR | AAV | 2.2 × 10¹⁰ GC | 1.2 × 10¹⁰ GC |
| | | Implant | | |
| Bioburden | Aerobic, | Placebo | — | Placebo: NOC |
| | Anaerobic, | Implant | | AAV: NOC |
| | Yeast/Mold | AAV | | |
| | and Heat | Implant | | |
| | Shock | | | |
| Endotoxin | Kinetic | Placebo | — | Placebo: <0.005 |
| | Turbidimetric | Fiber | | EU/fiber |
| | | AAV Fiber | | AAV: <0.0025 |
| | | | | EU/fiber |

Ocular Examinations and Inflammation Scores: Full ocular evaluations were performed at pre-dose, Day 2, 4, 7, and Weeks 2, 4, 6, and 8 post AAV injection. The SPOT scoring system was used for ocular evaluations. Overall, all groups experienced none to minimal inflammation (average score of ≤1).

Peak inflammation for G2 (AAV2 liquid) was seen in the anterior chamber at week 4 and in the posterior chamber at weeks 4-6.

The incorporation of TA (G4 and G5) effectively eliminated inflammation through the course of the 8-week study. This is a proof-of-concept that inflammation is the major bottle-neck in AAV gene therapy.

Interestingly, G3 (AAV2 implant) experienced on average none to trace inflammation at all timepoints. This means that implants manufactured according to the present invention are able to control inflammation as compared to naked AAV2 delivery

In a comparison of AAV2 Liquid (G2) and AAV2 Liquid+TKI (G6), TKI pretreatment reduced inflammation scores.

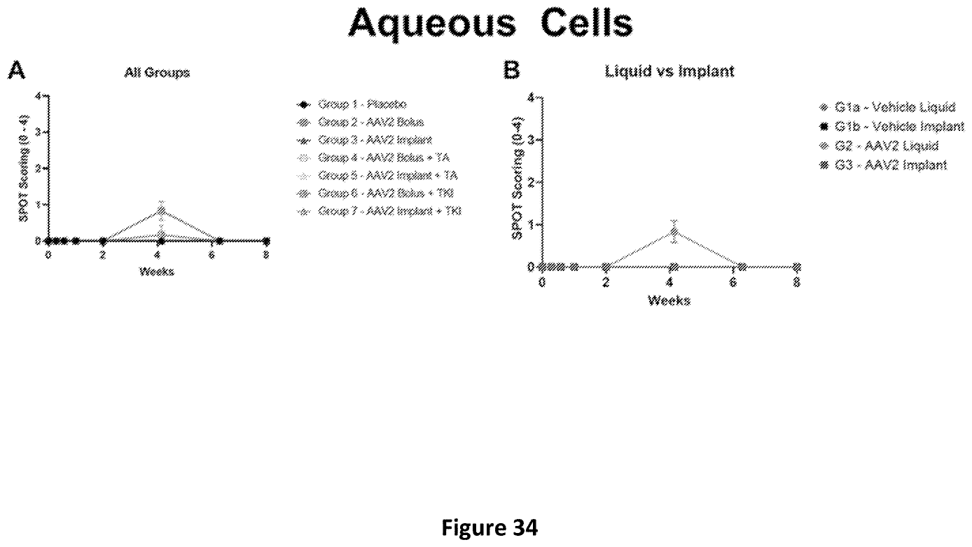

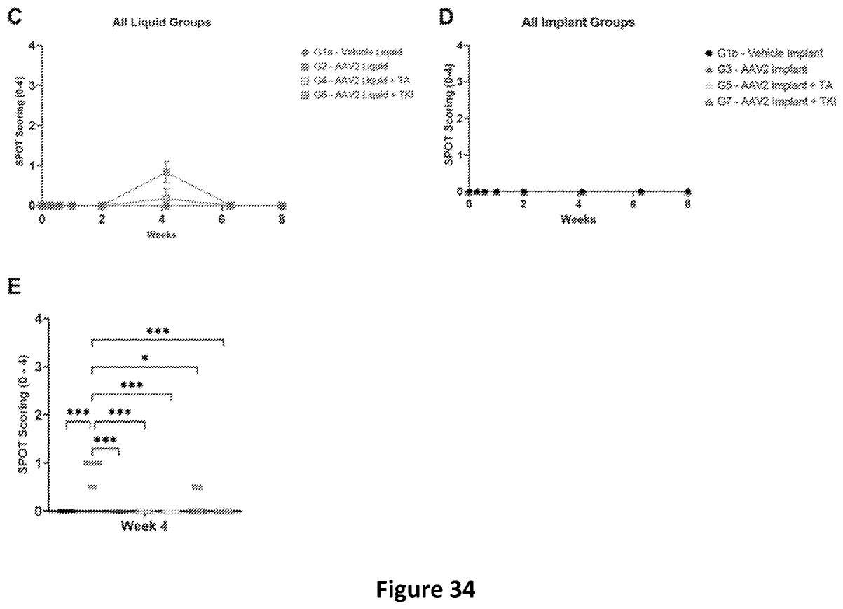

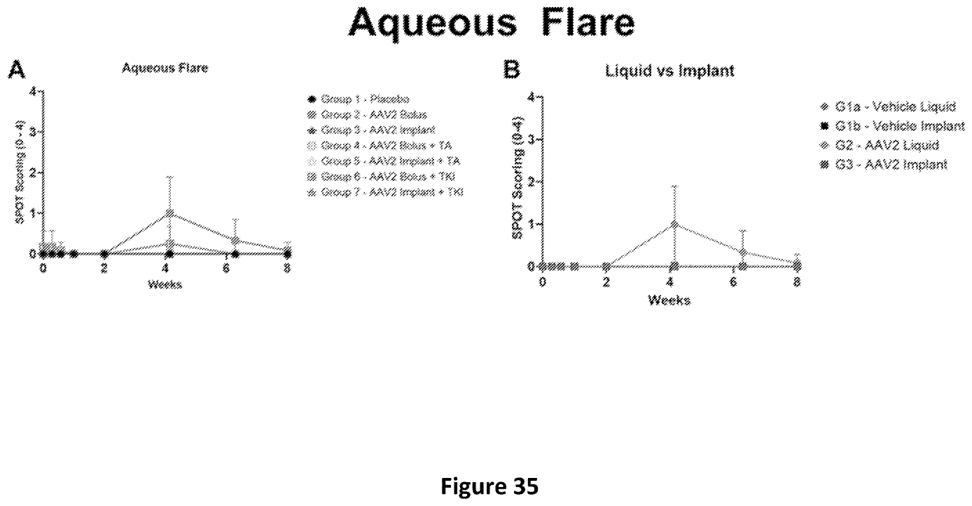

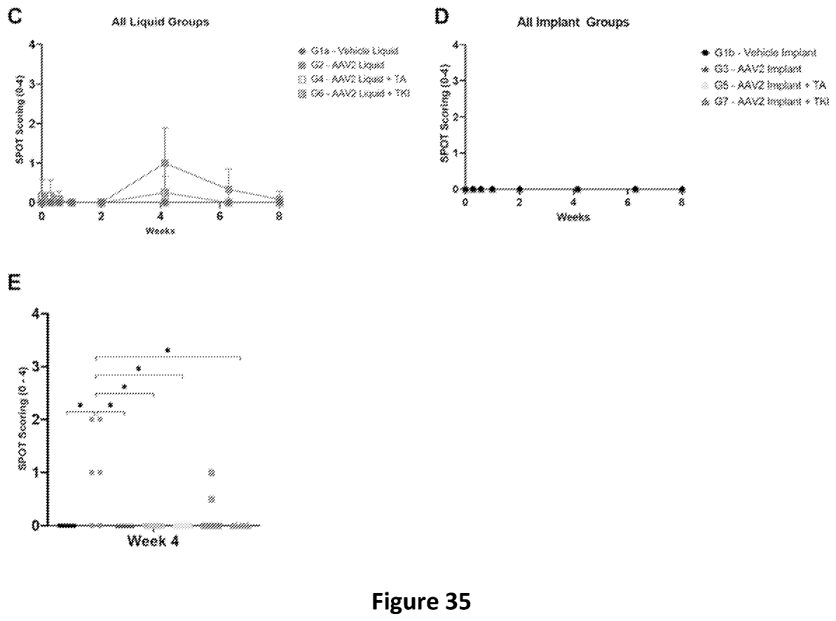

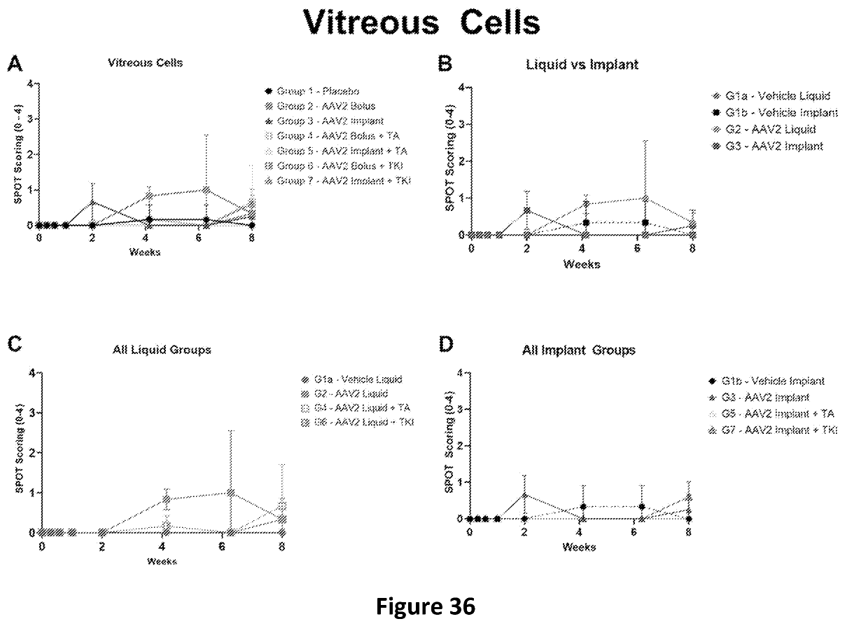

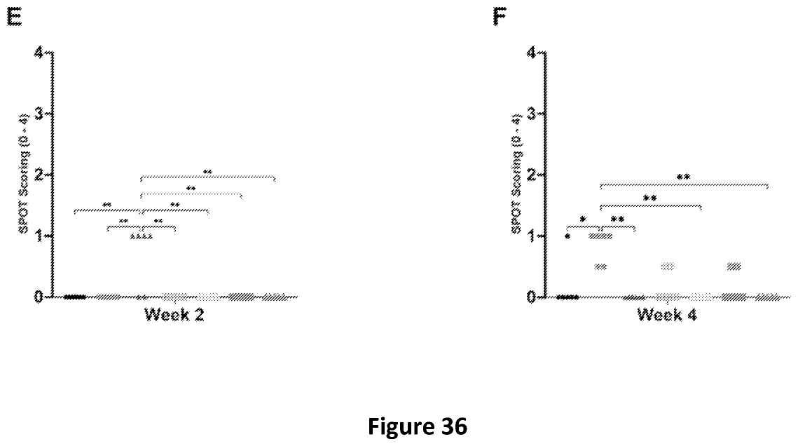

FIG. 34, FIG. 35, FIG. 36 show the Aqueous Cells, Aqueous Flare, Vitreous Cells scores for all groups, respectively. (A) incorporates all groups, (B) compares AAV2 liquid and AAV2 implant groups with no TA or TKI pretreatment, (C) and (D) highlights the impact of TA or TKI pretreatment, and (E-G) show interleaved scatter plot(s) comparing groups at weeks 2, 4, 6, or 8 with statistically different scores. * represents p<0.05, ** represents p<0.01, and *** represents p<0.001.

FIG. 34 highlights the Aqueous Cells scores. All implant groups have no aqueous cells through the 8-week study (D). The AAV2 liquid group peaked at week 4 with a score of 1 (B) and in a comparison of TA and TKI pretreatment, both compounds had a statistically significant reduction in aqueous cell scores compared to AAV2 liquid alone at week 4 (E).

FIG. 35 highlights the Aqueous Flare scores. Similar to aqueous cell scores, all implant groups have no aqueous flare through the 8-week study (D). The AAV2 liquid group peaked at week 4 with a score of 1 and reduced over the following 4 weeks (B). In a comparison of TA and TKI pretreatment for liquid groups (C), there was a reduction in aqueous flare compared to no pretreatment with statistical significance against the TA addition at week 4 (E).

FIG. 36 highlights the Vitreous Cells scores. The AAV2 implant group had a score of 1 in 2/3 rabbits at week 2, leading to a statistical difference from all other groups (E). Further investigation is needed to determine why. The AAV2 liquid groups experienced mild levels of vitreous cells from weeks 4-6 whereas all other groups had none-trace levels (A). At week 4, there was a statistically greater vitreous cell score for the AAV2 liquid group compared to all implant groups.

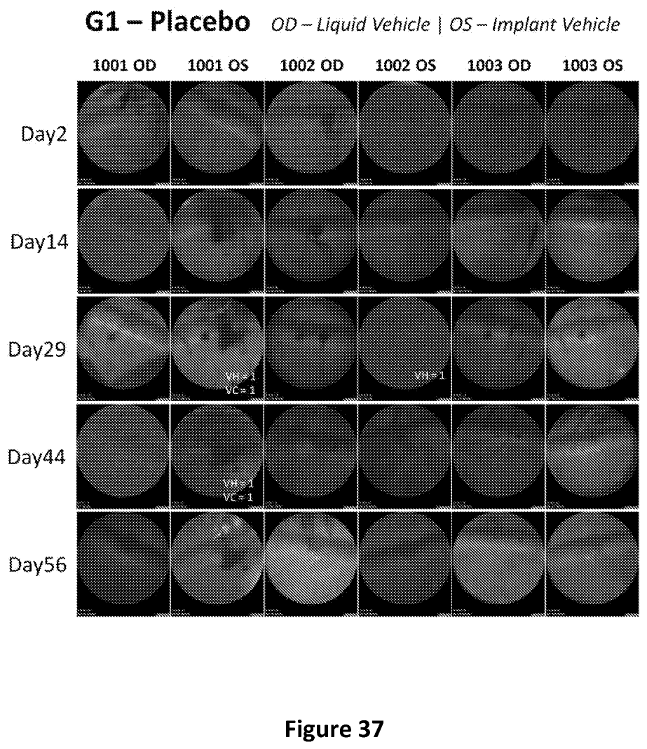

Fundus Autofluorescent Imaging: FAF imaging was performed at pre-dose, immediately post-dose, Day 2, 4, 7, and Weeks 2, 4, 6, and 8. Due to a technical issue with the Heidelberg Spectralis software, Day 4 and 7 images were unavailable. All AAV liquid and implant groups had signs of GFP expression over the course of 8 weeks. As expected, the Placebo group (G1) did not exhibit any GFP expression (FIG. 37). However, the change in GFP intensity from FAF imaging correlated with the presence of inflammation. Again, confirming that inflammation is that major bottle-neck in AAV mediated gene therapy.

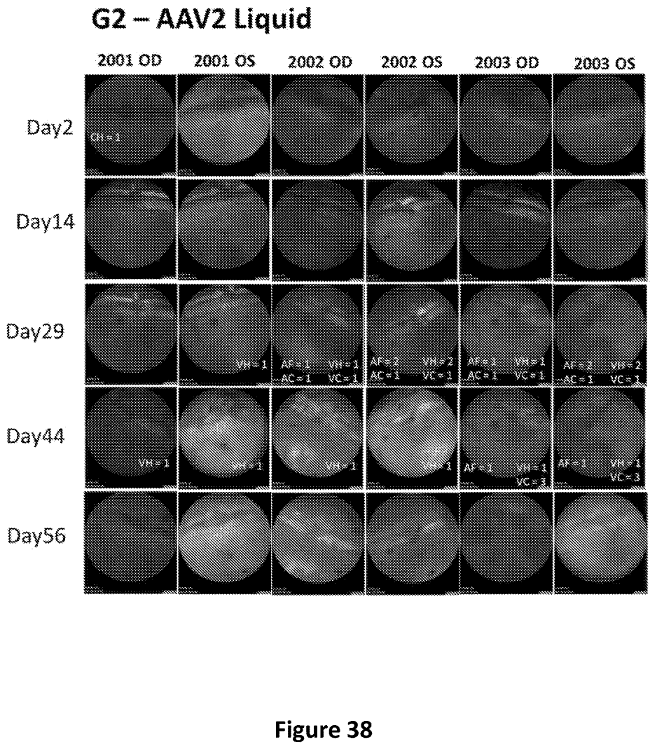

In G2 (AAV2 Liquid), GFP expression was seen in all eyes starting at D14 (FIG. 38). However, GFP expression was reduced by D29 and was difficult to visualize at D44 and D56. In examination of inflammation scores, the timing of expression reduction matches with the onset and peak of inflammation at D29.

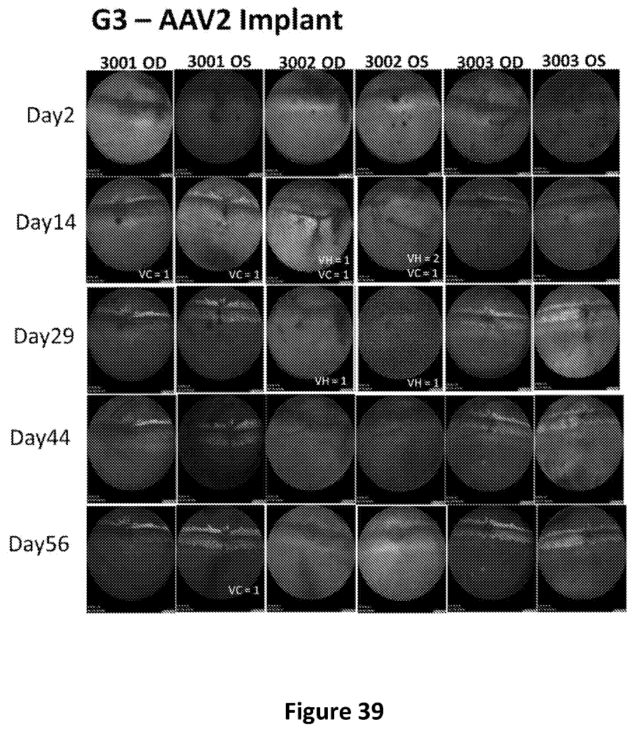

In G3 (AAV2 Implant), expression also initiated at D14 in 4/6 eyes (3001 and 3003 OU) and sustained or grew stronger at each following timepoint (FIG. 39). These eyes had none-minimal inflammation. One rabbit, 3002, did not exhibit GFP expression in either eye and had a mild inflammatory response from D14-D29. In comparison, rabbits 3001 and 3003 had sustained GFP expression with no inflammation.

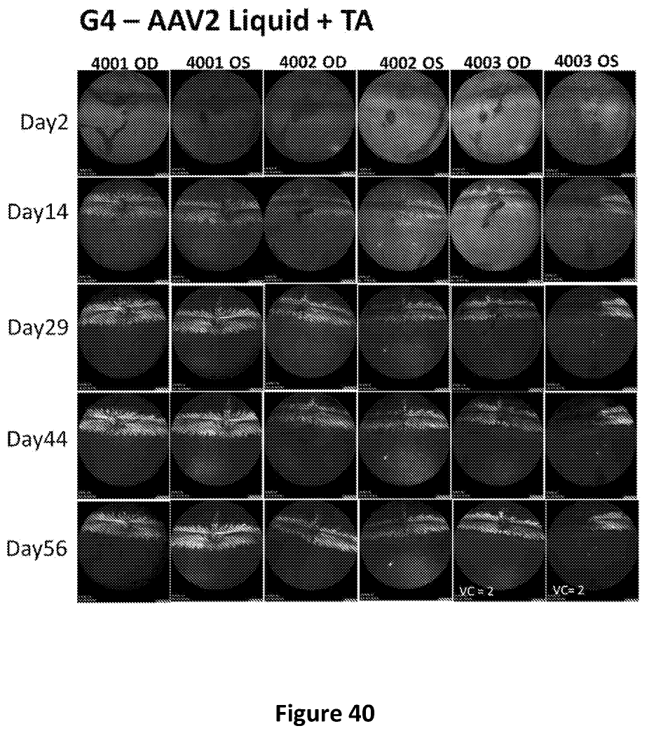

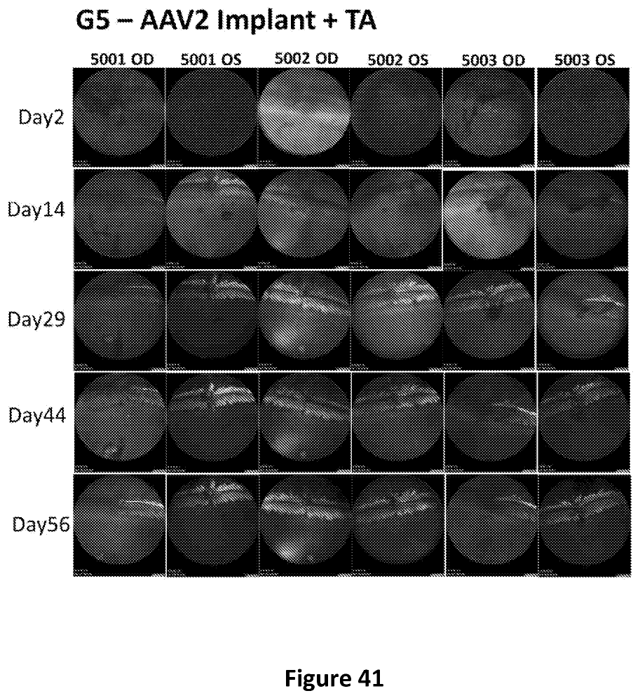

G4 (AAV2 Liquid+TA) and G5 (AAV2 Implant+TA) both had GFP expression in all eyes that sustained/grew in intensity over the course of the study (FIG. 40 and FIG. 41). Additionally, no inflammation was seen in these groups. These results highlight the use of steroids to treat inflammation and maintain transgene expression in ocular gene therapy studies. However, since steroids are known to be harmful in the long term, no means and methods are necessary for AAV mediated gene therapy without the use of steroids.

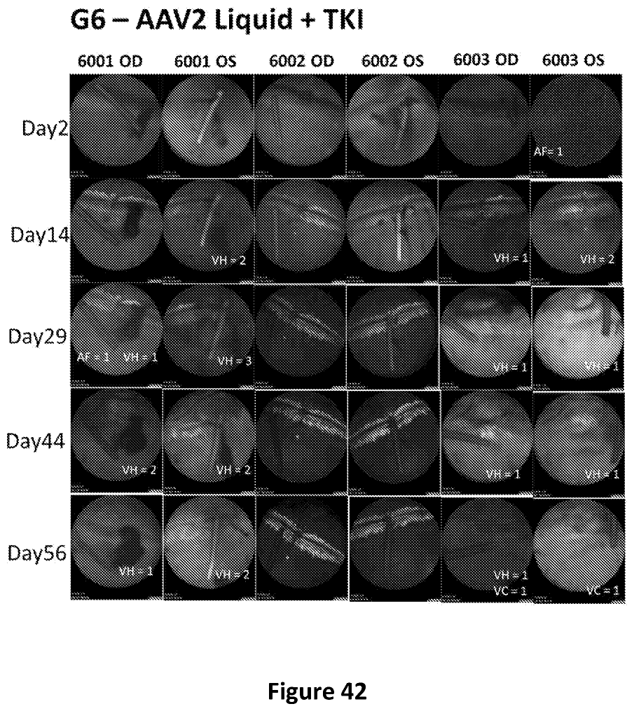

G6 (AAV2 Liquid+TKI) demonstrated a similar response to G2 for 2/3 rabbits (FIG. 42). Expression was seen at D14 in 6001 and 6003 and started to decrease at D29. This correlates with the observation of vitreous haze beginning at D14 and continuing through the end of the study in these eyes. In contrast, 6002 had strong GFP expression from D14-D56 and did not display any inflammation.

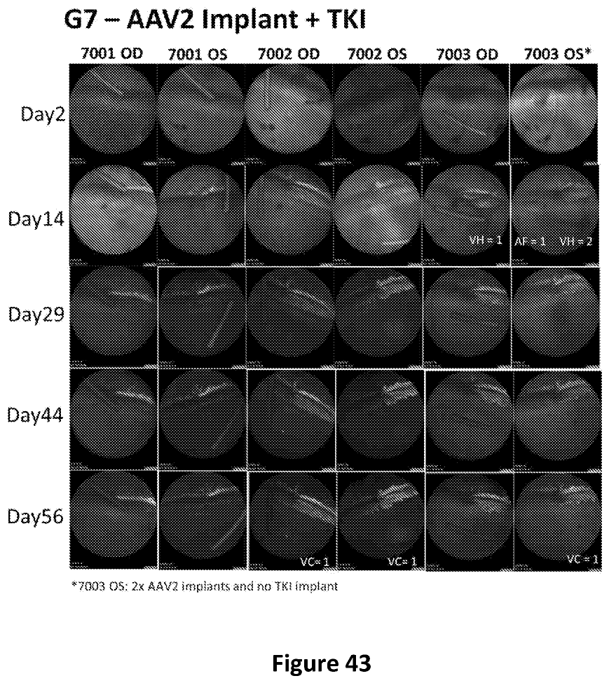

G7 (AAV2 Implant+TKI) also had sustained expression of GFP from D14-D56 in all three rabbits (FIG. 43). No inflammation was seen in the G7.

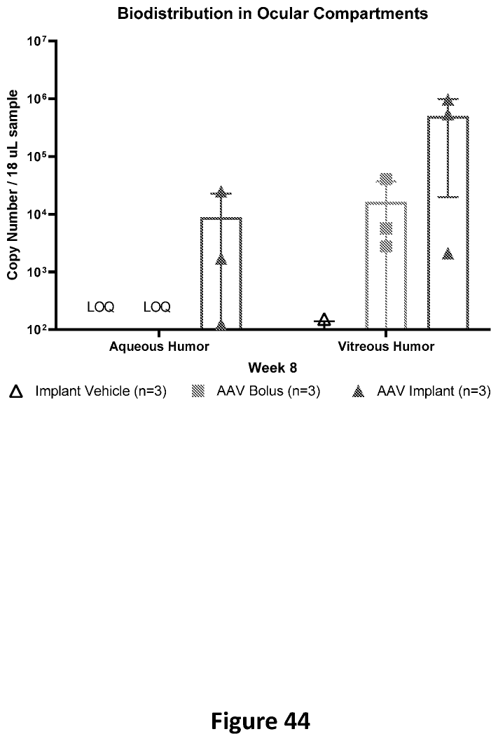

Ocular Biodistribution (OD): FIG. 44 demonstrates AAV concentrations (copy number/18 μL sample) in the eye at the end of the study (Week 8). These data demonstrate that intravitreal AAV implants are sustaining the release of AAV locally, and greater copy numbers are observed from the AAV implant group as compared to the AAV bolus group. The aqueous humor was selected for analysis because AAV vector will clear from the vitreous humor into the aqueous humor.

In the current study, a rabbit model was utilized to compare a single liquid injection of AAV2-CMV-GFP against an AAV2-loaded implant from an intravitreal injection. Primary outcome assessments were FAF imaging to examine GFP expression and ophthalmic examinations for inflammation scores. Additional groups were evaluated to assess the impact of triamcinolone acetate (4 mg) or OTX-TKI (200 ug) pretreatment on GFP expression and inflammation.

In the rabbit study, all groups experienced none to minimal inflammation (averaged score of ≤1). Although GFP expression was seen at week 2-4 in G2 (AAV2 liquid), a potential drop in expression in FAF images correlated with peak inflammation scores at weeks 4-6. In contrast, GFP expression was steady or grew stronger in G3 (AAV2 implant) from weeks 2-8 and experienced averaged none to trace inflammation at all timepoints.

This relationship between inflammation and GFP expression was evident in G4 and G5 where TA, a powerful anti-inflammatory steroid, was used. In these two groups, no inflammation was seen and visual GFP expression was the strongest among all groups.

In the TKI group, inflammation scores were overall lower and showed clear GFP expression. There is an indication that the combination of the AAV implant and TKI also helps to control inflammation.

### Example 6—In Vivo AAV2.7m8 Implants—GFP Expression and Inflammation Scores

In the first rabbit study in the previous example, low dose AAV2-CMV-GFP loaded implants were prepared and delivered intravitreally into rabbit vitreous. The impact of sustained AAV delivery on inflammation and GFP expression was investigated following IVT administration in rabbits. The study results showed that AAV loaded hydrogels had relatively lower inflammation scores and more consistent GFP expression compared to AAV2 bolus group throughout the study (8 weeks). Implants used in the first rabbit study had a lower AAV dose and a formulation that could release AAVs with near zero-order kinetics within 4 days. In the current study, higher dose AAV loaded implants were prepared with two different release formulations to explore the effect of release kinetics and AAV dose on ocular inflammation and GFP expression in rabbits. Additionally, a modified AAV2 vector-AAV2.7m8 and AAV8 were used to assess the versatility of our implants with different serotypes and/or variants. Table 37 outlines the experimental design for groups and study assessments.

### Materials and Methods

### Implant Manufacturing

Sterilization: All items were autoclaved or depyrogenated.

PEG Endotoxin Removal: 4a20k NH2+Fluorescein Sodium, 8k Linear PEG, 4a20k SS, and 4a40k SG were dissolved in aqueous medium, syringe filtered through Mustang E (Pall, MSTG25E3) filters to remove endotoxins, and lyophilized to remove water. Briefly, a 50 mg/mL 4a20k NH2+Fluorescein Sodium and 8k Linear PEG were each dissolved in WFI in 10 mL volumetric flasks. A 20:1 molar ratio of 4a20k NH2: Fluorescein Sodium was used. 50 mg/mL 4a20k SS and 4a40k SG solutions were made in cold WFI, pH=3.8 in 10 mL volumetric flasks and kept on ice to increase the PEG ester pot life. All four solutions were syringe filtered with Mustang E filters into a BSC using a syringe pump (Harvard Apparatus, Pump 11 Elite) at 2.5 ml/min. The filtered solutions were then transferred in aliquots of 3 mL into their respectively labeled 10 mL Wheaton glass vials. Depyrogenated aluminum heating blocks were removed from the −80° C. freezer and aseptically transferred into the BSC. The 10 mL Wheaton glass vials were then loaded into the aluminum heating blocks and the rubber stopper was set to the raised position, ensuring that the vial was still open and accessible for sublimation. The aluminum block containing the glass vials with PEG solutions and rubber stoppers was then double bagged with two autoclaved 7.5″×13″ autoclaved pouche

TABLE 37
Rabbit GFP Expression and Inflammation Study Outline

| | | | | | | Dose | | |
|---|---|---|---|---|---|---|---|---|
| | | Test Material | | Target | | Route/ | | |
| | No. | Right Eye | Left Eye | Dose | Dose | Reg- | | |
| GN | An. | (OD) | (OS) | (GC/eye) | Volume | imen | Assessments* | Study Endpoints |

| | | | | | | | |
|---|---|---|---|---|---|---|---|
| 1 | 3 | Vehicle, | Vehicle, | N/A | 50 μL for | Full ocular | Blood, feces, saliva, and |
| | | Liquid | Implant | | liquid in- | evaluations at | tears collected throughout |
| 2 | 3 | AAV8 Liquid | | 7.5E±10 | jection or | pre-dose, Day | the study to assess vector |
| 3 | 3 | AAV8 Liquid | | 2.3E±11 | 1 implant | 7, and at Week | shedding (Prior to dose, |
| 4 | 3 | AAV2.7m8 Liquid | | 7.5E±10 | bilateral | 2, 3, 4, 6, 8, | at Days 2, 4, 7, and |
| 5 | 3 | AAV8 Implant: | | 2.3E±11 | IVT dose | 10, and 13. | Weeks2, 4, and 8). |
| | | High Dose-Fast Release | | | on Day 0 | OCT and | Following final ophthalmic |
| 6 | 3 | AAV8 Implant: | | 2.3E±11 | | fluorescent | assessments and blood |
| | | High Dose-Medium Release | | | | imaging at | collection during Week |
| 7 | 3 | AAV8 Implant: | | 2.3E±11 | | pre-dose, | 13, animals will be |
| | | High Dose-Slow Release | | | | Day 7, and | euthanized on Week 14, |
| 8 | 3 | AAV8 Implant: | | 7.5E±10 | | Week 2, 4, 6, | and ocular tissues will |
| | | Medium-Dose Fast Release | | | | 8, and13. | be collected. |
| 9 | 3 | AAV8 Implant: | | 7.5E±10 | | An additional | |
| | | Medium Dose-Medium Release | | | | fluorescent | |
| 10 | 3 | AAV2.7m8 Implant: | | 7.5E±10 | | exam performed | |
| | | Medium Dose-Fast Release | | | | immediately | |
| 11 | 3 | AAV2.7m8 Implant: | | 7.5E±10 | | post dose. | |
| | | Medium Dose-Medium Release | | | | | |

*Animals in Group 4, 10, 11, and 5 received OCT and FAF images at Week 10.
Gr. = Group; No. = Number; An. = Animal; OD = Oculus dexter; OS = Oculus sinister; Conc. = Concentration; IVT = Intravitreal injection; OCT = optical coherence tomography

The assemblies of the filtered solutions in the aluminum heating blocks and double pouched 7.5″×13″ autoclaved pouches were placed into the lyophilizer (1-Shelf Viritis Advantage,). The lyophilization recipe was then confirmed to have the correct parameters (Table 38) and the cycle was started.

TABLE 38
PEG Endotoxin Removal Lyophilization Recipe

| | | Temp | Time | | Pressure |
|---|---|---|---|---|---|
| | Steps | (° C.) | (min) | Ramp/Hold | (mTorr) |
| Thermal | — | — | — | — | — |
| Treatment | | | | | |
| Freeze* | — | −40 | 120 | — | — |
| Primary | 1 | −30 | 600 | Hold | 100 |
| Drying** | 2 | −10 | 80 | Ramp | 100 |
| | 3 | −10 | 240 | Hold | 100 |
| | 4 | 25 | 140 | Ramp | 100 |
| | 5 | 25 | 960 | Hold | 100 |

*Shelf manually frozen to −70° C. prior to initiate cycle
**Condenser set at −70° C.

After the cycle was completed, the vials were stoppered under nitrogen at a pressure of roughly 500 Torr and transferred into the BSC. The vials were removed from the aluminum heating block and the lyophilized PEG cake was visually inspected for any abnormalities, crimp capped, and double bagged into autoclaved pouches consisting of one of each type of lyophilized PEG. They were then placed into a −20° C. freezer for storage.

After the cycle was completed, the vials were stoppered under nitrogen at a pressure of roughly 500 Torr and transferred into the BSC. The vials were removed from the aluminum heating block and the lyophilized PEG cake was visually inspected for any abnormalities, crimp capped, and double bagged into autoclaved pouches consisting of one of each type of lyophilized PEG. They were then placed into a −20° C. freezer for storage.

### AAV Lyophilization:

AAV stock solutions were combined with a concentrated sucrose solution to create mixtures at 10 mg/mL sucrose. The AAV+sucrose mixtures were then lyophilized in 1 mL syringes to remove water. The formulation and final composition of the groups lyophilized is outlined in Table 39.

TABLE 39
Formulation and Composition of Lyophilized AAVs

| | Cryo | Lyo Solution | | | | | | | | |
|---|---|---|---|---|---|---|---|---|---|---|
| | Solu- | | So- | Lyo Volume | | | % Mass Composition of Lyophi- | | | |
| | tion | | dium | | | Total | lized Product | | | |
| | Su- | Su- | Chlo- | AAV | Cryo | Lyo | | | PBS | |
| | crose | crose | ride | Solu- | Solu- | Solu- | | | Salts + | Plu- |
| | (mg/ | (mg/ | (mg/ | tion | tion | tion | | | Sodium | ronic |
| Group | mL) | mL) | mL) | (uL) | (uL) | (uL) | AAV | Sucrose | Chloride | F-68 |
| Pla- | 101.4 | 10 | 0 | 456.7 | 50 | 506.7 | 0.0% | 50.3% | 49.7% | 0.05% |
| cebo* | | | | | | | | | | |
| AAV2.7 | 15.7 | 10 | 4.2 | 181.3 | 325.5 | 506.8 | 0.6% | 41.3% | 58.1% | 0.04% |
| m8 | | | | | | | | | | |
| MDFR | | | | | | | | | | |
| AAV2.7 | 15.7 | 10 | 4.2 | 181.3 | 325.5 | 506.8 | 0.6% | 41.3% | 58.1% | 0.04% |
| m8 | | | | | | | | | | |
| MDMR | | | | | | | | | | |

*The placebo group has no AAV and used PBS + 0.001% Pluronic F68 as a surrogate.

Briefly, the sucrose solutions were endotoxin filtered into the BSC. AAV stock vials were thawed, centrifuged to collect all the solution, and brought into the BSC. After mixing the appropriate AAV stock solution and sucrose solution in 1.5 mL Protein LoBind tubes, the mixtures were loaded in 1 mL Soft-Ject syringes (Henke Sass Wolf, 8300018745) and placed in an aluminum syringe block cooled to −80° C. The loaded syringe block was then double bagged into autoclaved pouches and transferred into the lyophilizer.

The lyophilizer (1-Shelf Viritis Advantage,) was prepared for use by ensuring that the entire chamber was cleaned using Kimwipes and sterile 70% isopropanol. The shelf was frozen to a target temperature of −70° C. prior to starting the process. The appropriate lyo recipe was loaded and verified to the parameters outlined below in Table 40.

TABLE 40
AAV Lyophilization Recipe

| | | Temp | Time | | Pressure |
|---|---|---|---|---|---|
| | Steps | (° C.) | (min) | Ramp/Hold | (mTorr) |
| Thermal Treatment | 1 | −40 | 60 | Hold | — |
| | 2 | −20 | 40 | Ramp | — |
| | 3 | −20 | 180 | Hold | — |
| | 4 | −40 | 40 | Ramp | — |
| Freeze | — | −40 | 60 | — | — |
| Primary Drying* | 1 | −38 | 90 | Hold | 100 |
| | 2 | −35 | 25 | Ramp | 100 |
| | 3 | −35 | 1000 | Hold | 100 |
| | 4 | −35 | 1000 | Hold | 100 |
| | 5 | −10 | 200 | Ramp | 100 |
| | 6 | −10 | 240 | Hold | 100 |
| | 7 | 0 | 80 | Ramp | 100 |
| | 8 | 0 | 240 | Hold | 100 |
| | 9 | 25 | 200 | Ramp | 100 |
| | 10 | 25 | 480 | Hold | 100 |

*Condenser set at −70° C.

Fiber Casting: This section describes the methodology used for fabricating the strands loaded with the AAVs. The previously lyophilized 4a20kSS, 4a40kSG, 4a20kNH2+fluorescein sodium, and 8k linear PEG vials were removed from −20° C. storage and aseptically transferred into the BSC.

PEGs were reconstituted with sterile filtered, anhydrous DMC to make 10% w/w solutions. The PEG formulation for each group is listed in Table 41. The 8k linear PEG, 4a20k SS, and 4a20k SG were then combined in the appropriate volumetric ratios for each group in separate vials.

TABLE 41
PEG Formulation and Volumes for each Group

| | | | | | | | Ester Blend Volu- | | | PEG Volumes | |
|---|---|---|---|---|---|---|---|---|---|---|---|
| | PEG Formulation | | | | | | metric Ratio | | | for Casting | |
| | | % | | PEG | PEG | | | | 8k | | PEG |
| | | Lin- | | Ester | Ester | Ester | | | Lin- | PEG | Ester |
| | % | ear | | 1 | 2 | SS:SG | 4a20k | 4a40k | ear | NH2 | Blend |
| Group | maPEG | PEG | Amine | (SS) | (SG) | Ratio | SS | SG | PEG | (μL) | (μL) |
| Placebo | 8% | 2% | 4a20k | 4a20k | 4a40k | 60:40 | 1 | 1.33 | 1 | 64.8 | 129.6 |
| AAV2.7 | 6% | 4% | 4a20k | 4a20k | 4a40k | 70:30 | 1 | 0.86 | 2.19 | 50.7 | 143.7 |
| m8 | | | | | | | | | | | |
| MDFR | | | | | | | | | | | |
| AAV2.7 | 8% | 2% | 4a20k | 4a20k | 4a40k | 60:40 | 1 | 1.33 | 1 | 64.8 | 129.6 |
| m8 | | | | | | | | | | | |
| MDMR | | | | | | | | | | | |

AAV/Placebo lyophilized syringe were removed from the freeze-dryer and transferred aseptically to the BSC. The necessary amount of PEG amine solution was combined with the lyophilized powder. First, a plunger was reinstalled onto the lyophilized 1 mL HSW syringe, and the PEG amine solution was loaded using a pipette, dispensing the solution through the narrow front end of the syringe while pulling the plunger down. The syringe was then degassed three times by covering the open end of the syringe while pulling the plunger down. The mixed solution was then carefully pushed to the syringe tip and capped with a female luer cap.

The PEG ester blend syringe was prepared by adding the necessary amount of PEG ester blend solution into a new sterile 1 mL HSW syringe. The PEG ester blend solution was then carefully pushed to the syringe tip and capped with a female luer cap.

Casting the AAV/Placebo strands required the use of LDPE tubing of an inner diameter of 0.559 mm and a wall thickness of 0.127 mm cut into 45 cm long segments, white mini plastic tubing clamps, luer connectors, and 22G blunt needles. The tubing was prepared by carefully inserting the blunt 22G needle into one end of the tubing and threading two white mini plastic tubing clamps through the tube. One clamp was placed towards the distal end of the tube and the other was placed towards the proximal end.

The PEG ester and PEG amine syringes were mated with a luer connector and then mixed for 20 or 30 seconds. Before removing the luer connector, the syringe was degassed by pulling the plunger down to roughly the 0.3 mL mark to degas and the syringe was flicked to remove any remaining air bubbles. The luer connecter was then removed and the solution was pushed towards the tip of the syringe.

The syringe was then attached to the tube using the inserted 22G blunt needle and the plunger was slowly pushed to cast the solution into the tube. The distal end of the tubing was clipped once the necessary amount of solution was dispensed. The tube was then slightly pressurized, and the proximal end was then clipped. The remaining solution in the syringe was then used to record the gel time, listed below in Table 42.

TABLE 42
Gel Times for Casted Strands

| | Gel | Mix |
|---|---|---|
| | Time | Time |
| Fiber Formulation | (min:sec) | (s) |
| Placebo | 1:58 | 30 |
| AAV2.7m8 Medium Dose-Fast Release | 2:05 | 20 |
| AAV2.7m8 Medium Dose-Medium Release | 2:02 | 30 |

The casted tubes were then placed into a glass curing chamber in the presence of DMC vapor for 2 hours. The DMC vapor was present in the chamber to prevent premature drying of the casted gel prior to full curing. After curing, the tubing/strand was secured with tape onto an 18 inch long depyrogenated stainless steel ruler. The ruler with the tubing/strand was then placed and double pouched into 3.5″×22″ autoclaved pouches and transferred outside of the BSC and into a 37° C. oven with a nitrogen gas flow set to 30 SCFH to dry for 3 days.

Final composition of the dried AAV/Placebo implants are listed in Table 43.

TABLE 43
% Mass Composition of Dried Implants

| | | | PBS | | | | | |
|---|---|---|---|---|---|---|---|---|
| | | | Salts + | | | | LDP: | |
| | | | Sodium | Plu- | | | PEG | LDP: |
| | | Su- | Chlo- | ronic | Linear | | Ra- | maPEG |
| Group | AAV | crose | ride | F-68 | PEG | maPEG* | tio** | ratio |
| Placebo | 0% | 16.5% | 16.3% | 0.02% | 13.4% | 53.7% | 0.49 | 0.61 |
| AAV2.7m8 | 0.2% | 15.4% | 21.7% | 0.02% | 25.0% | 37.5% | 0.60 | 1.00 |
| MDFR | | | | | | | | |
| AAV2.7m8 | 0.2% | 15.4% | 21.7% | 0.02% | 12.5% | 50.1% | 0.60 | 0.75 |
| MDMR | | | | | | | | |

*maPEG = multi-arm PEG
**LDP:PEG Ratio = Lyophilized Product:PEG mass ratio

Fiber Cutting: The previously casted and dried fibers were removed from the 37° C. oven and aseptically transferred into the BSC. Working with one casted strand at a time, the casted tubing/strand was removed from the ruler and placed onto the depyrogenated stainless steel cutting apparatus.

Microtome blades were used to cut the strands into 8 mm fibers. To prevent dull fiber cuts, the blades were replaced after cutting five times in the beginning, middle, and end areas of the blade for a total of fifteen cuts per blade. After cutting, the fibers were kept in the tubing and transferred into the appropriate LoBind tubes designated for PCR, Bioburden, Endotoxin, Aggregation, and IV release, and needle-loading.

Using an 18G needle, four holes were punched into the top of the LoBind tubes containing the cut fibers. The LoBind tubes were then double pouched in autoclaved pouches and transferred into a nitrogen glove box for conditioning for a minimum of 3 days.

Needle Loading: The cut fibers were removed from the glove box and aseptically passed inside of the BSC, along with the forceps, glass slides, and 2″ nitinol wires. The 27G 1.25″ needle (NIPRO Hypodermic Needle, 27Gx1.25″, No. AH+2732) were transferred into the BSC one at a time and visually inspected for any damage to the tip of the needle. An implant was then grabbed with forceps and loaded into the needle through the tip. If the fiber was difficult to insert, it was placed between two glass slides and a gentle rolling motion was applied to the fiber to smoothen any bumps. After rolling the fiber, the insertion was retried and if still not successful the fiber was discarded.

Once the fiber was loaded into the needle a 2″ nitinol wire was inserted into the needle tip and a polypropylene hub cap was inserted into the needle hub thus securing the fiber in place for further handling. This assembly was then placed into a small, autoclaved Tyvek pouch and sealed. Once all needles were loaded and sealed in the autoclaved pouches, they were transferred outside of the BSC.

Outside of the BSC, the autoclaved pouches containing the loaded needles were placed into an aluminum pouch (Oliver Healthcare Packaging, Part No. 90-2003-002 REV C), labeled, and transferred into the glove box for conditioning for 3 days. After conditioning the aluminum pouches were sealed using a pouch sealer and the completed assemblies were sterile and ready for use.

### Implant Characterization

Bioburden and Endotoxin Testing: The same process was used for both endotoxin and bioburden sample preparation. 1×PBS, PH=8.4 buffer solution was endotoxin filtered into the BSC. Three fibers per formulation were placed into respectively labeled depyrogenated 2 mL glass vials (Duran Wheaton Kimble, 2 mL vial, Serum, Type I CLR Glass, Part No. 223683). 1 mL of the buffer solution was added to each vial, sealed with a rubber stopper, and crimped. The vials were swirled to ensure that all fibers were submerged. The vials were then transferred into a 37° C. incubator and placed onto a shaker plate for vigorous shaking. After full degradation, the samples were removed from the incubator and kept at room temperature until ready to ship. The vials were evaluated for endotoxin and bioburden analysis.

Dimensional Analysis: AuNP fibers with the same formulation were prepared for dimensional analysis. 8 mm fiber implants were cut from the strand and dry dimensions (length and diameter) were measured via the Starrett Vision Systems. 1× fiber placed in a 1.5 mL Eppendorf Lo-protein binding tube (VWR Cat #80077-232). 1 mL of buffer solution (1×PBS+0.001% Pluronic F-68 at pH=7.2) was added into each tube. 3 samples were prepared for each group (total=3 fibers per group). Samples were kept at 37° C. Swollen dimensions were measured daily via Starrett Vision Systems.

Dose Measurement: qPCR Titer: See Attachment 6-NB Scan-PCR sample prep: 4×8 mm AAV fiber implants were cut from the strand and placed in an autoclaved 1.5 mL Eppendorf Lo-protein binding tube (VWR Cat #80077-232). 0.5 mL of sterile filtered buffer solution (1×PBS+0.001% Pluronic F-68 at pH=8.4) were added into each tube. 3 samples were prepared for each group (total=12 fiber implants per group). Samples were kept at 37° C. under vigorous shaking. Upon full fiber degradation after 8 days at 37° C., samples spun down with the minifuge and pipette mixed. Total solution was transferred into 5×100 μL aliquots in 0.5 mL Lo-protein binding tubes (VWR Cat #8007-228). Samples were flash frozen in liquid nitrogen and analysed via PCR analysis.

IV Release Study—ELISA Analysis: 8 mm AAV fiber implants were cut from the strand and placed in a 1.5 mL Eppendorf Lo-protein binding tube (VWR Cat #80077-232). 1 mL of buffer solution (1×PBS+0.001% Pluronic F-68 at pH=7.2) added into each tube. 3 samples were prepared for each group (total=3 fiber implants per group). Samples were kept at 37° C. At each timepoint, 250 μL of PBS was removed and replaced with fresh PBS. The IV release sample was then flash frozen in liquid nitrogen and stored at −80° C. prior to ELISA analysis. AAV2 Xpress ELISA kits (Progen Cat #PRAAV2XP) were used to measure IV release. Initial internal testing conducted first and determined that AAV2 ELISA kits could be used for AAV2.7m8 samples (data not shown). % Cumulative release was calculated by each sample's maximally released dose.

In Vivo Study Design: A 13-week inflammation and expression study of AAV-loaded hydrogel implants was conducted in New Zealand White rabbits. Table 37 outlines the study design.

Both AAV8 and AAV2.7m8-loaded implants were prepared and tested.

### Bioanalytics

### GFP Quantification

After euthanasia, OD (right) eyes were enucleated with muscle and optic nerve trimmed. The OD tissues (Optic nerve, retina, RPE/choroid, aqueous and vitreous humor) were dissected out and immediately stored at −80° C. in Eppendorf LoBind® microcentrifuge tubes (1.5 mL/2.0 mL) with cap locks. To quantify the GFP in ocular tissues, a commercial ELISA method was optimized for use in rabbit ocular tissues via titration of reagents. Commercial Kit: Abcam GFP ELISA Kit (Catalog #: ab171581) was used for assay optimization. All ocular tissue samples underwent homogenization, lysis, and BCA to quantitate total protein prior to use during optimization of the ELISA method. After method optimization, rabbit tissue samples from retina, RPE/choroid, and optic nerve were analyzed for GFP with the optimized GFP ELISA method.

### Vector Shedding in Plasma

Whole blood was collected from non-sedated or sedated animals on Day 0 (predose), 2, 4, and 7 and Week 2, 4 and 8. Approximately 1.0 mL of whole blood was collected from each animal into tubes containing K2EDTA and processed into plasma. Plasma was divided into 200 UL aliquots and stored at nominally −80° C. To understand AAV shedding in plasma, a qPCR method was used to quantify the copy number of the eGFP target sequence in rabbit plasma samples.

### Anti-Drug Antibodies (ADA) Assay

Blood samples were collected from the central ear artery. Blood was collected from non-sedated or sedated animals. Samples were processed into serum. Serum was divided equally into 2 Eppendorf LoBind microcentrifuge with cap locks and stored at nominally −80° C. An anti-drug antibody ELISA was developed for the detection and determination of specific antibodies to AAV2.7m8 in rabbit serum samples. Rabbit serum samples were screened. The positive test samples were serially diluted from 1:30 to 1:21, 870. The sample titer value is the highest dilution at which a sample has a mean OD value equal to or above the assay cutoff, with the next highest dilution being below the assay cutoff.

### Results

Endotoxin: Table 44 lists endotoxin results of placebo, AAV2.7m8 Fast and Medium release fiber implants. All implants had very low endotoxin levels below 0.01 Endotoxin/implant that shows the sterility of our manufacturing process.

TABLE 44
Endotoxin Results

| | Total |
|---|---|
| Test Article | Endotoxin/Implant |
| Placebo | 0.003 |
| AAV2.7m8 Fast Release | 0.003 |
| AAV2.7m8 Medium Release | 0.003 |

Bioburden: Table 45 shows bioburden results of placebo, AAV2.7m8 Fast and Medium release fiber implants. There were not observable colonies in any implant groups.

TABLE 45
Bioburden Results

| | CFU/Plate | | | |
|---|---|---|---|---|
| | | | TSA | SDA |
| | TSA | TSA | (Heat | (Yeast & |
| Test Article | (Aerobic) | (Anaerobic) | Shocked) | Mold) |
| Placebo | NOC | NOC | NOC | NOC |
| AAV2.7m8 Fast Rel. | NOC | NOC | NOC | NOC |
| AAV2.7m8 Medium Rel. | NOC | NOC | NOC | NOC |

CFU: Colony Forming Units
NOC: No Observable Colonies

Dimensional Analysis: Since AAV fiber implants became transparent when they are immersed in water, AuNP fibers were used to measure dry and swollen dimensions. AuNP fibers were prepared with the same formulations (fast and medium release). Table 41 outlines the formulations of fast release and medium release fibers.

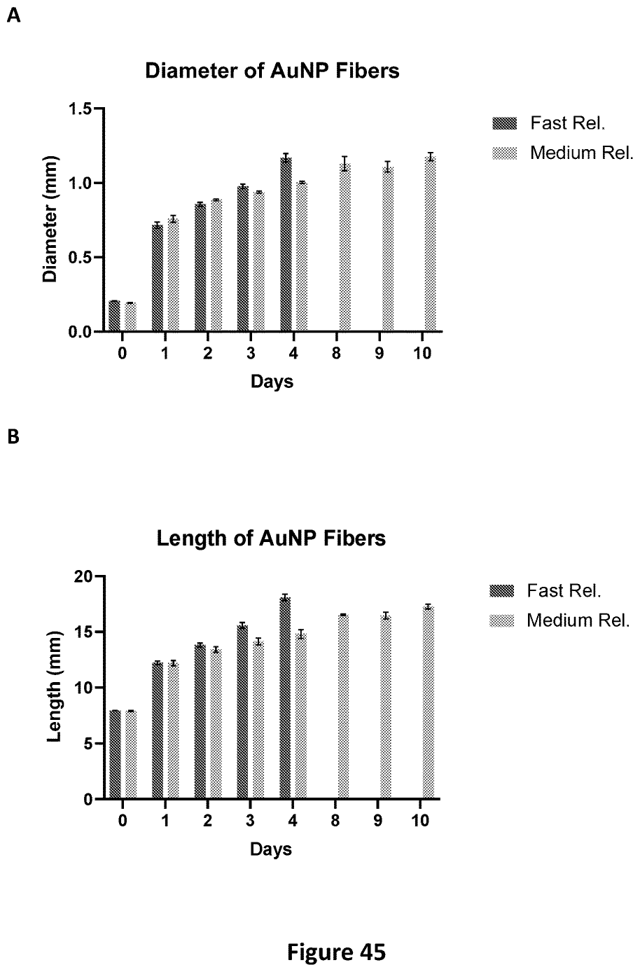

FIG. 45A shows the change in diameter over time and FIG. 45B shows the change in length over time. Diameter and length of both formulations were similar on Day 0. Both formulations showed high degree of swelling with longer length and thicker diameter on Day 1. Medium release fibers had shorter length and thinner diameter compared to fast release fibers. This can be attributed to the greater % multiarm PEG concentration in medium release formulation. A greater % multiarm PEG concentration increases crosslinking density and thus led to a smaller mesh size and limited hydrogel expansion. Finally, while fast release fibers degraded on day 6, medium release fibers degraded on day 14. Due to the higher SS content, the fast release fibers degraded earlier than the medium release fibers.

qPCR Titer Analysis:

Table 46 shows the PCR analysis results of AAV2.7m8 bolus, fast and medium Release samples. In the same way, ELISA results could have also been used as it was found in the previous example that the ELISA and PCR results are comparable. The implant titers were comparable however they were lower than the theoretical value (7.5 e10/implant).

TABLE 46
PCR Results of AAV2.7m8 Bolus, Fast, and Medium Release Implants

| Groups | Titer |
|---|---|
| AAV2.7m8 Bolus | 2.95E+10 GC |
| AAV2.7m8 Fast Release | 3.46E+10 GC/implant |
| AAV2.7m8 Medium Release | 3.67E+10 GC/implant |

In Vitro AAV Release-ELISA: Formulations listed in Table 41 were selected for this rabbit study. Fast release formulations were chosen by increasing the SS:SG ratio (increasing SS content). While the % multi-arm PEG concentration in DMC was maintained at 6% for fast release formulations, the % multi-arm PEG concentration in DMC was kept at 8% for medium release formulations.

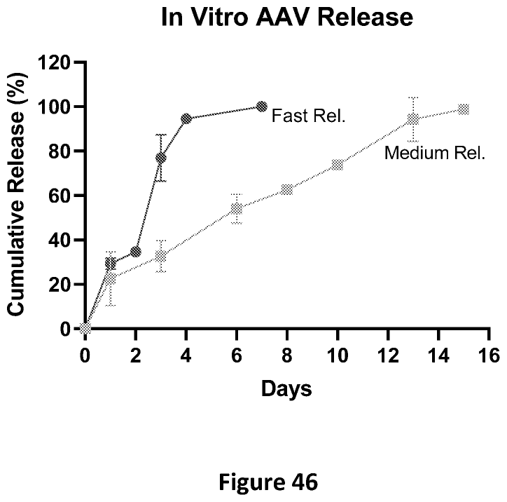

FIG. 46 shows the IV release curves of AAV2.7m8 Fast and Medium Release samples. From these results, it's seen that the SS:SG ratio and % multi-arm PEG concentration have an impact on release kinetics and fiber degradation. Increasing SS content led to a faster degrading fibers and faster release. While the fast release samples reached 100% release within 4 days, medium release samples reached 100% release in 15 days. Additionally, a greater % maPEG concentration results in slower release kinetics likely due to the increased crosslinking density and smaller mesh size.

### In Vivo Study Results:

In Vivo Fiber Degradation-Multicolor Imaging: Fluorescent imaging performed at pre-dose and post dose to visualize fiber implants and to confirmed successful deployment in rabbit vitreous. While the AAV2.7m8 fast release implant was visible on post dose IR image, it was no longer visible on the multicolor image on Day 9 due to fiber degradation. The AAV2.7m8 medium release implant was visible on post dose IR image and multicolor images on Day 9 and Day 14. Most of the AAV2.7m8 medium release implant degraded by Day 30, fibrous strand on Day 30 could be either leftover hydrogel or fibrin.

Ocular Examinations: Full ocular evaluations were performed at predose, Day 7, and at Week 2, 3, 4, 6, 8 and 10. The SPOT scoring system was used for ocular evaluations.

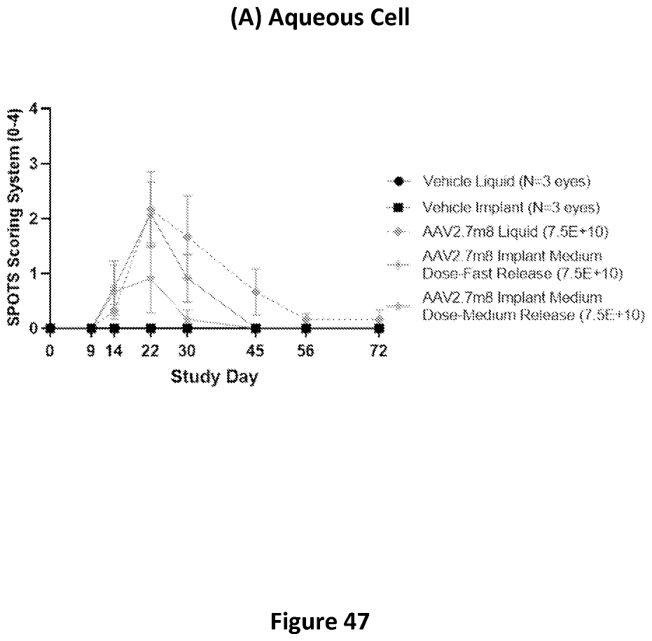

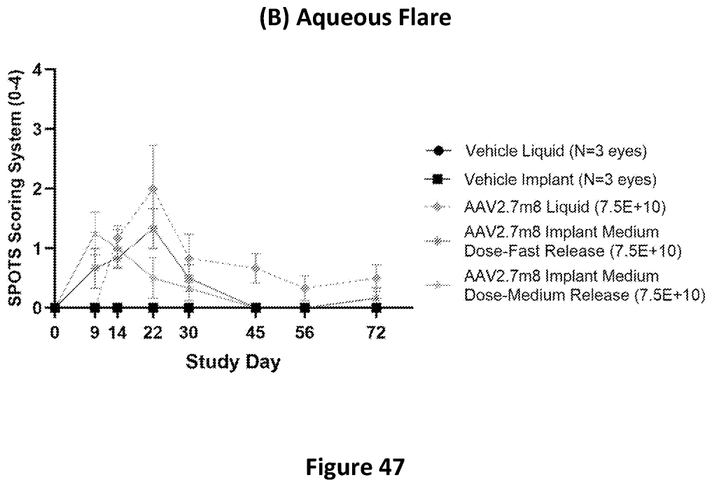

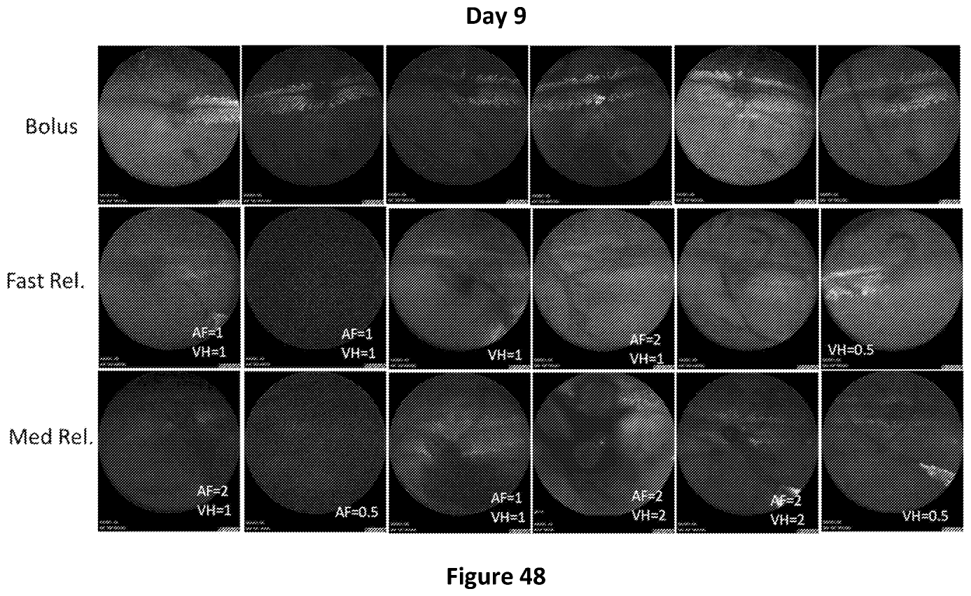

FIG. 47 shows the aqueous flare scores for all groups up to Day 72 (Week 10). Aqueous flare scores were zero throughout the study for both placebo fiber implants and liquid vehicle. Inflammation peak was seen on Day 22 for AAV2.7m8 bolus, fast, and medium release groups. After Day 22, aqueous flare scores subsided for all groups. Compared to AAV2.7m8 fast and medium release implants, AAV2.7m8 bolus had greater aqueous flare scores throughout the study except Day 9.

This is expected because any inflammation seen in the first two weeks could likely be due to the deployment procedure or a reaction to the implant biomaterial (and therefore, not a source of major concern), however the inflammation peak seen between week two and four is mostly likely due to the result of an antigen-specific immune response against vector capsid, vector genome, or expressed transgene (which should ideally be controlled). These data show that the implants of the present invention may be used to evade the antigen-specific immune response. Further, these data show that this immune response and thus, inflammation can be further controlled at higher dose by prolonging the number of days required for 100% release of the AAV dose. When AAV dose was low (as shown in Example 5-1^{st} rabbit study), the AAV release within 4 days reduced inflammation to none-trace and resulted in steady GFP expression compared to the bolus dose. However, higher doses can also be delivered if the number of days required for 100% release is extended. In this example, by increasing the number of days required for 100% release of the AAV dose over 2 weeks, the medium release formulation (3× higher dose) had the lowest inflammation scores and strongest GFP expression among all groups in the 2nd rabbit study.

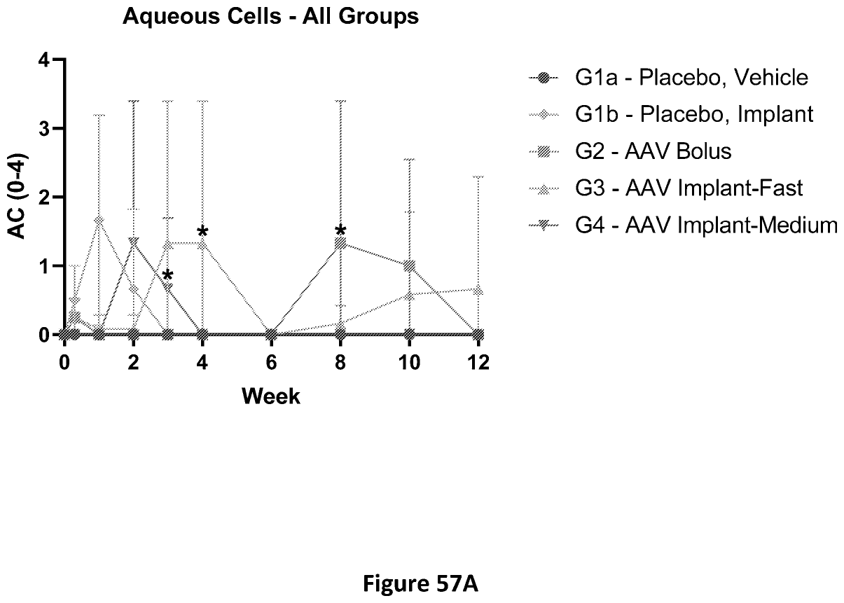

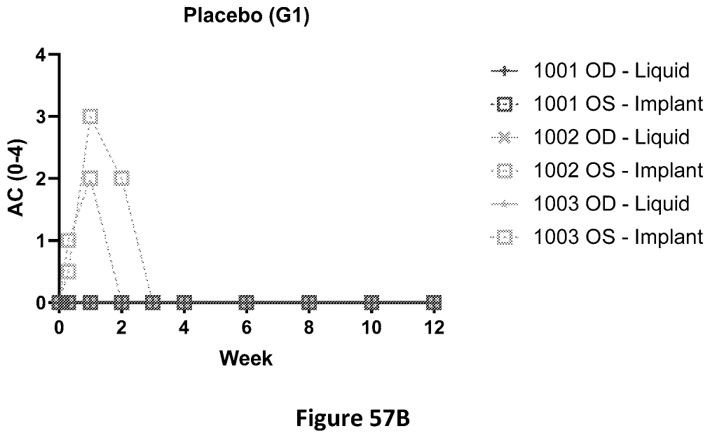

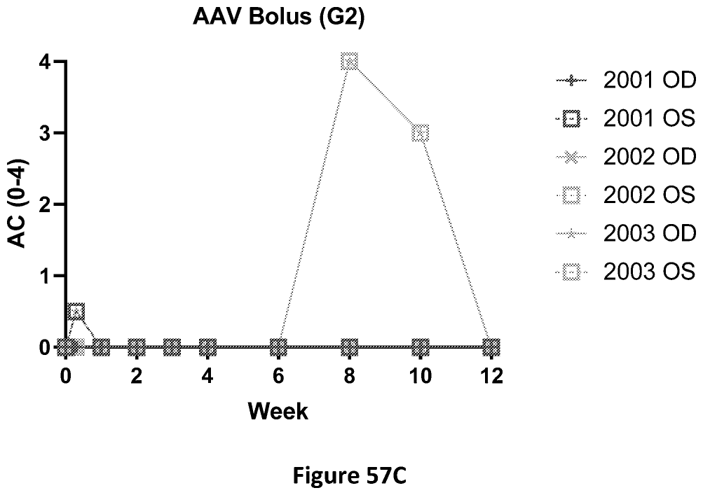

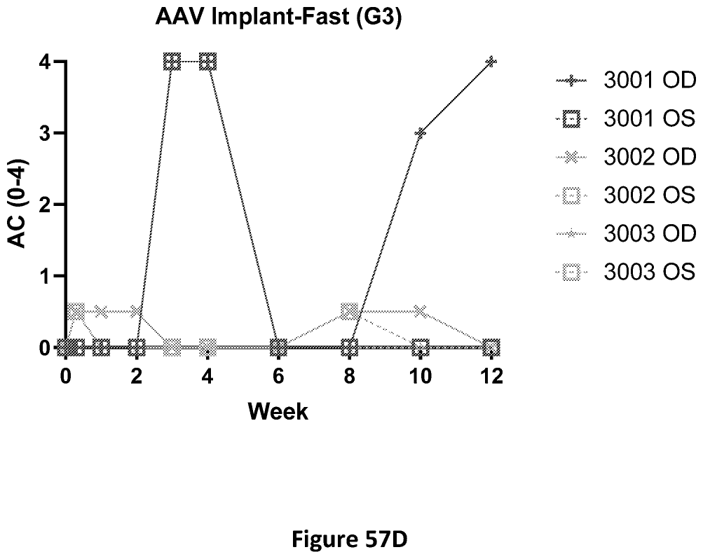

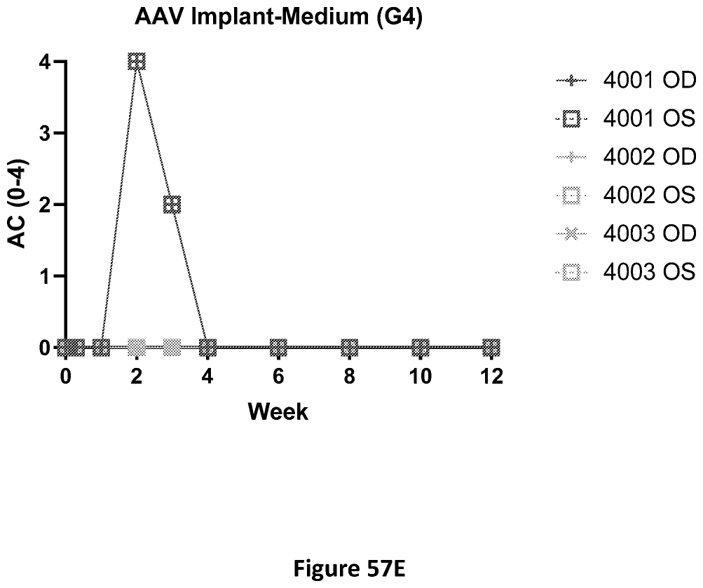

FIG. 57 shows the FAF images with inflammation scores on Day 9. The rabbit retina has myelinated nerve fibers radiating from the optical nerve head, termed the medullary rays. The majority of GFP signals was coming from this area. As expected, there was not any fluorescent signal from the placebo group throughout the study. The GFP expression intensity was significantly larger in the eyes that were injected with liquid AAV2.7m8 compared to the eyes treated with the AAV2.7m8 implants on Day 9. In the implant groups, GFP expression was weak across the medullary rays. While the fast release implant degraded already by Day 9, medium release implant was still visible on Day 9.

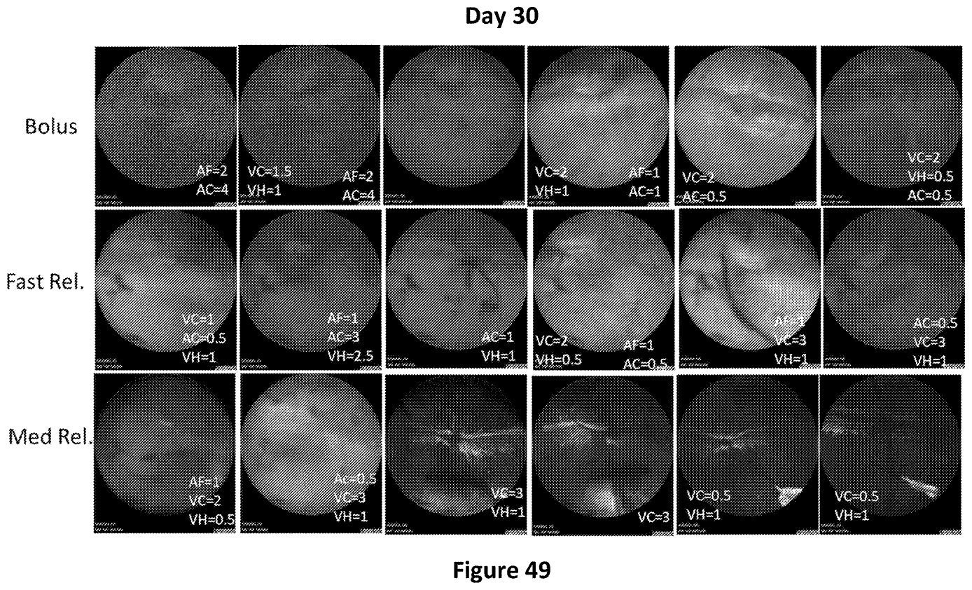

FIG. 49 shows the FAF images with inflammation scores on Day 30. The GFP expressions in the AAV2.7m8 bolus and fast release implant groups were weak after the inflammation peak on Day 22, however GFP expression intensity in the medium release group was even stronger than Day 14. This is attributed to the relationship between inflammation and GFP expression. AAV2.7m8 medium release implant group had lower inflammation scores on Day 22 compared to AAV2.7m8 bolus and fast release implant groups. There were still mild to moderate fibrin.

Like Day 30 results, GFP signal from the AAV2.7m8 bolus and fast release implant groups were still low on days 45 and 52, on the other hand GFP signal from the AAV2.7m8 medium release implant group was still visible and strong.

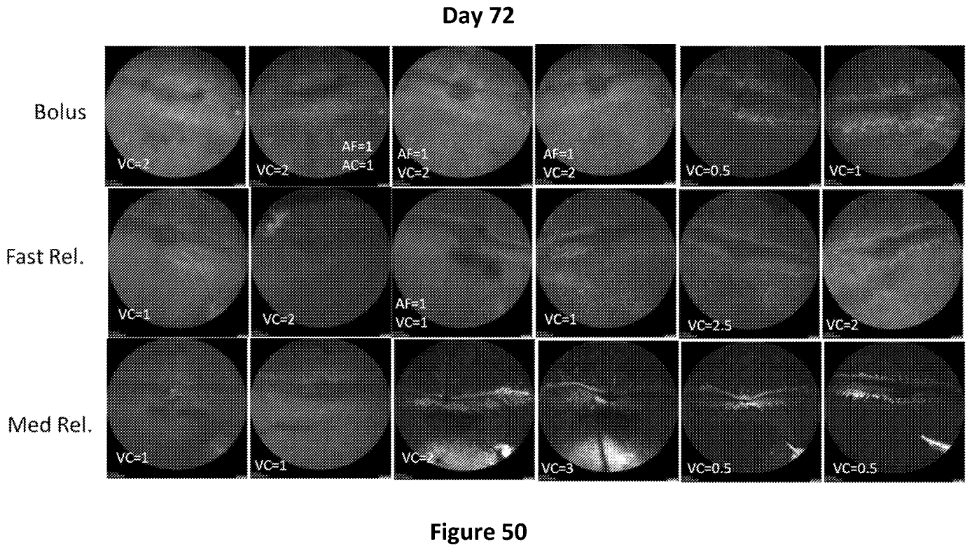

This study was initially planned for 8 weeks, week 10 timepoint was added later and FAF images were taken to investigate the GFP expression difference among AAV2.7m8 groups. FIG. 50 shows the FAF images and inflammation scores on Day 72. The GFP expression intensity was still clearer and stronger in the eyes that were injected with the AAV2.7m8 medium release implants compared to the eyes injected with the AAV2.7m8 bolus and fast release implants.

In the first rabbit study, the effect of sustained AAV2 delivery via hydrogels on inflammation and GFP expression following IVT administration in New Zealand white rabbits was investigated. In the second rabbit study, the AAV dose was increased (3× higher than the first rabbit study) and two release formulations were developed. While implants used in the first rabbit study had a formulation that could release AAVs within 4 days (fast release), implants used in the second rabbit study had two formulations that could release AAVs within 4 days (fast release) and within 15 days (medium release). In addition, a modified AAV2 vector-AAV2.7m8 in the second rabbit study was used.

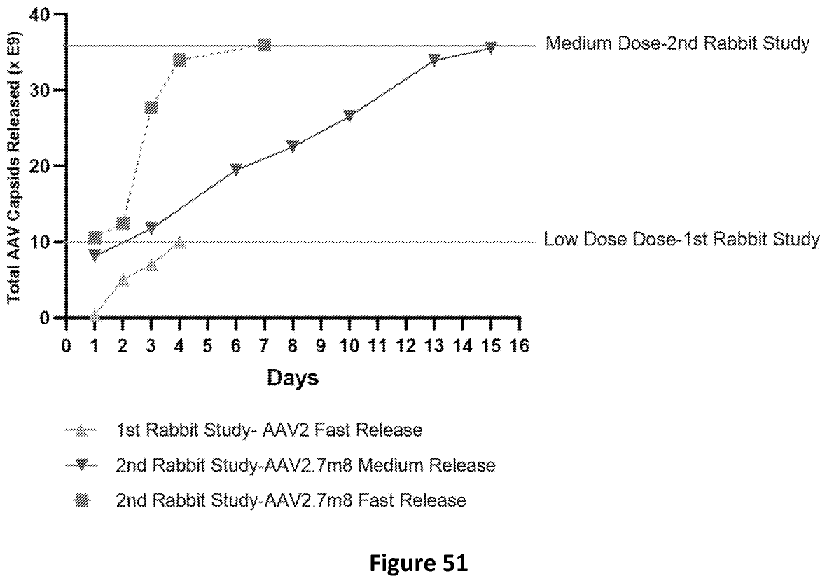

Both study results showed that the release of high titer AAVs in a short period of time resulted in inflammation and loss of GFP expression. FIG. 51 exhibits AAV release over time based on ELISA results from the first and second rabbit studies. When AAV dose was low (1st study), the AAV release within 4 days helped to control inflammation and thereby resulted in steady GFP expression compared to the bolus dose. On the other hand, the AAV release within 4 days in the 2nd study was not enough to manage inflammation since AAV dose was 3× higher than the first study. The medium release formulation had the lowest inflammation scores and strongest GFP expression among all groups in the 2nd rabbit study due to release of high titer AAVs over a longer period.

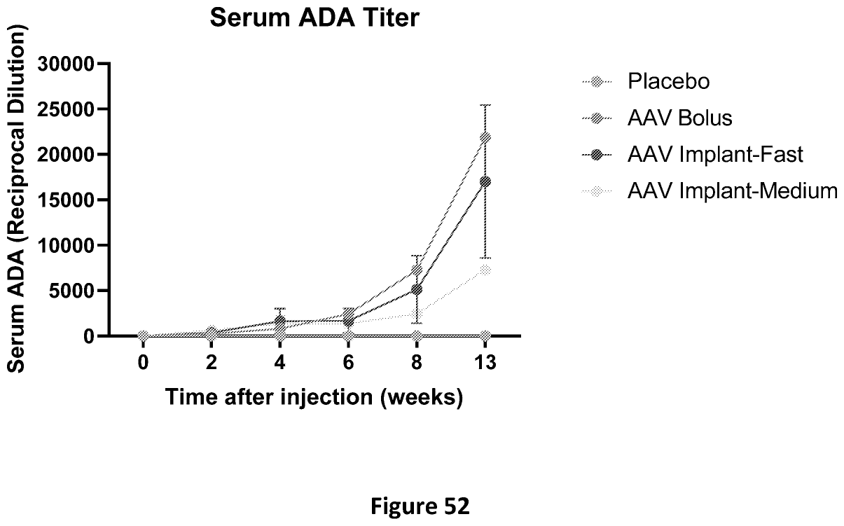

In line with inflammation scores discussed above, the lowest anti-drug antibody (ADA) levels were found in the serum of the medium release group as compared to the AAV bolus group that had the highest levels of ADA (FIG. 52). The placebo group and pre-dose samples generated negative screening results. This means that by controlling the release profile of the implants of the present invention irrespective of the dose, the immune response can be controlled when treating an ocular disorder using AAVs.

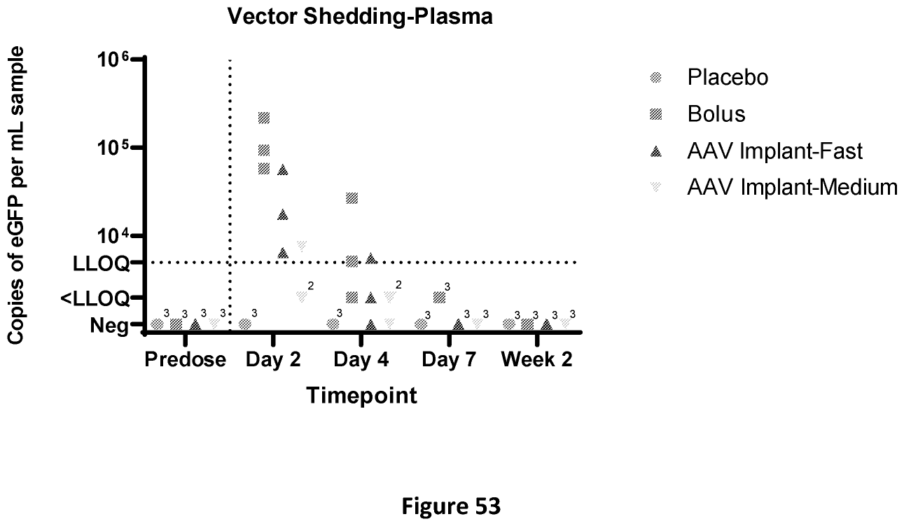

Interestingly, the vector shedding data showed a similar trend. Vector shedding in the plasma can be quantified in a number of ways such as by quantifying a nucleic acid sequence of the AAV genome, or by quantifying the AAV proteins, or by quantifying the heterologous nucleic acid sequence comprised in the AAV. In this example, vector shedding was quantified by the copies of heterologous nucleic acid sequence in the plasma of the animals. The placebo group, pre-dose samples and all samples beyond Day 4 generated copy numbers below the limit of detection (negative). All day 2 samples from AAV Bolus and AAV Implant-Fast groups generated copy numbers greater than the lower limit of quantification (LLOQ). AAV-Implant Medium group had the lowest amount of eGFP copies in the plasma compared to AAV Bolus and AAV-Implant-Fast groups. The highest copies of eGFP per mL of sample could be seen in the AAV bolus group followed by AAV (fast release) and AAV (medium release) groups (FIG. 53).

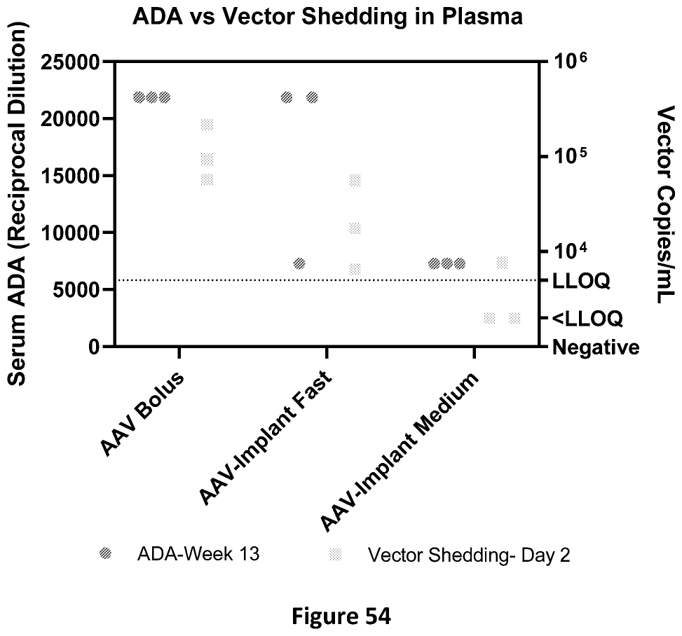

These results may indicate that by controlling the release profile of the implants of the present invention, irrespective of the dose, the AAV can be retained locally in the ocular tissues to a large extent as compared to the bolus group. The results also show that the AAVs in the medium release group do not reach the systemic circulation at the same levels as the bolus group. This could be a reason as to why the implants of the invention do not initiate an immune response (quantified by serum ADA titers) up to the level that is seen in AAV bolus group. FIG. 54 depicts the serum ADA titer versus vector shedding.

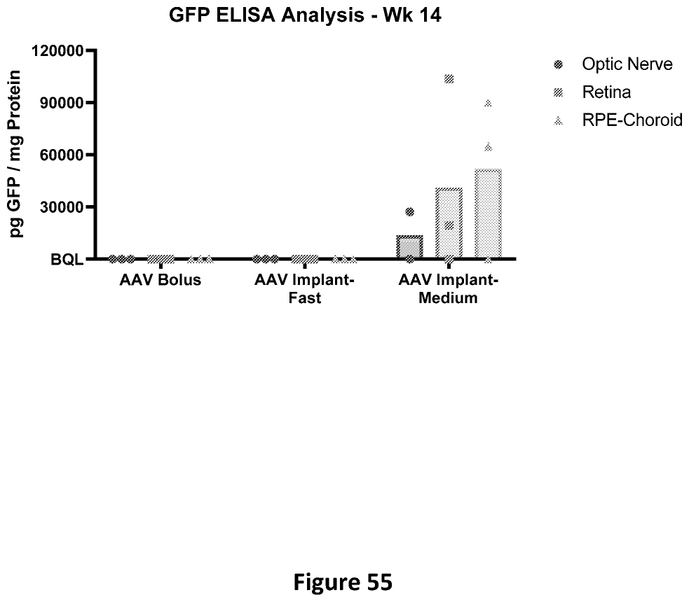

In line with the inflammation scores, and ADA assay, quantitative analysis of GFP expression showed strongest expression in the medium release group (FIG. 55).

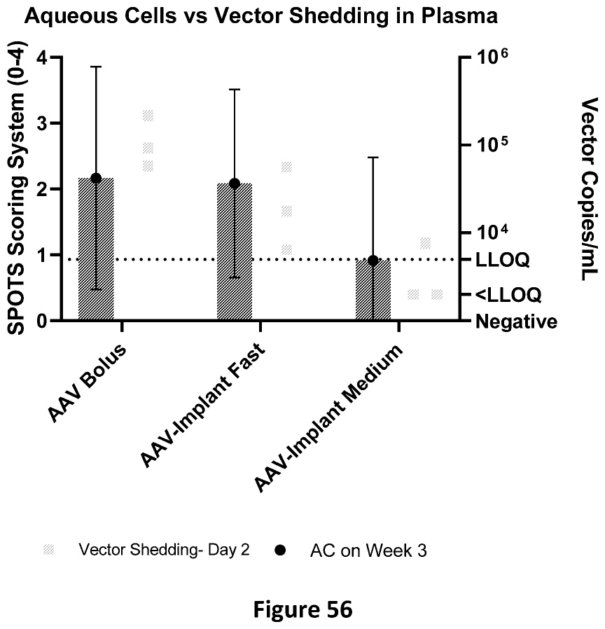

FIG. 56 depicts vector shedding versus aqueous cell inflammation scores at week 3.

From the above data, it can be concluded that there is a negative correlation between the amount of detectable transgene copies in the plasma and GFP quantification in the ocular tissues of the animals. Furthermore, there is a positive correlation between the amount of detectable transgene copies in the plasma and the detectable serum ADA titer against the AAV. Furthermore, there is a negative correlation between inflammation and GFP quantification in the eyes of the animals.

An effective treatment of an ocular disorder with AAV based gene therapy would require expression of the transgene in ocular tissues. An indirect indication of this can be the total detectable copy number of the transgene in the plasma. This is because the data shows that the highest GFP quantification in ocular tissues were seen in the medium release group and at the same time the lowest detectable eGFP copies in the plasma were seen in the medium release group (compare FIG. 55 and FIG. 53)

An effective treatment of ocular disorder would also require controlled immune response as also seen in the medium release group (FIG. 52).

An effective treatment of ocular disorder would also require controlled inflammation as also seen in the medium release group (FIG. 56).

In line with this observation, it can be concluded that the implant of the present invention may provide an effective treatment of an ocular disorder using AAV based gene therapy.

### Example 7—Non-Human Primate Study—Controlled Inflammation by Controlling Release of AAV2.7m8

In the first rabbit study (Example 5), the AAV implant groups had relatively lower inflammation scores and more consistent GFP expression compared to AAV bolus group throughout the study (8 weeks). In the second rabbit study (Example 6), we used AAV implants (with three times higher dose as compared to the first rabbit study). Furthermore, we tuned the release profile of the implants and investigated fast release implants, and medium release implants. We demonstrated that inflammation can be controlled even by delivering AAVs at a much higher dose. We also demonstrated that immune response can be controlled even by delivering AAVs at a much higher dose. We also demonstrated that there is a negative correlation between e detectable copy number of the transgene in the plasma and GFP expression. We also demonstrated that the implants of the invention can be used to provide an effective treatment of an ocular disorder using an AAV based gene therapy.

To test this further, we used a non-human primate (NHP) model. The summary of the groups are shown in Table 47 below.

TABLE 47
Study groups of NHP study

| | | Test Material | | Target | | Dose | | |
|---|---|---|---|---|---|---|---|---|
| | | Right | Left | Dose | Dose | route/ | | |
| | No. | Eye | Eye | (GC/ | vol- | Regi- | | |
| GN | An. | (OD) | (OS) | eye) | ume | men | Assessments | Study Endpoints |

| | | | | | | | |
|---|---|---|---|---|---|---|---|
| 1 | 3 | Vehicle, | Vehicle, | N/A | 50 μL for liquid in- | Full ocular eval- | Blood, urine, saliva, |
| | | liquid | implant | | jection or 1 implant | uations at pre- | nasal secretions, and |

| | | | | | | |
|---|---|---|---|---|---|---|
| 2 | 3 | AAV2.7m8 Liquid | 1E+11 | bilateral IVT dose | dose, Day 2, 7, | tears collected |
| 3 | 3 | AAV2.7m8 Im- | 1E+11 | on Day 0 | and at Week 2, | throughout the study |
| | | plant: | | | 3, 4, 6, 8, 10, | to assess vector shed- |
| | | Fast Release | | | and 12. | ding (Prior to dose, at |
| 4 | 3 | AAV2.7m8 Im- | 1E+11 | | OCT and fluo- | Days 2, 4, 7, 10, and |
| | | plant: | | | rescent imaging | Weeks 2, 3, 4, 6, 8, |
| | | Medium Release | | | at pre-dose, | and 12). |
| | | | | | post-dose, Day | Following final ophthal- |
| | | | | | 7, and Week 2, | mic assessments and |
| | | | | | 3, 4, 6, 8, and | blood collection, ani- |
| | | | | | 12. | mals will be euthanized |
| | | | | | | on Week 12, and ocu- |
| | | | | | | lar tissues will be col- |
| | | | | | | lected. |

GN = Group Number; No. = Number; An. = Animal; OD = Oculus dexter; OS = Oculus sinister; GC = Genome copies.
IVT = Intravitreal injection; OCT = optical coherence tomography

### Assessments and Study Points

Full ocular evaluations carried out at pre-dose, Day 2, 7, and at Week 2, 3, 4, 6, 8, 10, and 12. OCT and fluorescent imaging at pre-dose, post-dose were carried out at Day 7, and Week 2, 3, 4, 6, 8, and 12. At week 2, animal 4001 was given dexamethasone subconjunctival injection followed by an IVT injection of triamcinolone acetonide (TA) at week 3. At week 3, animal 3001 was given subconjunctival injection of TA followed by IVT injection of TA at week 4. Animal 2003 was given an IVT injection of TA at week 8. For all the above cases, the TA dose was 0.1 mL. No other rescue steps were required for any of the animals in any group. Blood, urine, saliva, nasal secretions, and tears collected throughout the study to assess vector shedding (Prior to dose, at days 2, 4, 7, 10, and weeks 2, 3, 4, 6, 8, and 12). Following final ophthalmic assessments and blood collection, animals were euthanized on Week 12, and ocular tissues collected.

Further information on the implants is given in Tables 48, 49 and 50 below.

TABLE 48
Formulation and Composition of Lyophilized AAVs

| | Cryo | Lyo Solution | | | | | | | | |
|---|---|---|---|---|---|---|---|---|---|---|
| | Solu- | | So- | Lyo Volume | | | % Mass Composition of Ly- | | | |
| | tion | | dium | | | Total | ophilized Product | | | |
| | Su- | | Chlo- | AAV | Cryo | Lyo | | | PBS | |
| | crose | Sucrose | ride | Solu- | Solu- | Solu- | | | Salts + | |
| | (mg/ | (mg/ | (mg/ | tion | tion | tion | | | Sodium | Pluronic |
| Group | mL) | mL) | mL) | (μL) | (μL) | (μL) | AAV | Sucrose | Chloride | F-68 |
| Placebo | 10 | 10 | 11.69 | 0 | 500 | 500 | 0.0% | 31.7% | 68.3% | 0.03% |
| AAV | 110 | 11 | 11.69 | 450 | 50 | 500 | 0.6% | 33.6% | 65.8% | 0.03% |
| Implant- | | | | | | | | | | |
| Fast | | | | | | | | | | |
| AAV | 110 | 11 | 11.69 | 450 | 50 | 500 | 0.6% | 33.6% | 65.8% | 0.03% |
| Implant- | | | | | | | | | | |
| Medium | | | | | | | | | | |

TABLE 49
PEG Formulation and Volumes for each Group

| | | | | | | | | | | PEG Volumes | |
|---|---|---|---|---|---|---|---|---|---|---|---|
| | PEG Formulation | | | | | | Ester Blend | | | for Casting | |
| | | | | PEG | PEG | Ester | Volumetric Ratio | | | | PEG |
| | | % | | Ester | Ester | SS: | | | 8k | PEG | Ester |
| | % | Linear | PEG | 1 | 2 | SG | 4a20k | 4a40k | Linear | NH2 | Blend |
| Group | maPEG | PEG | Amine | (SS) | (SG) | Ratio | SS | SG | PEG | (μL) | (μL) |
| Placebo | 6% | 0% | 4a20k | 4a20k | 4a40k | 70:30 | 1 | 0.86 | 0 | 116.5 | 151.4 |
| AAV | 6% | 0% | 4a20k | 4a20k | 4a40k | 70:30 | 1 | 0.86 | 0 | 116.5 | 151.4 |
| Implant- | | | | | | | | | | | |
| Fast | | | | | | | | | | | |
| AAV | 6% | 0% | 4a20k | 4a20k | 4a40k | 55:45 | 1 | 1.64 | 0 | 109.4 | 158.6 |
| Implant- | | | | | | | | | | | |
| Medium | | | | | | | | | | | |

TABLE 50
% Mass Composition of Dried Implants

| | | | PBS | | | | | |
|---|---|---|---|---|---|---|---|---|
| | | | Salts + | | | | | LDP: |
| | | | Sodium | Pluronic | Linear | | LDP:PEG | maPEG |
| Group | AAV | Sucrose | Chloride | F-68 | PEG | maPEG* | Ratio** | ratio |
| Placebo | 0.0% | 15.2% | 32.8% | 0.02% | 0.0% | 52.0% | 0.92 | 0.92 |
| AAV Implant-Fast | 0.3% | 16.4% | 32.2% | 0.01% | 0.0% | 51.1% | 0.96 | 0.96 |
| AAV Implant-Medium | 0.3% | 16.4% | 32.2% | 0.01% | 0.0% | 51.1% | 0.96 | 0.96 |

*maPEG = multi-arm PEG
**LDP:PEG Ratio = Lyophilized Product:PEG mass ratio

### Results

qPCR Titer Analysis:

Table 51 shows the PCR analysis results of AAV2.7m8 bolus, fast and medium Release samples.

TABLE 51
PCR Results of AAV2.7m8 Bolus, Fast, and Medium Release Implants

| Groups | Titer |
|---|---|
| AAV2.7m8 Bolus | 3.0E+10 GC |
| AAV2.7m8 Fast Release | 8.9E+10 GC/implant |
| AAV2.7m8 Medium Release | 6.8E+10 GC/implant |

### In Vivo Results:

FIG. 57 shows aqueous cell scores for all groups up to Week 12. Compared to AAV2.7m8 fast release group, AAV2.7m8 medium release group had lower scores. The medium release group had lower aqueous cell scores than the bolus and fast release groups between week 4 to week 12.

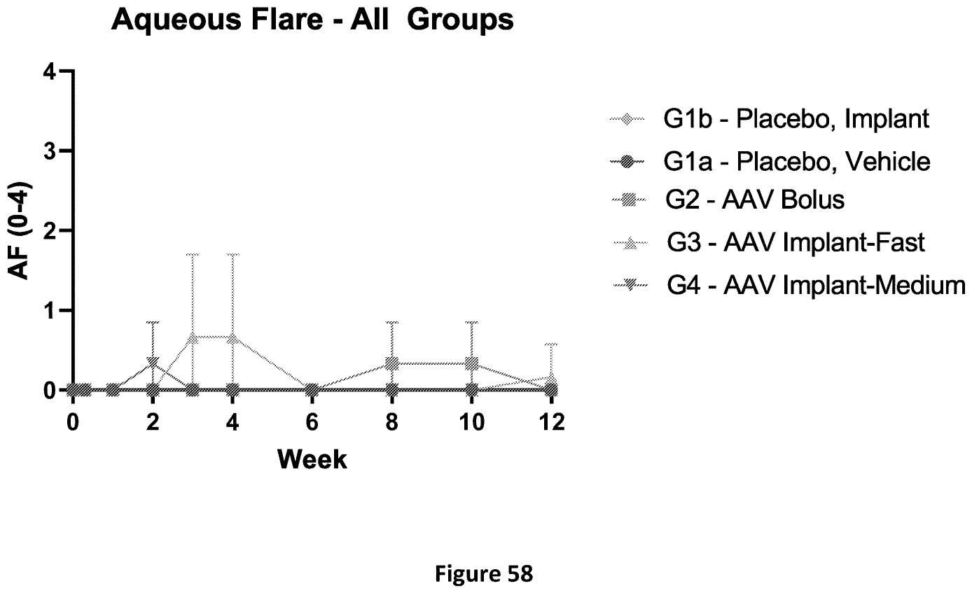

FIG. 58 shows the aqueous flare scores for all groups up to Week 12. Compared to AAV2.7m8 fast release group, AAV2.7m8 medium release group had lower scores. The medium release group had lower aqueous flare scores than the bolus and fast release groups between week 3 to week 12.

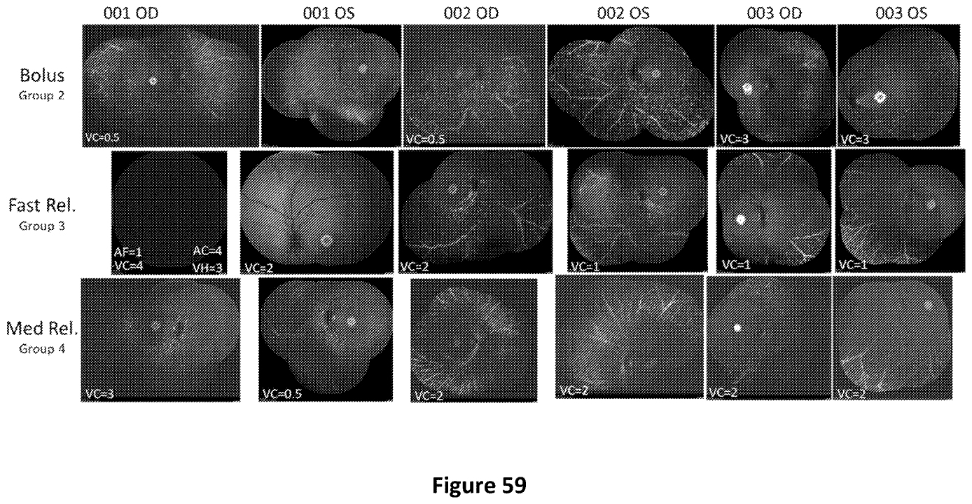

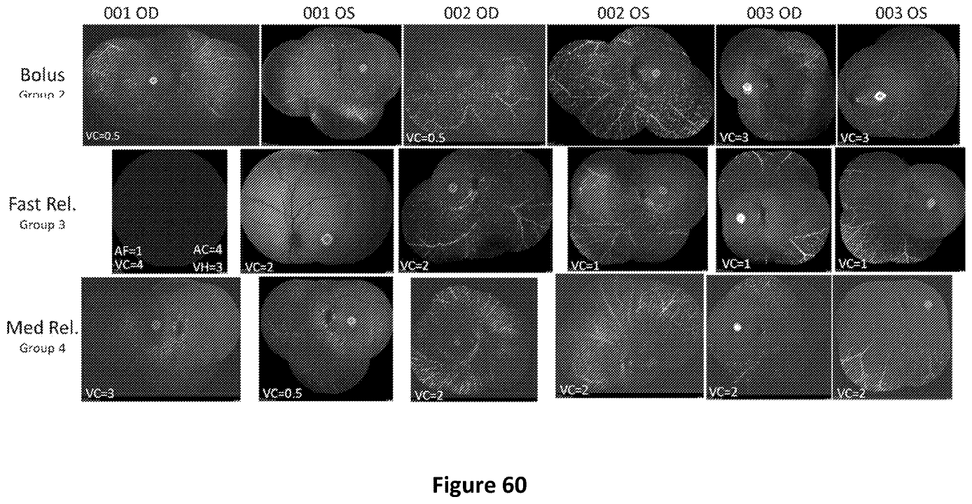

These data, similar to the previous examples show that inflammation can be controlled even when delivering higher dose of AAV. When AAV dose was low (as shown in Example 5—1^{st} rabbit study), the AAV release within 4 days reduced inflammation to none-trace and resulted in steady GFP expression compared to the bolus dose. However, with the implants of the present invention, higher doses can also be delivered. Similar to the 2^{nd} rabbit study (Example 6), higher doses could be delivered to non-human primates with controlled inflammation. This was further supported by the fact that the medium release implant showed GFP expression also in the nonhuman primates similar to rabbits. FIG. 59 and FIG. 60 show FAF images at 8 weeks and 12 weeks, respectively.

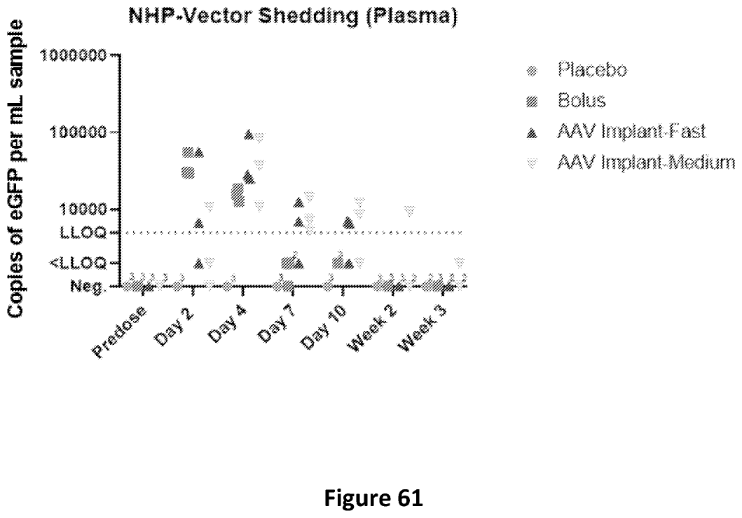

The vector shedding (quantified as copies of eGFP) in the plasma of the animals of each group is shown in FIG. 61.

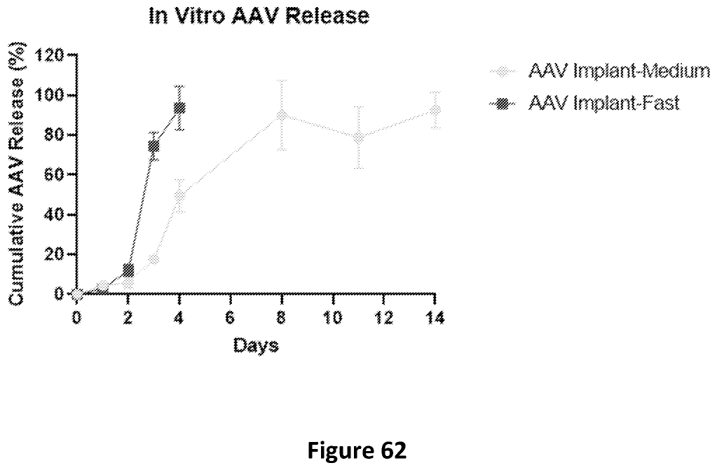

FIG. 62 shows in vitro release profile of AAV2.7m8 from the implants used in the NHP study in this Example (Tables 48 to 50).

### Example 8 (Prophetic)—Use of In-Situ Gel for Sustained Delivery of AAV

An in-situ gel is injected into the suprachoroidal space for sustained delivery of adeno associated viruses (AAVs). The in-situ gel comprises a mixture of AAVs and multi-arm PEG precursors dissolved in phosphate buffered saline prior to gelation, and AAVs entrapped within the PEG hydrogel following gelation. Similar in concept to a two-part epoxy, one PEG precursor solution is mixed with a second PEG precursor solution that initiates the crosslinking reaction between the precursors and forms a hydrogel. The kit for injection into the suprachoroidal space comprises one vial of AAV8-CMV-eGFP solution, a second vial containing a multi-arm PEG with nucleophilic end groups, and a third vial containing a multi-arm PEG with electrophilic end groups. The three solutions are mixed in a 1:1:1 ratio, and 100 μL is injected 4 mm from the limbus in the superior-temporal region into the suprachoroidal space of New Zealand White rabbits. Following injection, the needle is held in place for 30 s to prevent efflux. The liquid mixture spreads throughout the suprachoroidal space following injection prior to gelation. Within minutes, a thin hydrogel film is formed following gelation. Optical coherence tomography imaging post-injection demonstrates a thin hydrogel film formed in the suprachoroidal space. The gel formulation releases AAV over 2 weeks. At 1-month post-injection, fundus autofluorescent imaging reveals GFP expression in the back of the eye and immunohistochemistry demonstrates biodistribution of AAV8-CMV-eGFP with GFP expression in the sclera and retinal pigmented epithelium cells.

### Example 9 (Prophetic)—Use of In-Situ Gel Delivering AAV to Assess in a Preclinical Model of Wet-AMD

An in-situ gel is injected into the suprachoroidal space to evaluate the sustained delivery of AAV in a preclinical model of wet-AMD. The kit for injection into the suprachoroidal space comprises one vial of AAV8 expressing aflibercept using a ubiquitous CMV promoter, a second vial containing a multi-arm PEG with nucleophilic end groups, and a third vial containing a multi-arm PEG with electrophilic end groups. The three solutions are mixed in a 1:1:1 ratio and 100 μL is injected 4 mm from the limbus in the superior-temporal region into the suprachoroidal space of a laser-induced choroidal neovascularization model in non-human primates. Following injection, the needle is held in place for 30 s to prevent efflux. Optical coherence tomography imaging post-injection demonstrates a thin hydrogel film formed in the suprachoroidal space.

All of the references cited above, as well as all references cited herein, are incorporated herein by reference in their entireties.

1. An implant, such as a pharmaceutically acceptable implant, comprising a xerogel, a biologic, and at least one dehydration stabilizer.

2. The pharmaceutically acceptable implant of claim 1, wherein the xerogel comprises a matrix comprising covalently crosslinked multi-arm precursors within which particles comprising a mixture of the biologic, and at least one dehydration stabilizer and optionally at least one further stabilizer such as a buffer and/or a surfactant are dispersed.

3. The pharmaceutically acceptable implant according to claims 1 and 2, wherein the at least one dehydration stabilizer is a carbohydrate, a sugar alcohol, or a combination thereof.

4. The pharmaceutically acceptable implant of claim 2 or 3, wherein the multiarm precursors comprise at least two multi-arm precursors comprising a first multiarm precursor comprising a first functional group, and a second multi-arm precursor comprising a second functional group.

5. The pharmaceutically acceptable implant of claim 4, wherein the multi-arm precursors comprise a third multi-arm precursor comprising the same functional group as the second multi-arm precursor.

6. The pharmaceutically acceptable implant of claim 4 or 5, wherein each of the first functional group and the second functional group is selected from a group consisting of an electrophile and a nucleophile, and the reaction between the first functional group and second functional group is an electrophile-nucleophile reaction that forms the covalent bond.

7. The pharmaceutically acceptable implant of claim 6, wherein the nucleophile comprises an amine such as a primary amine, a thiol, or a hydrazide.

8. The pharmaceutically acceptable implant of claim 6 or 7, wherein the electrophile comprises succinimidyl esters, succinimidyl carbonates, nitrophenyl carbonates, aldehydes, ketones, acrylates, acrylamides, maleimides, vinylsulfones, iodoacetamides, alkenes, alkynes, norbornenes, epoxides, mesylates, tosylates, tresyls, cyanurates, orthopyridyl disulfides, or halides preferably, wherein the succinimidyl ester comprises a reactive group selected from succinimidyl succinate, succinimidyl glutarate, succinimidyl adipate, succinimidyl azelate, and succinimidyl glutaramide.

9. The pharmaceutically acceptable implant of any one of claims 3-8, wherein the carbohydrate is selected from a monosaccharide, a disaccharide, an oligosaccharide, a water-soluble polysaccharide, or any combination thereof.

10. The pharmaceutically acceptable implant of claim 9, wherein the carbohydrate is a sugar.

11. The pharmaceutically acceptable implant of claim 10, wherein the sugar is a non-reducing sugar.

12. The pharmaceutically acceptable implant of claim 10, wherein the sugar is selected from a group consisting of sucrose, trehalose, raffinose, stachyose, verbascose, hydrates thereof and any combination thereof, preferably sucrose, trehalose, trehalose dihydrate, and a combination thereof.

13. The pharmaceutically acceptable implant of any one of claims 3-12, wherein the sugar alcohol is selected from a group consisting of erythritol, glycerol, isomalt, lactitol, maltitol, mannitol, sorbitol, xylitol, and a combination thereof.

14. The pharmaceutically acceptable implant of any one of the preceding claims, wherein the xerogel forms a hydrogel after exposure to an aqueous solution.

15. The pharmaceutically acceptable implant of any one of the preceding claims, wherein the implant is in the form of a fiber, wherein the fiber is characterized by a diameter of about 0.1 mm or more and/or a length of about 2.0 mm or more.

16. The pharmaceutically acceptable implant according to any one of the preceding claims, wherein the implant is for controlled release of a total amount of the biologic, and wherein the controlled release is characterized by:
(A) the amount of the biologic released on day 1 is from 0 to 50% of the total amount of the biologic,
(B) the amount of the biologic released per day from day 2 until the last day of release is from 0 to 50% of the total amount of the biologic, and/or
(C) the number of days required for 100% release of the total amount of the biologic is at least 2 days.

17. The pharmaceutically acceptable implant according to any one of the preceding claims, wherein the implant is for controlled release of a total amount of the biologic, and wherein the controlled release is characterized by:
(A) the amount of the biologic released on day 1 is from 0 to 25%, 0 to 20% 0 to 10%, 0 to 5%, or about 0% of the total amount of the biologic,
(B) the amount of the biologic released per day from day 2 until the last day of release is from 0 to 50% of the total amount of the biologic, and/or
(C) the number of days required for 100% release of the total amount of the biologic is at least 3 days but no greater than 6 weeks, or no greater than 5 weeks, or no greater than 30 days, or no greater than 25 days, or no greater than 16 days.

18. The pharmaceutically acceptable implant for controlled release of claim 16 or 17, wherein the controlled release is measured from the time and under conditions wherein the implant is first immersed in an aqueous solution under physiological conditions such as at pH 7.2-7.4 and temperature 37° C.

19. The pharmaceutically acceptable implant for controlled release according to any one of claims 16-18, wherein the total (w/w) % of the carbohydrate, sugar alcohol, or combination thereof in the implant provides for the controlled release as defined in claim 16 item (A or C) or claim 17 item (A or C), and/or
the D₉₀ particle size such as Dv₉₀ particle size, wherein particles comprise the mixture of the biologic and at least one dehydration stabilizer such as a carbohydrate, a sugar alcohol, or a combination thereof, and optionally at least one further stabilizer such as a buffer and/or a surfactant,
provides for the controlled release as defined in claim 16 item (A or C) or claim 17 item (A or C).

20. The pharmaceutically acceptable implant for controlled release of any one of claims 16-18, wherein the molecular weight between crosslinks in the xerogel provides for the controlled release as defined in claim 16 or 17.

21. The pharmaceutically acceptable implant for controlled release according to any one of claims 16-18, wherein
(i) the (w/w) % of the total particles comprising the mixture of the biologic and the carbohydrate, sugar alcohol, or combination thereof, and optionally at least one further stabilizer such as a buffer and/or a surfactant,
(ii) the (w/w) % of the total number of multi-arm precursors,
(iii) the ratio of (i) and (ii), and/or
(iv) the D90 particle size such as Dv₉₀ particle size, wherein particles comprise the mixture of the biologic and at least one dehydration stabilizer such as a carbohydrate, a sugar alcohol, or a combination thereof, and optionally at least one further stabilizer such as a buffer and/or a surfactant,
provides for the controlled release as defined in claim 16 or 17, and wherein the (w/w) % is based on the weight of the pharmaceutically acceptable implant.

22. The pharmaceutically acceptable implant for controlled release according to any one of claims 16-18, wherein the multi-arm precursors comprise at least three multiarm precursors comprising
(i) a first multi-arm precursor comprising a nucleophile,
(ii) a second multi-arm precursor comprising an electrophile comprising a first reactive group, and
(iii) a third multi-arm precursor comprising an electrophile comprising a second reactive group.

23. The pharmaceutically acceptable implant for controlled release according to claim 22, wherein the third multi-arm precursor has a longer hydrolysis half-life as compared to the second multi-arm precursor.

24. The pharmaceutically acceptable implant for controlled release of any one of claims 22 and 23, wherein the electrophile-nucleophile reaction between the first and the second multi-arm precursors, and the first and the third multi-arm precursors forms the covalent bond.

25. The pharmaceutically acceptable implant for controlled release of claim 24, wherein the nucleophile comprises an amine such as a primary amine, a thiol, or a hydrazide.

26. The pharmaceutically acceptable implant for controlled release of claim 24 or 25, wherein the electrophile comprises succinimidyl esters, succinimidyl carbonates, nitrophenyl carbonates, aldehyde, ketones, acrylates, acrylamides, maleimides, vinylsulfones, iodoacetamides, alkenes, alkynes, norbornenes, epoxides, mesylates, tosylates, tresyls, cyanurates, orthopyridyl disulfides, or halides, preferably, wherein the succinimidyl ester comprises a reactive group selected from succinimidyl succinate, succinimidyl glutarate, succinimidyl adipate, succinimidyl azelate, and succinimidyl glutaramide.

27. The pharmaceutically acceptable implant for controlled release of claims 25 and 26, wherein
(i) the first multi-arm precursor comprises a primary amine,
(ii) the second multi-arm precursor comprises a succinimidyl ester comprising a first reactive group, and,
(iii) the third multi-arm precursor comprises a succinimidyl ester comprising a second reactive group,
wherein the first reactive group is succinimidyl succinate and the second reactive group is succinimidyl glutarate.

28. The pharmaceutically acceptable implant for controlled release according to any one of claims 16-18 and 22-27, wherein the molar ratio of:
(i) the first reactive group comprised in the second multi-arm precursor, and
(ii) the second reactive group comprised in the third multi-arm precursor, provides for the controlled release as defined in claim 16 or 17.

29. The pharmaceutically acceptable implant for controlled release according to any one of claims 16-28, wherein the controlled release is achieved by
(a) the total (w/w) % of the carbohydrate, sugar alcohol, or combination thereof,
(b) the molecular weight between crosslinks in the xerogel,
(c) the (w/w) % of the total particles comprising the mixture of the biologic and the carbohydrate, sugar alcohol, or combination thereof, and optionally at least one further stabilizer such as a buffer and/or a surfactant,
(d) the (w/w) % of the total number of multi-arm precursors,
(e) the ratio of (c) and (d),
(f) the D₉₀ particle size such as Dv₉₀ particle size, wherein particles comprise the mixture of the biologic and at least one dehydration stabilizer such as a carbohydrate, a sugar alcohol, or a combination thereof, and optionally at least one further stabilizer such as a buffer and/or a surfactant, and/or
(g) the molar ratio of:
(g-i) the first reactive group comprised in the second multi-arm precursor and
(g-ii) the second reactive group comprised in the third multi-arm precursor, wherein the (w/w) % is based on the weight of the pharmaceutically acceptable implant.

30. The pharmaceutically acceptable implant according to any one of claims 3-29, wherein the total (w/w) % of the carbohydrate, sugar alcohol, or combination thereof is no greater than 40%, such as from 5 to 40%, wherein the (w/w) % is based on the weight of the pharmaceutically acceptable implant.

31. The pharmaceutically acceptable implant according to claim 30, wherein the total (w/w) % of the carbohydrate, sugar alcohol, or combination thereof is from 10 to 35%, wherein the (w/w) % is based on the weight of the pharmaceutically acceptable implant.

32. The pharmaceutically acceptable implant according to any one of the preceding claims, wherein the molecular weight between crosslinks in the xerogel is from 7 to 25 kDa.

33. The pharmaceutically acceptable implant according to claim 32, wherein the molecular weight between crosslinks in the xerogel is from 9 to 16 kDa.

34. The pharmaceutically acceptable implant according to any one of claims 3-33, wherein:
(i) the (w/w) % of the total particles comprising the mixture of the biologic and the carbohydrate, sugar alcohol, or combination thereof, and optionally at least one further stabilizer such as a buffer and/or a surfactant is no greater than 80%, or no greater than 70%, or no greater than 60%, or no greater than 50%,
(ii) the (w/w) % of the total number of multi-arm precursors is from 20% to 80% such as from 35% to 75%, and/or
(iii) the ratio of (i) and (ii) is from 0.3 to 4.0 such as from 0.3 to 2.0,
(iv) the Dv₉₀ particle size is from 10 μm to 200 μm such as 35 μm to 75 μm such as 35 μm to 100 μm, or 35 μm to 150 μm, wherein particles comprise the mixture of the biologic and at least one dehydration stabilizer such as a carbohydrate, a sugar alcohol, or a combination thereof, and optionally at least one further stabilizer such as a buffer and/or a surfactant,
wherein the (w/w) % is based on the weight of the pharmaceutically acceptable implant.

35. The pharmaceutically acceptable implant according to claim 34, wherein the ratio is from 0.5 to 2.0, from 0.5 to 1.0, from 0.7 to 1.3 such as from 0.6 to 0.9.

36. The pharmaceutically acceptable implant of any one of claims 8-35, wherein the molar ratio of:
(i) the first reactive group comprised in the second multi-arm precursor and
(ii) the second reactive group comprised in the third multi-arm precursor, is from 0:100 to 100:0.

37. The pharmaceutically acceptable implant according to claim 36, wherein the ratio is from 30-90:70-10.

38. The pharmaceutically acceptable implant according to claim 36 or 37, wherein the first reactive group is succinimidyl succinate and the second reactive group is succinimidyl glutarate.

39. The pharmaceutically acceptable implant according to any one of claims 3-38, wherein
(a) the total (w/w) % of the carbohydrate, sugar alcohol, or combination thereof is no greater than 40%, such as from 5 to 40%,
(b) the molecular weight between crosslinks in the xerogel is from 7 to 25 kDa,
(c) the (w/w) % of the total particles comprising the mixture of the biologic and the carbohydrate, sugar alcohol, or combination thereof, and optionally at least one further stabilizer such as a buffer and/or a surfactant is no greater than 80%, or no greater than 70%, or no greater than 60%, or no greater than 50%,
(d) the (w/w) % of the total number of multi-arm precursors is from 20% to 80% such as from 35% to 75%,
(e) the ratio of (c) and (d) is from 0.3 to 4.0 such as from 0.3 to 2.0, and/or
(f) the Dv₉₀ particle size is from 10 μm to 200 μm such as 35 μm to 75 μm such as 35 μm to 100 μm, or 35 μm to 150 μm, wherein particles comprise the mixture of the biologic and at least one dehydration stabilizer such as a carbohydrate, a sugar alcohol, or a combination thereof, and optionally at least one further stabilizer such as a buffer and/or a surfactant,
(g) the molar ratio of:
(g-i) the first reactive group comprised in the second multi-arm precursor and
(g-ii) the second reactive group comprised in the third multi-arm precursor,
is from is from 30-90:70:10, wherein the first reactive group is succinimidyl succinate and the second reactive group is succinimidyl glutarate,
wherein the (w/w) % is based on the weight of the pharmaceutically acceptable implant.

40. The pharmaceutically acceptable implant according to any one of the preceding claims, wherein the implant further comprises a polymer that does not participate in the cross-linking reaction between the multi-arm precursors, wherein the MW of the polymer is from 1,000 to 35,000 Da.

41. The pharmaceutically acceptable implant of claim 40, wherein the polymer is selected from a group consisting of polyalkylene oxide such as polyethylene glycol, polyvinyl pyrrolidinone, and polyvinyl alcohol.

42. The pharmaceutically acceptable implant for controlled release according to any one of claims 16-41, wherein the controlled release comprises a zero-order release or substantially zero-order release.

43. The pharmaceutically acceptable implant for controlled release of claim 42, wherein the zero-order release starts at least one day after the implant is first immersed in an aqueous solution under physiological conditions such as at pH 7.2-7.4 and temperature 37° C.

44. The pharmaceutically acceptable implant for controlled release according to any one of claims 16-43, wherein the controlled release is characterized by at least 3 days to 65 days.

45. The pharmaceutically acceptable implant for controlled release of any one of claims 16-44, wherein the implant is in the form of a fiber, and wherein during the controlled release,
(i) the diameter and/or the length of the fiber increases, such as at least by 1.5 folds,
(ii) the length of the fiber does not change, and/or
(iii) the diameter and/or length of the fiber decreases,
as measured after the implant is first immersed in an aqueous solution under physiological conditions such as at pH 7.2-7.4 and temperature 37° C.

46. The pharmaceutically acceptable implant of any one of the preceding claims, wherein the biologic comprises a plurality of the same or different biologics.

47. The pharmaceutically acceptable implant of claim 46, wherein the biologic is selected from a group consisting of a polypeptide, a virus or a virus-like particle, and a lipid encapsulating a nucleic acid(s).

48. The pharmaceutically acceptable implant of claim 47, wherein the polypeptide is any polypeptide having a primary, secondary, tertiary or quaternary structure.

49. The pharmaceutically acceptable implant of claim 48, wherein the polypeptide is a recombinant protein.

50. The pharmaceutically acceptable implant of claim 49, wherein the recombinant protein is selected from an antibody, an antigen binding fragment, a fusion protein, a cytokine, a transcription factor, an enzyme or a hormone.

51. The pharmaceutically acceptable implant of claim 47, wherein the lipid encapsulating nucleic acid(s) comprises one or more heterologous nucleic acid(s).

52. The pharmaceutically acceptable implant of claim 47, wherein the virus or virus-like particle comprises a viral nucleic acid and one or more heterologous nucleic acid(s).

53. The pharmaceutically acceptable implant of any one of claims 51 and 52, wherein each heterologous nucleic acid is selected from a group consisting of DNA and RNA.

54. The pharmaceutically acceptable implant of claim 53, wherein the heterologous nucleic acid is a non-coding nucleic acid selected from a group consisting of a ssDNA (single-strand DNA), dsDNA (double-stranded DNA), small interfering RNA (siRNA), micro-RNA, dsRNA, lncRNA, piRNA, rmRNA, sRNA, tiRNA, eRNA, snoRNA, snRNA, circRNA (circular RNA), RNA aptamer, antisense oligonucleotide, a guide RNA, a tRNA or any combination thereof.

55. The pharmaceutically acceptable implant of claim 53, wherein the heterologous nucleic acid comprises a coding nucleic acid sequence.

56. The pharmaceutically acceptable implant of claim 55, wherein the coding nucleic acid sequence codes for a therapeutic protein.

57. The pharmaceutically acceptable implant of any one of claim 49-50 or 56, wherein the recombinant protein or the therapeutic protein is selected from a group consisting of RPE65, REP1, RPGR, BEST1, anti-VEGF inhibitors such as aflibercept, ranibizumab, brolucizumab, or bevacizumab, pegatanib sodium, adalimumab, Infliximab, hRS1, hCNGB3, ABCR, MYO7A, endostatin, angiostatin, TNF [alpha] receptor, the TGF [beta]2 receptor, IRS-1, IGF-1, Angiogenin, Angiopoietin-1, DeM, acidic or basic Fibroblast Growth Factors (aFGF and bFGF), FGF-2, Follistatin, Granulocyte Colony-Stimulating factor (G-CSF), Hepatocyte Growth Factor (HGF), Scatter Factor (SF), Leptin, Midkine, Placental Growth Factor (PGF), Platelet-Derived Endothelial Cell Growth Factor (PD-ECGF), Platelet-Derived Growth Factor-BB (PDGFBB), Pleiotrophin (PTN), RdCVF (Rod-derived Cone Viability Factor), Progranulin, Proliferin, Transforming Growth Factor-alpha (TGF-alpha), PEDF, Transforming Growth Factor-beta (TGF-beta), Vascular Permeability Factor (VPF), CNTF, BDNF, GDNF, PEDF, NT3, BFGF, ephrin, EPO, NGF, GMF, aFGF, NT5, Gax, a growth hormone, [alpha]-1-antitrypsin, calcitonin, leptin, an apolipoprotein, an enzyme for the biosynthesis of vitamins, hormones or neuromediators, chemokines, cytokines such as IL-1, IL-8, IL-10, IL-12, IL-13, a receptor thereof, an antibody blocking any one of said receptors, TIMP such as TIMP-1, TIMP-2, TIMP-3, TIMP-4, angioarrestin, endostatin such as endostatin XVIII and endostatin XV, ATF, a fusion protein of endostatin and angiostatin, the C-terminal hemopexin domain of matrix metalloproteinase-2, the kringle 5 domain of human plasminogen, a fusion protein of endostatin and the kringle 5 domain of human plasminogen, the placental ribonuclease inhibitor, the plasminogen activator inhibitor, the Platelet Factor-4 (PF4), a prolactin fragment, the Proliferin-Related Protein (PRP), the antiangiogenic antithrombin III, the Cartilage-Derived Inhibitor (CDI), a CD59 complement fragment, C3a and C5a inhibitors, complex attack membrane inhibitors, Factor H, ICAM, VCAM, caveolin, PKC zeta, junction proteins, JAMs, CD36, MERTK vasculostatin, vasostatin (calreticulin fragment), thrombospondin, fibronectin, in particular fibronectin fragment gro-beta, an heparinase, human chorionic gonadotropin (hCG), interferon alpha/beta/gamma, interferon inducible protein (IP-10), the monokine-induced by interferon-gamma (Mig), the interferon-alpha inducible protein 10 (IP10), a fusion protein of Mig and IP10, soluble Fms-Like Tyrosine kinase 1 (FLT-1) receptor, Kinase insert Domain Receptor (KDR), regulators of apoptosis such as Bcl-2, Bad, Bak, Bax, Bik, BcI-X short isoform and Gax, alpha-1 antitrypsin, factor IX, factor VIII, CI-esterase inhibitor, β-globin or γ-globin.

58. The pharmaceutically acceptable implant of any one of claims 47, 52-57, wherein the virus is selected from a group consisting of retrovirus, adenovirus, adenoassociated virus (AAV), lentivirus and herpes simplex virus.

59. The pharmaceutically acceptable implant of claim 58, wherein the virus is adeno-associated virus (AAV).

60. The pharmaceutically acceptable implant of claim 59, wherein the adenoassociated virus (AAV) is selected from a group consisting of AAV1, AAV2 such as AAV2.7m8, AAV3, AAV4, AAV5, AAV6, AAV7, AAV8, AAV9, AAV10, AAV11 and a mutant, hybrid or variant thereof.

61. The pharmaceutically acceptable implant of any one of claims 1 to 47, and 52-60, wherein the biologic is a virus or virus-like particle, wherein the virus is adenoassociated virus (AAV), and wherein AAV is selected from a group consisting of AAV2, AAV2.7m8, and AAV8.

62. The pharmaceutically acceptable implant of claims 59-61, wherein the total amount of the AAV comprised in the implant is in the order of at least 10⁹ vg.

63. The pharmaceutically acceptable implant of claim 61 or 62, wherein the total amount of the AAV comprised in the implant is in the order from 10⁹ to 10¹⁵ vg such as 10¹⁰ to 10¹³ vg.

64. The pharmaceutically acceptable implant of claim 63, wherein the total concentration of the AAV comprised in the implant is at least 10¹³ vg/cm³.

65. The pharmaceutically acceptable implant for controlled release according to any one of claims 59-63, wherein the controlled release is characterized by
(A) no greater than 9.0×10⁹ to 1.5×10¹⁰ AAV vg released on day 1,
(B) no greater than 5.0×10⁹ to 1.5×10¹⁰ AAV vg released per day from day 2 until the last day of the controlled release, and/or
(C) the number of days required for 100% release of the AAV is not less than 4 days.

66. A pharmaceutically acceptable implant comprising
(i) a xerogel comprising a matrix comprising covalently crosslinked multi-arm precursors comprising,
(i-a) a first multi-arm precursor comprising a nucleophile,
(i-b) a second multi-arm precursor comprising an electrophile,
(i-c) optionally, a third multi-arm precursor comprising an electrophile, and
(ii) particles comprising a mixture of a biologic and at least one dehydration stabilizer selected from a carbohydrate such as a sugar, such as sucrose and trehalose, a sugar alcohol, or a combination thereof, and optionally at least one further stabilizer such as a buffer and/or a surfactant,
wherein the particles are dispersed within the xerogel,
and wherein the total (w/w) % of said dehydration stabilizer is no greater than 40%, such as from 5 to 40%, and/or
the Dv₉₀ particle size is from 10 μm to 200 μm such as 35 μm to 75 μm such as 35 μm to 100 μm, or 35 μm to 150 μm, wherein particles comprise said mixture of the biologic and said dehydration stabilizer, and optionally said further stabilizer,
wherein the (w/w) % is based on the weight of the pharmaceutically acceptable implant.

67. A pharmaceutically acceptable implant for controlled release of a biologic comprising
(i) a xerogel comprising a matrix comprising covalently crosslinked multi-arm precursors comprising,
(i-a) a first multi-arm precursor comprising a nucleophile,
(i-b) a second multi-arm precursor comprising an electrophile, and
(i-c) optionally, a third multi-arm precursor comprising an electrophile, and
(ii) particles comprising a mixture of a biologic and at least one dehydration stabilizer selected from a carbohydrate such as a sugar, such as sucrose and trehalose, a sugar alcohol, or a combination thereof,
wherein the particles are dispersed within the xerogel, and wherein the total (w/w) % of said dehydration stabilizer is no greater than 40%, such as from 5 to 40%, wherein the (w/w) % is based on the weight of the pharmaceutically acceptable implant,
and wherein the controlled release of the biologic is characterized in that the number of days required for 100% release is at least 2 days, wherein the controlled released is measured from the time and under conditions wherein the implant is first immersed in an aqueous solution under physiological conditions such as at pH 7.2-7.4 and temperature 37° C.,
and/or
A pharmaceutically acceptable implant for controlled release of a biologic comprising
(i) a xerogel comprising a matrix comprising covalently crosslinked multi-arm precursors comprising,
(i-a) a first multi-arm precursor comprising a nucleophile,
(i-b) a second multi-arm precursor comprising an electrophile, and
(i-c) optionally, a third multi-arm precursor comprising an electrophile, and
(ii) particles comprising a mixture of a biologic and at least one dehydration stabilizer selected from a carbohydrate such as a sugar, such as sucrose and trehalose, a sugar alcohol, or a combination thereof, and optionally at least one further stabilizer such as a buffer and/or a surfactant, wherein the particles are dispersed within the xerogel,
wherein the Dv₉₀ particle size is from 10 μm to 200 μm such as 35 μm to 75 μm such as 35 μm to 100 μm, or 35 μm to 150 μm, wherein particles comprise said mixture of the biologic and said dehydration stabilizer and optionally said further stabilizer, and wherein the controlled release of the biologic is characterized in that the percentage release of the biologic on day 1 is from 0 to 50% of the total amount of the biologic, wherein the controlled released is measured from the time and under conditions wherein the implant is first immersed in an aqueous solution under physiological conditions such as at pH 7.2-7.4 and temperature 37° C.

68. A pharmaceutically acceptable implant comprising,
(i) a xerogel comprising a matrix comprising covalently crosslinked multi-arm precursors comprising,
(i-a) a first multi-arm precursor comprising a nucleophile,
(i-b) a second multi-arm precursor comprising an electrophile comprising a first reactive group, and
(i-c) a third multi-arm precursor comprising an electrophile comprising a second reactive group,
and
(ii) particles comprising a mixture of a biologic and at least one dehydration stabilizer selected from a carbohydrate such as a sugar, such as sucrose and trehalose, a sugar alcohol, or a combination thereof,
wherein the third multi-arm precursor has a longer hydrolysis half-life as compared to the second multi-arm precursor, and wherein the molar ratio of:
(i) the first reactive group comprised in the second multi-arm precursor, and
(ii) the second reactive group comprised in the third multi-arm precursor,
is 30-90:70-10.

69. A pharmaceutically acceptable implant for controlled release of a biologic comprising,
(i) a xerogel comprising a matrix comprising covalently crosslinked multi-arm precursors comprising,
(i-a) a first multi-arm precursor comprising a nucleophile,
(i-b) a second multi-arm precursor comprising an electrophile comprising a first reactive group, and
(i-c) a third multi-arm precursor comprising an electrophile comprising a second reactive group,
and
(ii) particles comprising a mixture of a biologic and at least one dehydration stabilizer selected from a carbohydrate such as a sugar, such as sucrose and trehalose, a sugar alcohol, or a combination thereof,
wherein the third multi-arm precursor has a longer hydrolysis half-life as compared to the second multi-arm precursor, and wherein the molar ratio of:
(i) the first reactive group comprised in the second multi-arm precursor, and
(ii) the second reactive group comprised in the third multi-arm precursor, is 30-90:70-10,
and wherein the controlled release of the total amount of the biologic is characterized in that
(A) the percentage release of the biologic on day 1 is from 0 to 50% of the total amount of the biologic,
(B) the percentage release of the biologic per day from day 2 until the last day of release is from 0 to 50% of the total amount of the biologic, and/or
(C) number of days required for 100% release of the total amount of the biologic is at least 2 days,
wherein the controlled released is measured from the time and under conditions wherein the implant is first immersed in an aqueous solution under physiological conditions such as at pH 7.2-7.4 and temperature 37° C.

70. A pharmaceutically acceptable implant comprising,
(i) a xerogel comprising a matrix comprising covalently crosslinked multi-arm precursors comprising,
(i-a) a first multi-arm precursor comprising a primary amine,
(i-b) a second multi-arm precursor comprising succinimidyl succinate, and
(i-c) a third multi-arm precursor comprising succinimidyl glutarate,
and
(ii) particles comprising a mixture of a biologic and at least one dehydration stabilizer selected from a carbohydrate such as a sugar, such as sucrose and trehalose, a sugar alcohol, or a combination thereof,
wherein the molar ratio of:
(i) the succinimidyl succinate group comprised in the second multi-arm precursor, and
(ii) the succinimidyl glutarate group comprised in the third multi-arm precursor, is 30-90:70-10.

71. A pharmaceutically acceptable implant for controlled release of a biologic comprising,
(i) a xerogel comprising a matrix comprising covalently crosslinked multi-arm precursors comprising,
(i-a) a first multi-arm precursor comprising a primary amine,
(i-b) a second multi-arm precursor comprising succinimidyl succinate, and
(i-c) a third multi-arm precursor comprising succinimidyl glutarate,
and
(ii) particles comprising a mixture of a biologic and at least one dehydration stabilizer selected from a carbohydrate such as a sugar, such as sucrose and trehalose, a sugar alcohol, or a combination thereof,
wherein the molar ratio of:
(i) the succinimidyl succinate group comprised in the second multi-arm precursor, and
(ii) the succinimidyl glutarate group comprised in the third multi-arm precursor, is 30-90:70-10,
wherein the controlled release of the total amount of the biologic is characterized in that
(A) the percentage release of the biologic on day 1 is from 0 to 50% of the total amount of the biologic,
(B) the percentage release of the biologic per day from day 2 until the last day of release is from 0 to 50% of the total amount of the biologic, and/or
(C) number of days required for 100% release of the total amount of the biologic is at least 2 days,
wherein the controlled released is measured from the time and under conditions wherein the implant is first immersed in an aqueous solution under physiological conditions such as at pH 7.2-7.4 and temperature 37° C.

72. A pharmaceutically acceptable implant comprising,
(i) a xerogel comprising a matrix comprising covalently crosslinked multi-arm precursors comprising,
(i-a) a first multi-arm precursor comprising a nucleophile,
(i-b) a second multi-arm precursor comprising an electrophile, and
(i-c) optionally a third multi-arm precursor comprising an electrophile,
and
(ii) particles comprising a mixture of a biologic and at least one dehydration stabilizer selected from a carbohydrate such as a sugar, such as sucrose and trehalose, a sugar alcohol, or a combination thereof,
wherein the molecular weight between crosslinks in the xerogel is from 7 to 25 kDa.

73. A pharmaceutically acceptable implant for controlled release of a biologic comprising,
(i) a xerogel comprising a matrix comprising covalently crosslinked multi-arm precursors comprising,
(i-a) a first multi-arm precursor comprising a nucleophile,
(i-b) a second multi-arm precursor comprising an electrophile, and
(i-c) optionally a third multi-arm precursor comprising an electrophile,
and
(ii) particles comprising a mixture of a biologic and at least one dehydration stabilizer selected from a carbohydrate such as a sugar, such as sucrose and trehalose, a sugar alcohol, or a combination thereof,
wherein the molecular weight between crosslinks is from 7 to 25 kDa,
and wherein the controlled release of the total amount of the biologic is characterized in that
(A) the percentage release of the biologic on day 1 is from 0 to 50% of the total amount of the biologic,
(B) the percentage release of the biologic per day from day 2 until the last day of release is from 0 to 50% of the total amount of the biologic, and/or
(C) number of days required for 100% release of the total amount of the biologic is at least 2 days,
wherein the controlled released is measured from the time and under conditions wherein the implant is first immersed in an aqueous solution under physiological conditions such as at pH 7.2-7.4 and temperature 37° C.

74. A pharmaceutically acceptable implant comprising,
(i) a xerogel comprising a matrix comprising covalently crosslinked multi-arm precursors comprising,
(i-a) a first multi-arm precursor comprising a nucleophile,
(i-b) a second multi-arm precursor comprising an electrophile, and
(i-c) optionally a third multi-arm precursor comprising an electrophile,
and
(ii) particles comprising a mixture of a biologic and at least one dehydration stabilizer selected from a carbohydrate such as a sugar, such as sucrose and trehalose, a sugar alcohol, or a combination thereof, and optionally at least one further stabilizer such as a buffer and/or a surfactant,
wherein
(a) the (w/w) % of the total particles comprising a mixture of the biologic and said dehydration stabilizer, and optionally said further stabilizer is no greater than 80%, or no greater than 70%, or no greater than 60%, or no greater than 50%,
(b) the (w/w) % of the total number of multi-arm precursors is from 20% to 80% such as from 35% to 75%,
(c) the ratio of (a) and (b) is from 0.3 to 4.0 such as from 0.3 to 2.0, and/or
(d) the Dv₉₀ particle size is from 10 μm to 200 μm such as 35 μm to 75 μm such as 35 μm to 100 μm, or 35 μm to 150 μm, wherein particles comprise said mixture of the biologic and said dehydration stabilizer and optionally said further stabilizer, wherein the (w/w) % is based on the weight of the pharmaceutically acceptable implant.

75. A pharmaceutically acceptable implant comprising for controlled release of a biologic comprising,
(i) a xerogel comprising a matrix comprising covalently crosslinked multi-arm precursors comprising,
(i-a) a first multi-arm precursor comprising a nucleophile,
(i-b) a second multi-arm precursor comprising an electrophile, and
(i-c) optionally a third multi-arm precursor comprising an electrophile, and
(ii) particles comprising a mixture of a biologic and at least one dehydration stabilizer selected from a carbohydrate such as a sugar, such as sucrose and trehalose, a sugar alcohol, or a combination thereof, and optionally at least one further stabilizer such as a buffer and/or a surfactant,
wherein
(a) the (w/w) % of the total particles comprising a mixture of the biologic and said dehydration stabilizer and optionally said further stabilizer is no greater than 80%, or no greater than 70%, or no greater than 60%, or no greater than 50%,
(b) the (w/w) % of the total number of multi-arm precursors is from 20% to 80% such as from 35% to 75%,
(c) the ratio of (a) and (b) is from 0.3 to 4.0 such as from 0.3 to 2.0, and/or
(d) the Dv₉₀ particle size is from 10 μm to 200 μm such as 35 μm to 75 μm such as 35 μm to 100 μm, or 35 μm to 150 μm, wherein particles comprise said mixture of the biologic and said dehydration stabilizer and optionally said further stabilizer, wherein the (w/w) % is based on the weight of the pharmaceutically acceptable implant, and wherein the controlled release of the total amount of the biologic is characterized in that
(A) the percentage release of the biologic on day 1 is from 0 to 50% of the total amount of the biologic,
(B) the percentage release of the biologic per day from day 2 until the last day of release is from 0 to 50% of the total amount of the biologic, and/or
(C) number of days required for 100% release of the total amount of the biologic is at least 2 days,
wherein the controlled released is measured from the time and under conditions wherein the implant is first immersed in an aqueous solution under physiological conditions such as at pH 7.2-7.4 and temperature 37° C.,

76. A pharmaceutically acceptable implant comprising,
(i) a xerogel comprising a matrix comprising covalently crosslinked multi-arm precursors comprising,
(i-a) a first multi-arm precursor comprising a nucleophile,
(i-b) a second multi-arm precursor comprising an electrophile comprising a first reactive group, and
(i-c) a third multi-arm precursor comprising an electrophile comprising a second reactive group,
and
(ii) particles comprising a mixture of a biologic and at least one dehydration stabilizer selected from a carbohydrate such as a sugar, such as sucrose and trehalose, a sugar alcohol, or a combination thereof, and optionally at least one further stabilizer such as a buffer and/or a surfactant,
wherein,
(i) the total (w/w) % of said dehydration stabilizer is no greater than 40%, such as from 5 to 40%,
(ii) the molecular weight between crosslinks in the xerogel is from 7 to 25 kDa,
(iii) the (w/w) % of the total particles comprising a mixture of the biologic and said dehydration stabilizer, and optionally said further stabilizer is no greater than 80%, or no greater than 70%, or no greater than 60%, or no greater than 50%,
(iv) the (w/w) % of the total number of multi-arm precursors is from 20% to 80% such as from 35% to 75%,
(v) the ratio of (iii) and (iv) is from 0.3 to 4.0 such as from 0.3 to 2.0,
(vi) the Dv₉₀ particle size is from 10 μm to 200 μm such as 35 μm to 75 μm such as 35 μm to 100 μm, or 35 μm to 150 μm, wherein particles comprise said mixture of the biologic and said dehydration stabilizer and optionally said further stabilizer,
(vii) the molar ratio of:
(vii-a) the first reactive group comprised in the second multi-arm precursor, and
(vii-b) the second reactive group comprised in the third multi-arm precursor, is from is from 30-90:70-10, wherein the first reactive group is succinimidyl succinate and the second reactive group is succinimidyl glutarate,
wherein the (w/w) % is based on the weight of the pharmaceutically acceptable implant.

77. A pharmaceutically acceptable implant for controlled release of a biologic comprising,
(i) a xerogel comprising a matrix comprising covalently crosslinked multi-arm precursors comprising,
(i-a) a first multi-arm precursor comprising a nucleophile,
(i-b) a second multi-arm precursor comprising an electrophile comprising a first reactive group, and
(i-c) a third multi-arm precursor comprising an electrophile comprising a second reactive group,
and
(ii) particles comprising a mixture of a biologic and at least one dehydration stabilizer selected from a carbohydrate such as a sugar, such as sucrose and trehalose, a sugar alcohol, or a combination thereof, and optionally at least one further stabilizer such as a buffer and/or a surfactant,
wherein,
(i) the total (w/w) % of said dehydration stabilizer is no greater than 40%, such as from 5 to 40%,
(ii) the molecular weight between crosslinks in the xerogel is from 7 to 25 kDa,
(iii) the (w/w) % of the total particles comprising a mixture of the biologic and said dehydration stabilizer and optionally said further stabilizer are no greater than 80%, or no greater than 70%, or no greater than 60%, or no greater than 50%,
(iv) the (w/w) % of the total number of multi-arm precursors is from 20% to 80% such as from 35% to 75%,
(v) the ratio of (iii) and (iv), is from 0.3 to 4.0 such as from 0.3 to 2.0,
(vi) the Dv₉₀ particle size is from 10 μm to 200 μm such as 35 μm to 75 μm such as 35 μm to 100 μm, or 35 μm to 150 μm, wherein particles comprise said mixture of the biologic and said dehydration stabilizer and optionally said further stabilizer, and/or
(vii) the molar ratio of:
(vii-a) the first reactive group comprised in the second multi-arm precursor and
(vii-b) the second reactive group comprised in the third multi-arm precursor, is from 30-90:70-10, wherein the first reactive group is succinimidyl succinate and the second reactive group is succinimidyl glutarate,
wherein the (w/w) % is based on the weight of the pharmaceutically acceptable implant,
and wherein the controlled release of the total amount of the biologic is characterized in that
(A) the percentage release of the biologic on day 1 is from 0 to 50% of the total amount of the biologic,
(B) the percentage release of the biologic per day from day 2 until the last day of release is from 0 to 50% of the total amount of the biologic, and/or
(C) number of days required for 100% release of the total amount of the biologic is at least 2 days,
wherein the controlled released is measured from the time and under conditions wherein the implant is first immersed in an aqueous solution under physiological conditions such as at pH 7.2-7.4 and temperature 37° C.,

78. A pharmaceutically acceptable implant comprising
(i) a xerogel comprising a matrix comprising covalently crosslinked multi-arm precursors comprising,
(i-a) a first multi-arm precursor comprising a nucleophile,
(i-b) a second multi-arm precursor comprising an electrophile, and
(i-c) optionally, a third multi-arm precursor comprising an electrophile,
and
(ii) particles comprising a mixture of AAV selected from a group consisting of AAV1, AAV2 such as AAV2.7m8, AAV3, AAV4, AAV5, AAV6, AAV7, AAV8, AAV9, AAV10, AAV11 and a mutant, hybrid or variant thereof at a total amount in the order from 10⁹ to 10¹⁵ vg such as 10¹⁰ to 10¹³ vg and at least one dehydration stabilizer selected from a carbohydrate such as a sugar, such as sucrose or trehalose, a sugar alcohol, or a combination thereof, and optionally at least one further stabilizer such as a buffer and/or a surfactant, wherein the AAV comprises at least one heterologous nucleic acid sequence that codes for a therapeutic protein,
wherein the particles are dispersed within the xerogel,
and wherein the total (w/w) % of said dehydration stabilizer is no greater than 40%, such as from 5 to 40%, and/or
the Dv₉₀ particle size is from 10 μm to 200 μm such as 35 μm to 75 μm such as 35 μm to 100 μm, or 35 μm to 150 μm, wherein particles comprise said mixture of the AAV and said dehydration stabilizer and optionally said further stabilizer,
wherein the (w/w) % is based on the weight of the pharmaceutically acceptable implant.

79. A pharmaceutically acceptable implant for controlled release of AAV comprising
(i) a xerogel comprising a matrix comprising covalently crosslinked multi-arm precursors comprising,
(i-a) a first multi-arm precursor comprising a nucleophile,
(i-b) a second multi-arm precursor comprising an electrophile, and
(i-c) optionally, a third multi-arm precursor comprising an electrophile, and
(ii) particles comprising a mixture of AAV selected from a group consisting of AAV1, AAV2 such as AAV2.7m8, AAV3, AAV4, AAV5, AAV6, AAV7, AAV8, AAV9, AAV10, AAV11 and a mutant, hybrid or variant thereof at a total amount in the order from 10⁹ to 10¹⁵ vg such as 10¹⁰ to 10¹³ vg and at least one dehydration stabilizer selected from a carbohydrate such as a sugar, such as sucrose or trehalose, a sugar alcohol, or a combination thereof, and optionally at least one further stabilizer such as a buffer and/or a surfactant, wherein the AAV comprises at least one heterologous nucleic acid sequence that codes for a therapeutic protein,
wherein the particles are dispersed within the xerogel, and wherein the total (w/w) % of said dehydration stabilizer is no greater than 40% w/w, such as from 5 to 40%,
wherein the (w/w) % is based on the weight of the pharmaceutically acceptable implant, and wherein the controlled release of the AAV is characterized in that the number of days required for 100% release is at least 2 days, and wherein the controlled released is measured from the time and under conditions wherein the implant is first immersed in an aqueous solution under physiological conditions such as at pH 7.2-7.4 and temperature 37° C.,
and/or
A pharmaceutically acceptable implant for controlled release of AAV comprising
(i) a xerogel comprising a matrix comprising covalently crosslinked multi-arm precursors comprising,
(i-a) a first multi-arm precursor comprising a nucleophile,
(i-b) a second multi-arm precursor comprising an electrophile, and
(i-c) optionally, a third multi-arm precursor comprising an electrophile, and
(ii) particles comprising a mixture of AAV selected from a group consisting of AAV1, AAV2 such as AAV2.7m8, AAV3, AAV4, AAV5, AAV6, AAV7, AAV8, AAV9, AAV10, AAV11 and a mutant, hybrid or variant thereof at a total amount in the order from 10⁹ to 10¹⁵ vg such as 10¹⁰ to 10¹³ vg and at least one dehydration stabilizer selected from a carbohydrate such as a sugar, such as sucrose or trehalose, a sugar alcohol, or a combination thereof, and optionally at least one further stabilizer such as a buffer and/or a surfactant,
wherein the AAV comprises at least one heterologous nucleic acid sequence that codes for a therapeutic protein,
wherein the particles are dispersed within the xerogel,
wherein the Dv₉₀ particle size is from 10 μm to 200 μm such as 35 μm to 75 μm such as 35 μm to 100 μm, or 35 μm to 150 μm, wherein particles comprise said mixture of the AAV and said dehydration stabilizer and optionally said further stabilizer,
and wherein the controlled release of the total amount of the AAV is characterized in that no greater than 9.0×10⁹ to 1.5×10¹⁰ AAV vg released on day 1,
and wherein the controlled released is measured from the time and under conditions wherein the implant is first immersed in an aqueous solution under physiological conditions such as at pH 7.2-7.4 and temperature 37° C.

80. A pharmaceutically acceptable implant comprising,
(i) a xerogel comprising a matrix comprising covalently crosslinked multi-arm precursors comprising,
(i-a) a first multi-arm precursor comprising a nucleophile,
(i-b) a second multi-arm precursor comprising an electrophile comprising a first reactive group, and
(i-c) a third multi-arm precursor comprising an electrophile comprising a second reactive group,
and
(ii) particles comprising a mixture of AAV selected from a group consisting of AAV1, AAV2 such as AAV2.7m8, AAV3, AAV4, AAV5, AAV6, AAV7, AAV8, AAV9, AAV10, AAV11 and a mutant, hybrid or variant thereof at a total amount in the order from 10⁹ to 10¹⁵ vg such as 10¹⁰ to 10¹³ vg and at least one dehydration stabilizer selected from a carbohydrate such as a sugar, such as sucrose or trehalose, a sugar alcohol, or a combination thereof, wherein the AAV comprises at least one heterologous nucleic acid sequence that codes for a therapeutic protein,
wherein the third multi-arm precursor has a longer hydrolysis half-life as compared to the second multi-arm precursor and wherein the molar ratio of:
(i) the first reactive group comprised in the second multi-arm precursor, and
(ii) the second reactive group comprised in the third multi-arm precursor, is 30-90:70-10.

81. A pharmaceutically acceptable implant for controlled release of AAV comprising,
(i) a xerogel comprising a matrix comprising covalently crosslinked multi-arm precursors comprising,
(i-a) a first multi-arm precursor comprising a nucleophile,
(i-b) a second multi-arm precursor comprising an electrophile comprising a first reactive group, and
(i-c) a third multi-arm precursor comprising an electrophile comprising a second reactive group,
and
(ii) particles comprising a mixture of AAV selected from a group consisting of AAV1, AAV2 such as AAV2.7m8, AAV3, AAV4, AAV5, AAV6, AAV7, AAV8, AAV9, AAV10, AAV11 and a mutant, hybrid or variant thereof at a total amount in the order from 10⁹ to 10¹⁵ vg such as 10¹⁰ to 10¹³ vg and at least one dehydration stabilizer selected from a carbohydrate such as a sugar, such as sucrose or trehalose, a sugar alcohol, or a combination thereof, wherein the AAV comprises at least one heterologous nucleic acid sequence that codes for a therapeutic protein,
wherein the third multi-arm precursor has a longer hydrolysis half-life as compared to the second multi-arm precursor and wherein the molar ratio of:
(i) the first reactive group comprised in the second multi-arm precursor and
(ii) the second reactive group comprised in the third multi-arm precursor, is 30-90:70-10,
and wherein the controlled release of the total amount of the AAV is characterized in that
(A) the percentage release of the AAV on day 1 is from 0 to 50% of the total amount of the AAV,
(B) the percentage release of the AAV per day from day 2 until the last day of release is from 0 to 50% of the total amount of the AAV, and/or
(C) number of days required for 100% release of the total amount of the AAV is at least 2 days,
wherein the controlled released is measured from the time and under conditions wherein the implant is first immersed in an aqueous solution under physiological conditions such as at pH 7.2-7.4 and temperature 37° C.

82. A pharmaceutically acceptable implant comprising,
(i) a xerogel comprising a matrix comprising covalently crosslinked multi-arm precursors comprising,
(i-a) a first multi-arm precursor comprising a primary amine,
(i-b) a second multi-arm precursor comprising succinimidyl succinate, and (i-c) a third multi-arm precursor comprising succinimidyl glutarate,
and
(ii) particles comprising a mixture of AAV selected from a group consisting of AAV1, AAV2 such as AAV2.7m8, AAV3, AAV4, AAV5, AAV6, AAV7, AAV8, AAV9, AAV10, AAV11 and a mutant, hybrid or variant thereof at a total amount in the order from 10⁹ to 10¹⁵ vg such as 10¹⁰ to 10¹³ vg and at least one dehydration stabilizer selected from a carbohydrate such as a sugar, such as sucrose or trehalose, a sugar alcohol, or a combination thereof, wherein the AAV comprises at least one heterologous nucleic acid sequence that codes for a therapeutic protein,
wherein the molar ratio of:
(i) the succinimidyl succinate group comprised in the second multi-arm precursor, and
(ii) the succinimidyl glutarate group comprised in the third multi-arm precursor, is 30-90:70-10.

83. A pharmaceutically acceptable implant for controlled release of AAV comprising,
(i) a xerogel comprising a matrix comprising covalently crosslinked multi-arm precursors comprising,
(i-a) a first multi-arm precursor comprising a primary amine,
(i-b) a second multi-arm precursor comprising succinimidyl succinate, and
(i-c) a third multi-arm precursor comprising succinimidyl glutarate,
and
(ii) particles comprising a mixture of AAV selected from a group consisting of AAV1, AAV2 such as AAV2.7m8, AAV3, AAV4, AAV5, AAV6, AAV7, AAV8, AAV9, AAV10, AAV11 and a mutant, hybrid or variant thereof at a total amount in the order from 10⁹ to 10¹⁵ vg such as 10¹⁰ to 10¹³ vg and at least one dehydration stabilizer selected from a carbohydrate such as a sugar, such as sucrose or trehalose, a sugar alcohol, or a combination thereof, wherein the AAV comprises at least one heterologous nucleic acid sequence that codes for a therapeutic protein,
wherein the molar ratio of:
(i) the succinimidyl succinate group comprised in the second multi-arm precursor, and
(ii) the succinimidyl glutarate group comprised in the third multi-arm precursor, is 30-90:70-10,
and wherein the controlled release of the total amount of the AAV is characterized in that
(A) the percentage release of the AAV on day 1 is from 0 to 50% of the total amount of the AAV,
(B) the percentage release of the AAV per day from day 2 until the last day of release is from 0 to 50% of the total amount of the AAV, and/or
(C) number of days required for 100% release of the total amount of the AAV is at least 2 days,
wherein the controlled released is measured from the time and under conditions wherein the implant is first immersed in an aqueous solution under physiological conditions such as at pH 7.2-7.4 and temperature 37° C.

84. A pharmaceutically acceptable implant comprising,
(i) a xerogel comprising a matrix comprising covalently crosslinked multi-arm precursors comprising,
(i-a) a first multi-arm precursor comprising a nucleophile,
(i-b) a second multi-arm precursor comprising an electrophile, and
(i-c) optionally a third multi-arm precursor comprising an electrophile,
and
(ii) particles comprising a mixture of AAV selected from a group consisting of AAV1, AAV2 such as AAV2.7m8, AAV3, AAV4, AAV5, AAV6, AAV7, AAV8, AAV9, AAV10, AAV11 and a mutant, hybrid or variant thereof at a total amount in the order from 10⁹ to 10¹⁵ vg such as 10¹⁰ to 10¹³ vg and at least one dehydration stabilizer selected from a carbohydrate such as a sugar, such as sucrose or trehalose, a sugar alcohol, or a combination thereof, wherein the AAV comprises at least one heterologous nucleic acid sequence that codes for a therapeutic protein,
wherein the molecular weight between crosslinks in the xerogel is from 7 to 25 kDa.

85. A pharmaceutically acceptable implant for controlled release of AAV comprising,
(i) a xerogel comprising a matrix comprising covalently crosslinked multi-arm precursors comprising,
(i-a) a first multi-arm precursor comprising a nucleophile,
(i-b) a second multi-arm precursor comprising an electrophile, and
(i-c) optionally a third multi-arm precursor comprising an electrophile, and
(ii) particles comprising a mixture of AAV selected from a group consisting of AAV1, AAV2 such as AAV2.7m8, AAV3, AAV4, AAV5, AAV6, AAV7, AAV8, AAV9, AAV10, AAV11 and a mutant, hybrid or variant thereof at a total amount in the order from 10⁹ to 10¹⁵ vg such as 10¹⁰ to 10¹³ vg and at least one dehydration stabilizer selected from a carbohydrate such as a sugar, such as sucrose or trehalose, a sugar alcohol, or a combination thereof, wherein the AAV comprises at least one heterologous nucleic acid sequence that codes for a therapeutic protein,
wherein the molecular weight between crosslinks in the xerogel is from 7 to 25 kDa, and wherein the controlled release of the total amount of the AAV is characterized in that
(A) the percentage release of the AAV on day 1 is from 0 to 50% of the total amount of the AAV,
(B) the percentage release of the AAV per day from day 2 until the last day of release is from 0 to 50% of the total amount of the AAV, and/or
(C) number of days required for 100% release of the total amount of the AAV is at least 2 days,
wherein the controlled released is measured from the time and under conditions wherein the implant is first immersed in an aqueous solution under physiological conditions such as at pH 7.2-7.4 and temperature 37° C.

86. A pharmaceutically acceptable implant comprising,
(i) a xerogel comprising a matrix comprising covalently crosslinked multi-arm precursors comprising,
(i-a) a first multi-arm precursor comprising a nucleophile,
(i-b) a second multi-arm precursor comprising an electrophile, and
(i-c) optionally a third multi-arm precursor comprising an electrophile,
and
(ii) particles comprising a mixture of AAV selected from a group consisting of AAV1, AAV2 such as AAV2.7m8, AAV3, AAV4, AAV5, AAV6, AAV7, AAV8, AAV9, AAV10, AAV11 and a mutant, hybrid or variant thereof at a total amount in the order from 10⁹ to 10¹⁵ vg such as 10¹⁰ to 10¹³ vg and at least one dehydration stabilizer selected from a carbohydrate such as a sugar, such as sucrose or trehalose, a sugar alcohol, or a combination thereof, and optionally at least one further stabilizer such as a buffer and/or a surfactant, wherein the AAV comprises at least one heterologous nucleic acid sequence that codes for a therapeutic protein,
wherein the ratio of:
(a) the (w/w) % of the total particles comprising a mixture of the AAV and said dehydration stabilizer and optionally said further stabilizer is no greater than 80%, or no greater than 70%, or no greater than 60%, or no greater than 50%,
(b) the (w/w) % of the total number of multi-arm precursors is from 20% to 80% such as from 35% to 75%, and
(c) the ratio of (a) and (b) is from 0.3 to 4.0 such as from 0.3 to 2.0, and/or
(d) the Dv₉₀ particle size is from 10 μm to 200 μm such as 35 μm to 75 μm such as 35 μm to 100 μm, or 35 μm to 150 μm, wherein particles comprise said mixture of the AAV and said dehydration stabilizer and optionally said further stabilizer, wherein the (w/w) % is based on the weight of the pharmaceutically acceptable implant.

87. A pharmaceutically acceptable implant comprising for controlled release of AAV comprising,
(i) a xerogel comprising a matrix comprising covalently crosslinked multi-arm precursors comprising,
(i-a) a first multi-arm precursor comprising a nucleophile,
(i-b) a second multi-arm precursor comprising an electrophile, and
(i-c) optionally a third multi-arm precursor comprising an electrophile,
and
(ii) particles comprising a mixture of AAV selected from a group consisting of AAV1, AAV2 such as AAV2.7m8, AAV3, AAV4, AAV5, AAV6, AAV7, AAV8, AAV9, AAV10, AAV11 and a mutant, hybrid or variant thereof at a total amount in the order from 10⁹ to 10¹⁵ vg such as 10¹⁰ to 10¹³ vg and at least one dehydration stabilizer selected from a carbohydrate such as a sugar, such as sucrose or trehalose, a sugar alcohol, or a combination thereof, and optionally at least one further stabilizer such as a buffer and/or a surfactant, wherein the AAV comprises at least one heterologous nucleic acid sequence that codes for a therapeutic protein,
wherein the ratio of:
(a) the (w/w) % of the total particles comprising a mixture of the AAV and said dehydration stabilizer is no greater than 80%, or no greater than 70%, or no greater than 60%, or no greater than 50% and,
(b) the (w/w) % of the total number of multi-arm precursors
(c) the ratio of (a) and (b) is from 0.3 to 4.0 such as from 0.3 to 2.0, and/or
(d) the Dv₉₀ particle size is from 10 μm to 200 μm such as 35 μm to 75 μm such as 35 μm to 100 μm, or 35 μm to 150 μm, wherein particles comprise said mixture of the AAV and said dehydration stabilizer and said further stabilizer, wherein the (w/w) % is based on the weight of the pharmaceutically acceptable implant,
and wherein the controlled release of the total amount of the AAV is characterized in that
(A) the percentage release of the AAV on day 1 is from 0 to 50% of the total amount of the AAV,
(B) the percentage release of the AAV per day from day 2 until the last day of release is from 0 to 50% of the total amount of the AAV, and/or
(C) number of days required for 100% release of the total amount of the AAV is at least 2 days,
wherein the controlled released is measured from the time and under conditions wherein the implant is first immersed in an aqueous solution under physiological conditions such as at pH 7.2-7.4 and temperature 37° C.

88. A pharmaceutically acceptable implant comprising,
(i) a xerogel comprising a matrix comprising covalently crosslinked multi-arm precursors comprising,
(i-a) a first multi-arm precursor comprising a nucleophile,
(i-b) a second multi-arm precursor comprising an electrophile comprising a first reactive group, and
(i-c) a third multi-arm precursor comprising an electrophile comprising a second reactive group,
and
(ii) particles comprising a mixture of AAV selected from a group consisting of AAV1, AAV2 such as AAV2.7m8, AAV3, AAV4, AAV5, AAV6, AAV7, AAV8, AAV9, AAV10, AAV11 and a mutant, hybrid or variant thereof at a total amount in the order from 10⁹ to 10¹⁵ vg such as 10¹⁰ to 10¹³ vg and at least one dehydration stabilizer selected from a carbohydrate such as a sugar, such as sucrose or trehalose, a sugar alcohol, or a combination thereof, and optionally at least one further stabilizer such as a buffer and/or a surfactant, wherein the AAV comprises at least one heterologous nucleic acid sequence that codes for a therapeutic protein,
wherein,
(i) the total (w/w) % of said dehydration stabilizer is no greater than 40%, such as from 5 to 40%,
(ii) the molecular weight between crosslinks in the xerogel is from 7 to 25 kDa,
(iii) the (w/w) % of the total particles comprising a mixture of the AAV and said dehydration stabilizer and optionally said further stabilizer is no greater than 80%, or no greater than 70%, or no greater than 60%, or no greater than 50%,
(iv) the (w/w) % of the total number of multi-arm precursors is from 20% to 80% such as from 35% to 75%,
(v) the ratio of (iii) and (iv) is from 0.3 to 4.0 such as from 0.3 to 2.0,
(vi) the Dv₉₀ particle size is from 10 μm to 200 μm such as 35 μm to 75 μm such as 35 μm to 100 μm, or 35 μm to 150 μm, wherein particles comprise said mixture of the AAV and said dehydration stabilizer and optionally said further stabilizer, and/or
(vii) the molar ratio of:
(vii-a) the first reactive group comprised in the second multi-arm precursor, and
(vii-b) the second reactive group comprised in the third multi-arm precursor, is from 30-90:70-10, wherein the first reactive group is succinimidyl succinate and the second reactive group is succinimidyl glutarate,
wherein the (w/w) % is based on the weight of the pharmaceutically acceptable implant.

89. A pharmaceutically acceptable implant for controlled release of AAV comprising,
(i) a xerogel comprising a matrix comprising covalently crosslinked multi-arm precursors comprising,
(i-a) a first multi-arm precursor comprising a nucleophile,
(i-b) a second multi-arm precursor comprising an electrophile comprising a first reactive group, and
(i-c) a third multi-arm precursor comprising an electrophile comprising a second reactive group,
and
(ii) particles comprising a mixture of AAV selected from a group consisting of AAV1, AAV2 such as AAV2.7m8, AAV3, AAV4, AAV5, AAV6, AAV7, AAV8, AAV9, AAV10, AAV11 and a mutant, hybrid or variant thereof at a total amount in the order from 10⁹ to 10¹⁵ vg such as 10¹⁰ to 10¹³ vg and at least one dehydration stabilizer selected from a carbohydrate such as a sugar, such as sucrose or trehalose, a sugar alcohol, or a combination thereof, wherein the AAV comprises at least one heterologous nucleic acid sequence that codes for a therapeutic protein,
wherein,
(i) the total (w/w) % of said dehydration stabilizer is no greater than 40%, such as from 5 to 40%,
(ii) the molecular weight between crosslinks in the xerogel is from 7 to 25 kDa,
(iii) the (w/w) % of the total particles comprising a mixture of the AAV and said dehydration stabilizer and optionally at least one further stabilizer such as a buffer and/or a surfactant is no greater than 80%, or no greater than 70%, or no greater than 60%, or no greater than 50%, and
(iv) the (w/w) % of the total number of multi-arm precursors is from 20% to 80% such as from 35% to 75%,
(v) the ratio of (iii) and (iv) is from 0.3 to 4.0 such as from 0.3 to 2.0,
(vi) the Dv₉₀ particle size is from 10 μm to 200 μm such as 35 μm to 75 μm such as 35 μm to 100 μm, or 35 μm to 150 μm, wherein particles comprise said mixture of the AAV and said dehydration stabilizer and optionally said further stabilizer, and/or
(vii) the molar ratio of:
(vii-a) the first reactive group comprised in the second multi-arm precursor and
(vii-b) the second reactive group comprised in the third multi-arm precursor, is from 30-90:70-10, wherein the first reactive group is succinimidyl succinate and the second reactive group is succinimidyl glutarate,
wherein the (w/w) % is based on the weight of the pharmaceutically acceptable implant,
and wherein the controlled release of the total amount of the AAV is characterized in that
(A) the percentage release of the AAV on day 1 is from 0 to 50% of the total amount of the AAV,
(B) the percentage release of the AAV per day from day 2 until the last day of release is from 0 to 50% of the total amount of the AAV, and/or
(C) number of days required for 100% release of the total amount of the AAV is at least 2 days,
wherein the controlled released is measured from the time and under conditions wherein the implant is first immersed in an aqueous solution under physiological conditions such as at pH 7.2-7.4 and temperature 37° C.

90. A pharmaceutically acceptable implant for controlled release of AAV comprising
(i) a xerogel comprising a matrix comprising covalently crosslinked multi-arm precursors comprising,
(i-a) a first multi-arm precursor comprising a nucleophile,
(i-b) a second multi-arm precursor comprising an electrophile, and
(i-c) optionally, a third multi-arm precursor comprising an electrophile, and
(ii) particles comprising a mixture of AAV selected from a group consisting of AAV1, AAV2 such as AAV2.7m8, AAV3, AAV4, AAV5, AAV6, AAV7, AAV8, AAV9, AAV10, AAV11 and a mutant, hybrid or variant thereof at a total amount in the order from 10⁹ to 10¹⁵ vg such as 10¹⁰ to 10¹³ vg and at least one dehydration stabilizer selected from a carbohydrate such as a sugar, such as sucrose or trehalose, a sugar alcohol, or a combination thereof, and optionally at least one further stabilizer such as a buffer and/or a surfactant, wherein the AAV comprises at least one heterologous nucleic acid sequence that codes for a therapeutic protein,
wherein the particles are dispersed within the xerogel,
and wherein the total (w/w) % of said dehydration stabilizer is no greater than 40% w/w, such as from 5 to 40% w/w, wherein the (w/w) % is based on the weight of the pharmaceutically acceptable implant,
and wherein the controlled release of the AAV is characterized in that the number of days required for 100% release is at least 4 days, and wherein the controlled released is measured from the time and under conditions wherein the implant is first immersed in an aqueous solution under physiological conditions such as at pH 7.2-7.4 and temperature 37° C.
and/or
A pharmaceutically acceptable implant for controlled release of AAV comprising
(i) a xerogel comprising a matrix comprising covalently crosslinked multi-arm precursors comprising,
(i-a) a first multi-arm precursor comprising a nucleophile,
(i-b) a second multi-arm precursor comprising an electrophile, and
(i-c) optionally, a third multi-arm precursor comprising an electrophile, and
(ii) particles comprising a mixture of AAV selected from a group consisting of AAV1, AAV2 such as AAV2.7m8, AAV3, AAV4, AAV5, AAV6, AAV7, AAV8, AAV9, AAV10, AAV11 and a mutant, hybrid or variant thereof at a total amount in the order from 10⁹ to 10¹⁵ vg such as 10¹⁰ to 10¹³ vg and at least one dehydration stabilizer selected from a carbohydrate such as a sugar, such as sucrose or trehalose, a sugar alcohol, or a combination thereof, and optionally at least one further stabilizer such as a buffer and/or a surfactant, wherein the AAV comprises at least one heterologous nucleic acid sequence that codes for a therapeutic protein, wherein the particles are dispersed within the xerogel,
wherein the Dv₉₀ particle size is from 10 μm to 200 μm such as 35 μm to 75 μm such as 35 μm to 100 μm, or 35 μm to 150 μm, wherein particles comprise said mixture of the AAV and said dehydration stabilizer and optionally said further stabilizer, and wherein the controlled release of the total amount of the AAV is characterized in that no greater than 9.0×10⁹ to 1.5×10¹⁰ AAV vg is released on day 1,
and wherein the controlled released is measured from the time and under conditions wherein the implant is first immersed in an aqueous solution under physiological conditions such as at pH 7.2-7.4 and temperature 37° C.

91. A pharmaceutically acceptable implant for controlled release of AAV comprising,
(i) a xerogel comprising a matrix comprising covalently crosslinked multi-arm precursors comprising,
(i-a) a first multi-arm precursor comprising a nucleophile,
(i-b) a second multi-arm precursor comprising an electrophile comprising a first reactive group, and
(i-c) a third multi-arm precursor comprising an electrophile comprising a second reactive group,
and
(ii) particles comprising a mixture of AAV selected from a group consisting of AAV1, AAV2 such as AAV2.7m8, AAV3, AAV4, AAV5, AAV6, AAV7, AAV8, AAV9, AAV10, AAV11 and a mutant, hybrid or variant thereof at a total amount in the order from 10⁹ to 10¹⁵ vg such as 10¹⁰ to 10¹³ vg and at least one dehydration stabilizer selected from a carbohydrate such as a sugar, such as sucrose or trehalose, a sugar alcohol, or a combination thereof, wherein the AAV comprises at least one heterologous nucleic acid sequence that codes for a therapeutic protein,
wherein the third multi-arm precursor has a longer hydrolysis half-life as compared to the second multi-arm precursor and wherein the molar ratio of:
(i) the first reactive group comprised in the second multi-arm precursor and
(ii) the second reactive group comprised in the third multi-arm precursor, is from 30-90:70-10, and wherein the controlled release of the total amount of the AAV is characterized in that
(A) no greater than 9.0×10⁹ to 1.5×10¹⁰ AAV vg released on day 1,
(B) no greater than in the order 10¹¹ vg AAV per day such as in the order 10⁸, or 10⁹ or 10¹⁰ such as 5.0×10⁹ to 1.5×10¹⁰ AAV vg released per day from day 2 until the last day of the controlled release, and/or
(C) the number of days required for 100% release of the AAV is not less than 4 days, wherein the controlled released is measured from the time and under conditions wherein the implant is first immersed in an aqueous solution under physiological conditions such as at pH 7.2-7.4 and temperature 37° C.

92. A pharmaceutically acceptable implant for controlled release of AAV comprising,
(i) a xerogel comprising a matrix comprising covalently crosslinked multi-arm precursors comprising,
(i-a) a first multi-arm precursor comprising a primary amine,
(i-b) a second multi-arm precursor comprising succinimidyl succinate, and
(i-c) a third multi-arm precursor comprising succinimidyl glutarate,
and
(ii) particles comprising a mixture of AAV selected from a group consisting of AAV1, AAV2 such as AAV2.7m8, AAV3, AAV4, AAV5, AAV6, AAV7, AAV8, AAV9, AAV10, AAV11 and a mutant, hybrid or variant thereof at a total amount in the order from 10⁹ to 10¹⁵ vg such as 10¹⁰ to 10¹³ vg and at least one dehydration stabilizer selected from a carbohydrate such as a sugar, such as sucrose or trehalose, a sugar alcohol, or a combination thereof, wherein the AAV comprises at least one heterologous nucleic acid sequence that codes for a therapeutic protein,
wherein the molar ratio of:
(i) the succinimidyl succinate group comprised in the second multi-arm precursor, and
(ii) the succinimidyl glutarate group comprised in the third multi-arm precursor, is from 30-90:70-10,
and wherein the controlled release of the total amount of the AAV is characterized in that
(A) no greater than 9.0×10⁹ to 1.5×10¹⁰ AAV vg released on day 1,
(B) no greater in the order 10¹¹ vg AAV per day such as in the order 10⁸, or 10⁹ or 10¹⁰ such as than 5.0×10⁹ to 1.5×10¹⁰ AAV vg released per day from day 2 until the last day of the controlled release, and/or
(C) the number of days required for 100% release of the AAV is not less than 4 days, wherein the controlled released is measured from the time and under conditions wherein the implant is first immersed in an aqueous solution under physiological conditions such as at pH 7.2-7.4 and temperature 37° C.

93. A pharmaceutically acceptable implant for controlled release of AAV comprising,
(i) a xerogel comprising a matrix comprising covalently crosslinked multi-arm precursors comprising,
(i-a) a first multi-arm precursor comprising a nucleophile,
(i-b) a second multi-arm precursor comprising an electrophile, and
(i-c) optionally a third multi-arm precursor comprising an electrophile, and
(ii) particles comprising a mixture of AAV selected from a group consisting of AAV1, AAV2 such as AAV2.7m8, AAV3, AAV4, AAV5, AAV6, AAV7, AAV8, AAV9, AAV10, AAV11 and a mutant, hybrid or variant thereof at a total amount in the order from 10⁹ to 10¹⁵ vg such as 10¹⁰ to 10¹³ vg and at least one dehydration stabilizer selected from a carbohydrate such as a sugar, such as sucrose or trehalose, a sugar alcohol, or a combination thereof, wherein the AAV comprises at least one heterologous nucleic acid sequence that codes for a therapeutic protein,
wherein the molecular weight between crosslinks is from 7 to 25 kDa,
and wherein the controlled release of the total amount of the AAV is characterized in that
(A) no greater than 9.0×10⁹ to 1.5×10¹⁰ AAV vg released on day 1,
(B) no greater than in the order 10¹¹ vg AAV per day such as in the order 10⁸, or 10⁹ or 10¹⁰ such as 5.0×10⁹ to 1.5×10¹⁰ AAV vg released per day from day 2 until the last day of the controlled release, and/or
(C) the number of days required for 100% release of the AAV is not less than 4 days, wherein the controlled released is measured from the time and under conditions wherein the implant is first immersed in an aqueous solution under physiological conditions such as at pH 7.2-7.4 and temperature 37° C.

94. A pharmaceutically acceptable implant comprising for controlled release of AAV comprising,
(i) a xerogel comprising a matrix comprising covalently crosslinked multi-arm precursors comprising,
(i-a) a first multi-arm precursor comprising a nucleophile,
(i-b) a second multi-arm precursor comprising an electrophile, and
(i-c) optionally a third multi-arm precursor comprising an electrophile, and
(ii) particles comprising a mixture of AAV selected from a group consisting of AAV1, AAV2 such as AAV2.7m8, AAV3, AAV4, AAV5, AAV6, AAV7, AAV8, AAV9, AAV10, AAV11 and a mutant, hybrid or variant thereof at a total amount in the order from 10⁹ to 10¹⁵ vg such as 10¹⁰ to 10¹³ vg and at least one dehydration stabilizer selected from a carbohydrate such as a sugar, such as sucrose or trehalose, a sugar alcohol, or a combination thereof, and optionally at least one further stabilizer such as a buffer and/or a surfactant, wherein the AAV comprises at least one heterologous nucleic acid sequence that codes for a therapeutic protein,
wherein
(a) the (w/w) % of the total particles comprising a mixture of the AAV and said dehydration stabilizer and optionally said further stabilizer is no greater than 80%, or no greater than 70%, or no greater than 60%, or no greater than 50% and,
(b) the (w/w) % of the total number of multi-arm precursors is from 20% to 80% such as from 35% to 75%, and/or
(c) the molar ratio of (a) and (b) is from 0.3 to 2.0, and/or
(d) the Dv₉₀ particle size is from 10 μm to 200 μm such as 35 μm to 75 μm such as 35 μm to 100 μm, or 35 μm to 150 μm, wherein particles comprise said mixture of the AAV and said dehydration stabilizer and optionally said further stabilizer,
wherein the (w/w) % is based on the weight of the pharmaceutically acceptable implant,
and wherein the controlled release of the total amount of the AAV is characterized in that
(A) no greater than 9.0×10⁹ to 1.5×10¹⁰ AAV vg released on day 1,
(B) no greater than 5.0×10⁹ to 1.5×10¹⁰ AAV vg released per day from day 2 until the last day of the controlled release, and/or
(C) the number of days required for 100% release of the AAV is not less than 4 days, wherein the controlled released is measured from the time and under conditions wherein the implant is first immersed in an aqueous solution under physiological conditions such as at pH 7.2-7.4 and temperature 37° C.

95. A pharmaceutically acceptable implant for controlled release of AAV comprising,
(i) a xerogel comprising a matrix comprising covalently crosslinked multi-arm precursors comprising,
(i-a) a first multi-arm precursor comprising a nucleophile,
(i-b) a second multi-arm precursor comprising an electrophile comprising a first reactive group, and
(i-c) a third multi-arm precursor comprising an electrophile comprising a second reactive group,
and
(ii) particles comprising a mixture of AAV selected from a group consisting of AAV1, AAV2 such as AAV2.7m8, AAV3, AAV4, AAV5, AAV6, AAV7, AAV8, AAV9, AAV10, AAV11 and a mutant, hybrid or variant thereof at a total amount in the order from 10⁹ to 10¹⁵ vg such as 10¹⁰ to 10¹³ vg and at least one dehydration stabilizer selected from a carbohydrate such as a sugar, such as sucrose or trehalose, a sugar alcohol, or a combination thereof, and optionally at least one further stabilizer such as a buffer and/or a surfactant, wherein the AAV comprises at least one heterologous nucleic acid sequence that codes for a therapeutic protein,
wherein,
(i) the total (w/w) % of said dehydration stabilizer is no greater than 40%, such as from 5 to 40%, wherein the (w/w) % is based on the weight of the pharmaceutically acceptable implant,
(ii) the molecular weight between crosslinks in the xerogel is from 7 to 25 kDa,
(iii) the (w/w) % of the total particles comprising a mixture of the AAV and said dehydration stabilizer and optionally said further stabilizer is no greater than 80%, or no greater than 70%, or no greater than 60%, or no greater than 50%,
(iv) the (w/w) % of the total number of multi-arm precursors is from 20% to 80% such as from 35% to 75%, and/or
(v) the ratio of (iii) and (iv) is from 0.3 to 4.0 such as from 0.3 to 2.0,
(vi) the Dv₉₀ particle size is from 10 μm to 200 μm such as 35 μm to 75 μm such as 35 μm to 100 μm, or 35 μm to 150 μm, wherein particles comprise said mixture of the AAV and said dehydration stabilizer, and optionally said further stabilizer, and/or
(vii) the molar ratio of:
(vii-a) the first reactive group comprised in the second multi-arm precursor and
(vii-b) the second reactive group comprised in the third multi-arm precursor, is from 30-90:70-10,
wherein the first reactive group is succinimidyl succinate and the second reactive group is succinimidyl glutarate,
wherein the (w/w) % is based on the weight of the pharmaceutically acceptable implant,
and wherein the controlled release of the total amount of the AAV is characterized in that
(A) no greater than 9.0×10⁹ to 1.5×10¹⁰ AAV vg released on day 1,
(B) no greater than in the order 10¹¹ vg AAV2 per day such as in the order 10⁸, or 10⁹ or 10¹⁰ such as 5.0×10⁹ to 1.5×10¹⁰ AAV vg released per day from day 2 until the last day of the controlled release, and/or
(C) the number of days required for 100% release of the AAV is not less than 4 days, wherein the controlled released is measured from the time and under conditions wherein the implant is first immersed in an aqueous solution under physiological conditions such as at pH 7.2-7.4 and temperature 37° C.

96. A method of treating an ocular disorder such as an ocular genetic disorder comprising administering to a subject a pharmaceutically acceptable implant according to any one of the preceding claims.

97. A pharmaceutically acceptable implant according to any one of the preceding claims for use in a method of treating an ocular disorder such as an ocular genetic disorder comprising administering to a subject the pharmaceutically acceptable implant.

98. The method or use according to claim 96 or 97, wherein the pharmaceutically acceptable implant is for controlled release of a total amount of the biologic.

99. The method or use according to claim 98, wherein the controlled release is characterized by:
(A) the amount of the biologic released on day 1 is from 0 to 50% of the total amount of the biologic,
(B) the amount of the biologic released per day from day 2 until the last day of release is from 0 to 50% of the total amount of the biologic, and/or
(C) the number of days required for 100% release of the total amount of the biologic is at least 2 days.

100. A method of controlling inflammation when treating an ocular disorder such as an ocular genetic disorder comprising administering to a subject a pharmaceutically acceptable implant according to any one of the preceding claims, wherein the implant comprises a total amount of a biologic, and wherein controlling inflammation is characterized by obtaining a lower inflammation in the eye of the subject as compared to another composition comprising the same biologic at the same amount administered at the same time to a comparison subject such as a bolus.

101. A pharmaceutically acceptable implant according to any one of the preceding claims for use in controlling inflammation when treating an ocular disorder such as an ocular genetic disorder, wherein the implant comprises a total amount of a biologic, and wherein controlling inflammation is characterized by obtaining a lower inflammation in the eye of the subject as compared to another composition comprising the same biologic at the same amount administered at the same time to a comparison subject such as a bolus.

102. The method or use according to claim 100 or 101, wherein inflammation refers to inflammation evaluated using an ocular scoring system, such as the Standardization of Uveitis Nomenclature (SUN), semiquantitative preclinical ocular toxicology scoring (SPOTS), McDonald-Shadduck, or the Hackett-McDonald systems such as at the inflammation peak.

103. The method or use according to claim 102, wherein the inflammation peak is characterized as the period between the second week and four months, or second week and three months, or second week and two months following administration of the pharmaceutically acceptable implant to a subject, such as between the second week and one month following administration of the pharmaceutically acceptable implant to a subject.

104. The method or use according to claims 102 and 103, wherein the inflammation peak is characterized as the third week following administration of the pharmaceutically acceptable implant to a subject, wherein the subject is rabbit, non-human primate or human.

105. The method or use according to any one of claims 100 to 104, wherein the pharmaceutically acceptable implant comprises a virus selected from a group consisting of retrovirus, adenovirus, adeno-associated virus (AAV), lentivirus and herpes simplex virus.

106. The method or use according to claim 105, wherein the virus is adeno-associated virus (AAV).

107. The method or use according to claim 106, wherein the adeno-associated virus (AAV) is selected from a group consisting of AAV1, AAV2 such as AAV2.7m8, AAV3, AAV4, AAV5, AAV6, AAV7, AAV8, AAV9, AAV10, AAV11 and a mutant, hybrid or variant thereof, wherein the AAV comprises at least one heterologous nucleic acid sequence that codes for a therapeutic protein preferably wherein the heterologous nucleic acid(s) code(s) for a therapeutic protein, which is absent or present at a reduced level in the subject in need thereof as compared to the same but healthy subject.

108. The method or use according to claim 107, wherein the pharmaceutically acceptable implant comprises a virus, wherein the virus is adeno-associated virus (AAV), and wherein AAV is selected from a group consisting of AAV2, AAV2.7m8, and AAV8.

109. The method or use according to any one of claims 100-108, wherein the pharmaceutically acceptable implant is for controlled release of a total amount of AAV.

110. The method or use according to claim 109, wherein the controlled release is characterized by:
(A) the amount of the AAV released on day 1 is from 0 to 50% of the total amount of the biologic,
(B) the amount of the AAV released per day from day 2 until the last day of release is from 0 to 50% of the total amount of the biologic, and/or
(C) the number of days required for 100% release of the total amount of the AAV is at least 4 days.

111. The method or use according to any one of claims 109-110, wherein the controlled release is characterized by
(A) no greater than 9.0×10⁹ to 1.5×10¹⁰ vg AAV released on day 1,
(B) no greater than in the order 10¹¹ vg AAV2 per day such as in the order 10⁸, or 10⁹ or 10¹⁰ such as 5.0×10⁹ to 1.5×10¹⁰ vg AAV released per day from day 2 until the last day of the controlled release, and/or
(C) the number of days required for 100% release of the AAV is not less than 4 days.

112. The method or use according to any one of claims 109-110, wherein the controlled release is characterized by
(A) no greater than 9.0×10⁹ to 1.5×10¹⁰ vg AAV2 released on day 1,
(B) no greater than in the order 10¹¹ vg AAV2 per day such as in the order 10⁸, or 10⁹, or 10¹⁰ such as 5.0×10⁹ to 1.5×10¹⁰ vg AAV2 released per day from day 2 until the last day of the controlled release, and/or
(C) the number of days required for 100% release of the AAV2 is not less than 4 days.

113. The method or use according to any one of claims 109-110, wherein the controlled release is characterized by
(A) no greater than 9.0×10⁹ to 1.5×10¹⁰ vg AAV2.7m8 released on day 1,
(B) no greater than in the order 10¹¹ vg AAV2.7m8 per day such as in the order 10⁸, or 10⁹ or 10¹⁰ such as 5.0×10⁹ to 1.5×10¹⁰ vg AAV2.7m8 released per day from day 2 until the last day of the controlled release, and/or
(C) the number of days required for 100% release of the AAV2.7m8 is not less than 4 days.

114. The method or use according to claims 96 to 113, wherein the method comprises intravitreal injection of the pharmaceutically acceptable implant to the subject in need thereof.

115. The method or use of any one of claims 96 to 114, wherein the controlled release is characterized by
(A) no greater than 6×10⁹ to 1.0×10¹⁰ vg AAV per mL of the vitreous volume of the subject released on day 1,
(B) no greater than 3.5×10⁹ to 1.0×10¹⁰ vg AAV per mL of the vitreous volume of the subject released per day from day 2 until the last day of the controlled release, and/or
(C) the number of days required for 100% release of the AAV is not less than 4 days.

116. The method or use of any one of claims 96 to 114, wherein the controlled release is characterized by
(A) no greater than 6×10⁹ to 1.0×10¹⁰ vg AAV2 per mL of the vitreous volume of the subject released on day 1,
(B) no greater than 3.5×10⁹ to 1.0×10¹⁰ vg AAV2 per mL of the vitreous volume of the subject released per day from day 2 until the last day of the controlled release, and/or
(C) the number of days required for 100% release of the AAV2 is not less than 4 days.

117. The method or use of any one of claims 96 to 114, wherein the controlled release is characterized by
(A) no greater than 6×10⁹ to 1.0×10¹⁰ vg AAV2.7m8 per mL of the vitreous volume of the subject released on day 1,
(B) no greater than 3.5×10⁹ to 1.0×10¹⁰ vg AAV2.7m8 per mL of the vitreous volume of the subject released per day from day 2 until the last day of the controlled release, and/or
(C) the number of days required for 100% release of the AAV2.7m8 is not less than 4 days.

118. The method or use according to any one of claims 96-117, wherein when the total dose of AAV comprised in the pharmaceutically acceptable implant is in the order less than 2.0×10¹⁰ vg the number of days required for 100% release of the AAV is at least 4 days.

119. The method or use according to claim 118, wherein when the total dose of AAV comprised in the pharmaceutically acceptable implant is in the order greater than 2.0×10¹⁰ vg, the number of days required for 100% release of the AAV is at least 7 days, such as greater than 7 days, such as greater than 10 days or more.

120. The method or use according to any one of claims 96-119, wherein the treatment comprises a combination therapy.

121. The method or use according to claim 120, wherein combination therapy is characterized in that
(i) the AAV comprises more than one heterologous nucleotide sequences, wherein each of the more than one heterologous nucleotide sequences codes for a different therapeutic protein, and/or
(ii) the pharmaceutically acceptable implant is administered in combination with one or more additional therapeutic agent(s) either on the same or different day.

122. The method or use according to claim 121, wherein the therapeutic agent is selected from a group consisting of a small molecule, a large molecule, a protein, a nanoparticle, or another virus.

123. The method or use according to any one of claims 121 and 122, wherein the therapeutic agent is selected from an immunosuppressant such as triamcinolone, prednisolone, or cyclosporin, cyclophsphamide, sirolimus, or tacrolimus.

124. The method or use according to any one of claims 121 and 122, wherein the therapeutic agent is an anti-NAb, an anti-T-cell antibody such as anti-CD40L, or a viral transduction enhancer.

125. The method or use according to any one of claims 121 and 122, wherein the therapeutic agent is a tyrosine kinase inhibitor, such as axitinib, sunitinib, sorafenib, paxopanib, or tivozanib.

126. A method of determining the concentration threshold of an AAV serotype that causes inflammation when treating an ocular genetic disorder comprising
(i) administering to a first subject the pharmaceutically acceptable implant according to claims 58-65 comprising AAV at a dose ranging from 10⁹ to 10¹⁵ vg,
(ii) assessing inflammation using an ocular scoring system, such as the Standardization of Uveitis Nomenclature (SUN), semiquantitative preclinical ocular toxicology scoring (SPOTS), McDonald-Shadduck, or the Hackett-McDonald systems,
(iii) administering to a second subject or second eye of the same subject as (i), a pharmaceutically acceptable implant with substantially the same characteristics as the implant of (i) and comprising AAV at a specific dose ranging from 10⁹ to 10¹⁵ vg that is different from the dose of (i) by at least 3-10 times,
(iv) assessing inflammation using an ocular scoring system, such as the Standardization of Uveitis Nomenclature (SUN), semiquantitative preclinical ocular toxicology scoring (SPOTS), McDonald-Shadduck, or the Hackett-McDonald systems,
(v) repeating steps (i) to (iv) until either of steps (ii) or (iv) provides for an acceptable inflammation score,
wherein the AAV serotype is selected from a group consisting of AAV1, AAV2 such as AAV2.7m8, AAV3, AAV4, AAV5, AAV6, AAV7, AAV8, AAV9, AAV10, AAV11 and a mutant, hybrid or variant thereof.

127. A pharmaceutically acceptable implant according to claims 58-65 for use in a method of determining the concentration threshold of an AAV serotype that causes inflammation when treating an ocular genetic disorder comprising
(i) administering to a first subject the pharmaceutically acceptable implant according to claims 58-65 comprising AAV at a dose ranging from 10⁹ to 10¹⁵ vg,
(ii) assessing inflammation using an ocular scoring system, such as the Standardization of Uveitis Nomenclature (SUN), semiquantitative preclinical ocular toxicology scoring (SPOTS), McDonald-Shadduck, or the Hackett-McDonald systems,
(iii) administering to a second subject or second eye of the same subject as (i), a pharmaceutically acceptable implant with substantially the same characteristics as the implant of (i) and comprising AAV at a specific dose ranging from 10⁹ to 10¹⁵ vg that is different from the dose of (i) by at least 3-10 times,
(iv) assessing inflammation using an ocular scoring system, such as the Standardization of Uveitis Nomenclature (SUN), semiquantitative preclinical ocular toxicology scoring (SPOTS), McDonald-Shadduck, or the Hackett-McDonald systems,
(v) repeating steps (i) to (iv) until either of steps (ii) or (iv) provides for an acceptable inflammation score,
wherein the AAV serotype is selected from a group consisting of AAV1, AAV2 such as AAV2.7m8, AAV3, AAV4, AAV5, AAV6, AAV7, AAV8, AAV9, AAV10, AAV11 and a mutant, hybrid or variant thereof.

128. The method or use according to claim 126 or 127, wherein the method comprises intravitreal injection of the pharmaceutically acceptable implant to the subject in need thereof.

129. The method or use according to claim 126 or 127, wherein inflammation refers to inflammation evaluated using an ocular scoring system, such as the Standardization of Uveitis Nomenclature (SUN), semiquantitative preclinical ocular toxicology scoring (SPOTS), McDonald-Shadduck, or the Hackett-McDonald systems at the inflammation peak.

130. The method or use according to claim 129, wherein the inflammation peak is characterized as the period between the second week and two months following administration of the pharmaceutically acceptable implant to a subject, such as between the second week and one month following administration of the pharmaceutically acceptable implant to a subject.

131. The method or use according to any one of claims 129 and 130, wherein the inflammation peak is characterized as the third week following administration of the pharmaceutically acceptable implant to a rabbit.

132. The method of claims 96 to 131, wherein the ocular disorder is selected from a group consisting of retinal neovascularisation, choroidal neovascularisation, Wet AMD, Dry AMD, retinal vein occlusion, diabetic macular edema, retinal degeneration, corneal graft rejection, retinoblastoma, melanoma, glaucoma, autoimmune uveitis, uveitis, proliferative vitreoretinopathy, thyroid eye disease, neurotrophic keratitis, and corneal degeneration.

133. The method of claims 96 to 131, wherein the ocular genetic disorder is selected from a group consisting of retinitis pigmentosa, Leber's Congenital Amaurosis, Choroideremia, X-linked retinitis pigmentosa, best vitelliform macular dystrophy, x-linked retinoschisis, achromatopsia CNGA3, achromotopsia CNGB3, LHON, Stargardt disease, Usher syndrome, Norrie disease, Bardet-Biedl syndrome, enhanced S-Cone Syndrom/Goldman Favre, and red-green colour blindness.

134. A method of treating inflammation in the eye of a subject by administering a therapeutically effective amount of a tyrosine kinase inhibitor to the eye of the subject in need thereof.

135. The method of claim 134, wherein the inflammation is caused by an innate immune response.

136. The method of claim 134, wherein the administration of the tyrosine kinase inhibitor is conducted within 24 hours of the inflammation causing event.

137. The method of claim 136, wherein the tyrosine kinase inhibitor is co-administered with the inflammation causing event.

138. The method of any one of claims 134-137, wherein the tyrosine kinase inhibitor is selected from a group consisting of axitinib, sunitinib, sorafenib, paxopanib, or tivozanib.

139. The method of claim 138, wherein the tyrosine kinase inhibitor is axitinib.

140. Use of at least one dehydration stabilizer for protecting a biologic against damage during a process wherein the biologic is directly exposed to an organic solvent.

141. The use of claim 140, wherein the dehydration stabilizer and/or the biologic are substantially insoluble in the organic solvent, such as a solubility of 0.1 mg/ml or less.

142. The use of claim 140 or 141, wherein the total concentration of the dehydration stabilizer intermixed with the biologic is from 5 mg/ml to 200 mg/mL

143. The use of any one of claims 140-142, wherein the at least one dehydration stabilizer is selected from a group consisting of a carbohydrate, a sugar alcohol and combination thereof

144. The use of claim 143, wherein the carbohydrate is selected from a monosaccharide, a disaccharide, an oligosaccharide, a water-soluble polysaccharide, or a combination thereof.

145. The use of claim 143 or 144, wherein the carbohydrate is a sugar.

146. The use of claim 145, wherein the sugar is a non-reducing sugar.

147. The use of claim 145 or 146, wherein the sugar is selected from a group consisting of sucrose, trehalose, raffinose, stachyose, verbascose, hydrates thereof and a combination thereof, preferably sucrose, trehalose, trehalose dihydrate and a combination thereof.

148. The use of any one of claims 143-147, wherein the sugar alcohol is selected from a group consisting of erythritol, glycerol, isomalt, lactitol, maltitol, mannitol, sorbitol, xylitol, and a combination thereof.

149. The use of any one of claims 140-149, wherein the process in which the biologic is directly exposed to an organic solvent comprises forming an organogel comprising forming a matrix within which the biologic is dispersed.

150. The use of claim 149, wherein the matrix comprises covalently crosslinked multiarm precursors formed in the presence of the organic solvent.

151. The use of claim 150, wherein the multi-arm precursors comprise at least two multi-arm precursors comprising a first multi-arm precursor comprising a first functional group, and a second multi-arm precursor comprising a second functional group.

152. The use of claim 151, wherein the multi-arm precursors comprise a third multiarm precursor comprising the same functional group as the second multi-arm precursor.

153. The use of claim 151 or 152, wherein each of the first functional group and the second functional group is selected from a group consisting of an electrophile and a nucleophile, and the reaction between the first functional group and second functional group is an electrophile-nucleophile reaction that forms the covalent bond.

154. The use of claim 153, wherein the nucleophile comprises an amine such as, a primary amine, a thiol, an or a hydrazide.

155. The use of any one of claims 153-154, wherein the electrophile comprises succinimidyl esters, succinimidyl carbonates, nitrophenyl carbonates, aldehyde, ketones, acrylates, acrylamides, maleimides, vinylsulfones, iodoacetamides, alkenes, alkynes, norbornenes, epoxides, mesylates, tosylates, tresyls, cyanurates, orthopyridyl disulfides, or halides preferably, wherein the succinimidyl ester comprises a reactive group selected from succinimidyl succinate, succinimidyl glutarate, succinimidyl adipate, succinimidyl azelate, and succinimidyl glutaramide.

156. The use of any one of claims 140-155, wherein the process further comprises forming a xerogel.

157. The use of claim 156, wherein forming a xerogel comprises removing the organic solvent from the organogel.

158. The use of claim 157, wherein the process further comprises forming a pharmaceutically acceptable implant.

159. The use of claim 158, wherein the pharmaceutically acceptable implant comprises a xerogel, and particles comprising a mixture of the biologic, and at least one dehydration stabilizer and optionally at least one further stabilizer such as a buffer and/or a surfactant.

160. The use of claim 159, wherein the xerogel comprises a matrix comprising covalently crosslinked multi-arm precursors within which the particles comprising the mixture of the biologic, and at least one dehydration stabilizer and optionally at least one further stabilizer such as a buffer and/or a surfactant are dispersed, preferably wherein the at least one dehydration stabilizer is selected from carbohydrate, a sugar alcohol and any combination thereof.

161. The use of any one of claims 140-160, wherein a further dehydration stabilizer is selected from a group consisting of polyalkylene oxide such as polyethylene glycol, polyvinyl pyrrolidinone, and polyvinyl alcohol.

162. The use of any one of claims 156 to 161, wherein the xerogel is a hydrogel upon exposure to an aqueous solution.

163. The use of any one of claims 158 to 162, wherein the pharmaceutically acceptable implant is for a controlled release of the biologic.

164. The use of claim 163, wherein the total (w/w) % of the carbohydrate, sugar alcohol or combination thereof is no greater than 40%, such as from 5 to 40%, wherein the (w/w) % is based on the weight of the pharmaceutically acceptable implant.

165. The use of claim 164, wherein the total (w/w) % of the carbohydrate, sugar alcohol or combination thereof is from 10 to 25%, wherein the (w/w) % is based on the weight of the pharmaceutically acceptable implant.

166. The use of any one of claims 163-165, wherein the implant is for controlled release of a total amount of the biologic, and wherein the controlled release is characterized in that the number of days required for 100% release of the total amount of the biologic is at least 2 days.

167. The use of any one of claims 140-166, wherein the organic solvent is selected from a group consisting of methylene chloride, dimethyl carbonate, acetone, acetonitrile, ethyl acetate, and tetrahydrofuran.

168. The use of claims 140-167, wherein the biologic comprises a plurality of the same or different biologics.

169. The use of claim 168, wherein the biologic is selected from a group consisting of a polypeptide, a virus or virus-like particle, and a lipid encapsulating a nucleic acid(s).

170. The use of claim 169, wherein the polypeptide is any polypeptide having a primary, secondary, tertiary or quaternary structure.

171. The use of any one of claims 169-170, wherein the polypeptide is a recombinant protein.

172. The use of claim 171, wherein the recombinant protein is selected from an antibody, an antigen binding fragment, a fusion protein, a cytokine, a transcription factor, an enzyme or a hormone.

173. The use of claim 169, wherein the lipid encapsulating nucleic acid(s) comprises one or more heterologous nucleic acid(s).

174. The use of claim 169, wherein the virus or virus-like particle comprises a viral nucleic acid and one or more heterologous nucleic acid(s).

175. The use of any one of claims 173 and 174, wherein each heterologous nucleic acid is selected from a group consisting of DNA and RNA.

176. The use of any one of claims 173-175, wherein the synthetic nucleic acid is a non-coding nucleic acid selected from a group consisting of a ssDNA (single-strand DNA), dsDNA (double-stranded DNA), small interfering RNA (siRNA), micro-RNA, dsRNA, lncRNA, piRNA, rmRNA, sRNA, tiRNA, eRNA, snoRNA, snRNA, circRNA (circular RNA), RNA aptamer, antisense oligonucleotide, a guide RNA, a tRNA or any combination thereof.

177. The use of any one of claims 173-175, wherein the heterologous nucleic acid comprises a coding region.

178. The use of claim 177, wherein the coding region codes for a therapeutic protein.

179. The use of any one of claim 171-172, or 178, wherein the recombinant protein or the therapeutic protein is selected from a group consisting of RPE65, REP1, RPGR, BEST1, anti-VEGF inhibitors such as aflibercept, ranibizumab, brolucizumab, or bevacizumab, pegatanib sodium, adalimumab, Infliximab, hRS1, hCNGB3, ABCR, MYO7A, endostatin, angiostatin, TNF [alpha] receptor, the TGF [beta]2 receptor, IRS1, IGF-1, Angiogenin, Angiopoietin-1, DeM, acidic or basic Fibroblast Growth Factors (aFGF and bFGF), FGF-2, Follistatin, Granulocyte Colony-Stimulating factor (G-CSF), Hepatocyte Growth Factor (HGF), Scatter Factor (SF), Leptin, Midkine, Placental Growth Factor (PGF), Platelet-Derived Endothelial Cell Growth Factor (PD-ECGF), Platelet-Derived Growth Factor-BB (PDGF-BB), Pleiotrophin (PTN), RdCVF (Rodderived Cone Viability Factor), Progranulin, Proliferin, Transforming Growth Factoralpha (TGF-alpha), PEDF, Transforming Growth Factor-beta (TGF-beta), Vascular Permeability Factor (VPF), CNTF, BDNF, GDNF, PEDF, NT3, BFGF, ephrin, EPO, NGF, GMF, aFGF, NT5, Gax, a growth hormone, [alpha]-1-antitrypsin, calcitonin, leptin, an apolipoprotein, an enzyme for the biosynthesis of vitamins, hormones or neuromediators, chemokines, cytokines such as IL-1, IL-8, IL-10, IL-12, IL-13, a receptor thereof, an antibody blocking any one of said receptors, TIMP such as TIMP1, TIMP-2, TIMP-3, TIMP-4, angioarrestin, endostatin such as endostatin XVIII and endostatin XV, ATF, a fusion protein of endostatin and angiostatin, the C-terminal hemopexin domain of matrix metalloproteinase-2, the kringle 5 domain of human plasminogen, a fusion protein of endostatin and the kringle 5 domain of human plasminogen, the placental ribonuclease inhibitor, the plasminogen activator inhibitor, the Platelet Factor-4 (PF4), a prolactin fragment, the Proliferin-Related Protein (PRP), the antiangiogenic antithrombin III, the Cartilage-Derived Inhibitor (CDI), a CD59 complement fragment, C3a and C5a inhibitors, complex attack membrane inhibitors, Factor H, ICAM, VCAM, caveolin, PKC zeta, junction proteins, JAMs, CD36, MERTK vasculostatin, vasostatin (calreticulin fragment), thrombospondin, fibronectin, in particular fibronectin fragment gro-beta, an heparinase, human chorionic gonadotropin (hCG), interferon alpha/beta/gamma, interferon inducible protein (IP-10), the monokine-induced by interferon-gamma (Mig), the interferon-alpha inducible protein 10 (IP10), a fusion protein of Mig and IP10, soluble Fms-Like Tyrosine kinase 1 (FLT1) receptor, Kinase insert Domain Receptor (KDR), regulators of apoptosis such as Bcl2, Bad, Bak, Bax, Bik, BcI-X short isoform and Gax, alpha-1 antitrypsin, factor IX, factor VIII, CI-esterase inhibitor, β-globin or γ-globin.

180. The use of any one of claims 169, 174 to 179, wherein the virus is selected from a group consisting of a retrovirus, adenovirus, adeno-associated virus (AAV), lentiviruses and herpes simplex virus.

181. The use of claim 180, wherein the virus is adeno-associated virus (AAV).

182. The use of claim 181, wherein the adeno-associated virus (AAV) is selected from a group consisting of AAV1, AAV2 such as AAV2.7m8, AAV3, AAV4, AAV5, AAV6, AAV7, AAV8, AAV9, AAV10, AAV11 and a mutant, hybrid or variant thereof.

183. The use of any one of claims 140-169, and 174-182, wherein the biologic is a virus or virus-like particle, wherein the virus is adeno-associated virus (AAV), and wherein AAV is selected from a group consisting of AAV2, AAV2.7m8, and AAV8.

184. The use of any one of claims 181 to 183, wherein the total amount of the AAV comprised in the implant is in the order of at least 10⁹ vg.

185. The use of claim 184, wherein the total amount of the AAV comprised in the implant is in the order from 10⁹ to 10¹⁵ vg such as 10¹⁰ to 10¹³ vg.

186. The use of claim 185, wherein the total concentration of the AAV comprised in the implant is at least 10¹³ vg/cm³.

187. A method for protecting a biologic against damage during a process wherein the biologic is directly exposed to an organic solvent, the method comprising mixing the biologic with at least one dehydration stabilizer before directly exposing the biologic to an organic solvent.

188. The method of claim 187, wherein the dehydration stabilizer and/or the biologic are substantially insoluble in the organic solvent such as a solubility of 0.1 mg/ml or less.

189. The method of any one of claims 187-188, wherein the total concentration of the dehydration stabilizer intermixed with the biologic is from 5 mg/ml to 200 mg/mL

190. The method of any one of claims 187-189, wherein the at least one dehydration stabilizer is selected from a group consisting of a carbohydrate, a sugar alcohol, and a combination thereof.

191. The method of claim 190, wherein the carbohydrate is selected from a monosaccharide, a disaccharide, an oligosaccharide, a water-soluble polysaccharide or a combination thereof.

192. The method of claim 190 or 191, wherein the carbohydrate is a sugar.

193. The method of claim 192, wherein the sugar is a non-reducing sugar.

194. The method of claim 192 or 193, wherein the sugar is selected from a group consisting of sucrose, trehalose, raffinose, stachyose, verbascose, hydrates thereof and a combination thereof, preferably sucrose, trehalose, trehalose dihydrate and a combination thereof.

195. The method of any one of claims 190-192, wherein the sugar alcohol is selected from a group consisting of erythritol, glycerol, isomalt, lactitol, maltitol, mannitol, sorbitol, xylitol, and a combination thereof.

196. The method of any one of claims 187-195, wherein the process in which the biologic is directly exposed to an organic solvent comprises forming an organogel comprising forming a matrix within which the biologic is dispersed.

197. The method of claim 196, wherein the matrix comprises covalently crosslinked multi-arm precursors formed in the presence of the organic solvent.

198. The method of claim 197, wherein the multi-arm precursors comprise at least two multi-arm precursors comprising a first multi-arm precursor comprising a first functional group, and a second multi-arm precursor comprising a second functional group.

199. The method of claim 198, wherein the multi-arm precursors comprise a third multi-arm precursor comprising the same functional group as the second multi-arm precursor.

200. The method of any one of claims 198 and 199, wherein each of the first functional group and the second functional group is selected from a group consisting of an electrophile and a nucleophile, and the reaction between the first functional group and second functional group is an electrophile-nucleophile reaction that forms the covalent bond.

201. The method of claim 200, wherein the nucleophile comprises an amine such as a primary amine, a thiol, an or a hydrazide.

202. The method of any one of claims 200 and 201, wherein the electrophile comprises succinimidyl esters, succinimidyl carbonates, nitrophenyl carbonates, aldehyde, ketones, acrylates, acrylamides, maleimides, vinylsulfones, iodoacetamides, alkenes, alkynes, norbornenes, epoxides, mesylates, tosylates, tresyls, cyanurates, orthopyridyl disulfides, or halides preferably, wherein the succinimidyl ester comprises a reactive group selected from succinimidyl succinate, succinimidyl glutarate, succinimidyl adipate, succinimidyl azelate, and succinimidyl glutaramide.

203. The method of any one of claims 187-202, wherein the process further comprises forming a xerogel.

204. The method of claim 203, wherein forming a xerogel comprises removing the organic solvent from the organogel.

205. The method of any one of claims 187-204, wherein the process further comprises forming a pharmaceutically acceptable implant.

206. The method of claim 205, wherein the pharmaceutically acceptable implant comprises a xerogel, and particles comprising a mixture of the biologic, and at least one dehydration stabilizer and optionally at least one further stabilizer such as a buffer and/or a surfactant.

207. The method of claim 206, wherein the xerogel comprises a matrix comprising covalently crosslinked multi-arm precursors within which the particles comprising the mixture of the biologic, and at least one dehydration stabilizer and optionally at least one further stabilizer such as a buffer and/or a surfactant are dispersed, preferably wherein the at least one dehydration stabilizer is a carbohydrate, sugar alcohol or a combination thereof.

208. The method of any one of claims 203 to 207, wherein the xerogel is a hydrogel upon exposure to an aqueous solution.

209. The method of any one of claims 187-208, wherein a further dehydration stabilizer is selected from a group consisting of polyalkylene oxide such as polyethylene glycol, polyvinyl pyrrolidinone, polyvinyl alcohol.

210. The method of any one of claims 205 to 209, wherein the pharmaceutically acceptable implant is for a controlled release of the biologic.

211. The method of claim 210, wherein the total (w/w) % of the carbohydrate, sugar alcohol or combination thereof is no greater than 40%, such as from 5 to 40%, wherein the (w/w) % is based on the weight of the pharmaceutically acceptable implant.

212. The method of claim 211, wherein the total (w/w) % of the carbohydrate, sugar alcohol or combination thereof is from 10 to 25%, wherein the (w/w) % is based on the weight of the pharmaceutically acceptable implant.

213. The method of any one of claims 210-212, wherein the pharmaceutically acceptable implant is for controlled release of a total amount of the biologic, and wherein the controlled release is characterized in that the number of days required for 100% release of the total amount of the biologic is at least 2 days.

214. The method of any one of claims 187-213, wherein the organic solvent is selected from a group consisting of methylene chloride, dimethyl carbonate, acetone, acetonitrile, ethyl acetate, and tetrahydrofuran.

215. The method of any one of claims 187-214, wherein the biologic comprises a plurality of the same or different biologics.

216. The method of claim 215, wherein the biologic is selected from a group consisting of a polypeptide, a virus or a virus-like particle, and a lipid encapsulating a nucleic acid(s).

217. The method of claim 216, wherein the polypeptide is any polypeptide having a primary, secondary, tertiary or quaternary structure.

218. The method of any one of claims 216-217, wherein the polypeptide is a recombinant protein.

219. The method of claim 218, wherein the recombinant protein is selected from an antibody, an antigen binding fragment, a fusion protein, a cytokine, a transcription factor, an enzyme or a hormone.

220. The method of claim 216, wherein the lipid encapsulating nucleic acid(s) comprises one or more heterologous nucleic acid(s).

221. The method of claim 216, wherein the virus or virus-like particle comprises a viral nucleic acid and one or more heterologous nucleic acid(s).

222. The method of any one of claims 220 and 221, wherein each heterologous nucleic acid is selected from a group consisting of DNA and RNA.

223. The method of any one of claims 220-222, wherein the heterologous nucleic acid is a non-coding nucleic acid selected from a group consisting of a ssDNA (singlestrand DNA), dsDNA (double-stranded DNA), small interfering RNA (siRNA), microRNA, dsRNA, lncRNA, piRNA, rmRNA, sRNA, tiRNA, eRNA, snoRNA, snRNA, circRNA (circular RNA), RNA aptamer, antisense oligonucleotide, a guide RNA, a tRNA or any combination thereof.

224. The method of any one of claims 220-223, wherein the heterologous nucleic acid comprises a coding nucleic acid sequence.

225. The method of claim 224, wherein the coding nucleic acid sequence codes for a therapeutic protein.

226. The method of any one of claim 218-219, or 225, wherein the recombinant protein or the therapeutic protein is selected from a group consisting of RPE65, REP1, RPGR, BEST1, anti-VEGF inhibitors such as aflibercept, ranibizumab, brolucizumab, or bevacizumab, pegatanib sodium, adalimumab, Infliximab, hRS1, hCNGB3, ABCR, MYO7A, endostatin, angiostatin, TNF [alpha] receptor, the TGF [beta]2 receptor, IRS1, IGF-1, Angiogenin, Angiopoietin-1, DeM, acidic or basic Fibroblast Growth Factors (aFGF and bFGF), FGF-2, Follistatin, Granulocyte Colony-Stimulating factor (G-CSF), Hepatocyte Growth Factor (HGF), Scatter Factor (SF), Leptin, Midkine, Placental Growth Factor (PGF), Platelet-Derived Endothelial Cell Growth Factor (PD-ECGF), Platelet-Derived Growth Factor-BB (PDGF-BB), Pleiotrophin (PTN), RdCVF (Rodderived Cone Viability Factor), Progranulin, Proliferin, Transforming Growth Factoralpha (TGF-alpha), PEDF, Transforming Growth Factor-beta (TGF-beta), Vascular Permeability Factor (VPF), CNTF, BDNF, GDNF, PEDF, NT3, BFGF, ephrin, EPO, NGF, GMF, aFGF, NT5, Gax, a growth hormone, [alpha]-1-antitrypsin, calcitonin, leptin, an apolipoprotein, an enzyme for the biosynthesis of vitamins, hormones or neuromediators, chemokines, cytokines such as IL-1, IL-8, IL-10, IL-12, IL-13, a receptor thereof, an antibody blocking any one of said receptors, TIMP such as TIMP1, TIMP-2, TIMP-3, TIMP-4, angioarrestin, endostatin such as endostatin XVIII and endostatin XV, ATF, a fusion protein of endostatin and angiostatin, the C-terminal hemopexin domain of matrix metalloproteinase-2, the kringle 5 domain of human plasminogen, a fusion protein of endostatin and the kringle 5 domain of human plasminogen, the placental ribonuclease inhibitor, the plasminogen activator inhibitor, the Platelet Factor-4 (PF4), a prolactin fragment, the Proliferin-Related Protein (PRP), the antiangiogenic antithrombin III, the Cartilage-Derived Inhibitor (CDI), a CD59 complement fragment, C3a and C5a inhibitors, complex attack membrane inhibitors, Factor H, ICAM, VCAM, caveolin, PKC zeta, junction proteins, JAMs, CD36, MERTK vasculostatin, vasostatin (calreticulin fragment), thrombospondin, fibronectin, in particular fibronectin fragment gro-beta, an heparinase, human chorionic gonadotropin (hCG), interferon alpha/beta/gamma, interferon inducible protein (IP-10), the monokine-induced by interferon-gamma (Mig), the interferon-alpha inducible protein 10 (IP10), a fusion protein of Mig and IP10, soluble Fms-Like Tyrosine kinase 1 (FLT1) receptor, Kinase insert Domain Receptor (KDR), regulators of apoptosis such as Bcl2, Bad, Bak, Bax, Bik, BcI-X short isoform and Gax, alpha-1 antitrypsin, factor IX, factor VIII, CI-esterase inhibitor, β-globin or γ-globin.

227. The method of any one of claims 216 to 226, wherein the virus is selected from a group consisting of a retrovirus, adenovirus, adeno-associated virus (AAV), lentivirus and herpes simplex virus.

228. The method of claim 227, wherein the virus is adeno-associated virus (AAV).

229. The method of claim 228, wherein the adeno-associated virus (AAV) is selected from a group consisting of AAV1, AAV2 such as AAV2.7m8, AAV3, AAV4, AAV5, AAV6, AAV7, AAV8, AAV9, AAV10, AAV11 and a mutant, hybrid or variant thereof.

230. The method of any one of claims 187-216, and 221-229, wherein the biologic is a virus or virus-like particle, wherein the virus is adeno-associated virus (AAV), and wherein AAV is selected from a group consisting of AAV2, AAV2.7m8, and AAV8.

231. The method of any one of claims 228-230 wherein the total amount of the AAV comprised in the implant is in the order of at least 10⁹ vg.

232. The method of claim 231, wherein the total amount of the AAV comprised in the implant is in the order from 10⁹ to 10¹⁵ vg such as 10¹⁰ to 10¹³ vg.

233. The method of claim 232, wherein the total concentration of the AAV comprised in the implant is at least 10¹³ vg/cm³.

233. A method for manufacturing a pharmaceutically acceptable implant comprising a biologic comprising
(A) forming an organogel including the biologic comprising forming a matrix comprising at least two multi-arm precursors that are covalently crosslinked in an organic solvent in the presence of the biologic,
(B) forming a xerogel comprising removing the organic solvent.

234. The method of claim 233, wherein step (A) comprises the following steps:
(a) providing a mixture of the biologic and at least one dehydration stabilizer, and optionally at least one further stabilizer such as a buffer and/or a surfactant,
(b) providing a first multi-arm precursor,
(c) providing a second multi-arm precursor or a mixture of the second multi-arm precursor and a third multi-arm precursor.

235. The method of claim 234, wherein each of (a), (b) and (c) are processed to obtain:
(d) particles comprising the mixture of the biologic and at least one dehydration stabilizer, and optionally at least one further stabilizer such as a buffer and/or a surfactant,
(e) processed first multi-arm precursor,
(f) processed second multi-arm precursor or a processed mixture of the second multiarm precursor and a third multi-arm precursor.

236. The method of claim 235, wherein processing of (a) and optionally of (b) and (c) comprises forming dried particulates such as by lyophilization or spray drying.

237. The method of any one of claims 235-236, wherein processing of (b) and (c) to obtain (e) and (f) comprises sterilization such as gamma sterilization, e-beam sterilization, or ethylene oxide sterilization.

238. The method of any one of claims 235-237, wherein step (A) comprises:
(A-1) adding an organic solvent to each of (e) and (f) to obtain (g) and (h),
(A-2) mixing (d) and (g) to obtain (i),
(A-3) mixing (h) and (i)
to form an organogel.

239. The method of claims 233-238, wherein step (B) comprises drying to form a xerogel from the organogel.

240. The method of claim 239, wherein drying is from 1 to 5 days, such as 3 days.

241. The method of claim 240, wherein the temperature during drying is from 33 to 38° C., such as 35-37° C.

242. The method of any one of claims 233-241, wherein the pharmaceutically acceptable implant is in the form of a fiber, wherein the fiber is characterized by a diameter of about 0.1 mm or more and/or a length of about 2.0 mm or more.

243. The method of any one of claims 233-242, wherein the at least one dehydration stabilizer protects the biologic from damage upon direct exposure to the organic solvent in step (A).

244. The method of any one of claims 234-244, wherein the at least one dehydration stabilizer is selected from a group consisting of carbohydrates, a sugar alcohols and combination thereof.

245. The method of claim 244, wherein the carbohydrate is selected from a monosaccharide, a disaccharide, an oligosaccharide, a water-soluble polysaccharide or a combination thereof.

246. The method of claim 245, wherein the carbohydrate is a sugar.

247. The method of claim 246, wherein the sugar is a non-reducing sugar.

248. The method of any one of claims 246-247, wherein the sugar is selected from a group consisting of sucrose, trehalose, raffinose, stachyose, verbascose, hydrates thereof and a combination thereof, preferably sucrose, trehalose, trehalose dihydrate and a combination thereof.

249. The method of any one of claims 244-248, wherein the sugar alcohol is selected from a group consisting of erythritol, glycerol, isomalt, lactitol, maltitol, mannitol, sorbitol, xylitol, and a combination thereof.

250. The method of any one of claims 235-249, wherein a further dehydration stabilizer is selected from a group consisting of polyalkylene oxide such as polyethylene glycol, polyvinyl pyrrolidinone, and polyvinyl alcohol.

251. The method of claim 250, wherein the further dehydration stabilizer is the first multi-arm precursor.

252. The method of any one of claims 234-251, wherein (b) is mixed with (a).

253. The method of any one of claims 235-252, wherein (e) is part of (d).

254. The method of any one of claims 234-253, wherein claim 234 (b) or (c) further comprises a polymer that does not participate in the cross-linking reaction between the multi-arm precursors, wherein the MW of the polymer is from 1,000 to 35,000 Da, and wherein the polymer is selected from a group consisting of polyalkylene oxide such as polyethylene glycol, polyvinyl pyrrolidinone, polyvinyl alcohol.

255. The method of any one of claims 233-254, wherein the pharmaceutically acceptable implant comprises a xerogel, and particles comprising the mixture of the biologic, and at least one dehydration stabilizer and optionally at least one further stabilizer such as a buffer and/or a surfactant.

256. The method of claim 255, wherein the xerogel comprises a matrix comprising covalently crosslinked multi-arm precursors within which the particles comprising the mixture of the biologic, and at least one dehydration stabilizer and optionally at least one further stabilizer such as a buffer and/or a surfactant are dispersed, preferably wherein the at least one dehydration stabilizer is selected from a carbohydrate, sugar alcohol, or combination thereof.

257. The method of any one of claims 233-256, wherein the xerogel is a hydrogel upon exposure to an aqueous solution.

258. The method of any one of claims 233-257, wherein the multi-arm precursors comprise a first multi-arm precursor comprising a first functional group, and a second multi-arm precursor comprising a second functional group.

259. The method of claim 258, wherein the multi-arm precursors comprise a third multi-arm precursor comprising the same functional group as the second multi-arm precursor.

260. The method of claim 258 or 259, wherein each of the first functional group and the second functional group is each selected from a group consisting of an electrophile and a nucleophile, and the reaction between the first functional group and second functional group is an electrophile-nucleophile reaction that forms the covalent bond.

261. The method of claim 260, wherein the nucleophile comprises an amine such as a primary amine, a thiol, an or a hydrazide.

262. The method of claim 260 or 261, wherein the electrophile comprises succinimidyl esters, succinimidyl carbonates, nitrophenyl carbonates, aldehyde, ketones, acrylates, acrylamides, maleimides, vinylsulfones, iodoacetamides, alkenes, alkynes, norbornenes, epoxides, mesylates, tosylates, tresyls, cyanurates, orthopyridyl disulfides, or halides preferably, wherein the succinimidyl ester comprises a reactive group selected from succinimidyl succinate, succinimidyl glutarate, succinimidyl adipate, succinimidyl azelate, and succinimidyl glutaramide.

263. The method of any one of claims 233-262, wherein the method is for manufacturing a pharmaceutically acceptable implant for controlled release of the total amount of the biologic, and wherein the controlled release is characterized by:
(A) the amount of the biologic released on day 1 is from 0 to 50% of the total amount of the biologic,
(B) the amount of the biologic released per day from day 2 until the last day of release is from 0 to 50% of the total amount of the biologic, and/or
(C) the number of days required for 100% release of the total amount of the biologic is at least 2 days.

264. The method of claim 263, wherein the controlled release is characterized by:
(A) the amount of the biologic released on day 1 is from 0 to 25%, 0 to 20% 0 to 10%, 0 to 5%, or about 0% of the total amount of the biologic,
(B) the amount of the biologic released per day from day 2 until the last day of release is from 0 to 50% of the total amount of the biologic, and/or
(C) the number of days required for 100% release of the total amount of the biologic is at least 3 days but no greater than 30 days, 25 days, or no greater than 16 days.

265. The method of claim 263 or 264, wherein the controlled release is measured from the time and under conditions wherein the implant is first immersed in an aqueous solution under physiological conditions such as at pH 7.2-7.4 and temperature 37° C.

266. The method of any one of claims 263-265, wherein the total (w/w) % of the carbohydrate, sugar alcohol or combination thereof is selected to provide for the controlled release as defined in claim 263 item (C) or claim 264 item (C).

267. The method of any one of claims 263-265, wherein the molecular weight between crosslinks in the xerogel is selected to provide for the controlled release as defined in claims 30 and 31.

268. The method of any one of claims 263-265, wherein
(i) the (w/w) % of the total particles comprising the mixture of the biologic and the carbohydrate, sugar alcohol or combination thereof, and optionally at least one further stabilizer such as a buffer and/or a surfactant,
(ii) the (w/w) % of the total number of multi-arm precursors,
(iii) the ratio of (i) and (ii), and/or
(iv) the D90 particle size such as DV₉₀ particle size, wherein particles comprise the mixture of the biologic and at least one dehydration stabilizer such as a carbohydrate, a sugar alcohol, or a combination thereof, and optionally at least one further stabilizer such as a buffer and/or a surfactant,
is selected to provide for the controlled release as defined in claim 263 or 264, and wherein the (w/w) % is based on the weight of the pharmaceutically acceptable implant.

269. The method of any one of claims 263-265, wherein the precursors comprise at least three multi-arm precursors comprising
(i) a first multi-arm precursor comprising a nucleophile,
(ii) a second multi-arm precursor comprising an electrophile comprising a first reactive group, and
(iii) a third multi-arm precursor comprising an electrophile comprising a second reactive group.

270. The method of claim 269, wherein the third multi-arm precursor has a longer hydrolysis half-life as compared to the second multi-arm precursor.

271. The method of claim 269 or 270, wherein the electrophile-nucleophile reaction between the first and the second multi-arm precursors, and the first and the third multi-arm precursors forms the covalent bond.

272. The method of any one of claims 269-271, wherein the nucleophile comprises an amine such as a primary amine, a thiol, or a hydrazide.

273. The method of any one of claims 269-272, wherein the electrophile comprises succinimidyl esters, succinimidyl carbonates, nitrophenyl carbonates, aldehyde, ketones, acrylates, acrylamides, maleimides, vinylsulfones, iodoacetamides, alkenes, alkynes, norbornenes, epoxides, mesylates, tosylates, tresyls, cyanurates, orthopyridyl disulfides, or halides preferably, wherein the succinimidyl ester comprises a reactive group selected from succinimidyl succinate, succinimidyl glutarate, succinimidyl adipate, succinimidyl azelate, and succinimidyl glutaramide.

274. The method of any one of claims 272 and 273, wherein
(i) the first multi-arm precursor comprises a primary amine,
(ii) the second multi-arm precursor comprises a succinimidyl ester comprising a first reactive group, and,
(iii) the third multi-arm precursor comprises a succinimidyl ester comprising a second reactive group,
wherein the first reactive group is succinimidyl succinate and the second reactive group is succinimidyl glutarate.

275. The method according to any one of claims 263-265 and 269-274, wherein the molar ratio of:
(i) the first reactive group comprised in the second multi-arm precursor, and
(ii) the second reactive group comprised in the third multi-arm precursor, is selected to provide for the controlled release as defined in claims 30 and 31.

276. The method according to claims 263-275, wherein the controlled release is achieved by
(a) selecting the total (w/w) % of the carbohydrate, sugar alcohol or combination thereof,
(b) selecting the molecular weight between crosslinks in the xerogel,
(c) selecting the (w/w) % of the total particles comprising the mixture of the biologic and the carbohydrate, sugar alcohol or combination thereof, and optionally at least one further stabilizer such as a buffer and/or a surfactant,
(d) selecting the (w/w) % of the total number of multi-arm precursors,
(e) selecting the ratio of (c) and (d),
(f) selecting the D₉₀ particle size such as Dv₉₀ particle size, wherein particles comprise the mixture of the biologic and at least one dehydration stabilizer such as a carbohydrate, a sugar alcohol, or a combination thereof, and optionally at least one further stabilizer such as a buffer and/or a surfactant, and/or
(g) selecting the molar ratio of:
(g-i) the first reactive group comprised in the second multi-arm precursor, and
(g-ii) the second reactive group comprised in the third multi-arm precursor,
wherein the (w/w) % is based on the weight of the pharmaceutically acceptable implant.

277. The method of claim 276, wherein the total (w/w) % of carbohydrate, sugar alcohol or combination thereof is selected to be no greater than 40%, such as from 5 to 40%, wherein the (w/w) % is based on the weight of the pharmaceutically acceptable implant.

278. The method of claim 277, wherein the total (w/w) % of the carbohydrate, sugar alcohol or combination thereof is selected from no greater than 10 to 35%, wherein the (w/w) % is based on the weight of the pharmaceutically acceptable implant.

279. The method of any one of claims 276-278, wherein the molecular weight between crosslinks in the xerogel is selected from 7 to 25 kDa.

280. The method of claim 279, wherein the molecular weight between crosslinks in the xerogel is selected from 9 to 16 kDa.

281. The method of any one of claims 276-280, wherein,
(i) the (w/w) % of the total particles comprising the mixture of the biologic and the carbohydrate, sugar alcohol or combination thereof, and optionally at least one further stabilizer such as a buffer and/or a surfactant is selected to be no greater than 80%, or no greater than 70%, or no greater than 60%, or no greater than 50%,
(ii) the (w/w) % of the total number of multi-arm precursors is selected from 20% to 80% such as from 35% to 75%,
(iii) the ratio of (i) and (ii) is selected from 0.3 to 4.0 such as from 0.3 to 2.0,
(iv) the Dv₉₀ particle size is selected to be from 10 μm to 200 μm such as 35 μm to 75 μm such as 35 μm to 100 μm, or 35 μm to 150 μm, wherein particles comprise the mixture of the biologic and at least one dehydration stabilizer such as a carbohydrate, a sugar alcohol, or a combination thereof, and optionally at least one further stabilizer such as a buffer and/or a surfactant,
wherein the (w/w) % is based on the weight of the pharmaceutically acceptable implant.

282. The method of claim 281, wherein the ratio is selected from 0.5 to 2.0, from 0.5 to 1.0, from 0.7 to 1.3 such as from 0.6 to 0.9.

283. The method of any one of claims 276-282, wherein the molar ratio of:
(i) the first reactive group comprised in the second multi-arm precursor, and
(ii) the second reactive group comprised in the third multi-arm precursor, is selected from 0:100 to 100:0.

284. The method of claim 283, wherein the ratio is selected from 30-90:70-10.

285. The method of any one of claims 283-284, wherein the first reactive group is succinimidyl succinate and the second reactive group is succinimidyl glutarate.

286. The method of any one of claims 263-285, wherein
(a) the total (w/w) % of the carbohydrate, sugar alcohol or combination thereof is selected to be no greater than 40%, such as from 5 to 40%, wherein the (w/w) % is based on the weight of the pharmaceutically acceptable implant,
(b) the molecular weight between crosslinks in the xerogel is selected from 7 to 25 kDa,
(c) the (w/w) % of the total particles comprising the mixture of the biologic and the carbohydrate, sugar alcohol or combination thereof, and optionally at least one further stabilizer such as a buffer and/or a surfactant is selected to be no greater than 80%, or no greater than 70%, or no greater than 60%, or no greater than 50%,
(d) the (w/w) % of the total number of multi-arm precursors is selected from 20% to 80% such as from 35% to 75%,
(e) the ratio of (c) and (d) is selected from 0.3 to 4.0 such as from 0.3 to 2.0,
(f) the Dv₉₀ particle size is selected to be from 10 μm to 200 μm such as 35 μm to 75 μm such as 35 μm to 100 μm, or 35 μm to 150 μm, wherein particles comprise the mixture of the biologic and at least one dehydration stabilizer such as a carbohydrate, a sugar alcohol, or a combination thereof, and optionally at least one further stabilizer such as a buffer and/or a surfactant, and/or
(g) the molar ratio of:
(g-i) the first reactive group comprised in the second multi-arm precursor and
(g-ii) the second reactive group comprised in the third multi-arm precursor,
is selected from 30-90:70-10, wherein the first reactive group is succinimidyl succinate and the second reactive group is succinimidyl glutarate,
wherein the (w/w) % is based on the weight of the pharmaceutically acceptable implant.

287. The method of any one of claims 263-286, wherein the controlled release comprises a zero-order release or substantially a zero-order release.

288. The method of claim 287, wherein the zero-order release starts at least one day after the implant is first immersed in an aqueous solution under physiological conditions such as at pH 7.2-7.4 and temperature 37° C.

289. The method of any one of claims 263-288, wherein the controlled release is characterized by at least 3 days to 65 days.

290. The method of any one of claims 263-289, wherein during the controlled release,
(i) the diameter and/or the length of the fiber increases, such as at least by 1.5 folds,
(ii) the length of the fiber does not change, and/or
(iii) the diameter and/or length of the fiber decreases,
as measured after the implant is first immersed in an aqueous solution under physiological conditions such as at pH 7.2-7.4 and temperature 37° C.

291. The method of claims 233-290, wherein the biologic comprises a plurality of the same or different biologics.

292. The method of claim 291, wherein the biologic is selected from a group consisting of a polypeptide, a virus or a virus-like particle, and a lipid encapsulating a nucleic acid(s).

293. The method of claim 292, wherein the polypeptide is any polypeptide having a primary, secondary, tertiary or quaternary structure.

294. The method of any one of claims 292-293, wherein the polypeptide is a recombinant protein.

295. The method of claim 294, wherein the recombinant protein is selected from an antibody, an antigen binding fragment, a fusion protein, a cytokine, a transcription factor, an enzyme or a hormone.

296. The method of claim 292, wherein the lipid encapsulating nucleic acid(s) comprises one or more heterologous nucleic acid(s).

297. The method of claim 292, wherein the virus or virus-like particle comprises a viral nucleic acid and one or more heterologous nucleic acid(s).

298. The method of any one of claims 296-297, wherein each heterologous nucleic acid is selected from a group consisting of DNA and RNA.

299. The method of claim 298, wherein the heterologous nucleic acid is a non-coding nucleic acid selected from a group consisting of a ssDNA (single-strand DNA), dsDNA (double-stranded DNA), small interfering RNA (siRNA), micro-RNA, dsRNA, lncRNA, piRNA, rmRNA, sRNA, tiRNA, eRNA, snoRNA, snRNA, circRNA (circular RNA), RNA aptamer, antisense oligonucleotide, a guide RNA, a tRNA or any combination thereof.

300. The method of any one of claims 296-298, wherein the heterologous nucleic acid comprises a coding nucleic acid sequence.

301. The method of claim 300, wherein the coding nucleic acid sequence codes for a therapeutic protein.

302. The method of any one of claim 294-295, or 301, wherein the recombinant protein or therapeutic protein is selected from a group consisting of RPE65, REP1, RPGR, BEST1, anti-VEGF inhibitors such as aflibercept, ranibizumab, brolucizumab, or bevacizumab, pegatanib sodium, adalimumab, Infliximab, hRS1, hCNGB3, ABCR, MYO7A, endostatin, angiostatin, TNF [alpha] receptor, the TGF [beta]2 receptor, IRS1, IGF-1, Angiogenin, Angiopoietin-1, DeM, acidic or basic Fibroblast Growth Factors (aFGF and bFGF), FGF-2, Follistatin, Granulocyte Colony-Stimulating factor (G-CSF), Hepatocyte Growth Factor (HGF), Scatter Factor (SF), Leptin, Midkine, Placental Growth Factor (PGF), Platelet-Derived Endothelial Cell Growth Factor (PD-ECGF), Platelet-Derived Growth Factor-BB (PDGF-BB), Pleiotrophin (PTN), RdCVF (Rod-derived Cone Viability Factor), Progranulin, Proliferin, Transforming Growth Factor-alpha (TGF-alpha), PEDF, Transforming Growth Factor-beta (TGF-beta), Vascular Permeability Factor (VPF), CNTF, BDNF, GDNF, PEDF, NT3, BFGF, ephrin, EPO, NGF, GMF, aFGF, NT5, Gax, a growth hormone, [alpha]-1-antitrypsin, calcitonin, leptin, an apolipoprotein, an enzyme for the biosynthesis of vitamins, hormones or neuromediators, chemokines, cytokines such as IL-1, IL-8, IL-10, IL-12, IL-13, a receptor thereof, an antibody blocking any one of said receptors, TIMP such as TIMP-1, TIMP-2, TIMP-3, TIMP4, angioarrestin, endostatin such as endostatin XVIII and endostatin XV, ATF, a fusion protein of endostatin and angiostatin, the C-terminal hemopexin domain of matrix metalloproteinase-2, the kringle 5 domain of human plasminogen, a fusion protein of endostatin and the kringle 5 domain of human plasminogen, the placental ribonuclease inhibitor, the plasminogen activator inhibitor, the Platelet Factor-4 (PF4), a prolactin fragment, the Proliferin-Related Protein (PRP), the antiangiogenic antithrombin III, the Cartilage-Derived Inhibitor (CDI), a CD59 complement fragment, C3a and C5a inhibitors, complex attack membrane inhibitors, Factor H, ICAM, VCAM, caveolin, PKC zeta, junction proteins, JAMs, CD36, MERTK vasculostatin, vasostatin (calreticulin fragment), thrombospondin, fibronectin, in particular fibronectin fragment gro-beta, an heparinase, human chorionic gonadotropin (hCG), interferon alpha/beta/gamma, interferon inducible protein (IP-10), the monokine-induced by interferon-gamma (Mig), the interferon-alpha inducible protein 10 (IP10), a fusion protein of Mig and IP10, soluble FmsLike Tyrosine kinase 1 (FLT-1) receptor, Kinase insert Domain Receptor (KDR), regulators of apoptosis such as Bcl-2, Bad, Bak, Bax, Bik, BcI-X short isoform and Gax, alpha-1 antitrypsin, factor IX, factor VIII, CI-esterase inhibitor, β-globin or γ-globin.

303. The method of any one of claims 297 to 302, wherein the virus is selected from a group consisting of retrovirus, adenovirus, adeno-associated virus (AAV), lentivirus and herpes simplex virus.

304. The method of claim 303, wherein the virus is adeno-associated virus (AAV).

305. The method of claim 304, wherein the adeno-associated virus (AAV) is selected from a group consisting of AAV1, AAV2 such as AAV2.7m8, AAV3, AAV4, AAV5, AAV6, AAV7, AAV8, AAV9, AAV10, AAV11 and a mutant, hybrid or variant thereof.

306. The method of any one of claims 233-292, and 297-305, wherein the biologic is a virus, wherein the virus is adeno-associated virus (AAV), and wherein AAV is selected from a group consisting of AAV1, AAV2 such as AAV2.7m8, AAV3, AAV4, AAV5, AAV6, AAV7, AAV8, AAV9, AAV10, AAV11 and a mutant, hybrid or variant thereof.

307. The method of any one of claims 304-306, wherein the total amount of the AAV comprised in the implant is in the order of at least 10⁹ vg.

308. The method of claim 307, wherein the total amount of the AAV comprised in the implant is in the order from 10⁹ to 10¹⁵ vg such as 10¹⁰ to 10¹³ vg.

309. The method of claim 308, wherein the total concentration of the AAV comprised in the implant is at least 10¹³ vg/cm³.

310. The method of any one of claims 304-309, wherein the controlled release is characterized by
(A) no greater than 9.0×10⁹ to 1.5×10¹⁰ vg AAV released on day 1,
(B) no greater than in the order 10¹¹ vg AAV per day such as in the order 10⁸, or 10⁹ or 10¹⁰ such as 5.0×10⁹ to 1.5×10¹⁰ vg AAV released per day from day 2 until the last day of the controlled release, and/or
(C) the number of days required for 100% release of the AAV is not less than 4 days.

311. The pharmaceutically acceptable implant of any one of claims 1 to 95, which is alternatively an in-situ gel such as in-situ implant.

312. A method of delivering an AAV to a subject in need thereof, comprising injecting the in-situ gel such as in-situ implant of claim 311 into a suprachoroidal space of the subject.

313. A method of treating a wet-AMD in a subject in need thereof, comprising injecting the in-situ gel such as in-situ implant of claim 311 into a suprachoroidal space of the subject.

314. The pharmaceutically acceptable implant of claim 311, for use in a method of delivering an AAV to a subject in need thereof the method comprising injecting the insitu gel such as in-situ implant into a suprachoroidal space of the subject

315. The pharmaceutically acceptable implant of claim 311 for use in a method of treating a wet-AMD in a subject in need thereof, the method comprising injecting the in-situ gel such as an in-situ implant into a suprachoroidal space of the subject.
Product characterization claims

316. The pharmaceutically acceptable implant according to any one of claims 1 to 95, characterized in that the implant induces an immune response such as an adaptive immune response such as a humoral immune response as measured by detectable serum titer of ADA in a rabbit against the biologic comprised in the implant, wherein the serum titer of ADA is no greater than 20,000, or 15,000, or 10,000, or 8,000, or 7,000, or 5,000, or 2,000, or 1,000 or below detection limit as compared to the serum titer of the ADA at baseline in the rabbit, wherein the implant is administered to the eye of the rabbit such as an intravitreal administration.

317. The pharmaceutically acceptable implant according to claim 316, wherein the serum titer of ADA pertains to
(i) any time point from week 8 to week 13 post-administration such as at week 8 or at week 13 post-administration, or
(ii) a corresponding time point when compared to (i), wherein (i) pertains to the biologic being AAV2.7m8.

318. The pharmaceutically acceptable implant according to any one of claims 1 to 95, characterized in that it provides a total detectable amount of the biologic in the plasma per mL of a rabbit that is at least four log-less, five log-less, six log-less, seven log-less, eight log-less, nine log less or below detection limit as compared to the total amount of the biologic comprised in the implant, wherein the implant is administered to the eye of the rabbit such as an intravitreal administration.

319. The pharmaceutically acceptable implant according to claim 318, wherein the total detectable concentration of the biologic pertains to
(i) any time point from day 1 to day 3 post-administration such as at day 2 postadministration, or
(ii) a corresponding time point when compared to (i) wherein (i) pertains to the biologic being AAV2.7m8.

320. The pharmaceutically acceptable implant according to any one of claims 316 to 319, wherein the pharmaceutically acceptable implant is for controlled release of a total amount of the biologic.
Method of controlling an immune response

321. A method of controlling an immune response such as an adaptive immune response such as humoral immune response when treating an ocular disorder such as an ocular genetic disorder comprising administering to a subject such as human a pharmaceutically acceptable implant according to any one of claims 1 to 95 or 316 to 320.

322. The pharmaceutically acceptable implant according to any one of claims 1 to 95, or 316 to 320 for use in controlling an immune response such as an adaptive immune response or humoral immune response when treating an ocular disorder such as an ocular genetic disorder.

323. The method or use of claim 321 or 322, wherein the administration is to the eye of the subject such as intravitreal injection.

324. The method or use of any one of claims 321 to 323, wherein controlling an immune response such as an adaptive immune response such as humoral immune response is characterized by inducing an ADA titer against a biologic comprised in the implant after administration of the implant to the subject, wherein the ADA titer in said subject is no greater than 20,000, or 15,000, or 10,000, or 8,000, or 7,000, or 5,000, or 2,000, or 1,000 or below detection limit as compared to the ADA titer at baseline.

325. The method or use of claim 324, wherein the ADA titer pertains to the ADA titer at any time from week 8 to week 13 post-administration such as at week 8 or at week 13 post-administration.

326. The method or use of any one of claims 321 to 323, wherein the implant comprises a total amount of a biologic, and wherein controlling an immune response such as an adaptive immune response such as humoral immune response is characterized by inducing an ADA titer against the biologic after administration of the implant to the subject, wherein the ADA titer is lower than the ADA titer against the same biologic obtained after administering another composition such as a bolus comprising the same total amount of the biologic and administered to a comparison subject at the same time.

327. The method or use of any one of claims 321 to 323, wherein the implant comprises a total amount of a biologic, and wherein controlling an immune response such as an adaptive immune response such as humoral immune response is characterized by inducing an ADA titer against the biologic after administration of the implant to the subject, wherein the ADA titer is lower by at least 10%, or at least 20%, or at least, 30%, or at least 40%, or at least 50%, or at least 60%, or at least 70%, or at least 80%, or at least 90%, or 100% than the ADA titer against the same biologic obtained after administering another composition such as a bolus comprising the same total amount of the biologic and administered to a comparison subject at the same time.

328. The method or use of any one of claims 321 to 323, wherein the implant comprises a total amount of a biologic, and wherein controlling an immune response such as an adaptive immune response such as humoral immune response is characterized by inducing an ADA titer against the biologic comprised in the implant after administration of the implant to the subject, wherein the ADA titer is lower by at least 20,000, or 15,000, or 10,000, or 8,000, or 7,000, or 5,000, or 2,000, or 1,000 than the ADA titer against the same biologic obtained after administering another composition such as a bolus comprising the same total amount of the biologic and administered to a comparison subject at the same time.

329. The method or use of any one of claims 326 to 328 wherein the ADA titer pertains to a time point post administration in which the ADA titer due to the other composition such as a bolus is at least 5,000.

330. The method or use of any one of claims 326 to 329, wherein the ADA titer pertains to at any time from week 8 to week 13 post-administration such as at week 8 or at week 13 post-administration.
Method effective treatment claims

331. A method of effective treatment of an ocular disorder such as an ocular genetic disorder comprising administering to a subject such as human the pharmaceutically acceptable implant according to any one of claims 1 to 95 or 316 to 320.

332. The pharmaceutically acceptable implant according to any one of claims 1 to 95, or 316 to 320 for use in an effective treatment of an ocular disorder such as an ocular genetic disorder in a subject such as human.

333. The method or use of claim 331 or 332, wherein administration is to the eye of the subject such as intravitreal administration.

334. The method or use of anyone of claims 331 to 333, wherein the effective treatment is characterized by the total detectable amount of the biologic in the systemic circulation of the subject such as at day 1, 2 or 3 post administration that is lower than the total amount of the biologic comprised in the implant.

335. The method or use of anyone of claims 331 to 333, wherein the biologic is AAV and wherein the effective treatment is characterized by the total detectable amount of an endogenous nucleic acid sequence of said AAV in the systemic circulation of the subject such as at day 1, 2 or 3 post administration that is at least four log-less, five log-less, six log-less, seven log-less, eight log-less, nine log less or below detection limit as compared to the total amount of the AAV in gc comprised in the implant.

336. The method or use of anyone of claims 331 to 333, wherein the biologic is AAV comprising a heterologous nucleic acid sequence, and wherein the effective treatment is characterized by the total detectable amount of the heterologous nucleic acid sequence in the systemic circulation of the subject such as at day 1, 2 or 3 post administration that is at least four log-less, five log-less, six log-less, seven log-less, eight log-less, nine log less or below detection limit as compared to the total amount of the AAV in gc comprised in the implant.

337. The method or use of anyone of claims 331 to 333, wherein the implant comprises a total amount of a biologic and wherein the effective treatment is characterized by the total detectable amount of the biologic in the systemic circulation of the subject such as at day 1, 2 or 3 post administration that is lower as compared to the total detectable amount of the same biologic in the systemic circulation of the subject obtained after administering another composition such as a bolus comprising the same total amount of the biologic and administered to a comparison subject at the same time.

338. The method or use of anyone of claims 331 to 333, wherein the implant comprises a total amount of a biologic and wherein the effective treatment is characterized by a lower Cₘₐₓ in the subject as compared to the Cₘₐₓ obtained in a subject after administering another composition such as a bolus comprising the same total amount of the biologic and administered to a comparison subject at the same time, wherein Cₘₐₓ pertains to the Cₘₐₓ at any time during the entire period of the treatment.

339. The method or use of anyone of claims 331 to 333, wherein the biologic is AAV comprised in the implant at a total amount, and wherein the effective treatment is characterized by the total detectable amount of an endogenous nucleic acid sequence of said AAV in the systemic circulation of the subject that is at least one log-less, two log-less, three log-less, four log-less, five log-less, such as at day 1, day 2, or day 3 post administration as compared to the total detectable amount of the same nucleic acid sequence of the genome of said AAV in the systemic circulation of a subject obtained after administering another composition such as a bolus comprising the same total amount of said AAV and administered to a comparison subject at the same time.

340. The method or use of anyone of claims 331 to 333, wherein the biologic is AAV comprised in the implant at a total amount, and wherein the effective treatment is characterized by the Cₘₐₓ of the endogenous nucleic acid sequence of said AAV in the subject that is at least one log-less, two log-less, three log-less, four log-less, five loglessas compared to the Cₘₐₓ of the same nucleic acid sequence of the genome of said AAV obtained after administering another composition such as a bolus comprising the same total amount of said AAV and administered to a comparison subject at the same time, wherein Cₘₐₓ pertains to the Cₘₐₓ at any time during the entire period of the treatment.

341. The method or use of anyone of claims 331 to 333, wherein the biologic is AAV comprised in the implant at a total amount, wherein the AAV comprises a heterologous nucleic acid sequence, and wherein the effective treatment is characterized by the total detectable amount of the heterologous nucleic acid sequence in the systemic circulation of the subject that is at least one log-less, two log-less, three log-less, four log-less, five log-less, such as at day 1, day 2, or day 3 post administration as compared to the total detectable amount of the heterologous nucleic acid sequence of said AAV in the systemic circulation of a subject obtained after administering another composition such as a bolus comprising the same total amount of said AAV and administered to a comparison subject at the same time.

342. The method or use of anyone of claims 331 to 333, wherein the biologic is AAV comprised in the implant at a total amount, wherein the AAV comprises a heterologous nucleic acid sequence, and wherein the effective treatment is characterized by the Cₘₐₓ of the heterologous nucleic acid sequence in the subject that is at least one log-less, two log-less, three log-less, four log-less, five log-less as compared to the Cₘₐₓ of the of the same heterologous nucleic acid sequence comprised in the AAV obtained after administering another composition such as a bolus comprising the same total amount of said AAV and administered to a comparison subject at the same time, wherein Cₘₐₓ pertains to the Cₘₐₓ at any time during the entire period of the treatment.

343. The method of any one of claims 331 to 342, wherein the effective treatment is further characterized by a controlled immune response according to any one of claims 320 to 329.

344. The method of any one of claims 331 to 343, wherein the effective treatment is further characterized by a controlled inflammation according to any one of claims 100 to 104.

345. The method of any one of claims 326 to 328, or 337 to 342, wherein the other composition is selected from
(i) a bolus comprising the same biologic at the same total amount,
(ii) a pharmaceutically acceptable implant according to any one of claims 1 to 95 comprising the same biologic at the same total amount characterized in that the total number of days required for 100% release of the total amount of the biologic is fewer,
(ii) a pharmaceutically acceptable implant according to any one of claims 1 to 95 comprising the same biologic at a higher total amount,
(iii) a pharmaceutically acceptable implant according to any one of claims 1 to 95 comprising the same biologic at a higher total amount and characterized in that the total number of days required for 100% release of the total amount of the biologic is fewer,
wherein the release is measured from the time and under conditions wherein the implant is first immersed in an aqueous solution under physiological conditions such as at pH 7.2-7.4 and temperature 37° C.

346. The method or use according to any one of claims 321 to 345, wherein the pharmaceutically acceptable implant is for controlled release of a total amount of the biologic.

347. The method or use according to claim any one of claims 321 to 346, wherein the controlled release is characterized by:
(A) the amount of the biologic released on day 1 is from 0 to 50% of the total amount of the biologic,
(B) the amount of the biologic released per day from day 2 until the last day of release is from 0 to 50% of the total amount of the biologic, and/or
(C) the number of days required for 100% release of the total amount of the biologic is at least 2 days.

348. The method or use according to claim any one of claims 321 to 347, wherein the pharmaceutically acceptable implant comprises a virus selected from a group consisting of retrovirus, adenovirus, adeno-associated virus (AAV), lentivirus and herpes simplex virus.

349. The method or use according to claim 348, wherein the virus is adeno-associated virus (AAV)

350. The method or use according to claim 349, wherein the adeno-associated virus (AAV) is selected from a group consisting of AAV1, AAV2 such as AAV2.7m8, AAV3, AAV4, AAV5, AAV6, AAV7, AAV8, AAV9, AAV10, AAV11 and a mutant, hybrid or variant thereof, wherein the AAV comprises at least one heterologous nucleic acid sequence that codes for a therapeutic protein.

351. The method or use according to claim 350, wherein the pharmaceutically acceptable implant comprises a virus, wherein the virus is adeno-associated virus (AAV), and wherein AAV is selected from a group consisting of AAV2, AAV2.7m8, and AAV8.

352. The method or use according to any one of claims 321 to 351, wherein the pharmaceutically acceptable implant is for controlled release of a total amount of AAV.

353. The method or use according to claim 352, wherein the controlled release is characterized by:
(A) the amount of the AAV released on day 1 is from 0 to 50% of the total amount of the AAV,
(B) the amount of the AAV released per day from day 2 until the last day of release is from 0 to 50% of the total amount of the AAV, and/or
(C) the number of days required for 100% release of the total amount of the AAV is at least 4 days.

354. The method or use according to any one of claims 352 to 353, wherein the controlled release is characterized by
(A) no greater than 9.0×10⁹ to 1.5×10¹⁰ vg AAV released on day 1,
(B) no greater than in the order 10¹¹ vg AAV per day such as in the order 10⁸, or 10⁹ or 10¹⁰ such as 5.0×10⁹ to 1.5×10¹⁰ vg AAV released per day from day 2 until the last day of the controlled release, and/or
(C) the number of days required for 100% release of the AAV is not less than 4 days.

355. The method or use according to any one of claims 352 to 353, wherein the controlled release is characterized by
(A) no greater than 9.0×10⁹ to 1.5×10¹⁰ vg AAV2 released on day 1,
(B) no greater than in the order 10¹¹ vg AAV2 per day such as in the order 10⁸, or 10⁹ or 10¹⁰ such as 5.0×10⁹ to 1.5×10¹⁰ vg AAV2 released per day from day 2 until the last day of the controlled release, and/or
(C) the number of days required for 100% release of the AAV2 is not less than 4 days.

356. The method or use according to any one of claims 352 to 353, wherein the controlled release is characterized by
(A) no greater than 9.0×10⁹ to 1.5×10¹⁰ vg AAV2.7m8 released on day 1,
(B) no greater than in the order 10¹¹ vg AAV2.7m8 per day such as in the order 10⁸, or 10⁹ or 10¹⁰ such as 5.0×10⁹ to 1.5×10¹⁰ vg AAV2.7m8 released per day from day 2 until the last day of the controlled release, and/or
(C) the number of days required for 100% release of the AAV2.7m8 is not less than 4 days.

357. The method or use according to claims 320 to 356, wherein the method comprises intravitreal injection of the pharmaceutically acceptable implant to the subject in need thereof.

358. The method or use of any one of claim 352 to 353, or 357, wherein the controlled release is characterized by
(A) no greater than 6×10⁹ to 1.0×10¹⁰ vg AAV per mL of the vitreous volume of the subject released on day 1,
(B) no greater than 3.5×10⁹ to 1.0×10¹⁰ vg AAV per mL of the vitreous volume of the subject released per day from day 2 until the last day of the controlled release, and/or
(C) the number of days required for 100% release of the AAV is not less than 4 days.

359. The method or use of any one of claim 352 to 353, or 357, wherein the controlled release is characterized by
(A) no greater than 6×10⁹ to 1.0×10¹⁰ vg AAV2 per mL of the vitreous volume of the subject released on day 1,
(B) no greater than 3.5×10⁹ to 1.0×10¹⁰ vg AAV2 per mL of the vitreous volume of the subject released per day from day 2 until the last day of the controlled release, and/or
(C) the number of days required for 100% release of the AAV2 is not less than 4 days.

360. The method or use of any one of claim 352 to 353, or 357, wherein the controlled release is characterized by
(A) no greater than 6×10⁹ to 1.0×10¹⁰ vg AAV2.7m8 per mL of the vitreous volume of the subject released on day 1,
(B) no greater than 3.5×10⁹ to 1.0×10¹⁰ vg AAV2.7m8 per mL of the vitreous volume of the subject released per day from day 2 until the last day of the controlled release, and/or
(C) the number of days required for 100% release of the AAV2.7m8 is not less than 4 days.

361. The method or use according to any one of claims 321-360, wherein when the total dose of AAV comprised in the pharmaceutically acceptable implant is in the order less than 2.0×10¹⁰ vg the number of days required for 100% release of the AAV is at least 4 days.

362. The method or use according to claims 321 to 361, wherein when the total dose of AAV comprised in the pharmaceutically acceptable implant is in the order greater than 2.0×10¹⁰ vg, the number of days required for 100% release of the AAV is at least 7 days, such as greater than 7 days, such as greater than 10 days or more.

363. A method of prophylactic inflammation treatment in a subject when treating an ocular disorder or an ocular genetic disorder comprising the following sequential steps:
(A) administering a composition to the eye of the subject comprising a total amount of a virus such as AAV comprising at least one heterologous nucleic acid sequence,
(B) assessing the total detectable amount of an endogenous nucleic acid sequence of the virus such as AAV or the heterologous nucleic acid sequence comprised in the virus such as AAV in the systemic circulation of the subject such as on day 1, day 2 or day 3 post administration,
(D) providing to the subject a prophylactic inflammation treatment if the detectable amount is higher than five log-less such as four log-less, three log-less, two log-less or one log-less as compared to the total amount of the AAV in the composition, wherein the prophylactic inflammation treatment comprises administering an anti-inflammatory agent to the eye of the subject.

364. The method or use according to claims 321 to 330 or 345 to 362, wherein ADA titer refers to ADA titer in the serum.

365. The method or use according to claims 331 to 362, wherein systemic circulation refers to blood, plasma, serum or lymph.

366. A pharmaceutically acceptable implant, which is an in-situ gel such as in-situ implant.

367. A method of delivering an AAV to a subject in need thereof, comprising injecting the in-situ gel such as in-situ implant of claim 365 into a suprachoroidal space of the subject.

368. A method of treating a wet-AMD in a subject in need thereof, comprising injecting the in-situ gel such as in-situ implant of claim 365 into a suprachoroidal space of the subject.

369. The pharmaceutically acceptable implant of claim 365, for use in a method of delivering an AAV to a subject in need thereof the method comprising injecting the insitu gel such as in-situ implant into a suprachoroidal space of the subject

370. The pharmaceutically acceptable implant of claim 365 for use in a method of treating a wet-AMD in a subject in need thereof, the method comprising injecting the in-situ gel such as an in-situ implant into a suprachoroidal space of the subject.
